(19) United States
(12) Patent Application Publication
(10) Pub. No.: US 2020/0248274 A1
(43) Pub. Date: Aug. 6, 2020

(54) DIAGNOSTIC FOR LUNG DISORDERS USING CLASS PREDICTION

(71) Applicant: Trustees of Boston University, Boston, MA (US)

(72) Inventors: Jerome S. Brody, Boston, MA (US); Avrum Spira, Newton, MA (US); Jennifer E. Beane-Ebel, Fort Collins, CO (US); Marc E. Lenburg, Brookline, MA (US); Jeffrey S. Berman, Cambridge, MA (US); Anthony W. O'Regan, Galway (IE)

(21) Appl. No.: 16/810,827

(22) Filed: Mar. 5, 2020

Related U.S. Application Data
(63) Continuation-in-part of application No. 15/888,831, filed on Feb. 5, 2018.
Application No. 15/888,831 is a continuation of application No. 14/613,210, filed on Feb. 3, 2015, now Pat. No. 9,920,374.
Application No. 14/613,210 is a continuation of application No. 13/524,749, filed on Jun. 15, 2012, now abandoned.
Application No. 13/524,749 is a continuation of application No. 12/869,525, filed on Aug. 26, 2010, now abandoned.
Application No. 12/869,525 is a continuation of application No. 11/918,588, filed on Feb. 8, 2008, now abandoned.
(60) Provisional application No. 60/671,243, filed on Apr. 14, 2005.

(51) Int. Cl. C12Q 1/6886

(52) U.S. Cl. CPC C12Q 1/6886; C12Q 2600/16; C12Q 2600/158

(57) ABSTRACT

The present invention provides methods for diagnosis and prognosis of lung cancer using expression analysis of one or more groups of genes, and a combination of expression analysis with bronchoscopy or via nasal epithelial cells. The methods of the invention provide far superior detection accuracy for lung cancer when compared to any other currently available method for lung cancer diagnostic or prognosis. The invention also provides methods of diagnosis and prognosis of other lung diseases, particularly in individuals who are exposed to air pollutants, such as cigarette or cigar smoke, smog, asbestos and the like air contaminants or pollutants via more accessible clinical samples from a bronchoscope or nasal sample.

### CROSS-REFERENCE TO RELATED APPLICATIONS

The present application is a continuation-in-part of U.S. application Ser. No. 15/888,831, filed on Feb. 5, 2018, which is a continuation of U.S. application Ser. No. 14/613,210, filed on Feb. 3, 2015, which is a continuation of U.S. application Ser. No. 13/524,749, filed on Jun. 15, 2012, which is a continuation of U.S. application Ser. No. 12/869,525, filed on Aug. 26, 2010, which is a continuation of U.S. application Ser. No. 11/918,588, filed Feb. 8, 2008, which is a national stage filing under 35 U.S.C. 371 of International Application PCT/US2006/014132, filed Apr. 14, 2006, which claims the benefit of priority under 35 U.S.C. 119(e) to U.S. provisional application Ser. No. 60/671,243, filed on Apr. 14, 2005, the contents of which are herein incorporated by reference in their entirety. International Application PCT/US2006/014132 was published under PCT Article 21(2) in English.

### GOVERNMENT SUPPORT

The present invention was made, in part, by support from the National Institutes of Health grant No. HL077498 and grant No. 071771. The United States Government has certain rights to the invention.

### BACKGROUND OF THE INVENTION

### Field of the Invention

The present invention is directed to diagnostic and prognostic methods by using analysis of gene group expression patterns in a subject. More specifically, the invention is directed to diagnostic and prognostic methods for detecting lung diseases, particularly lung cancer in subjects, preferably humans that have been exposed to air pollutants.

### Background

Lung disorders represent a serious health problem in the modern society. For example, lung cancer claims more than 150,000 lives every year in the United States, exceeding the combined mortality from breast, prostate and colorectal cancers. Cigarette smoking is the most predominant cause of lung cancer. Presently, 25% of the U.S. population smokes, but only 10% to 15% of heavy smokers develop lung cancer. There are also other disorders associated with smoking such as emphysema. There are also health questions arising from people exposed to smokers, for example, second hand smoke. Former smokers remain at risk for developing such disorders including cancer and now constitute a large reservoir of new lung cancer cases. In addition to cigarette smoke, exposure to other air pollutants such as asbestos, and smog, pose a serious lung disease risk to individuals who have been exposed to such pollutants.

Approximately 85% of all subjects with lung cancer die within three years of diagnosis. Unfortunately survival rates have not changed substantially of the past several decades. This is largely because there are no effective methods for identifying smokers who are at highest risk for developing lung cancer and no effective tools for early diagnosis.

The methods that are currently employed to diagnose lung cancer include chest X-ray analysis, bronchoscopy or sputum cytological analysis, computer tomographic analysis of the chest, and positron electron tomographic (PET) analysis. However, none of these methods provide a combination of both sensitivity and specificity needed for an optimal diagnostic test.

Classification of human lung cancer by gene expression profiling has been described in several recent publications (M. Garber, “Diversity of gene expression in adenocarcinoma of the lung,” PNAS, 98(24): 13784-13789 (2001); A. Bhattacharjee, “Classification of human lung carcinomas by mRNA expression profiling reveals distinct adenocarcinoma subclasses,” PNAS, 98(24):13790-13795 (2001)), but no specific gene set is used as a classifier to diagnose lung cancer in bronchial epithelial tissue samples.

Moreover, while it appears that a subset of smokers are more susceptible to, for example, the carcinogenic effects of cigarette smoke and are more likely to develop lung cancer, the particular risk factors, and particularly genetic risk factors, for individuals have gone largely unidentified. Same applies to lung cancer associated with, for example, asbestos exposure.

Therefore, there exists a great need to develop sensitive diagnostic methods that can be used for early diagnosis and prognosis of lung diseases, particularly in individuals who are at risk of developing lung diseases, particularly individuals who are exposed to air pollutants such as cigarette/cigar smoke, asbestos and other toxic air pollutants.

### SUMMARY OF THE INVENTION

The present invention provides compositions and methods for diagnosis and prognosis of lung diseases which provides a diagnostic test that is both very sensitive and specific.

We have found a group of gene transcripts that we can use individually and in groups or subsets for enhanced diagnosis for lung diseases, such as lung cancer, using gene expression analysis. We provide detailed guidance on the increase and/or decrease of expression of these genes for diagnosis and prognosis of lung diseases, such as lung cancer.

One example of the gene transcript groups useful in the diagnostic/prognostic tests of the invention are set forth in Table 6. We have found that taking groups of at least 20 of the Table 6 genes provides a much greater diagnostic capability than chance alone.

Preferably one would use more than 20 of these gene transcript, for example about 20-100 and any combination between, for example, 21, 22, 23, 24, 25, 26, 27, 28, 29, 30, and so on. Our preferred groups are the groups of 96 (Table 1), 84 (Table 2), 50 (Table 3), 36 (Table 4), 80 (Table 5), 535 (Table 6) and 20 (Table 7). In some instances, we have found that one can enhance the accuracy of the diagnosis by adding certain additional genes to any of these specific groups. When one uses these groups, the genes in the group are compared to a control or a control group. The control groups can be non-smokers, smokers, or former smokers. Preferably, one compares the gene transcripts or their expression product in the biological sample of an individual against a similar group, except that the members of the control groups do not have the lung disorder, such as emphysema or lung cancer. For example, comparing can be performed in the biological sample from a smoker against a control group of smokers who do not have lung cancer. When one compares the transcripts or expression products against the control for increased expression or decreased expression, which depends upon the particular gene and is set forth in the tables—not all the genes surveyed will show an increase or decrease. However, at least 50% of the genes surveyed must provide the described pattern. Greater reliability if obtained as the percent approaches 100%. Thus, in one embodiment, one wants at least 55%, 60%, 65%, 70%, 75%, 80%, 85%, 90%, 95%, 98%, or 99% of the genes surveyed to show the altered pattern indicative of lung disease, such as lung cancer, as set forth in the tables, infra.

In one embodiment, the invention provides a group of genes the expression of which is altered in individuals who are at risk of developing lung diseases, such as lung cancer, because of the exposure to air pollutants. The invention also provides groups of genes the expression of which is consistently altered as a group in individuals who are at risk of developing lung diseases because of the exposure to air pollutants.

The present invention provides gene groups the expression pattern or profile of which can be used in methods to diagnose lung diseases, such as lung cancer and even the type of lung cancer, in more than 60%, preferably more than 65%, still more preferably at least about 70%, still more preferably about 75%, or still more preferably about 80%-95% accuracy from a sample taken from airways of an individual screened for a lung disease, such as lung cancer.

In one embodiment, the invention provides a method of diagnosing a lung disease such as lung cancer using a combination of bronchoscopy and the analysis of gene expression pattern of the gene groups as described in the present invention.

Accordingly, the invention provides gene groups that can be used in diagnosis and prognosis of lung diseases. Particularly, the invention provides groups of genes the expression profile of which provides a diagnostic and or prognostic test to determine lung disease in an individual exposed to air pollutants. For example, the invention provides groups of genes the expression profile of which can distinguish individuals with lung cancer from individuals without lung cancer.

In one embodiment, the invention provides an early asymptomatic screening system for lung cancer by using the analysis of the disclosed gene expression profiles. Such screening can be performed, for example, in similar age groups as colonoscopy for screening colon cancer. Because early detection in lung cancer is crucial for efficient treatment, the gene expression analysis system of the present invention provides a vastly improved method to detect tumor cells that cannot yet be discovered by any other means currently available.

The probes that can be used to measure expression of the gene groups of the invention can be nucleic acid probes capable of hybridizing to the individual gene/transcript sequences identified in the present invention, or antibodies targeting the proteins encoded by the individual gene group gene products of the invention. The probes are preferably immobilized on a surface, such as a gene or protein chip so as to allow diagnosis and prognosis of lung diseases in an individual.

In one embodiment, the invention provides a group of genes that can be used as individual predictors of lung disease. These genes were identified using probabilities with a t-test analysis and show differential expression in smokers as opposed to non-smokers. The group of genes comprise ranging from 1 to 96, and all combinations in between, for example 5, 10, 15, 20, 25, 30, for example at least 36, at least about, 40, 45, 50, 60, 70, 80, 90, or 96 gene transcripts, selected from the group consisting of genes identified by the following GenBank sequence identification numbers (the identification numbers for each gene are separated by “;” while the alternative GenBank ID numbers are separated by “///”): NM_003335; NM_000918; NM_006430.1; NM_001416.1; NM_004090; NM_006406.1; NM_003001.2; NM_001319; NM_006545.1; NM_021145.1; NM_002437.1; NM_006286; NM_001003698///NM_001003699///NM_002955; NM_001123///NM_006721; NM_024824; NM_004935.1; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_001696; NM_005494///NM_058246; NM_006534///NM_181659; NM_006368; NM_002268///NM_032771; NM_014033; NM_016138; NM_007048///NM_194441; NM_006694; NM_000051///NM_138292///NM_138293; NM_000410///NM_139002///NM_139003///NM 139004///NM_139005///NM_139006///NM_139007///NM_139008///NM_139009///NM_139010///NM_139011; NM_004691; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_012394; NM_019011///NM_207111///NM_207116; NM_017646; NM_021800; NM_016049; NM_014395; NM_014336; NM_018097; NM_019014; NM_024804; NM_018260; NM_018118; NM_014128; NM_024084; NM_005294; AF077053; NM_138387; NM_024531; NM_000693; NM_018509; NM_033128; NM_020706; AI523613; and NM_014884, the expression profile of which can be used to diagnose lung disease, for example lung cancer, in lung cell sample from a smoker, when the expression pattern is compared to the expression pattern of the same group of genes in a smoker who does not have or is not at risk of developing lung cancer.

In another embodiment, the gene/transcript analysis comprises a group of about 10-20, 20-30, 30-40, 40-50, 50-60, 60-70, 70-80, 80, 80-90, 90-100, 100-120, 120-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, 490-500, 500-510, 510-520, 520-530, and up to about 535 genes selected from the group consisting of genes or transcripts as shown in the Table 6.

In one embodiment, the genes are selected from the group consisting of genes or transcripts as shown in Table 5.

In another embodiment, the genes are selected from the genes or transcripts as shown in Table 7.

In one embodiment, the transcript analysis gene group comprises a group of individual genes the change of expression of which is predictive of a lung disease either alone or as a group, the gene transcripts selected from the group consisting of NM_007062.1; NM_001281.1; BC002642.1; NM_000346.1; NM_006545.1; BG034328; NM_019067.1; NM_017925.1; NM_017932.1; NM_030757.1; NM_030972.1; NM_002268///NM_032771; NM_007048///NM_194441; NM_006694; U85430.1; NM_004691; AB014576.1; BF218804; BE467941; R83000; AL161952.1; AK023843.1; AK021571.1; AK023783.1; AL080112.1; AW971983; AI683552; NM_024006.1; AK026565.1; NM_014182.1; NM_021800.1; NM_016049.1; NM_021971.1; NM_014128.1; AA133341; AF198444.1.

In one embodiment, the gene group comprises a probe set capable of specifically hybridizing to at least all of the 36 gene products. Gene product can be mRNA which can be recognized by an oligonucleotide or modified oligonucleotide probe, or protein, in which case the probe can be, for example an antibody specific to that protein or an antigenic epitope of the protein.

In yet another embodiment, the invention provides a gene group, wherein the expression pattern of the group of genes provides diagnostic for a lung disease. The gene group comprises gene transcripts encoded by a gene group consisting of at least for example 5, 10, 15, 20, 25, 30, preferably at least 36, still more preferably 40, still more preferably 45, and still more preferably 46, 47, 48, 49, or all 50 of the genes selected from the group consisting of and identified by their GenBank identification numbers: NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U 93240.1; U90552.1; AF151056.1; U85430.1; U51007.1; BC005969.1; NM_002271.1; AL566172; AB014576.1; BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1. In one preferred embodiment, one can use at least 20 of the 36 genes that overlap with the individual predictors and, for example, 5-9 of the non-overlapping genes and combinations thereof.

In another embodiment, the invention provides a group of about 30-180, preferably, a group of about 36-150 genes, still more preferably a group of about 36-100, and still more preferably a group of about 36-50 genes, the expression profile of which is diagnostic of lung cancer in individuals who smoke.

In one embodiment, the invention provides a group of genes the expression of which is decreased in an individual having lung cancer. In one embodiment, the group of genes comprises at least 5-10, 10-15, 15-20, 20-25 genes selected from the group consisting of NM_000918; NM_006430.1; NM_001416.1; NM_004090; NM_006406.1; NM_003001.2; NM_006545.1; NM_002437.1; NM_006286; NM_001123///NM_006721; NM_024824; NM_004935.1; NM_001696; NM_005494///NM_058246; NM_006368; NM_002268///NM_032771; NM_006694; NM_004691; NM_012394; NM_021800; NM_016049; NM_138387; NM_024531; and NM_018509. One or more other genes can be added to the analysis mixtures in addition to these genes.

In another embodiment, the group of genes comprises genes selected from the group consisting of NM_014182.1; NM_001281.1; NM_024006.1; AF135421.1; L76200.1; NM_000346.1; BC008710.1; BC000423.2; BC008710.1; NM_007062; BC075839.1///BC073760.1; BC072436.1///BC004560.2; BC001016.2; BC005023.1; BC000360.2; BC007455.2; BC023528.2///BC047680.1; BC064957.1; BC008710.1; BC066329.1; BC023976.2; BC008591.2///BC050440.1///BC048096.1; and BC028912.1.

In yet another embodiment, the group of genes comprises genes selected from the group consisting of NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; and AB014576.1.

In one embodiment, the invention provides a group of genes the expression of which is increased in an individual having lung cancer. In one embodiment, the group of genes comprises genes selected from the group consisting of NM_003335; NM_001319; NM_021145.1; NM_001003698///NM_001003699///; NM_002955; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_006534///NM_181659; NM_014033; NM_016138; NM_007048///NM_194441; NM_000051///NM_138292///NM_138293; NM_000410///NM_139002///NM_139003///NM_139004///NM_139005///NM_139006///NM_139007///NM_139008///NM_139009///NM_139010///NM_139011; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_019011///NM_207111///NM_207116; NM_017646; NM_014395; NM_014336; NM_018097; NM_019014; NM_024804; NM_018260; NM_018118; NM_014128; NM_024084; NM_005294; AF077053; NM_000693; NM_033128; NM_020706; AI523613; and NM_014884.

In one embodiment, the group of genes comprises genes selected from the group consisting of NM_030757.1; R83000; AK021571.1; NM_17932.1; U85430.1; AI683552; BC002642.1; AW024467; NM_030972.1; BC021135.1; AL161952.1; AK026565.1; AK023783.1; BF218804; AK023843.1; BC001602.1; BC034707.1; BC064619.1; AY280502.1; BC059387.1; BC061522.1; U50532.1; BC006547.2; BC008797.2; BC000807.1; AL080112.1; BC033718.1///BC046176.1///; BC038443.1; Hs.288575 (UNIGENE ID); AF020591.1; BC002503.2; BC009185.2; Hs.528304 (UNIGENE ID); U50532.1; BC013923.2; BC031091; Hs.249591 (Unigene ID); Hs.286261 (Unigene ID); AF348514.1; BC066337.1///BC058736.1///BC050555.1; Hs.216623 (Unigene ID); BC072400.1; BC041073.1; U43965.1; BC021258.2; BC016057.1; BC016713.1///BC014535.1///AF237771.1; BC000701.2; BC010067.2; Hs.156701 (Unigene ID); BC030619.2; U43965.1; Hs.438867 (Unigene ID); BC035025.2///BC050330.1; BC074852.2///BC074851.2; Hs.445885 (Unigene ID); AF365931.1; and AF257099.1.

In one embodiment, the group of genes comprises genes selected from the group consisting of BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1.

In another embodiment, the invention provides a method for diagnosing a lung disease comprising obtaining a nucleic acid sample from lung, airways or mouth of an individual exposed to an air pollutant, analyzing the gene transcript levels of one or more gene groups provided by the present invention in the sample, and comparing the expression pattern of the gene group in the sample to an expression pattern of the same gene group in an individual, who is exposed to similar air pollutant but not having lung disease, such as lung cancer or emphysema, wherein the difference in the expression pattern is indicative of the test individual having or being at high risk of developing a lung disease. The decreased expression of one or more of the genes, preferably all of the genes including the genes listed on Tables 1-4 as “down” when compared to a control, and/or increased expression of one or more genes, preferably all of the genes listed on Tables 1-4 as “up” when compared to an individual exposed to similar air pollutants who does not have a lung disease, is indicative of the person having a lung disease or being at high risk of developing a lung disease, preferably lung cancer, in the near future and needing frequent follow ups to allow early treatment of the disease.

In one preferred embodiment, the lung disease is lung cancer. In one embodiment, the air pollutant is cigarette smoke.

Alternatively, the diagnosis can separate the individuals, such as smokers, who are at lesser risk of developing lung diseases, such as lung cancer by analyzing the expression pattern of the gene groups of the invention provides a method of excluding individuals from invasive and frequent follow ups.

Accordingly, the invention provides methods for prognosis, diagnosis and therapy designs for lung diseases comprising obtaining an airway sample from an individual who smokes and analyzing expression profile of the gene groups of the present invention, wherein an expression pattern of the gene group that deviates from that in a healthy age, race, and gender matched smoker, is indicative of an increased risk of developing a lung disease. Tables 1-4 indicate the expression pattern differences as either being down or up as compared to a control, which is an individual exposed to similar airway pollutant but not affected with a lung disease.

The invention also provides methods for prognosis, diagnosis and therapy designs for lung diseases comprising obtaining an airway sample from a non-smoker individual and analyzing expression profile of the gene groups of the present invention, wherein an expression pattern of the gene group that deviates from that in a healthy age, race, and gender matched smoker, is indicative of an increased risk of developing a lung disease.

In one embodiment, the analysis is performed from a biological sample obtained from bronchial airways.

In one embodiment, the analysis is performed from a biological sample obtained from buccal mucosa.

In one embodiment, the analysis is performed using nucleic acids, preferably RNA, in the biological sample.

In one embodiment, the analysis is performed analyzing the amount of proteins encoded by the genes of the gene groups of the invention present in the sample.

In one embodiment the analysis is performed using DNA by analyzing the gene expression regulatory regions of the groups of genes of the present invention using nucleic acid polymorphisms, such as single nucleic acid polymorphisms or SNPs, wherein polymorphisms known to be associated with increased or decreased expression are used to indicate increased or decreased gene expression in the individual. For example, methylation patterns of the regulatory regions of these genes can be analyzed.

In one embodiment, the present invention provides a minimally invasive sample procurement method for obtaining airway epithelial cell RNA that can be analyzed by expression profiling of the groups of genes, for example, by array-based gene expression profiling. These methods can be used to diagnose individuals who are already affected with a lung disease, such as lung cancer, or who are at high risk of developing lung disease, such as lung cancer, as a consequence of being exposed to air pollutants. These methods can also be used to identify further patterns of gene expression that are diagnostic of lung disorders/diseases, for example, cancer or emphysema, and to identify subjects at risk for developing lung disorders.

The invention further provides a gene group microarray consisting of one or more of the gene groups provided by the invention, specifically intended for the diagnosis or prediction of lung disorders or determining susceptibility of an individual to lung disorders.

In one embodiment, the invention relates to a method of diagnosing a disease or disorder of the lung comprising obtaining a sample, nucleic acid or protein sample, from an individual to be diagnosed; and determining the expression of group of identified genes in said sample, wherein changed expression of such gene compared to the expression pattern of the same gene in a healthy individual with similar life style and environment is indicative of the individual having a disease of the lung.

In one embodiment, the invention relates to a method of diagnosing a disease or disorder of the lung comprising obtaining at least two samples, nucleic acid or protein samples, in at least one time interval from an individual to be diagnosed; and determining the expression of the group of identified genes in said sample, wherein changed expression of at least about for example 5, 10, 15, 20, 25, 30, preferably at least about 36, 40, 50, 60, 70, 80, 90, 100, 110, 120, 130, 140, 150, 160, 170, or 180 of such genes in the sample taken later in time compared to the sample taken earlier in time is diagnostic of a lung disease.

In one embodiment, the disease of the lung is selected from the group consisting of asthma, chronic bronchitis, emphysema, primary pulmonary hypertension, acute respiratory distress syndrome, hypersensitivity pneumonitis, eosinophilic pneumonia, persistent fungal infection, pulmonary fibrosis, systemic sclerosis, idiopathic pulmonary hemosiderosis, pulmonary alveolar proteinosis, and lung cancer, such as adenocarcinoma, squamous cell carcinoma, small cell carcinoma, large cell carcinoma, and benign neoplasm of the lung (e.g., bronchial adenomas and hamartomas).

In a particular embodiment, the nucleic acid sample is RNA.

In a preferred embodiment, the nucleic acid sample is obtained from an airway epithelial cell. In one embodiment, the airway epithelial cell is obtained from a bronchoscopy or buccal mucosal scraping.

In one embodiment, individual to be diagnosed is an individual who has been exposed to tobacco smoke, an individual who has smoked, or an individual who currently smokes.

The invention also provides an array, for example, a microarray for diagnosis of a disease of the lung having immobilized thereon a plurality of oligonucleotides which hybridize specifically to genes of the gene groups which are differentially expressed in airways exposed to air pollutants, such as cigarette smoke, and have or are at high risk of developing lung disease, as compared to those individuals who are exposed to similar air pollutants and airways which are not exposed to such pollutants. In one embodiment, the oligonucleotides hybridize specifically to one allelic form of one or more genes which are differentially expressed for a disease of the lung. In a particular embodiment, the differentially expressed genes are selected from the group consisting of the genes shown in tables 1-4; preferably the group of genes comprises genes selected from the Table 3. In one preferred embodiment, the group of genes comprises the group of at least 20 genes selected from Table 3 and additional 5-10 genes selected from Tables 1 and 2. In one preferred embodiment, at least about 10 genes are selected from Table 4.

Although sampling epithelial cells from bronchial tissue while less invasive than many other methods has some drawbacks. For example, the patient may not eat or drink for about 6-12 hours prior to the test. Also, if the procedure is performed using a rigid bronchoscope the patient needs general anesthesia involving related risks to the patient. When the method is performed using a flexible bronchoscope, the procedure is performed using local anesthesia. However, several patients experience uncomfortable sensations, such as a sensation of suffocating during such a procedure and thus are relatively resistant for going through the procedure more than once. Also, after the bronchoscopy procedure, the throat may feel uncomfortably scratchy for several days.

While it has been previously described, that RNA can be isolated from mouth epithelial cells for gene expression analysis (U.S. Ser. No. 10/579,376), it has not been clear if such samples routinely reflect the same gene expression changes as bronchial samples that can be used in accurate diagnostic and prognostic methods.

Thus, there is significant interest and need in developing simple non-invasive screening methods for assessing an individual's lung disease, such as lung cancer or risk for developing lung cancer, including primary lung malignancies. It would be preferable if such a method would be more accurate than the traditional chest x-ray or PET analysis or cytological analysis, for example by identifying marker genes which have their expression altered at various states of disease progression.

Thus, some aspects of the invention provide a much less invasive method for diagnosing lung diseases, such as lung cancer based on analysis of gene expression in nose epithelial cells.

We have found surprisingly that the gene expression changes in nose epithelial cells closely mirrors the gene expression changes in the lung epithelial cells. Accordingly, the invention provides methods for diagnosis, prognosis and follow up of progression or success of treatment for lung diseases using gene expression analysis from nose epithelial cells.

We have also found that the gene expression pattern in the bronchial epithelial cells and nasal epithelial cells very closely correlated. This is in contrast with epithelial cell expression pattern in any other tissue we have studies thus far. The genes the expression of which is particularly closely correlated between the lung and the nose are listed in tables 18, 19 and 20.

The method provides an optimal means for screening for changes indicating malignancies in individuals who, for example are at risk of developing lung diseases, particularly lung cancers because they have been exposed to pollutants, such as cigarette or cigar smoke or asbestos or any other known pollutant. The method allows screening at a routine annual medical examination because it does not need to be performed by an expert trained in bronchoscopy and it does not require sophisticated equipment needed for bronchoscopy.

We discovered that there is a significant correlation between the epithelial cell gene expression in the brinchial tissue and in the nasal passages. We discovered this by analyzing samples from individuals with cancer as well as by analyzing samples from smokers compared to non-smokers.

We discovered a strong correlation between the gene expression profile in the bronchial and nasal epithelial cell samples when we analyzed genes that distinguish individuals with known sarcoidosis from individuals who do not have sarcoidosis.

We also discovered that the same is true, when one compares the changes in the gene expression pattern between smokers and individuals who have never smoked.

Accordingly, we have found a much less invasive method of sampling for prognostic, diagnostic and follow-up purposes by taking epithelial samples from the nasal passages as opposed to bronchial tissue, and that the same genes that have proven effective predictors for lung diseases, such as lung cancer, in smokers and non-smokers, can be used in analysis of epithelial cells from the nasal passages.

The gene expression analysis can be performed using genes and/or groups of genes as described in tables 18, 19 and 20 and, for example, in other tables disclosed herein. Naturally, other diagnostic genes may also be used, as they are identified.

Accordingly, the invention provides a substantially less invasive method for diagnosis, prognosis, and follow-up of lung diseases using samples from nasal epithelial cells. To provide an improved analysis, one preferably uses gene expression analysis.

One can use analysis of gene transcripts individually and in groups or subsets for enhanced diagnosis for lung diseases, such as lung cancer.

Similarly, as the art continues to identify the gene expression changes associated with other lung diseases wherein the disease causes a field effect, namely, wherein the disease-causing agent, i.e. a pollutant, or a microbe or other airway irritant, the analysis and discoveries presented herein allow us to conclude that those gene expression changes can also be analyzed from nasal epithelial cells thus providing a much less invasive and more accurate method for diagnosing lung diseases in general. For example, using the methods as described, one can diagnose any lung disease that results in detectable gene expression changes, including, but not limited to acute pulmonary eosinophilia (Loeffler's syndrome), CMV pneumonia, chronic pulmonary coccidioidomycosis, cryptococcosis, disseminated tuberculosis (infectious), chronic pulmonary histoplasmosis, pulmonary actinomycosis, pulmonary aspergilloma (mycetoma), pulmonary aspergillosis (invasive type), pulmonary histiocytosis X (eosinophilic granuloma), pulmonary nocardiosis, pulmonary tuberculosis, and sarcoidosis. In fact, one of the examples shows a group of genes the expression of which changes when the individual is affected with sarcoidosis.

One example of the gene transcript groups useful in the diagnostic/prognostic tests of the invention using nasal epithelial cells are set forth in Table 16. We have found that taking groups of at least 20 of the Table 16 genes provides a much greater diagnostic capability than chance alone.

Preferably one would use more than 20 of these gene transcript, for example about 20-100 and any combination between, for example, 21, 22, 23, 24, 25, 26, 27, 28, 29, 30, and so on. Our preferred groups are the groups of 361 (Table 18), 107 (Table 19), 70 (Table 20), 96 (Table 11), 84 (Table 12), 50 (Table 13), 36 (Table 14), 80 (Table 15), 535 (Table 16) and 20 (Table 17).

In some instances, we have found that one can enhance the accuracy of the diagnosis by adding certain additional genes to any of these specific groups. When one uses these groups, the genes in the group are compared to a control or a control group. The control groups can be individuals who have not been exposed to a particular airway irritant, such as non-smokers, smokers, or former smokers, or individuals not exposed to viruses or other substance that can cause a “filed effect” in the airways thus resulting in potential for lung disease. Typically, when one wishes to diagnose a disease, the control sample should be from an individual who does not have the diseases and alternatively include one or more samples with individuals who have similar or different lung diseases. Thus, one can match the sample one wishes to diagnose with a control wherein the expression pattern most closely resembles the expression pattern in the sample. Preferably, one compares the gene transcripts or their expression product in the biological sample of an individual against a similar group, except that the members of the control groups do not have the lung disorder, such as emphysema or lung cancer. For example, comparing can be performed in the biological sample from a smoker against a control group of smokers who do not have lung cancer. When one compares the transcripts or expression products against the control for increased expression or decreased expression, which depends upon the particular gene and is set forth in the tables—not all the genes surveyed will show an increase or decrease. However, at least 50% of the genes surveyed must provide the described pattern. Greater reliability is obtained as the percent approaches 100%. Thus, in one embodiment, one wants at least 55%, 60%, 65%, 70%, 75%, 80%, 85%, 90%, 95%, 98%, or 99% of the genes surveyed to show the altered pattern indicative of lung disease, such as lung cancer, as set forth in the tables, infra.

In one embodiment, the nasal epithelial cell sample is analyzed for a group of genes the expression of which is altered in individuals who are at risk of developing lung diseases, such as lung cancer, because of the exposure to air pollutants or other airway irritant such as microbes that occur in the air and are inhaled. The method can also be used for analysis of groups of genes the expression of which is consistently altered as a group in individuals who are at risk of developing lung diseases because of the exposure to such air pollutants including microbes and viruses present in the air.

One can analyze the nasal epithelial cells according to the methods of the present invention using gene groups the expression pattern or profile of which can be used to diagnose lung diseases, such as lung cancer and even the type of lung cancer, in more than 60%, preferably more than 65%, still more preferably at least about 70%, still more preferably about 75%, or still more preferably about 80%-95% accuracy from a sample taken from airways of an individual screened for a lung disease, such as lung cancer.

In one embodiment, the invention provides a method of diagnosing a lung disease such as lung cancer using a combination of nasal epithelial cells and the analysis of gene expression pattern of the gene groups as described in the present invention.

Accordingly, the invention provides methods for analyzing gene groups from nasal epithelial cells, wherein the gene expression pattern that can be directly used in diagnosis and prognosis of lung diseases. Particularly, the invention provides analysis from nasal epithelial cells groups of genes the expression profile of which provides a diagnostic and or prognostic test to determine lung disease in an individual exposed to air pollutants. For example, the invention provides analysis from nasal epithelial cells, groups of genes the expression profile of which can distinguish individuals with lung cancer from individuals without lung cancer.

In one embodiment, the invention provides an early asymptomatic screening system for lung cancer by using the analysis of nasal epithelial cells for the disclosed gene expression profiles. Such screening can be performed, for example, in similar age groups as colonoscopy for screening colon cancer. Because early detection in lung cancer is crucial for efficient treatment, the gene expression analysis system of the present invention provides an improved method to detect tumor cells. Thus, the analysis can be made at various time intervals, such as once a year, once every other year for screening purposes. Alternatively, one can use a more frequent sampling if one wishes to monitor disease progression or regression in response to a therapeutic intervention. For example, one can take samples from the same patient once a week, once or two times a month, every 3, 4, 5, or 6 months.

The probes that can be used to measure expression of the gene groups of the invention can be nucleic acid probes capable of hybridizing to the individual gene/transcript sequences identified in the present invention, or antibodies targeting the proteins encoded by the individual gene group gene products of the invention. The probes are preferably immobilized on a surface, such as a gene or protein chip so as to allow diagnosis and prognosis of lung diseases in an individual.

In one preferred embodiment, the invention provides a group of genes that can be used in diagnosis of lung diseases from the nasal epithelial cells. These genes were identified using

In one embodiment, the invention provides a group of genes that can be used as individual predictors of lung disease. These genes were identified using probabilities with a t-test analysis and show differential expression in smokers as opposed to non-smokers. The group of genes comprise ranging from 1 to 96, and all combinations in between, for example 5, 10, 15, 20, 25, 30, for example at least 36, at least about, 40, 45, 50, 60, 70, 80, 90, or 96 gene transcripts, selected from the group consisting of genes identified by the following GenBank sequence identification numbers (the identification numbers for each gene are separated by “;” while the alternative GenBank ID numbers are separated by “///”): NM_003335; NM_000918; NM_006430.1; NM_001416.1; NM_004090; NM_006406.1; NM_003001.2; NM_001319; NM_006545.1; NM_021145.1; NM_002437.1; NM_006286; NM_001003698///NM_001003699///NM_002955; NM_001123///NM_006721; NM_024824; NM_004935.1; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_001696; NM_005494///NM_058246; NM_006534///NM_181659; NM_006368; NM_002268///NM_032771; NM_014033; NM_016138; NM_007048///NM_194441; NM_006694; NM_000051///NM_138292///NM_138293; NM_000410///NM_139002///NM_139003///NM_139004///NM_139005///NM_139006///NM_139007///NM_139008///NM_139009///NM_139010///NM_139011; NM_004691; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_012394; NM_019011///NM_207111///NM_207116; NM_017646; NM_021800; NM_016049; NM_014395; NM_014336; NM_018097; NM_019014; NM_024804; NM_018260; NM_018118; NM_014128; NM_024084; NM_005294; AF077053; NM_138387; NM_024531; NM_000693; NM_018509; NM_033128; NM_020706; AI523613; and NM_014884, the expression profile of which can be used to diagnose lung disease, for example lung cancer, in lung cell sample from a smoker, when the expression pattern is compared to the expression pattern of the same group of genes in a smoker who does not have or is not at risk of developing lung cancer.

In another embodiment, the gene/transcript analysis comprises a group of about 10-20, 20-30, 30-40, 40-50, 50-60, 60-70, 70-80, 80, 80-90, 90-100, 100-120, 120-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, 490-500, 500-510, 510-520, 520-530, and up to about 535 genes selected from the group consisting of genes or transcripts as shown in the Table 16.

In one embodiment, the genes are selected from the group consisting of genes or transcripts as shown in Table 15.

In another embodiment, the genes are selected from the genes or transcripts as shown in Table 17.

In one embodiment, the transcript analysis gene group comprises a group of individual genes the change of expression of which is predictive of a lung disease either alone or as a group, the gene transcripts selected from the group consisting of NM_007062.1; NM_001281.1; BC002642.1; NM_000346.1; NM_006545.1; BG034328; NM_019067.1; NM_017925.1; NM_017932.1; NM_030757.1; NM_030972.1; NM_002268///NM_032771; NM_007048///NM_194441; NM_006694; U85430.1; NM_004691; AB014576.1; BF218804; BE467941; R83000; AL161952.1; AK023843.1; AK021571.1; AK023783.1; AL080112.1; AW971983; AI683552; NM_024006.1; AK026565.1; NM_014182.1; NM_021800.1; NM_016049.1; NM_021971.1; NM_014128.1; AA133341; AF198444.1.

In one embodiment, the gene group comprises a probe set capable of specifically hybridizing to at least all of the 36 gene products. Gene product can be mRNA which can be recognized by an oligonucleotide or modified oligonucleotide probe, or protein, in which case the probe can be, for example an antibody specific to that protein or an antigenic epitope of the protein.

In yet another embodiment, the invention provides a gene group, wherein the expression pattern of the group of genes provides diagnostic for a lung disease. The gene group comprises gene transcripts encoded by a gene group consisting of at least for example 5, 10, 15, 20, 25, 30, preferably at least 36, still more preferably 40, still more preferably 45, and still more preferably 46, 47, 48, 49, or all 50 of the genes selected from the group consisting of and identified by their GenBank identification numbers: NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U 93240.1; U90552.1; AF151056.1; U85430.1; U51007.1; BC005969.1; NM_002271.1; AL566172; AB014576.1; BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1. In one preferred embodiment, one can use at least 20 of the 36 genes that overlap with the individual predictors and, for example, 5-9 of the non-overlapping genes and combinations thereof.

In another embodiment, the invention provides a group of about 30-180, preferably, a group of about 36-150 genes, still more preferably a group of about 36-100, and still more preferably a group of about 36-50 genes, the expression profile of which is diagnostic of lung cancer in individuals who smoke.

In one embodiment, the invention provides a group of genes the expression of which is decreased in an individual having lung cancer. In one embodiment, the group of genes comprises at least 5-10, 10-15, 15-20, 20-25 genes selected from the group consisting of NM_000918; NM_006430.1; NM_001416.1; NM_004090; NM_006406.1; NM_003001.2; NM_006545.1; NM_002437.1; NM_006286; NM_001123///NM_006721; NM_024824; NM_004935.1; NM_001696; NM_005494///NM_058246; NM_006368; NM_002268///NM_032771; NM_006694; NM_004691; NM_012394; NM_021800; NM_016049; NM_138387; NM_024531; and NM_018509. One or more other genes can be added to the analysis mixtures in addition to these genes.

In another embodiment, the group of genes comprises genes selected from the group consisting of NM_014182.1; NM_001281.1; NM_024006.1; AF135421.1; L76200.1; NM_000346.1; BC008710.1; BC000423.2; BC008710.1; NM_007062; BC075839.1///BC073760.1; BC072436.1///BC004560.2; BC001016.2; BC005023.1; BC000360.2; BC007455.2; BC023528.2///BC047680.1; BC064957.1; BC008710.1; BC066329.1; BC023976.2; BC008591.2///BC050440.1///BC048096.1; and BC028912.1.

In yet another embodiment, the group of genes comprises genes selected from the group consisting of NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; and AB014576.1.

In one embodiment, the invention provides a group of genes the expression of which is increased in an individual having lung cancer. In one embodiment, the group of genes comprises genes selected from the group consisting of NM_003335; NM_001319; NM_021145.1; NM_001003698///NM_001003699///; NM_002955; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_006534///NM_181659; NM_014033; NM_016138; NM_007048///NM_194441; NM_000051///NM_138292///NM_138293; NM_000410///NM_139002///NM_139003///NM_139004///NM_139005///NM_139006///NM_139007///NM_139008///NM_139009///NM_139010///NM_139011; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_019011///NM_207111///NM_207116; NM_017646; NM_014395; NM_014336; NM_018097; NM_019014; NM_024804; NM_018260; NM_018118; NM_014128; NM_024084; NM_005294; AF077053; NM_000693; NM_033128; NM_020706; AI523613; and NM_014884.

In one embodiment, the group of genes comprises genes selected from the group consisting of NM_030757.1; R83000; AK021571.1; NM_17932.1; U85430.1; AI683552; BC002642.1; AW024467; NM_030972.1; BC021135.1; AL161952.1; AK026565.1; AK023783.1; BF218804; AK023843.1; BC001602.1; BC034707.1; BC064619.1; AY280502.1; BC059387.1; BC061522.1; U50532.1; BC006547.2; BC008797.2; BC000807.1; AL080112.1; BC033718.1///BC046176.1///; BC038443.1; Hs.288575 (UNIGENE ID); AF020591.1; BC002503.2; BC009185.2; Hs.528304 (UNIGENE ID); U50532.1; BC013923.2; BC031091; Hs.249591 (Unigene ID); Hs.286261 (Unigene ID); AF348514.1; BC066337.1///BC058736.1///BC050555.1; Hs.216623 (Unigene ID); BC072400.1; BC041073.1; U43965.1; BC021258.2; BC016057.1; BC016713.1///BC014535.1///AF237771.1; BC000701.2; BC010067.2; Hs.156701 (Unigene ID); BC030619.2; U43965.1; Hs.438867 (Unigene ID); BC035025.2///BC050330.1; BC074852.2///BC074851.2; Hs.445885 (Unigene ID); AF365931.1; and AF257099.1.

In one embodiment, the group of genes comprises genes selected from the group consisting of BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1.

In another embodiment, the invention provides a method for diagnosing a lung disease comprising obtaining a nucleic acid sample from lung, airways or mouth of an individual exposed to an air pollutant, analyzing the gene transcript levels of one or more gene groups provided by the present invention in the sample, and comparing the expression pattern of the gene group in the sample to an expression pattern of the same gene group in an individual, who is exposed to similar air pollutant but not having lung disease, such as lung cancer or emphysema, wherein the difference in the expression pattern is indicative of the test individual having or being at high risk of developing a lung disease. The decreased expression of one or more of the genes, preferably all of the genes including the genes listed on Tables 11-14 as “down” when compared to a control, and/or increased expression of one or more genes, preferably all of the genes listed on Tables 11-14 as “up” when compared to an individual exposed to similar air pollutants who does not have a lung disease, is indicative of the person having a lung disease or being at high risk of developing a lung disease, preferably lung cancer, in the near future and needing frequent follow ups to allow early treatment of the disease.

In one preferred embodiment, the lung disease is lung cancer. In one embodiment, the air pollutant is tobacco or tobacco smoke.

Alternatively, the diagnosis can separate the individuals, such as smokers, who are at lesser risk of developing lung diseases, such as lung cancer by analyzing from the nasal epithelial cells the expression pattern of the gene groups of the invention provides a method of excluding individuals from invasive and frequent follow ups.

Accordingly, in one embodiment, the invention provides methods for prognosis, diagnosis and therapy designs for lung diseases comprising obtaining an nasal epithelial cell sample from an individual who smokes and analyzing expression profile of the gene groups of the present invention, wherein an expression pattern of the gene group that deviates from that in a healthy age, race, and gender matched smoker, is indicative of an increased risk of developing a lung disease. Tables 11-14 indicate the expression pattern differences as either being down or up as compared to a control, which is an individual exposed to similar airway pollutant but not affected with a lung disease.

The invention also provides methods for prognosis, diagnosis and therapy designs for lung diseases comprising obtaining an nasal epithelial cell sample from a non-smoker individual and analyzing expression profile of the gene groups of the present invention, wherein an expression pattern of the gene group that deviates from that in a healthy age, race, and gender matched smoker, is indicative of an increased risk of developing a lung disease.

In one embodiment, the analysis is performed using nucleic acids, preferably RNA, in the biological sample.

In one embodiment, the analysis is performed analyzing the amount of proteins encoded by the genes of the gene groups of the invention present in the sample.

In one embodiment the analysis is performed using DNA by analyzing the gene expression regulatory regions of the groups of genes of the present invention using nucleic acid polymorphisms, such as single nucleic acid polymorphisms or SNPs, wherein polymorphisms known to be associated with increased or decreased expression are used to indicate increased or decreased gene expression in the individual. For example, methylation patterns of the regulatory regions of these genes can be analyzed.

In one embodiment, the present invention provides a minimally invasive sample procurement method for obtaining nasal epithelial cell RNA that can be analyzed by expression profiling of the groups of genes, for example, by array-based gene expression profiling. These methods can be used to diagnose individuals who are already affected with a lung disease, such as lung cancer, or who are at high risk of developing lung disease, such as lung cancer, as a consequence of being exposed to air pollutants. These methods can also be used to identify further patterns of gene expression that are diagnostic of lung disorders/diseases, for example, cancer or emphysema, and to identify subjects at risk for developing lung disorders.

The invention further provides a method of analyzing nasal epithelial cells using gene group microarray consisting of one or more of the gene groups provided by the invention, specifically intended for the diagnosis or prediction of lung disorders or determining susceptibility of an individual to lung disorders.

In one embodiment, the invention relates to a method of diagnosing a disease or disorder of the lung comprising obtaining a sample from nasal epithelial cells, wherein the sample is a nucleic acid or protein sample, from an individual to be diagnosed; and determining the expression of group of identified genes in said sample, wherein changed expression of such gene compared to the expression pattern of the same gene in a healthy individual with similar life style and environment is indicative of the individual having a disease of the lung.

In one embodiment, the invention relates to a method of diagnosing a disease or disorder of the lung comprising obtaining at least two nasal epithelial samples, wherein the samples are either nucleic acid or protein samples, in at least one, two, 3, 4, 5, 6, 7, 8, 9, or more time intervals from an individual to be diagnosed; and determining the expression of the group of identified genes in said sample, wherein changed expression of at least about for example 5, 10, 15, 20, 25, 30, preferably at least about 36, 40, 50, 60, 70, 80, 90, 100, 110, 120, 130, 140, 150, 160, 170, or 180 of such genes in the sample taken later in time compared to the sample taken earlier in time is diagnostic of a lung disease.

In one embodiment, the disease of the lung is selected from the group consisting of asthma, chronic bronchitis, emphysema, primary pulmonary hypertension, acute respiratory distress syndrome, hypersensitivity pneumonitis, eosinophilic pneumonia, persistent fungal infection, pulmonary fibrosis, systemic sclerosis, idiopathic pulmonary hemosiderosis, pulmonary alveolar proteinosis, and lung cancer, such as adenocarcinoma, squamous cell carcinoma, small cell carcinoma, large cell carcinoma, and benign neoplasm of the lung (e.g., bronchial adenomas and hamartomas).

In a particular embodiment, the nucleic acid sample is RNA.

In one embodiment, individual to be diagnosed is an individual who has been exposed to tobacco smoke, an individual who has smoked, or an individual who currently smokes.

Some aspects of the present invention are directed to a method for determining whether a subject has or is at risk of developing a lung disorder, comprising: (a) obtaining a biological sample from a nasal passage of said subject; (b) assaying nucleic acid molecules derived from said biological sample to identify a level of gene expression in said biological sample; (c) processing said level of gene expression against a control to determine a deviation in said level of expression; and (d) based on said deviation in (c), determining that said subject has or is at risk of developing said lung disorder.

The invention also provides analysis of nasal epithelial cells using an array, for example, a microarray for diagnosis of a disease of the lung having immobilized thereon a plurality of oligonucleotides which hybridize specifically to genes of the gene groups which are differentially expressed in airways exposed to air pollutants, such as cigarette smoke, and have or are at high risk of developing lung disease, as compared to those individuals who are exposed to similar air pollutants and airways which are not exposed to such pollutants. In one embodiment, the oligonucleotides hybridize specifically to one allelic form of one or more genes which are differentially expressed for a disease of the lung. In a particular embodiment, the differentially expressed genes are selected from the group consisting of the genes shown in tables 11-14; preferably the group of genes comprises genes selected from the Table 22. In one preferred embodiment, the group of genes comprises the group of at least 20 genes selected from Table 13 and additional 5-10 genes selected from Tables 11 and 12. In one preferred embodiment, at least about 10 genes are selected from Table 14.

### BRIEF DESCRIPTION OF FIGURES

FIG. 1 shows Table 1, which sets forth a listing a group of 96 genes, their expression profile in lung cancer as compared to an individual not having lung cancer but being exposed to similar environmental stress, i.e. air pollutant, in this example, cigarette smoke. These genes were identified using Student's t-test.

FIG. 2 shows Table 2, listing a group of 84 genes, their expression profile in lung cancer as compared to an individual not having lung cancer but being exposed to similar environmental stress, i.e. air pollutant, in this example, cigarette smoke. These genes were identified using Student's t-test.

FIG. 3 shows Table 3, listing a group of 50 genes, and their expression profile in lung cancer as compared using a class-prediction model to an individual not having lung cancer but being exposed to similar environmental stress, i.e. air pollutant, in this example, cigarette smoke.

FIG. 4 shows Table 4, listing a group of 36 genes, their expression profile in lung cancer as compared to an individual not having lung cancer but being exposed to similar environmental stress, i.e. air pollutant, in this example, cigarette smoke. This group of genes is a combination of predictive genes identified using both Student's t-test and class-prediction model.

FIG. 5 shows an example of the results using class prediction model as obtained in Example 1. Training set included 74 samples, and the test set 24 samples. The mean age for the training set was 55 years, and the mean pack years smoked by the training set was 38. The mean age for the test set was 56 years, and the mean pack years smoked by the test set was 41.

FIG. 6 shows an example of the 50 gene class prediction model obtained in Example 1. Each square represents expression of one transcript. The transcript can be identified by the probe identifier on the y-axis according to the Affymetrix Human Genome Gene chip U133 probe numbers (see Appendix). The individual samples are identified on the x-axis. The samples are shown in this figure as individuals with lung cancer (“cancer”) and individuals without lung cancer (“no cancer”). The gene expression is shown as higher in darker squares and lower in lighter squares. One can clearly see the differences between the gene expression of these 50 genes in these two groups just by visually observing the pattern of lighter and darker squares.

FIG. 7 shows a comparison of sample-quality metrics. The graph plots the Affymetrix MAS 5.0 percent present (y-axis) versus the z-score derived filter (x-axis). The two metrics have a correlation (R2) of 0.82.

FIG. 8 shows distribution of accuracies for real vs. random 1000 runs. Histogram comparing test set class prediction accuracies of 1000 “sample randomized” classifiers generated by randomly assigning samples into training and test sets with true class labels (unshaded) versus 1000 “sample and class randomized” classifiers where the training set class labels were randomized following sample assignment to the training or test set (shaded).

FIG. 9 shows classification accuracy as a function of the average prediction strength over the 1000 runs of the algorithm with different training/test sets.

FIG. 10A shows the number of times each of the 80-predictive probe sets from the actual biomarker was present in the predictive lists of 80 probe sets derived from 1000 runs of the algorithm.

FIG. 10B shows the Number of times a probe set was present in the predictive lists of 80 probe sets derived from 1000 random runs of the algorithm described in Supplemental Table 7.

FIG. 11 shows Boxplot of the Prediction Strength values of the test set sample predictions made by the Weighted Voting algorithm across the 1000 runs with different training and test sets. The black boxplots (first two boxes from the left) are derived from the actual training and test set data with correct sample labels, the grey boxplots (last two boxes on the right) are derived from the test set predictions based on training sets with randomized sample labels.

FIG. 12 shows homogeneity of gene expression in large airway samples from smokers with lung cancer of varying cell types. Principal Component Analysis (PCA) was performed on the gene-expression measurements for the 80 genes in our predictor and all of the airway epithelium samples from patients with lung cancer. Gene expression measurements were Z(0,1) normalized prior to PCA. The graph shows the sample loadings for the first two principal components which together account for 58% of the variation among samples from smokers with cancer. There is no apparent separation of the samples with regard to lung tumor subtype.

FIG. 13 shows real time RT-PCR and microarray data for selected genes distinguishing smokers with and without cancer. Fold change for each gene is shown as the ratio of average expression level of cancer group (n=3) to the average expression of non-cancer group (n=3). Four genes (IL8, FOS, TPD52, and RAB1A) were found to be up-regulated in cancer group on both microarray and RT-PCR platforms; three genes (DCLRE1C, BACH2, and DUOX1) were found to be down-regulated in cancer group on both platforms.

FIG. 14 shows the class prediction methodology used. 129 samples (69 from patients without cancer; 60 from patients with lung cancer) were separated into a training (n=77) and a test set (n=52). The most frequently chosen 40 up- and 40 down-regulated genes from internal cross validation on the training set were selected for the final gene committee. The weighted voted algorithm using this committee of 80 genes was then used to predict the class of the test set samples.

FIG. 15 shows hierarchical clustering of class-predictor genes. Z-score-normalized gene-expression measurements of the eighty class-predictor genes in the 52 test-set samples are shown in a false-color scale and organized from top to bottom by hierarchical clustering. The Affymetrix U133A probeset ID and HUGO symbol are given to the right of each gene. The test-set samples are organized from left to right first by whether the patient had a clinical diagnosis of cancer. Within these two groups, the samples are organized by the accuracy of the class-predictor diagnosis (samples classified incorrectly are on the right shown in dark green). 43/52 (83%) test samples are classified correctly. The sample ID is given at the top of each column. The prediction strength of each of the diagnoses made by the class-prediction algorithm is indicated in a false-color scale immediately below the prediction accuracy. Prediction strength is a measure of the level of diagnostic confidence and varies on a continuous scale from 0 to 1 where 1 indicates a high degree of confidence.

FIG. 16 shows a Comparison of Receiver Operating Characteristic (ROC) curves. Sensitivity (y-axis) and 1-Specificity (x-axis) were calculated at various prediction strength thresholds where a prediction of no cancer was assigned a negative prediction strength value and a prediction of cancer was assigned a positive prediction strength value. The solid black line represents the ROC curve for the airway gene expression classifier. The dotted black line represents the average ROC curve for 1000 classifiers derived by randomizing the training set class labels (“class randomized”). The upper and lower lines of the gray shaded region represent the average ROC curves for the top and bottom half of random biomarkers (based on area under the curve). There is a significant difference between the area under the curve of the actual classifier and the random classifiers (p=0.004; empiric p-value based on permutation)

FIG. 17 shows the Principal Component Analysis (PCA) of biomarker gene expression in lung tissue samples. The 80 biomarker probesets were mapped to 64 probesets in the Bhattacharjee et al. HGU95Av2 microarray dataset of lung cancer and normal lung tissue. The PCA is a representation of the overall variation in expression of the 64 biomarker probesets. The normal lung samples (NL) are represented in green, the adenocarcinomas (AD) in red, the small cells (SC) in blue, and the squamous (SQ) lung cancer samples in yellow. The normal lung samples separate from the lung cancer samples along the first principal component (empirically derived p-value=0.023, see supplemental methods).

FIGS. 18A-18C show data obtained in this study. FIG. 18A shows bronchoscopy results for the 129 patients in the study. Only 32 of the 60 patients that had a final diagnosis of cancer had bronchoscopies that were diagnostic of lung cancer. The remaining 97 samples had bronchoscopies that were negative for lung cancer including 5 that had a definitive alternate benign diagnosis. This resulted in 92 patients with non-diagnostic bronchoscopy that required further tests and/or clinical follow-up. FIG. 18B shows biomarker prediction results. 36 of the 92 patients with non-diagnostic bronchoscopies exhibited a gene expression profile that was positive for lung cancer. This resulted in 25 of 28 cancer patients with non-diagnostic bronchoscopies being predicted to have cancer. FIG. 18C shows combined test results. In a combined test where a positive test result from either bronchoscopy or gene expression is considered indicative of lung cancer a sensitivity of 95% (57 of 60 cancer patients) with only a 16% false positive rate (11 of 69 non-cancer patients) is achieved. The shading of each contingency table is reflective of the overall fraction of each sample type in each quadrant.

FIGS. 19A-19B show a comparison of bronchoscopy and biomarker prediction by A) cancer stage or B) cancer subtype. Each square symbolizes one patient sample. The upper half represents the biomarker prediction accuracy and the lower half represents the bronchoscopy accuracy. Not all cancer samples are represented in this figure. FIG. 19A includes only Non Small Cell cancer samples that could be staged using the TMN system (48 of the 60 total cancer samples). FIG. 19B includes samples that could be histologically classified as Adenocarcinoma, Squamous Cell Carcinoma and Small Cell Carcinoma (45 of the 60 total cancer samples).

FIGS. 20A-20F show hierarchical clustering of bronchial airway epithelial samples from current (striped box) and never (white box) smokers according to the expression of 60 genes whose expression levels are altered by smoking in the nasal epithelium. Airway samples tend to group with their appropriate class. Dark grey indicates higher level of expression and light grey lower level of expression.

FIG. 21 shows hierarchical clustering of nasal epithelial samples from patients with sarcoid (stiped box) and normal healthy volunteers (white box) according to the expression of top 20 t-test genes that differ between the 2 groups (P<0.00005). With few exceptions, samples group into their appropriate classes. Light grey=low level of expression, black=mean level of expression, dark grey=high level of expression.

FIG. 22 shows smoking related genes in mouth, nose and bronchus. Principal component analysis (PCA) shows the variation in expression of genes affected by tobacco exposure in current smokers (dark grey) and never smokers (black). Airway epithelium type is indicated by the symbol shape: bronchial (circle), nasal (triangle) and mouth (square). Samples largely separate by smoking status across the first principal component, with the exception of samples from mouth. This indicates a common gene expression host response that can be seen both in the bronchial epithelial tissue and the nasal epithelial tissue.

FIG. 23 shows a supervised hierarchical clustering analysis of cancer samples. Individuals with sarcoidosis and individuals with no sarcoids were sampled from both lung tissues and nasal tissues. Gene expression analysis showed that expression of 37 genes can be used to differentiate the cancer samples and non-cancer sampled either from bronchial or nasal epithelial cells. Light grey in the clustering analysis indicates low level of expression and dark grey high level of expression. Asterisk next to the circles indicates that these samples were from an individual with stage 0-1 sarcoidosis. The dot next to the circle indicates that these samples were from an individual with a stage 4 sarcoidosis.

FIG. 24 shows airway t-test genes projected on nose data including the 107 leading edge genes as shown in Table 19. Enrichment of differentially expressed bronchial epithelial genes among genes highly changed in the nasal epithelium in response to smoking. Results from GSEA analysis shows the leading edge of the set of 361 differentially expressed bronchial epithelial genes being overrepresented among the top ranked list of genes differentially expressed in nasal epithelium cells in response to smoking. There are 107 genes that comprise the “leading edge subset” (p<0.001).

FIG. 25 shows 107 Leading Edge Genes from Airway—PCA on Nose Samples. Asterisk next to the circle indicates current smokers. Dark circles represent samples from never smokers. Principal component analysis of 107 “leading edge” genes from bronchial epithelial cells enriched in the nasal epithelial gene expression profile. Two dimensional PCA of the 107 “leading edge” genes from the bronchial epithelial signature that are enriched in the nasal epithelial cell expression profile.

FIG. 26 shows a Bronch projection from 10 tissues. From this figure one can see, that the samples from bronchial epithelial cells (dotted squares) and the samples from nose epithelial cells (crossed squares) overlapped closely and were clearly distinct from samples from other tissues, including mouth. Principal component analysis of 2382 genes from normal airway transcriptome across 10 tissues. Principal component analysis (PCA) of 2382 genes from the normal airway transcriptome across 10 different tissue types. Samples separate based on expression of transcriptome genes.

FIGS. 27A-27C show a hierarchical clustering of 51 genes across epithelial cell functional categories. Supervised hierarchical clustering of 51 genes spanning mucin, dynein/microtubule, cytochrome P450, glutathione, and keratin functional gene categories. The 51 genes were clustered across the 10 tissue types separately for each functional group.

### DETAILED DESCRIPTION OF THE INVENTION

The present invention is directed in part to gene/transcript groups and methods of using the expression profile of these gene/transcript groups in diagnosis and prognosis of lung diseases.

We provide a method that significantly increases the diagnostic accuracy of lung diseases, such as lung cancer. When one combines the gene expression analysis of the present invention with bronchoscopy, the diagnosis of lung cancer is dramatically better by detecting the cancer in an earlier stage than any other available method to date, and by providing far fewer false negatives and/or false positives than any other available method.

We have found a group of gene transcripts that we can use individually and in groups or subsets for enhanced diagnosis for lung diseases, such as lung cancer, using gene expression analysis. We provide detailed guidance on the increase and/or decrease of expression of these genes for diagnosis and prognosis of lung diseases, such as lung cancer.

One example of the gene transcript groups useful in the diagnostic/prognostic tests of the invention is set forth in Table 6. We have found that taking any group that has at least 20 of the Table 6 genes provides a much greater diagnostic capability than chance alone.

Preferably one would use more than 20 of these gene transcript, for example about 20-100 and any combination between, for example, 21, 22, 23, 24, 25, 26, 27, 28, 29, 30, and so on. Our preferred groups are the groups of 96 (Table 1), 84 (Table 2), 50 (Table 3), 36 (Table 4), 80 (Table 5), 535 (Table 6) and 20 (Table 7). In some instances, we have found that one can enhance the accuracy of the diagnosis by adding additional genes to any of these specific groups.

Naturally, following the teachings of the present invention, one may also include one or more of the genes and/or transcripts presented in Tables 1-7 into a kit or a system for a multicancer screening kit. For example, any one or more genes and or transcripts from Table 7 may be added as a lung cancer marker for a gene expression analysis.

When one uses these groups, the genes in the group are compared to a control or a control group. The control groups can be non-smokers, smokers, or former smokers. Preferably, one compares the gene transcripts or their expression product in the biological sample of an individual against a similar group, except that the members of the control groups do not have the lung disorder, such as emphysema or lung cancer. For example, comparing can be performed in the biological sample from a smoker against a control group of smokers who do not have lung cancer. When one compares the transcripts or expression products against the control for increased expression or decreased expression, which depends upon the particular gene and is set forth in the tables—not all the genes surveyed will show an increase or decrease. However, at least 50% of the genes surveyed must provide the described pattern. Greater reliability if obtained as the percent approaches 100%. Thus, in one embodiment, one wants at least 55%, 60%, 65%, 70%, 75%, 80%, 85%, 90%, 95%, 98%, 99% of the genes surveyed to show the altered pattern indicative of lung disease, such as lung cancer, as set forth in the tables as shown below.

The presently described gene expression profile can also be used to screen for individuals who are susceptible for lung cancer. For example, a smoker, who is over a certain age, for example over 40 years old, or a smoker who has smoked, for example, a certain number of years, may wish to be screened for lung cancer. The gene expression analysis as described herein can provide an accurate very early diagnosis for lung cancer. This is particularly useful in diagnosis of lung cancer, because the earlier the cancer is detected, the better the survival rate is.

For example, when we analyzed the gene expression results, we found, that if one applies a less stringent threshold, the group of 80 genes as presented in Table 5 are part of the most frequently chosen genes across 1000 statistical test runs (see Examples below for more details regarding the statistical testing). Using random data, we have shown that no random gene shows up more than 67 times out of 1000. Using such a cutoff, the 535 genes of Table 6 in our data show up more than 67 times out of 1000. All the 80 genes in Table 5 form a subset of the 535 genes. Table 7 shows the top 20 genes which are subset of the 535 list. The direction of change in expression is shown using signal to noise ratio. A negative number in Tables 5, 6, and 7 means that expression of this gene or transcript is up in lung cancer samples. Positive number in Table 5, 6, and 7, indicates that the expression of this gene or transcript is down in lung cancer.

Accordingly, any combination of the genes and/or transcripts of Table 6 can be used. In one embodiment, any combination of at least 5-10, 10-20, 20-30, 30-40, 40-50, 50-60, 60-70, 70-80, 80, 80-90, 90-100, 100-120, 120-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, 490-500, 500-510, 510-520, 520-530, and up to about 535 genes selected from the group consisting of genes or transcripts as shown in the Table 6.

Table 7 provides 20 of the most frequently variably expressed genes in lung cancer when compared to samples without cancer. Accordingly, in one embodiment, any combination of about 3-5, 5-10, 11, 12, 13, 14, 15, 16, 17, 18, 19, or all 20 genes and/or transcripts of Table 7, or any sub-combination thereof are used.

In one embodiment, the invention provides a gene group the expression profile of which is useful in diagnosing lung diseases and which comprises probes that hybridize ranging from 1 to 96 and all combinations in between for example 5, 10, 15, 20, 25, 30, 35, at least about 36, at least to 40, at least to 50, at least to 60, to at least 70, to at least 80, to at least 90, or all of the following 96 gene sequences: NM_003335; NM_000918; NM_006430.1; NM_001416.1; NM_004090; NM_006406.1; NM_003001.2; NM_001319; NM_006545.1; NM_021145.1; NM_002437.1; NM_006286; NM_001003698///NM_001003699///NM_002955; NM_001123///NM_006721; NM_024824; NM_004935.1; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_001696; NM_005494///NM_058246; NM_006534///NM_181659; NM_006368; NM_002268///NM_032771; NM_014033; NM_016138; NM_007048///NM_194441; NM_006694; NM_000051///NM_138292///NM_138293; NM_000410///NM_139002///NM_139003///NM_139004///NM_139005///NM_139006///NM_139007///NM_139008///NM_139009///NM_139010///NM_139011; NM_004691; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_012394; NM_019011///NM_207111///NM_207116; NM_017646; NM_021800; NM_016049; NM_014395; NM_014336; NM_018097; NM_019014; NM_024804; NM_018260; NM_018118; NM_014128; NM_024084; NM_005294; AF077053; NM_138387; NM_024531; NM_000693; NM_018509; NM_033128; NM_020706; AI523613; and NM_014884

In one embodiment, the invention provides a gene group the expression profile of which is useful in diagnosing lung diseases and comprises probes that hybridize to at least, for example, 5, 10, 15, 20, 25, 30, 35, at least about 36, at least to 40, at least to 50, at least to 60, to at least 70, to at least 80, to all of the following 84 gene sequences: NM_030757.1; R83000; AK021571.1; NM_014182.1; NM_17932.1; U85430.1; AI683552; BC002642.1; AW024467; NM_030972.1; BC021135.1; AL161952.1; AK026565.1; AK023783.1; BF218804; NM_001281.1; NM_024006.1; AK023843.1; BC001602.1; BC034707.1; BC064619.1; AY280502.1; BC059387.1; AF135421.1; BC061522.1; L76200.1; U50532.1; BC006547.2; BC008797.2; BC000807.1; AL080112.1; BC033718.1///BC046176.1///BC038443.1; NM_000346.1; BC008710.1; Hs.288575 (UNIGENE ID); AF020591.1; BC000423.2; BC002503.2; BC008710.1; BC009185.2; Hs.528304 (UNIGENE ID); U50532.1; BC013923.2; BC031091; NM_007062; Hs.249591 (Unigene ID); BC075839.1///BC073760.1; BC072436.1///BC004560.2; BC001016.2; Hs.286261 (Unigene ID); AF348514.1; BC005023.1; BC066337.1///BC058736.1///BC050555.1; Hs.216623 (Unigene ID); BC072400.1; BC041073.1; U43965.1; BC021258.2; BC016057.1; BC016713.1///BC014535.1///AF237771.1; BC000360.2; BC007455.2; BC000701.2; BC010067.2; BC023528.2///BC047680.1; BC064957.1; Hs.156701 (Unigene ID); BC030619.2; BC008710.1; U43965.1; BC066329.1; Hs.438867 (Unigene ID); BC035025.2///BC050330.1; BC023976.2; BC074852.2///BC074851.2; Hs.445885 (Unigene ID); BC008591.2///BC050440.1///; BC048096.1; AF365931.1; AF257099.1; and BC028912.1.

In one embodiment, the invention provides a gene group the expression profile of which is useful in diagnosing lung diseases and comprises probes that hybridize to at least, for example 5, 10, 15, 20, 25, 30, preferably at least about 36, still more preferably at least to 40, still more preferably at least to 45, still more preferably all of the following 50 gene sequences, although it can include any and all members, for example, 20, 21, 22, up to and including 36: NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; AB014576.1; BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1. In one preferred embodiment, one can use at least 20-30, 30-40, of the 50 genes that overlap with the individual predictor genes identified in the analysis using the t-test, and, for example, 5-9 of the non-overlapping genes, identified using the t-test analysis as individual predictor genes, and combinations thereof.

In one embodiment, the invention provides a gene group the expression profile of which is useful in diagnosing lung diseases and comprises probes that hybridize to at least for example 5, 10, 15, 20, preferably at least about 25, still more preferably at least to 30, still more preferably all of the following 36 gene sequences: NM_007062.1; NM_001281.1; BC002642.1; NM_000346.1; NM_006545.1; BG034328; NM_019067.1; NM_017925.1; NM_017932.1; NM_030757.1; NM_030972.1; NM_002268///NM_032771; NM_007048///NM_194441; NM_006694; U85430.1; NM_004691; AB014576.1; BF218804; BE467941; R83000; AL161952.1; AK023843.1; AK021571.1; AK023783.1; AL080112.1; AW971983; AI683552; NM_024006.1; AK026565.1; NM_014182.1; NM_021800.1; NM_016049.1; NM_021971.1; NM_014128.1; AA133341; and AF198444.1. In one preferred embodiment, one can use at least 20 of the 36 genes that overlap with the individual predictors and, for example, 5-9 of the non-overlapping genes, and combinations thereof.

The expression of the gene groups in an individual sample can be analyzed using any probe specific to the nucleic acid sequences or protein product sequences encoded by the gene group members. For example, in one embodiment, a probe set useful in the methods of the present invention is selected from the nucleic acid probes of between 10-15, 15-20, 20-180, preferably between 30-180, still more preferably between 36-96, still more preferably between 36-84, still more preferably between 36-50 probes, included in the Affymetrix Inc. gene chip of the Human Genome U133 Set and identified as probe ID Nos: 208082_x_at, 214800_x_at, 215208_x_at, 218556_at, 207730_x_at, 210556_at, 217679_x_at, 202901_x_at, 213939_s_at, 208137_x_at, 214705_at, 215001_s_at, 218155_x_at, 215604_x_at, 212297_at, 201804_x_at, 217949_s_at, 215179_x_at, 211316_x_at, 217653_x_at, 266_s_at, 204718_at, 211916_s_at, 215032_at, 219920_s_at, 211996_s_at, 200075_s_at, 214753_at, 204102_s_at, 202419_at, 214715_x_at, 216859_x_at, 215529_x_at, 202936_s_at, 212130_x_at, 215204_at, 218735_s_at, 200078_s_at, 203455_s_at, 212227_x_at, 222282_at, 219678x_at, 208268_at, 221899_at, 213721_at, 214718_at, 201608_s_at, 205684_s_at, 209008_x_at, 200825_s_at, 218160_at, 57739_at, 211921_x_at, 218074_at, 200914_x_at, 216384_x_at, 214594_x_at, 222122_s_at, 204060_s_at, 215314_at, 208238_x_at, 210705_s_at, 211184_s_at, 215418_at, 209393_s_at, 210101_x_at, 212052_s_at, 215011_at, 221932_s_at, 201239_s_at, 215553_x_at, 213351_s_at, 202021_x_at, 209442_x_at, 210131_x_at, 217713_x_at, 214707_x_at, 203272_s_at, 206279_at, 214912_at, 201729_s_at, 205917_at, 200772_x_at, 202842_s_at, 203588_s_at, 209703_x_at, 217313_at, 217588_at, 214153_at, 222155_s_at, 203704_s_at, 220934_s_at, 206929_s_at, 220459_at, 215645_at, 217336_at, 203301_s_at, 207283_at, 222168_at, 222272_x_at, 219290_x_at, 204119_s_at, 215387_x_at, 222358_x_at, 205010_at, 1316_at, 216187_x_at, 208678_at, 222310_at, 210434_x_at, 220242_x_at, 207287_at, 207953_at, 209015_s_at, 221759_at, 220856_x_at, 200654_at, 220071_x_at, 216745_x_at, 218976_at, 214833_at, 202004_x_at, 209653_at, 210858_x_at, 212041_at, 221294_at, 207020_at, 204461_x_at, 205367_at, 219203_at, 215067_x_at, 212517_at, 220215_at, 201923_at, 215609_at, 207984_s_at, 215373_x_at, 216110_x_at, 215600_x_at, 216922_x_at, 215892_at, 201530_x_at, 217371_s_at, 222231_s_at, 218265_at, 201537_s_at, 221616_s_at, 213106_at, 215336_at, 209770_at, 209061_at, 202573_at, 207064_s_at, 64371_at, 219977_at, 218617_at, 214902_x_at, 207436_x_at, 215659_at, 204216_s_at, 214763_at, 200877_at, 218425_at, 203246_s_at, 203466_at, 204247_s_at, 216012_at, 211328_x_at, 218336_at, 209746_s_at, 214722_at, 214599_at, 220113_x_at, 213212_x_at, 217671_at, 207365_x_at, 218067_s_at, 205238_at, 209432_s_at, and 213919_at. In one preferred embodiment, one can use at least, for example, 10-20, 20-30, 30-40, 40-50, 50-60, 60-70, 70-80, 80-90, 90-100, 110, 120, 130, 140, 150, 160, or 170 of the 180 genes that overlap with the individual predictors genes and, for example, 5-9 of the non-overlapping genes and combinations thereof.

Sequences for the Affymetrix probes are provided in the Appendix to the specification, all the pages of which are herein incorporated by reference in their entirety.

One can analyze the expression data to identify expression patters associated with any lung disease that is caused by exposure to air pollutants, such as cigarette smoke, asbestos or any other lung disease. For example, the analysis can be performed as follows. One first scans a gene chip or mixture of beads comprising probes that are hybridized with a study group samples. For example, one can use samples of non-smokers and smokers, non-asbestos exposed individuals and asbestos-exposed individuals, non-smog exposed individuals and smog-exposed individuals, smokers without a lung disease and smokers with lung disease, to obtain the differentially expressed gene groups between individuals with no lung disease and individuals with lung disease. One must, of course select appropriate groups, wherein only one air pollutant can be selected as a variable. So, for example, one can compare non-smokers exposed to asbestos but not smog and non-smokers not exposed to asbestos or smog.

The obtained expression analysis, such as microarray or microbead raw data consists of signal strength and detection p-value. One normalizes or scales the data, and filters the poor quality chips/bead sets based on images of the expression data, control probes, and histograms. One also filters contaminated specimens which contain non-epithelial cells. Lastly, one filters the genes of importance using detection p-value. This results in identification of transcripts present in normal airways (normal airway transcriptome). Variability and multiple regression analysis can be used. This also results in identification of effects of smoking on airway epithelial cell transcription. For this analysis, one can use T-test and Pearson correlation analysis. One can also identify a group or a set of transcripts that are differentially expressed in samples with lung disease, such as lung cancer and samples without cancer. This analysis was performed using class prediction models.

For analysis of the data, one can use, for example, a weighted voting method. The weighted voting method ranks, and gives a weight “p” to all genes by the signal to noise ration of gene expression between two classes: P=mean_{(class 1)}−mean_{(class 2)}/sd_{(class 1)}=sd_{(class 2)}. Committees of variable sizes of the top ranked genes are used to evaluate test samples, but genes with more significant p-values can be more heavily weighed. Each committee genes in test sample votes for one class or the other, based on how close that gene expression level is to the class 1 mean or the class 2 mean. V_{(gene A)}=P_{(gene A)}, i.e. level of expression in test sample less the average of the mean expression values in the two classes. Votes for each class are tallied and the winning class is determined along with prediction strength as PS=V_{win}−Vₗₒₛₑ/V_{win}+Vₗₒₛₑ. Finally, the accuracy can be validated using cross-validation+/−independent samples.

Table 1 shows 96 genes that were identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer. In one embodiment, the exemplary probes shown in the column “Affymetrix Id in the Human Genome U133 chip” can be used. Sequences for the Affymetrix probes are provided in the Appendix.

TABLE 1
96 Gene Group

| Affymetrix | | | Gene | Direction |
|---|---|---|---|---|
| Id | GenBank ID | Gene Description | Name | in Cancer |
| 1316_at | NM_003335 | ubiquitin-activated | UBE1L | down |
| | | enzyme E1-like | | |
| 200654_at | NM_000918 | procollagen-proline, | P4HB | up |
| | | 2-oxoglutarate | | |
| | | 4-dioxygenase | | |
| | | (proline 4-hydroxylase), | | |
| | | beta polypeptide (protein | | |
| | | disulfide isomerase; | | |
| | | thyroid hormone | | |
| | | binding protein p55) | | |
| 200877_at | NM_006430.1 | chaperonin containing | CCT4 | up |
| | | TCP1, subunit 4 (delta) | | |
| 201530_x_at | NM_001416.1 | eukaryotic translation | EIF4A1 | up |
| | | factor 4A, isoform 1 | | |
| 201537_s_at | NM_004090 | dual specificity | DUSP3 | up |
| | | phosphatase 3 | | |
| | | (vaccinia virus | | |
| | | phosphatase | | |
| | | VH1-related) | | |
| 201923_at | NM_006406.1 | peroxiredoxin 4 | PRDX4 | up |
| 202004_x_at | NM_003001.2 | succinate | SDHC | up |
| | | dehydrogenase | | |
| | | complex, subunit C, | | |
| | | integral membrane | | |
| | | protein 15kDa | | |
| 202573_at | NM_001319 | casein kinase 1, gamma 2 | CSNKIG2 | down |
| 203246_s_at | NM_006545.1 | tumor suppressor | TUSC4 | up |
| | | candidate 4 | | |
| 20330l_s_at | NM_021145.1 | cyclin D binding | DMTF1 | down |
| | | myb-like transcription | | |
| | | factor 1 | | |
| 203466_at | NM_002437.1 | MpV17 transgene, | MPV17 | up |
| | | murine homolog, | | |
| | | glomerusclerosis | | |
| 203588_s_at | NM_006286 | transcription factor Dp-2 | TFDP2 | up |
| | | (E2F dimerization | | |
| | | partner 2) | | |
| 203704_s_at | NM_001003698 /// | ras responsive clement | RREB1 | down |
| | NM_001003699 /// | binding protein 1 | | |
| | NM_002955 | | | |
| 204119_s_at | NM_001123 /// | adenosine kinase | ADK | up |
| | NM_006721 | | | |
| 204216_s_at | NM_024824 | nuclear protein UKp68 | FLJ11806 | up |
| 204247_s_at | NM_004935.1 | cyclin-dependent kinase 5 | CDK5 | up |
| 20446l_x_at | NM_002853.1 | RADI homolog | RADI | down |
| 205010_at | NM_019067.1 | hypothetical protein | FLJ10613 | down |
| | | FLJ10613 | | |
| 205238_at | NM_024917.1 | chromosome X open | CXorf34 | down |
| | | reading frame 34 | | |
| 205367_at | NM_020979.1 | adaptor protein with | APS | down |
| | | pleckstrin homology | | |
| | | and src homology 2 | | |
| | | domains | | |
| 206929_s_at | NM_005597.1 | nuclear factor I/c | NFIC | down |
| | | (CCAAT-binding | | |
| | | transcription factor) | | |
| 207020_at | NM_007031.1 | heat shock transcription | HSF2BP | down |
| | | factor 2 binding protein | | |
| 207064_s_at | NM_009590.1 | amine oxidase, | AOC2 | down |
| | | copper containing 2 | | |
| | | (retina-specific) | | |
| 207283_at | NM_020217.1 | hypothetical protein | DKFZp547I014 | down |
| | | DKFZp547I0l4 | | |
| 207287_at | NM_025026.1 | hypothetical protein | FLJI4107 | down |
| | | FLJ14107 | | |
| 207365_x_at | NM_014709.1 | ubiquitin specific | USF34 | down |
| | | protease 34 | | |
| 207436_x_at | NM_014896.1 | KIAA0894 protein | KIAA0894 | down |
| 207953_at | AF010144 | — | — | down |
| 207984_s_at | NM_005374.1 | membrane protein, | MPP2 | down |
| | | palmitoylated 2 | | |
| | | (MAGUK p55 | | |
| | | subfamily member2 | | |
| 208678_at | NM_001696 | ATPase, H+ | ATP6V1E1 | up |
| | | transporting, lysosomal | | |
| | | 31kDa, V1 subunit E, | | |
| | | isoform 1 | | |
| 209015_s_at | NM_005494 /// | DnaJ (Hsp40) homolog, | DNAJB6 | up |
| | NM_058246 | subfamily B, member 6 | | |
| 20906l_at | NM_006534 /// | nuclear receptor | NCOA3 | down |
| | NM_181659 | coactivator 3 | | |
| 209432_s_at | NM_006368 | cAMP responsive element | CREB3 | up |
| | | binding protein 3 | | |
| 209653_at | NM_002268 /// | karyopherin alpha 4 | KPNA4 | up |
| | NM_032771 | (importin alpha 3) | | |
| 209703_x_at | NM_014033 | DKFZP586A0522 protein | DKFZP586A0522 | down |
| 209746_s_at | NM_016138 | coenzyme Q7 homolog, | COQ7 | down |
| | | ubiquinone | | |
| 209770_at | NM_007048 /// | butyrophilin, subfamily 3, | BTN3A1 | down |
| | NM_194441 | member A1 | | |
| 210434_x_at | NM_006694 | jumping translocation | JTB | up |
| | | breakpoint | | |
| 210858_x_at | NM_000051 /// | ataxia telangiectasia | ATM | down |
| | NM_138292 /// | mutated (includes | | |
| | NM_138293 | complementation | | |
| | | groups A, C, and D | | |
| 211328_x_at | NM_000410 /// | hemochromatosis | HFE | down |
| | NM_139002 /// | | | |
| | NM_139003 /// | | | |
| | NM_139004 /// | | | |
| | NM_139005 /// | | | |
| | NM_139006 /// | | | |
| | NM_139007 /// | | | |
| | NM_139008 /// | | | |
| | NM_139009 /// | | | |
| | NM_139010 /// | | | |
| | NM_139011 | | | |
| 212041_at | NM_004691 | ATPase, H+ transporting, | ATP6V0D1 | up |
| | | lysosomal 38kDa, | | |
| | | V0 subunit d | | |
| | | isoform 1 | | |
| 212517_at | NM_012070 /// | attractin | ATRN | down |
| | NM_139321 /// | | | |
| | NM_039322 | | | |
| 213106_at | NM_006095 | ATPase, | ATP8A1 | down |
| | | aminophospholipid | | |
| | | transporter (APLT), | | |
| | | Class I, type 8A, | | |
| | | member 1 | | |
| 213212_x_at | AI632181 | Similar to FLJ40113 | — | down |
| | | protein | | |
| 213919_at | AW024467 | — | — | down |
| 214153_at | NM_021814 | ELOVL family member 5, | ELOVL5 | down |
| | | elongation of long | | |
| | | chain fatty acids | | |
| | | (FEN1/Elo2, SUR4/ | | |
| | | Elo3-like, yeast) | | |
| 214599_at | NM_005547.1 | involucrin | IVL | down |
| 214722_at | NM_203458 | similar to NOTCH2 | N2N | down |
| | | protein | | |
| 214763_at | NM_015547 /// | thiosterase, adipose | THEA | down |
| | NM_147161 | associated | | |
| 214833_at | AB007958.1 | KIAA0792 gene product | KIAA0792 | down |
| 214902_x_at | NM_207488 | FLJ42393 protein | FLJ42393 | down |
| 215067_x_at | NM_005809 /// | peroxiredoxin 2 | PRDX2 | down |
| | NM_181737 /// | | | |
| | NM_181738 | | | |
| 215336_at | NM_016248 /// | A kinase (PRKA) | AKAP11 | down |
| | NM_144490 | anchor protein | | |
| 215373_x_at | AK022213.1 | hypothetical protein | FLJ12151 | down |
| | | FLJ12151 | | |
| 215387_x_at | NM_005708 | Glypican 6 | GPC6 | down |
| 215600_x_at | NM_207102 | F-box and WD-40 | FBXW12 | down |
| | | domain protein 12 | | |
| 215609_at | AK023895 | — | — | down |
| 215645_at | NM_144606 /// | Hypothetical protein | FLCN | down |
| | NM_144997 | MGC13008 | | |
| 215659_at | NM_018530 | Gasdermin-like | GSDML | down |
| 215892_at | AK021474 | — | — | down |
| 216012_at | U43604.1 | human unidentified mRNA, | — | down |
| | | partial sequence | | |
| 216110_x_at | AU147017 | — | — | down |
| 216187_x_at | AF222691.1 | Homo sapiens Alu repeat | LNX1 | down |
| 216745_x_at | NM_015116 | Leucine-rich repeats and | LRCH1 | down |
| | | calponin homology (CH) | | |
| | | domain containing 1 | | |
| 216922_x_at | NM_001005375 /// | deleted in azoospermia | DAZ2 | down |
| | NM_001005785 /// | | | |
| | NM_001005786 /// | | | |
| | NM_004081 /// | | | |
| | NM_020363 /// | | | |
| | NM_020364 /// | | | |
| | NM_020420 | | | |
| 217313_at | AC004692 | — | ... | down |
| 217336_al | NM_001014 | ribosomal protein S10 | RPS10 | down |
| 217371_s_at | NM_000585 /// | interleukin 15 | IL15 | down |
| | NM_172174 /// | | | |
| | NM_172175 | | | |
| 217588_at | NM_054020 /// | cation channel, | CATSPER2 | down |
| | NM_172095 /// | sperm associated 2 | | |
| | NM_172096 /// | | | |
| | NM_172097 | | | |
| 217671_at | BE466926 | — | — | down |
| 218067_s_at | NM_018011 | hypothetical protein | FLJ10154 | down |
| | | FLJ10154 | | |
| 218265_at | NM_024077 | SECIS binding protein 2 | SECISBP2 | down |
| 218336_at | NM_012394 | prefoldin 2 | PFDN2 | up |
| 218425_at | NM_019011 /// | TRIAD3 protein | TRIAD3 | down |
| | NM_207111 /// | | | |
| | NM_207116 | | | |
| 218617_at | NM_017646 | tRNA isopentenyltransferase 1 | TRIT1 | down |
| 218976_at | NM_021800 | DnaJ (Hsp40) homolog, | DNAJC12 | up |
| | | subfamily C, member 12 | | |
| 219203_at | NM_016049 | chromosome 14 open | C14orf122 | up |
| | | reading frame 122 | | |
| 219290_x_at | NM_014395 | dual adaptor of | DAPP1 | down |
| | | phosphotyrosine and 3- | | |
| | | phosphoinositides | | |
| 219977_at | NM_014336 | aryl hydrocarbon | AIPL1 | down |
| | | receptor interacting | | |
| | | protein-like 1 | | |
| 220071_x_at | NM_018097 | chromosome 15 open | C15orf25 | down |
| | | reading frame 25 | | |
| 220113_x_at | NM_019014 | polymerase (RNA) I | POLR1B | down |
| | | polypeptide B, 128 kDa | | |
| 220215_at | NM_024804 | hypothetical protein | FLJ12606 | down |
| | | FLJ12606 | | |
| 220242_x_at | NM_018260 | hypothetical protein | FLJ10891 | down |
| | | FLJ10891 | | |
| 220459_at | NM_018118 | MCM3 minichromosome | MCM3APAS | down |
| | | maintenace deficient 3 | | |
| | | (s. cerevisiae) associated | | |
| | | protein, antisense | | |
| 220856_x_at | NM_014128 | — | | down |
| 220934_s_at | NM_024084 | hypothetical protein MGC3196 | MGC3196 | down |
| 221294_at | NM_005294 | G protein-coupled receptor 21 | GPR21 | down |
| 221616_s_at | AF077053 | Phosphoglycerate kinase 1 | PGK1 | down |
| 221759_at | NM_138387 | glucose-6-phosphatase | G6PC3 | up |
| | | catalytic subunit-related | | |
| 222155_s_at | NM_024531 | G protein-coupled | GPR172A | up |
| | | receptor 172 A | | |
| 222168_at | NM_000693 | Aldehyde | ALDH1A3 | down |
| | | dehydrogenase 1 | | |
| | | family, member A3 | | |
| 222231_s_at | NM_018509 | hypothetical protein | PRO1855 | up |
| | | PRO 1855 | | |
| 222272_x_at | NM_033128 | scinderin | SCIN | down |
| 222310_at | NM.020706 | splicing factor, | SFRS15 | down |
| | | arginine/serine-rich 15 | | |
| 222358_x_at | A1523613 | — | — | down |
| 64371_at | NM_014884 | splicing factor, | SFRS14 | down |
| | | arginine/serine-rich 14 | | |

Table 2 shows one preferred 84 gene group that was identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer. These genes were identified using traditional Student's t-test analysis.

In one embodiment, the exemplary probes shown in the column “Affymetrix Id in the Human Genome U133 chip” can be used in the expression analysis.

TABLE 2
84 Gene Group

| GenBank ID | | | | |
|---|---|---|---|---|
| (unless otherwise | | | Direction in | Affymetrix |
| mentioned) | Gene Name | Description | Cancer | ID |
| NM_030757.1 | MKRN4 | makorin, ring finger | down | 208082_x_at |
| | | protein, 4///makorin, | | |
| | | ring finger protein, 4 | | |
| R83000 | BTF3 | basic transcription | down | 214800_x_at |
| | | factor 3 | | |
| AK021571.1 | MUC20 | mucin 20 | down | 215208_x_at |
| NM_014182.1 | ORMDL2 | ORM1-like 2 (S. | up | 218556_at |
| | | cerevisiae) | | |
| NM_17932.1 | FLJ20700 | hypothetical protein | down | 207730_x_at |
| | | FLJ20700 | | |
| U85430.1 | NFATC3 | nuclear factor of | down | 210556_at |
| | | activated T-cells, | | |
| | | cytoplasmic, | | |
| | | calcineurin-dependent 3 | | |
| AI683552 | — | — | down | 217679_x_at |
| BC002642.1 | CTSS | cathepsin S | down | 202901_x_at |
| AW024467 | RIPX | rap2 interacting protein | down | 213939_s_at |
| | | x | | |
| NM_030972.1 | MGC5384 | hypothetical protein | down | 208137_x_at |
| | | MGC5384/// | | |
| | | hypothetical protein | | |
| | | MGC5384 | | |
| BC021135.1 | INADL | InaD-like protein | down | 214705_at |
| AL161952.1 | GLUL | glutamate-ammonia | down | 215001_s_at |
| | | ligase (glutamine | | |
| | | synthase) | | |
| AK026565.1 | FLJ10534 | hypothetical protein | down | 218155_x_at |
| | | FLJ10534 | | |
| AK023783.1 | — | Homo sapiens cDNA | down | 215604_x_at |
| | | FLJ13721 fis, clone | | |
| | | PLACE2000450. | | |
| BF218804 | AFURS1 | ATPase family homolog | down | 212297_at |
| | | up-regulated in | | |
| | | senescence cells | | |
| NM_001281.1 | CKAP1 | cytoskeleton associated | up | 201804_x_at |
| | | protein 1 | | |
| NM_024006.1 | IMAGE3455200 | hypothetical protein | up | 217949_s_at |
| | | IMAGE3455200 | | |
| AK023843.1 | PGF | placental growth factor, | down | 215179_x_at |
| | | vascular endothelial | | |
| | | growth factor-related | | |
| | | protein | | |
| BC001602.1 | CFLAR | CASP8 and FADD-like | down | 211316_x_at |
| | | apoptosis regulator | | |
| BC034707.1 | — | Homo sapiens | down | 217653_x_at |
| | | transcribed sequence | | |
| | | with weak similarity to | | |
| | | protein | | |
| | | ref:NP_060312.1 | | |
| | | (H. sapiens) | | |
| | | hypothetical protein | | |
| | | FLJ20489 [Homo | | |
| | | sapiens] | | |
| BC064619.1 | CD24 | CD24 antigen (small | down | 266_s_at |
| | | cell lung carcinoma | | |
| | | cluster 4 antigen) | | |
| AY280502.1 | EPHB6 | EphB6 | down | 204718_at |
| BC059387.1 | MYO1A | myosin IA | down | 211916_s_at |
| | — | Homo sapiens | down | 215032_at |
| | | transcribed sequences | | |
| AF135421.1 | GMPPB | GDP-mannose | up | 219920_s_at |
| | | pyrophosphorylase B | | |
| BC061522.1 | MGC70907 | similar to MGC9515 | down | 211996_s_at |
| | | protein | | |
| L76200.1 | GUK1 | guanylate kinase 1 | up | 200075_s_at |
| U50532.1 | CG005 | hypothetical protein | down | 214753_at |
| | | from BCRA2 region | | |
| BC006547.2 | EEF2 | eukaryotic translation | down | 204102_s_at |
| | | elongation factor 2 | | |
| BC008797.2 | FVT1 | follicular lymphoma | down | 202419_at |
| | | variant translocation 1 | | |
| BC000807.1 | ZNF160 | zinc finger protein 160 | down | 214715_x_at |
| AL080112.1 | — | — | down | 216859_x_at |
| BC033718.1/// | C21orf106 | chromosome 21 open | down | 215529_x_at |
| BC046176.1/// | | reading frame 106 | | |
| BC038443.1 | | | | |
| NM_000346.1 | SOX9 | SRY (sex determining | up | 202936_s_at |
| | | region Y)-box 9 | | |
| | | (campomelic dysplasia, | | |
| | | autosomal sex-reversal) | | |
| BC008710.1 | SUI1 | putative translation | up | 212130_x_at |
| | | initiation factor | | |
| Hs.288575 | — | Homo sapiens cDNA | down | 215204_at |
| (UNIGENE ID) | | FLJ14090 fis, clone | | |
| | | MAMMA1000264. | | |
| AF020591.1 | AF020591 | zinc finger protein | down | 218735_s_at |
| BC000423.2 | ATP6V0B | ATPase, H+ | up | 200078_s_at |
| | | transporting, lysosomal | | |
| | | 21 kDa, V0 subunit c″/// | | |
| | | ATPase, H+ | | |
| | | transporting, lysosomal | | |
| | | 21 kDa, V0 subunit c″ | | |
| BC002503.2 | SAT | spermidine/spermine | down | 203455_s_at |
| | | N1-acetyltransferase | | |
| BC008710.1 | SUI1 | putative translation | up | 212227_x_at |
| | | initiation factor | | |
| | — | Homo sapiens | down | 222282_at |
| | | transcribed sequences | | |
| BC009185.2 | DCLRE1C | DNA cross-link repair | down | 219678_x_at |
| | | 1C (PSO2 homolog, S. | | |
| | | cerevisiae) | | |
| Hs.528304 | ADAM28 | a disintegrin and | down | 208268_at |
| (UNIGENE ID) | | metalloproteinase | | |
| | | domain 28 | | |
| U50532.1 | CG005 | hypothetical protein | down | 221899_at |
| | | from BCRA2 region | | |
| BC013923.2 | SOX2 | SRY (sex determining | down | 213721_at |
| | | region Y)-box 2 | | |
| BC031091 | ODAG | ocular development- | down | 214718_at |
| | | associated gene | | |
| NM_007062 | PWP1 | nuclear phosphoprotein | up | 201608_s_at |
| | | similar to S. cerevisiae | | |
| | | PWP1 | | |
| Hs.249591 | FLJ20686 | hypothetical protein | down | 205684_s_at |
| (Unigene ID) | | FLJ20686 | | |
| BC075839.1/// | KRT8 | keratin 8 | up | 209008_x_at |
| BC073760.1 | | | | |
| BC072436.1/// | HYOU1 | hypoxia up-regulated 1 | up | 200825_s_at |
| BC004560.2 | | | | |
| BC001016.2 | NDUFA8 | NADH dehydrogenase | up | 218160_at |
| | | (ubiquinone) 1 alpha | | |
| | | subcomplex, 8, 19 kDa | | |
| Hs.286261 | FLJ20195 | hypothetical protein | down | 57739_at |
| (Unigene ID) | | FLJ20195 | | |
| AF348514.1 | — | Homo sapiens fetal | down | 211921_x_at |
| | | thymus prothymosin | | |
| | | alpha mRNA, complete | | |
| | | cds | | |
| BC005023.1 | CGI-128 | CGI-128 protein | up | 218074_at |
| BC066337.1/// | KTN1 | kinectin 1 (kinesin | down | 200914_x_at |
| BC058736.1/// | | receptor) | | |
| BC050555.1 | | | | |
| | — | — | down | 216384_x_at |
| Hs.216623 | ATP8B1 | ATPase, Class I, type | down | 214594_x_at |
| (Unigene ID) | | 8B, member 1 | | |
| BC072400.1 | THOC2 | THO complex 2 | down | 222122 s at |
| BC041073.1 | PRKX | protein kinase, X-linked | down | 204060_s_at |
| U43965.1 | ANK3 | ankyrin 3, node of | down | 215314_at |
| | | Ranvier (ankyrin G) | | |
| | — | — | down | 208238_x_at |
| BC021258.2 | TRIM5 | tripartite motif- | down | 210705_s_at |
| | | containing 5 | | |
| BC016057.1 | USH1C | Usher syndrome 1C | down | 211184_s_at |
| | | (autosomal recessive, | | |
| | | severe) | | |
| BC016713.1/// | PARVA | parvin, alpha | down | 215418_at |
| BC014535.1/// | | | | |
| AF237771.1 | | | | |
| BC000360.2 | EIF4EL3 | eukaryotic translation | up | 209393_s_at |
| | | initiation factor 4E-like | | |
| | | 3 | | |
| BC007455.2 | SH3GLB1 | SH3-domain GRB2-like | up | 210101_x_at |
| | | endophilin B1 | | |
| BC000701.2 | KIAA0676 | KIAA0676 protein | down | 212052_s_at |
| BC010067.2 | CHC1 | chromosome | down | 215011_at |
| | | condensation 1 | | |
| BC023528.2/// | C14orf87 | chromosome 14 open | up | 221932_s_at |
| BC047680.1 | | reading frame 87 | | |
| BC064957.1 | KIAA0102 | KIAA0102 gene | up | 201239_s_at |
| | | product | | |
| Hs.156701 | — | Homo sapiens cDNA | down | 215553_x_at |
| (Unigene ID) | | FLJ14253 fis, clone | | |
| | | OVARC1001376. | | |
| BC030619.2 | KIAA0779 | KIAA0779 protein | down | 213351_s_at |
| BC008710.1 | SUI1 | putative translation | up | 202021_x_at |
| | | initiation factor | | |
| U43965.1 | ANK3 | ankyrin 3, node of | down | 209442_x_at |
| | | Ranvier (ankyrin G) | | |
| BC066329.1 | SDHC | succinate | up | 210131_x_at |
| | | dehydrogenase | | |
| | | complex, subunit C, | | |
| | | integral membrane | | |
| | | protein, 15 kDa | | |
| Hs.438867 | — | Homo sapiens | down | 217713_x_at |
| (Unigene ID) | | transcribed sequence | | |
| | | with weak similarity to | | |
| | | protein | | |
| | | ref:NP_060312.1 | | |
| | | (H. sapiens) | | |
| | | hypothetical protein | | |
| | | FLJ20489 [Homo | | |
| | | sapiens] | | |
| BC035025.2/// | ALMS1 | Alstrom syndrome 1 | down | 214707_x_at |
| BC050330.1 | | | | |
| BC023976.2 | PDAP2 | PDGFA associated | up | 203272_s_at |
| | | protein 2 | | |
| BC074852.2/// | PRKY | protein kinase, Y-linked | down | 206279_at |
| BC074851.2 | | | | |
| Hs.445885 | KIAA1217 | Homo sapiens cDNA | down | 214912_at |
| (Unigene ID) | | FLJ12005 fis, clone | | |
| | | HEMBB1001565. | | |
| BC008591.2/// | KIAA0100 | KIAA0100 gene | up | 201729_s_at |
| BC050440.1/// | | product | | |
| BC048096.1 | | | | |
| AF365931.1 | ZNF264 | zinc finger protein 264 | down | 205917_at |
| AF257099.1 | PTMA | prothymosin, alpha | down | 200772_x_at |
| | | (gene sequence 28) | | |
| BC028912.1 | DNAJB9 | DnaJ (Hsp40) homolog, | up | 202842_s_at |
| | | subfamily B, member 9 | | |

Table 3 shows one preferred 50 gene group that was identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer.

This gene group was identified using the GenePattern server from the Broad Institute, which includes the Weighted Voting algorithm. The default settings, i.e., the signal to noise ratio and no gene filtering, were used.

In one embodiment, the exemplary probes shown in the column “Affymetrix Id in the Human Genome U133 chip” can be used in the expression analysis.

TABLE 3
50 Gene Group

| | | | Affymetrix Id in the |
|---|---|---|---|
| | | Direction in | Human Genome |
| GenBank ID | Gene Name | Cancer | U133 chip |
| NM_007062.1 | PWP1 | up in cancer | 201608_s_at |
| NM_001281.1 | CKAP1 | up in cancer | 201804_x_at |
| BC000120.1 | | up in cancer | 202355_s_at |
| NM_014255.1 | TMEM4 | up in cancer | 202857_at |
| BC002642.1 | CTSS | up in cancer | 202901_x_at |
| NM_000346.1 | SOX9 | up in cancer | 202936_s_at |
| NM_006545.1 | NPR2L | up in cancer | 203246_s_at |
| BG034328 | | up in cancer | 203588_s_at |
| NM_021822.1 | APOBEC3G | up in cancer | 204205_at |
| NM_021069.1 | ARGBP2 | up in cancer | 204288_s_at |
| NM_019067.1 | FLJ10613 | up in cancer | 205010_at |
| NM_017925.1 | FLJ20686 | up in cancer | 205684_s_at |
| NM_017932.1 | FLJ20700 | up in cancer | 207730_x_at |
| NM_030757.1 | MKRN4 | up in cancer | 208082_x_at |
| NM_030972.1 | MGC5384 | up in cancer | 208137_x_at |
| AF126181.1 | BCG1 | up in cancer | 208682_s_at |
| U93240.1 | | up in cancer | 209653_at |
| U90552.1 | | up in cancer | 209770_at |
| AF151056.1 | | up in cancer | 210434_x_at |
| U85430.1 | NFATC3 | up in cancer | 210556_at |
| U51007.1 | | up in cancer | 211609_x_at |
| BC005969.1 | | up in cancer | 211759_x_at |
| NM_002271.1 | | up in cancer | 211954_s_at |
| AL566172 | | up in cancer | 212041_at |
| AB014576.1 | KIAA0676 | up in cancer | 212052_s_at |
| BF218804 | AFURS1 | down in cancer | 212297_at |
| AK022494.1 | | down in cancer | 212932_at |
| AA114843 | | down in cancer | 213884_s_at |
| BE467941 | | down in cancer | 214153_at |
| NM_003541.1 | HIST1H4K | down in cancer | 214463_x_at |
| R83000 | BTF3 | down in cancer | 214800_x_at |
| AL161952.1 | GLUL | down in cancer | 215001_s_at |
| AK023843.1 | PGF | down in cancer | 215179_x_at |
| AK021571.1 | MUC20 | down in cancer | 215208_x_at |
| AK023783.1 | — | down in cancer | 215604_x_at |
| AU147182 | | down in cancer | 215620_at |
| AL080112.1 | — | down in cancer | 216859_x_at |
| AW971983 | | down in cancer | 217588_at |
| AI683552 | — | down in cancer | 217679_x_at |
| NM_024006.1 | IMAGE3455200 | down in cancer | 217949_s_at |
| AK026565.1 | FLJ10534 | down in cancer | 218155_x_at |
| NM_014182.1 | ORMDL2 | down in cancer | 218556_at |
| NM_021800.1 | DNAJC12 | down in cancer | 218976_at |
| NM_016049.1 | CGI-112 | down in cancer | 219203_at |
| NM_019023.1 | PRMT7 | down in cancer | 219408_at |
| NM_021971.1 | GMPPB | down in cancer | 219920_s_at |
| NM_014128.1 | — | down in cancer | 220856_x_at |
| AK025651.1 | | down in cancer | 221648_s_at |
| AA133341 | C14orf87 | down in cancer | 221932_s_at |
| AF198444.1 | | down in cancer | 222168_at |

Table 4 shows one preferred 36 gene group that was identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer.

In one embodiment, the exemplary probes shown in the column “Affymetrix Id in the Human Genome U133 chip” can be used in the expression analysis.

TABLE 4
36 Gene Group

| GenBank ID | Gene Name | Gene Description | Affy ID |
|---|---|---|---|
| NM_007062.1 | PWP1 | nuclear phosphoprotein | 201608_s_at |
| | | similar to S. cerevisiae | |
| | | PWP1 | |
| NM_001281.1 | CKAP1 | cytoskeleton associated | 201804_x_at |
| | | protein 1 | |
| BC002642.1 | CTSS | cathepsin S | 202901_x_at |
| NM_000346.1 | SOX9 | SRY (sex determining | 202936_s_at |
| | | region Y)-box 9 | |
| | | (campomelic dysplasia, | |
| | | autosomal sex-reversal) | |
| NM_006545.1 | NPR2L | homologous to yeast | 203246_s_at |
| | | nitrogen permease | |
| | | (candidate tumor | |
| | | suppressor) | |
| BG034328 | | transcription factor | 203588_s_at |
| | | Dp-2 (E2F dimerization | |
| | | partner 2) | |
| NM_019067.1 | FLJ10613 | hypothetical protein | 205010_at |
| | | FLJ10613 | |
| NM_017925.1 | FLJ20686 | hypothetical protein | 205684_s_at |
| | | FLJ20686 | |
| NM_017932.1 | FLJ20700 | hypothetical protein | 207730_x_at |
| | | FLJ20700 | |
| NM_030757.1 | MKRN4 | makorin, ring finger | 208082_x_at |
| | | protein, 4///makorin, | |
| | | ring finger protein, 4 | |
| NM_030972.1 | MGC5384 | hypothetical protein | 208137_x_at |
| | | MGC5384 | |
| NM_002268/// | KPNA4 | karyopherin alpha 4 | 209653_at |
| NM_032771 | | (importin alpha 3) | |
| NM_007048/// | BTN3A1 | butyrophilin, subfamily | 209770_at |
| NM_194441 | | 3, member A1 | |
| NM_006694 | JBT | jumping translocation | 210434_x_at |
| | | breakpoint | |
| U85430.1 | NFATC3 | nuclear factor of | 210556_at |
| | | activated T-cells, | |
| | | cytoplasmic, | |
| | | calcineurin-dependent 3 | |
| NM_004691 | ATP6V0D1 | ATPase, H+ | 212041_at |
| | | transporting, | |
| | | lysosomal 38 kDa, V0 | |
| | | subunit d isoform 1 | |
| AB014576.1 | KIAA0676 | KIAA0676 protein | 212052_s_at |
| BF218804 | AFURS1 | ATPase family | 212297_at |
| | | homolog up-regulated | |
| | | in senescence cells | |
| BE467941 | | EVOVL family | 214153_at |
| | | member 5, elongation | |
| | | of long chain fatty acids | |
| | | (FEN1/Elo2, | |
| | | SUR4/Elo3-like, yeast) | |
| R83000 | BTF3 | basic transcription | 214800_x_at |
| | | factor 3 | |
| AL161952.1 | GLUL | glutamate-ammonia | 215001_s_at |
| | | ligase (glutamine | |
| | | synthase) | |
| AK023843.1 | PGF | placental growth factor, | 215179_x_at |
| | | vascular endothelial | |
| | | growth factor-related | |
| | | protein | |
| AK021571.1 | MUC20 | mucin 20 | 215208_x_at |
| AK023783.1 | — | Homo sapiens cDNA | 215604_x_at |
| | | FLJ13721 fis, clone | |
| | | PLACE2000450. | |
| AL080112.1 | — | — | 216859_x_at |
| AW971983 | | cation, sperm | 217588_at |
| | | associated 2 | |
| AI683552 | — | — | 217679_x_at |
| NM_024006.1 | IMAGE3455200 | hypothetical protein | 217949_s_at |
| | | IMAGE3455200 | |
| AK026565.1 | FLJ10534 | hypothetical protein | 218155_x_at |
| | | FLJ10534 | |
| NM_014182.1 | ORMDL2 | ORM1-like 2 (S. | 218556_at |
| | | cerevisiae) | |
| NM_021800.1 | DNAJC12 | J Domain containing | 218976_at |
| | | protein 1 | |
| NM_016049.1 | CGI-112 | comparative gene | 219203_at |
| | | identification transcript | |
| | | 112 | |
| NM_021971.1 | GMPPB | GDP-mannose | 219920_s_at |
| | | pyrophosphorylase B | |
| NM_014128.1 | — | — | 220856_x_at |
| AA133341 | C14orf87 | chromosome 14 open | 221932_s_at |
| | | reading frame 87 | |
| AF198444.1 | | Homo sapiens 10q21 | 222168_at |
| | | mRNA sequence | |

In one embodiment, the gene group of the present invention comprises at least, for example, 5, 10, 15, 20, 25, 30, more preferably at least 36, still more preferably at least about 40, still more preferably at least about 50, still more preferably at least about 60, still more preferably at least about 70, still more preferably at least about 80, still more preferably at least about 86, still more preferably at least about 90, still more preferably at least about 96 of the genes as shown in Tables 1-4.

In one preferred embodiment, the gene group comprises 36-180 genes selected from the group consisting of the genes listed in Tables 1-4.

In one embodiment, the invention provides group of genes the expression of which is lower in individuals with cancer.

Accordingly, in one embodiment, the invention provides of a group of genes useful in diagnosing lung diseases, wherein the expression of the group of genes is lower in individuals exposed to air pollutants with cancer as compared to individuals exposed to the same air pollutant who do not have cancer, the group comprising probes that hybridize at least 5, preferably at least about 5-10, still more preferably at least about 10-20, still more preferably at least about 20-30, still more preferably at least about 30-40, still more preferably at least about 40-50, still more preferably at least about 50-60, still more preferably at least about 60-70, still more preferably about 72 genes consisting of transcripts (transcripts are identified using their GenBank ID or Unigene ID numbers and the corresponding gene names appear in Table 1): NM_003335; NM_001319; NM_021145.1; NM_001003698///NM_001003699///; NM_002955; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_006534///NM_181659; NM_014033; NM_016138; NM_007048///NM_194441; NM_000051///NM_138292///NM_138293; NM_000410///NM_139002///NM_139003///NM_139004///NM_139005///NM_139006///NM_139007///NM_139008///NM_139009///NM_139010///NM_139011; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_019011///NM_207111///NM_207116; NM_017646; NM_014395; NM_014336; NM_018097; NM_019014; NM_024804; NM_018260; NM_018118; NM_014128; NM_024084; NM_005294; AF077053; NM_000693; NM_033128; NM_020706; AI523613; and NM_014884.

In another embodiment, the invention provides of a group of genes useful in diagnosing lung diseases wherein the expression of the group of genes is lower in individuals exposed to air pollutants with cancer as compared to individuals exposed to the same air pollutant who do not have cancer, the group comprising probes that hybridize at least 5, preferably at least about 5-10, still more preferably at least about 10-20, still more preferably at least about 20-30, still more preferably at least about 30-40, still more preferably at least about 40-50, still more preferably at least about 50-60, still more preferably about 63 genes consisting of transcripts (transcripts are identified using their GenBank ID or Unigene ID numbers and the corresponding gene names appear in Table 2): NM_030757.1; R83000; AK021571.1; NM_17932.1; U85430.1; AI683552; BC002642.1; AW024467; NM_030972.1; BC021135.1; AL161952.1; AK026565.1; AK023783.1; BF218804; AK023843.1; BC001602.1; BC034707.1; BC064619.1; AY280502.1; BC059387.1; BC061522.1; U50532.1; BC006547.2; BC008797.2; BC000807.1; AL080112.1; BC033718.1///BC046176.1///; BC038443.1; Hs.288575 (UNIGENE ID); AF020591.1; BC002503.2; BC009185.2; Hs.528304 (UNIGENE ID); U50532.1; BC013923.2; BC031091; Hs.249591 (Unigene ID); Hs.286261 (Unigene ID); AF348514.1; BC066337.1///BC058736.1///BC050555.1; Hs.216623 (Unigene ID); BC072400.1; BC041073.1; U43965.1; BC021258.2; BC016057.1; BC016713.1///BC014535.1///AF237771.1; BC000701.2; BC010067.2; Hs.156701 (Unigene ID); BC030619.2; U43965.1; Hs.438867 (Unigene ID); BC035025.2///BC050330.1; BC074852.2///BC074851.2; Hs.445885 (Unigene ID); AF365931.1; and AF257099.1

In another embodiment, the invention provides of a group of genes useful in diagnosing lung diseases wherein the expression of the group of genes is lower in individuals exposed to air pollutants with cancer as compared to individuals exposed to the same air pollutant who do not have cancer, the group comprising probes that hybridize at least 5, preferably at least about 5-10, still more preferably at least about 10-20, still more preferably at least about 20-25, still more preferably about 25 genes consisting of transcripts (transcripts are identified using their GenBank ID or Unigene ID numbers and the corresponding gene names appear in Table 3):BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1.

In another embodiment, the invention provides of a group of genes useful in diagnosing lung diseases wherein the expression of the group of genes is higher in individuals exposed to air pollutants with cancer as compared to individuals exposed to the same air pollutant who do not have cancer, the group comprising probes that hybridize at least to 5, preferably at least about 5-10, still more preferably at least about 10-20, still more preferably at least about 20-25, still more preferably about 25 genes consisting of transcripts (transcripts are identified using their GenBank ID or Unigene ID numbers and the corresponding gene names appear in Table 1): NM_000918; NM_006430.1; NM_001416.1; NM_004090; NM_006406.1; NM_003001.2; NM_006545.1; NM_002437.1; NM_006286; NM_001123///NM_006721; NM_024824; NM_004935.1; NM_001696; NM_005494///NM_058246; NM_006368; NM_002268///NM_032771; NM_006694; NM_004691; NM_012394; NM_021800; NM_016049; NM_138387; NM_024531; and NM_018509.

In another embodiment, the invention provides of a group of genes useful in diagnosing lung diseases wherein the expression of the group of genes is higher in individuals exposed to air pollutants with cancer as compared to individuals exposed to the same air pollutant who do not have cancer, the group comprising probes that hybridize at least to 5, preferably at least about 5-10, still more preferably at least about 10-20, still more preferably at least about 20-23, still more preferably about 23 genes consisting of transcripts (transcripts are identified using their GenBank ID or Unigene ID numbers and the corresponding gene names appear in Table 2): NM_014182.1; NM_001281.1; NM_024006.1; AF135421.1; L76200.1; NM_000346.1; BC008710.1; BC000423.2; BC008710.1; NM_007062; BC075839.1///BC073760.1; BC072436.1///BC004560.2; BC001016.2; BC005023.1; BC000360.2; BC007455.2; BC023528.2///BC047680.1; BC064957.1; BC008710.1; BC066329.1; BC023976.2; BC008591.2///BC050440.1///BC048096.1; and BC028912.1.

In another embodiment, the invention provides of a group of genes useful in diagnosing lung diseases wherein the expression of the group of genes is higher in individuals exposed to air pollutants with cancer as compared to individuals exposed to the same air pollutant who do not have cancer, the group comprising probes that hybridize at least to 5, preferably at least about 5-10, still more preferably at least about 10-20, still more preferably at least about 20-25, still more preferably about 25 genes consisting of transcripts (transcripts are identified using their GenBank ID or Unigene ID numbers and the corresponding gene names appear in Table 3): NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; and AB014576.1.

In one embodiment, the invention provides a method of diagnosing lung disease comprising the steps of measuring the expression profile of a gene group in an individual suspected of being affected or being at high risk of a lung disease (i.e. test individual), and comparing the expression profile (i.e. control profile) to an expression profile of an individual without the lung disease who has also been exposed to similar air pollutant than the test individual (i.e. control individual), wherein differences in the expression of genes when compared between the afore mentioned test individual and control individual of at least 10, more preferably at least 20, still more preferably at least 30, still more preferably at least 36, still more preferably between 36-180, still more preferably between 36-96, still more preferably between 36-84, still more preferably between 36-50, is indicative of the test individual being affected with a lung disease. Groups of about 36 genes as shown in table 4, about 50 genes as shown in table 3, about 84 genes as shown in table 2 and about 96 genes as shown in table 1 are preferred. The different gene groups can also be combined, so that the test individual can be screened for all, three, two, or just one group as shown in tables 1-4.

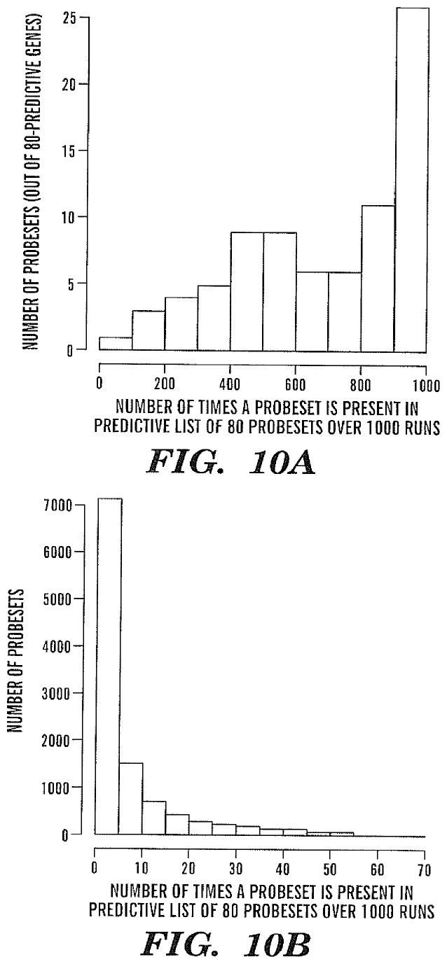

For example, if the expression profile of a test individual exposed to cigarette smoke is compared to the expression profile of the 50 genes shown in table 3, using the Affymetrix inc probe set on a gene chip as shown in table 3, the expression profile that is similar to the one shown in FIG. 10 for the individuals with cancer, is indicative that the test individual has cancer. Alternatively, if the expression profile is more like the expression profile of the individuals who do not have cancer in FIG. 10, the test individual likely is not affected with lung cancer.

The group of 50 genes was identified using the GenePattern server from the Broad Institute, which includes the Weighted Voting algorithm. The default settings, i.e., the signal to noise ratio and no gene filtering, were used. GenePattern is available through the World Wide Wed at location broad.mit.edu/cancer/software/genepattern. This program allows analysis of data in groups rather than as individual genes. Thus, in one preferred embodiment, the expression of substantially all 50 genes of Table 3, are analyzed together. The expression profile of lower that normal expression of genes selected from the group consisting of BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1, and the gene expression profile of higher than normal expression of genes selected from the group consisting of NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; and AB014576.1, is indicative of the individual having or being at high risk of developing lung disease, such as lung cancer. In one preferred embodiment, the expression pattern of all the genes in the Table 3 is analyzed. In one embodiment, in addition to analyzing the group of predictor genes of Table 3, 1, 2, 3, 4, 5, 6, 7, 8, 9, 10-15, 15-20, 20-30, or more of the individual predictor genes identified using the t-test analysis are analyzed. Any combination of, for example, 5-10 or more of the group predictor genes and 5-10, or more of the individual genes can also be used.

The term “expression profile” as used herein, refers to the amount of the gene product of each of the analyzed individual genes in the sample. The “expression profile” is like a signature expression map, like the one shown for each individual in FIG. 10, on the Y-axis.

The term “lung disease”, as used herein, refers to disorders including, but not limited to, asthma, chronic bronchitis, emphysema, bronchietasis, primary pulmonary hypertension and acute respiratory distress syndrome. The methods described herein may also be used to diagnose or treat lung disorders that involve the immune system including, hypersensitivity pneumonitis, eosinophilic pneumonias, and persistent fungal infections, pulmonary fibrosis, systemic sclerosis, idiopathic pulmonary hemosiderosis, pulmonary alveolar proteinosis, cancers of the lung such as adenocarcinoma, squamous cell carcinoma, small cell and large cell carcinomas, and benign neoplasm of the lung including bronchial adenomas and hamartomas. In one preferred embodiment, the lung disease is lung cancer.

The biological samples useful according to the present invention include, but are not limited to tissue samples, cell samples, and excretion samples, such as sputum or saliva, of the airways. The samples useful for the analysis methods according to the present invention can be taken from the mouth, the bronchial airways, and the lungs.

The term “air pollutants”, as used herein, refers to any air impurities or environmental airway stress inducing agents, such as cigarette smoke, cigar smoke, smog, asbestos, and other air pollutants that have suspected or proven association to lung diseases.

The term “individual”, as used herein, preferably refers to human. However, the methods are not limited to humans, and a skilled artisan can use the diagnostic/prognostic gene groupings of the present invention in, for example, laboratory test animals, preferably animals that have lungs, such as non-human primates, murine species, including, but not limited to rats and mice, dogs, sheep, pig, guinea pigs, and other model animals. Such laboratory tests can be used, for example in pre-clinical animal testing of drugs intended to be used to treat or prevent lung diseases.

The phrase “altered expression” as used herein, refers to either increased or decreased expression in an individual exposed to air pollutant, such as a smoker, with cancer when compared to an expression pattern of the lung cells from an individual exposed to similar air pollutant, such as smoker, who does not have cancer. Tables 1 and 2 show the preferred expression pattern changes of the invention. The terms “up” and “down” in the tables refer to the amount of expression in a smoker with cancer to the amount of expression in a smoker without cancer. Similar expression pattern changes are likely associated with development of cancer in individuals who have been exposed to other airway pollutants.

In one embodiment, the group of genes the expression of which is analyzed in diagnosis and/or prognosis of lung cancer are selected from the group of 80 genes as shown in Table 5. Any combination of genes can be selected from the 80 genes. In one embodiment, the combination of 20 genes shown in Table 7 is selected. In one embodiment, a combination of genes from Table 6 is selected.

TABLE 5
Group of 80 genes for prognostic and diagnostic testing of lung cancer.

| | | | Signal to noise in a |
|---|---|---|---|
| | | Number of | cancer sample. |
| | | runs the gene | Negative values |
| | | is indicated | indicate increase |
| Affymetrix probe | | in cancer | of expression in lung |
| ID No. that can be | | samples as | cancer, positive |
| used to identify | | differentially | values indicate |
| the gene/nucleic | | expressed out | decrease of |
| acid sequence in | Gene | of 1000 test | expression in lung |
| the next column | symbol | runs | cancer. |
| 200729_s_at | ACTR2 | 736 | −0.22284 |
| 200760_s_at | ARL6IP5 | 483 | −0.21221 |
| 201399_s_at | TRAM1 | 611 | −0.21328 |
| 201444_s_at | ATP6AP2 | 527 | −0.21487 |
| 201635_s_at | FXR1 | 458 | −0.2162 |
| 201689_s_at | TPD52 | 565 | −0.22292 |
| 201925_s_at | DAF | 717 | −0.25875 |
| 201926_s_at | DAF | 591 | −0.23228 |
| 201946_s_at | CCT2 | 954 | −0.24592 |
| 202118_s_at | CPNE3 | 334 | −0.21273 |
| 202704_at | TOB1 | 943 | −0.25724 |
| 202833_s_at | SERPINA1 | 576 | −0.20583 |
| 202935_s_at | SOX9 | 750 | −0.25574 |
| 203413_at | NELL2 | 629 | −0.23576 |
| 203881_s_at | DMD | 850 | −0.24341 |
| 203908_at | SLC4A4 | 887 | −0.23167 |
| 204006_s_at | FCGR3A/// | 207 | −0.20071 |
| | FCGR3B | | |
| 204403_x_at | KIAA0738 | 923 | 0.167772 |
| 204427_s_at | RNP24 | 725 | −0.2366 |
| 206056_x_at | SPN | 976 | 0.196398 |
| 206169_x_at | RoXaN | 984 | 0.259637 |
| 207730_x_at | HDGF2 | 969 | 0.169108 |
| 207756_at | — | 855 | 0.161708 |
| 207791_s_at | RAB1A | 823 | −0.21704 |
| 207953_at | AD7C-NTP | 1000 | 0.218433 |
| 208137_x_at | — | 996 | 0.191938 |
| 208246_x_at | TK2 | 982 | 0.179058 |
| 208654_s_at | CD164 | 388 | −0.21228 |
| 208892_s_at | DUSP6 | 878 | −0.25023 |
| 209189_at | FOS | 935 | −0.27446 |
| 209204_at | LMO4 | 78 | 0.158674 |
| 209267_s_at | SLC39A8 | 228 | −0.24231 |
| 209369_at | ANXA3 | 384 | −0.19972 |
| 209656_s_at | TMEM47 | 456 | −0.23033 |
| 209774_x_at | CXCL2 | 404 | −0.2117 |
| 210145_at | PLA2G4A | 475 | −0.26146 |
| 210168_at | C6 | 458 | −0.24157 |
| 210317_s_at | YWHAE | 803 | −0.29542 |
| 210397_at | DEFB1 | 176 | −0.22512 |
| 210679_x_at | — | 970 | 0.181718 |
| 211506_s_at | IL8 | 270 | −0.3105 |
| 212006_at | UBXD2 | 802 | −0.22094 |
| 213089_at | LOC153561 | 649 | 0.164097 |
| 213736_at | COX5B | 505 | 0.155243 |
| 213813_x_at | — | 789 | 0.178643 |
| 214007_s_at | PTK9 | 480 | −0.21285 |
| 214146_s_at | PPBP | 593 | −0.24265 |
| 214594_x_at | ATP8B1 | 962 | 0.284039 |
| 214707_x_at | ALMS1 | 750 | 0.164047 |
| 214715_x_at | ZNF160 | 996 | 0.198532 |
| 215204_at | SENP6 | 211 | 0.169986 |
| 215208_x_at | RPL35A | 999 | 0.228485 |
| 215385_at | FTO | 164 | 0.187634 |
| 215600_x_at | FBXW12 | 960 | 0.17329 |
| 215604_x_at | UBE2D2 | 998 | 0.224878 |
| 215609_at | STARD7 | 940 | 0.191953 |
| 215628_x_at | PPP2CA | 829 | 0.16391 |
| 215800_at | DUOX1 | 412 | 0.160036 |
| 215907_at | BACH2 | 987 | 0.178338 |
| 215978_x_at | LOC152719 | 645 | 0.163399 |
| 216834_at | — | 633 | −0.25508 |
| 216858_x_at | — | 997 | 0.232969 |
| 217446_x_at | — | 942 | 0.182612 |
| 217653_x_at | — | 976 | 0.270552 |
| 217679_x_at | — | 987 | 0.265918 |
| 217715_x_at | ZNF354A | 995 | 0.223881 |
| 217826_s_at | UBE2J1 | 812 | −0.23003 |
| 218155_x_at | FLJ10534 | 998 | 0.186425 |
| 218976_at | DNAJC12 | 486 | −0.22866 |
| 219392_x_at | FLJ11029 | 867 | 0.169113 |
| 219678_x_at | DCLRE1C | 877 | 0.169975 |
| 220199_s_at | FLJ12806 | 378 | −0.20713 |
| 220389_at | FLJ23514 | 102 | 0.239341 |
| 220720_x_at | FLJ14346 | 989 | 0.17976 |
| 221191_at | DKFZP434A0 | 616 | 0.185412 |
| | 131 | | |
| 221310_at | FGF14 | 511 | −0.19965 |
| 221765_at | — | 319 | −0.25025 |
| 222027_at | NUCKS | 547 | 0.171954 |
| 222104_x_at | GTF2H3 | 981 | 0.186025 |
| 222358_x_at | — | 564 | 0.194048 |

TABLE 6
Group of 535 genes useful in prognosis or diagnosis of lung cancer.

| Affymetrix | | Number of | Signal to noise in a |
|---|---|---|---|
| probe ID No. | | runs the gene | cancer sample. Negative |
| that can be | | is indicated in | values indicate |
| used to identify | | cancer samples | increase of expression |
| the gene/nucleic | | as differentially | in lung cancer, |
| acid sequence | | expressed out | positive values indicate |
| in the next | | of 1000 test | decrease of expression |
| column | Gene symbol | runs | in lung cancer. |
| 200729_s_at | ACTR2 | 736 | −0.22284 |
| 200760_s_at | ARL6IP5 | 483 | −0.21221 |
| 201399_s_at | TRAM1 | 611 | −0.21328 |
| 201444_s_at | ATP6AP2 | 527 | −0.21487 |
| 201635_s_at | FXR1 | 458 | −0.2162 |
| 201689_s_at | TPD52 | 565 | −0.22292 |
| 201925_s_at | DAF | 717 | −0.25875 |
| 201926_s_at | DAF | 591 | −0.23228 |
| 201946_s_at | CCT2 | 954 | −0.24592 |
| 202118_s_at | CPNE3 | 334 | −0.21273 |
| 202704_at | TOB1 | 943 | −0.25724 |
| 202833_s_at | SERPINA1 | 576 | −0.20583 |
| 202935_s_at | SOX9 | 750 | −0.25574 |
| 203413_at | NELL2 | 629 | −0.23576 |
| 203881_s_at | DMD | 850 | −0.24341 |
| 203908_at | SLC4A4 | 887 | −0.23167 |
| 204006_s_at | FCGR3A/// | 207 | −0.20071 |
| | FCGR3B | | |
| 204403_x_at | KIAA0738 | 923 | 0.167772 |
| 204427_s_at | RNP24 | 725 | −0.2366 |
| 206056_x_at | SPN | 976 | 0.196398 |
| 206169_x_at | RoXaN | 984 | 0.259637 |
| 207730_x_at | HDGF2 | 969 | 0.169108 |
| 207756_at | — | 855 | 0.161708 |
| 207791_s_at | RAB1A | 823 | −0.21704 |
| 207953_at | AD7C-NTP | 1000 | 0.218433 |
| 208137_x_at | — | 996 | 0.191938 |
| 208246_x_at | TK2 | 982 | 0.179058 |
| 208654_s_at | CD164 | 388 | −0.21228 |
| 208892_s_at | DUSP6 | 878 | −0.25023 |
| 209189_at | FOS | 935 | −0.27446 |
| 209204_at | LMO4 | 78 | 0.158674 |
| 209267_s_at | SLC39A8 | 228 | −0.24231 |
| 209369_at | ANXA3 | 384 | −0.19972 |
| 209656_s_at | TMEM47 | 456 | −0.23033 |
| 209774_x_at | CXCL2 | 404 | −0.2117 |
| 210145_at | PLA2G4A | 475 | −0.26146 |
| 210168_at | C6 | 458 | −0.24157 |
| 210317_s_at | YWHAE | 803 | −0.29542 |
| 210397_at | DEFB1 | 176 | −0.22512 |
| 210679_x_at | — | 970 | 0.181718 |
| 211506_s_at | IL8 | 270 | −0.3105 |
| 212006_at | UBXD2 | 802 | −0.22094 |
| 213089_at | LOC153561 | 649 | 0.164097 |
| 213736_at | COX5B | 505 | 0.155243 |
| 213813_x_at | — | 789 | 0.178643 |
| 214007_s_at | PTK9 | 480 | −0.21285 |
| 214146_s_at | PPBP | 593 | −0.24265 |
| 214594_x_at | ATP8B1 | 962 | 0.284039 |
| 214707_x_at | ALMS1 | 750 | 0.164047 |
| 214715_x_at | ZNF160 | 996 | 0.198532 |
| 215204_at | SENP6 | 211 | 0.169986 |
| 215208_x_at | RPL35A | 999 | 0.228485 |
| 215385_at | FTO | 164 | 0.187634 |
| 215600_x_at | FBXW12 | 960 | 0.17329 |
| 215604_x_at | UBE2D2 | 998 | 0.224878 |
| 215609_at | STARD7 | 940 | 0.191953 |
| 215628_x_at | PPP2CA | 829 | 0.16391 |
| 215800_at | DUOX1 | 412 | 0.160036 |
| 215907_at | BACH2 | 987 | 0.178338 |
| 215978_x_at | LOC152719 | 645 | 0.163399 |
| 216834_at | — | 633 | −0.25508 |
| 216858_x_at | — | 997 | 0.232969 |
| 217446_x_at | — | 942 | 0.182612 |
| 217653_x_at | — | 976 | 0.270552 |
| 217679_x_at | — | 987 | 0.265918 |
| 217715_x_at | ZNF354A | 995 | 0.223881 |
| 217826_s_at | UBE2J1 | 812 | −0.23003 |
| 218155_x_at | FLJ10534 | 998 | 0.186425 |
| 218976_at | DNAJC12 | 486 | −0.22866 |
| 219392_x_at | FLJ11029 | 867 | 0.169113 |
| 219678_x_at | DCLRE1C | 877 | 0.169975 |
| 220199_s_at | FLJ12806 | 378 | −0.20713 |
| 220389_at | FLJ23514 | 102 | 0.239341 |
| 220720_x_at | FLJ14346 | 989 | 0.17976 |
| 221191_at | DKFZP434A0 | 616 | 0.185412 |
| | 131 | | |
| 221310_at | FGF14 | 511 | −0.19965 |
| 221765_at | — | 319 | −0.25025 |
| 222027_at | NUCKS | 547 | 0.171954 |
| 222104_x_at | GTF2H3 | 981 | 0.186025 |
| 222358_x_at | — | 564 | 0.194048 |
| 202113_s_at | SNX2 | 841 | −0.20503 |
| 207133_x_at | ALPK1 | 781 | 0.155812 |
| 218989_x_at | SLC30A5 | 765 | −0.198 |
| 200751_s_at | HNRPC | 759 | −0.19243 |
| 220796_x_at | SLC35E1 | 691 | 0.158199 |
| 209362_at | SURB7 | 690 | −0.18777 |
| 216248_s_at | NR4A2 | 678 | −0.19796 |
| 203138_at | HAT1 | 669 | −0.18115 |
| 221428_s_at | TBL1XR1 | 665 | −0.19331 |
| 218172_s_at | DERL1 | 665 | −0.16341 |
| 215861_at | FLJ14031 | 651 | 0.156927 |
| 209288_s_at | CDC42EP3 | 638 | −0.20146 |
| 214001_x_at | RPS10 | 634 | 0.151006 |
| 209116_x_at | HBB | 626 | −0.12237 |
| 215595_x_at | GCNT2 | 625 | 0.136319 |
| 208891_at | DUSP6 | 617 | −0.17282 |
| 215067_x_at | PRDX2 | 616 | 0.160582 |
| 202918_s_at | PREI3 | 614 | −0.17003 |
| 211985_s_at | CALM1 | 614 | −0.20103 |
| 212019_at | RSL1D1 | 601 | 0.152717 |
| 216187_x_at | KNS2 | 591 | 0.14297 |
| 215066_at | PTPRF | 587 | 0.143323 |
| 212192_at | KCTD12 | 581 | −0.17535 |
| 217586_x_at | — | 577 | 0.147487 |
| 203582_s_at | RAB4A | 567 | −0.18289 |
| 220113_x_at | POLR1B | 563 | 0.15764 |
| 217232_x_at | HBB | 561 | −0.11398 |
| 201041_s_at | DUSP1 | 560 | −0.18661 |
| 211450_s_at | MSH6 | 544 | −0.15597 |
| 202648_at | RPS19 | 533 | 0.150087 |
| 202936_s_at | SOX9 | 533 | −0.17714 |
| 204426_at | RNP24 | 526 | −0.18959 |
| 206392_s_at | RARRES1 | 517 | −0.18328 |
| 208750_s_at | ARF1 | 515 | −0.19797 |
| 202089_s_at | SLC39A6 | 512 | −0.19904 |
| 211297_s_at | CDK7 | 510 | −0.15992 |
| 215373_x_at | FLJ12151 | 509 | 0.146742 |
| 213679_at | FLJ13946 | 492 | −0.10963 |
| 201694_s_at | EGR1 | 490 | −0.19478 |
| 209142_s_at | UBE2G1 | 487 | −0.18055 |
| 217706_at | LOC220074 | 483 | 0.11787 |
| 212991_at | FBXO9 | 476 | 0.148288 |
| 201289_at | CYR61 | 465 | −0.19925 |
| 206548_at | FLJ23556 | 465 | 0.141583 |
| 202593_s_at | MIR16 | 462 | −0.17042 |
| 202932_at | YES1 | 461 | −0.17637 |
| 220575_at | FLJ11800 | 461 | 0.116435 |
| 217713_x_at | DKFZP566N0 | 452 | 0.145994 |
| | 34 | | |
| 211953_s_at | RANBP5 | 447 | −0.17838 |
| 203827_at | WIPI49 | 447 | −0.17767 |
| 221997_s_at | MRPL52 | 444 | 0.132649 |
| 217662_x_at | BCAP29 | 434 | 0.116886 |
| 218519_at | SLC35A5 | 428 | −0.15495 |
| 214833_at | KIAA0792 | 428 | 0.132943 |
| 201339_s_at | SCP2 | 426 | −0.18605 |
| 203799_at | CD302 | 422 | −0.16798 |
| 211090_s_at | PRPF4B | 421 | −0.1838 |
| 220071_x_at | C15orf25 | 420 | 0.138308 |
| 203946_s_at | ARG2 | 415 | −0.14964 |
| 213544_at | ING1L | 415 | 0.137052 |
| 209908_s_at | — | 414 | 0.131346 |
| 201688_s_at | TPD52 | 410 | −0.18965 |
| 215587_x_at | BTBD14B | 410 | 0.139952 |
| 201699_at | PSMC6 | 409 | −0.13784 |
| 214902_x_at | FLJ42393 | 409 | 0.140198 |
| 214041_x_at | RPL37A | 402 | 0.106746 |
| 203987_at | FZD6 | 392 | −0.19252 |
| 211696_x_at | HBB | 392 | −0.09508 |
| 218025_s_at | PECI | 389 | −0.18002 |
| 215852_x_at | KIAA0889 | 382 | 0.12243 |
| 209458_x_at | HBA1/// | 380 | −0.09796 |
| | HBA2 | | |
| 219410_at | TMEM45A | 379 | −0.22387 |
| 215375_x_at | — | 379 | 0.148377 |
| 206302_s_at | NUDT4 | 376 | −0.18873 |
| 208783_s_at | MCP | 372 | −0.15076 |
| 211374_x_at | — | 364 | 0.131101 |
| 220352_x_at | MGC4278 | 364 | 0.152722 |
| 216609_at | TXN | 363 | 0.15162 |
| 201942_s_at | CPD | 363 | −0.1889 |
| 202672_s_at | ATF3 | 361 | −0.12935 |
| 204959_at | MNDA | 359 | −0.21676 |
| 211996_s_at | KIAA0220 | 358 | 0.144358 |
| 222035_s_at | PAPOLA | 353 | −0.14487 |
| 208808_s_at | HMGB2 | 349 | −0.15222 |
| 203711_s_at | HIBCH | 347 | −0.13214 |
| 215179_x_at | PGF | 347 | 0.146279 |
| 213562_s_at | SQLE | 345 | −0.14669 |
| 203765_at | GCA | 340 | −0.1798 |
| 214414_x_at | HBA2 | 336 | −0.08492 |
| 217497_at | ECGF1 | 336 | 0.123255 |
| 220924_s_at | SLC38A2 | 333 | −0.17315 |
| 218139_s_at | C14orf108 | 332 | −0.15021 |
| 201096_s_at | ARF4 | 330 | −0.18887 |
| 220361_at | FLJ12476 | 325 | −0.15452 |
| 202169_s_at | AASDHPPT | 323 | −0.15787 |
| 202527_s_at | SMAD4 | 322 | −0.18399 |
| 202166_s_at | PPP1R2 | 320 | −0.16402 |
| 204634_at | NEK4 | 319 | −0.15511 |
| 215504_x_at | — | 319 | 0.145981 |
| 202388_at | RGS2 | 315 | −0.14894 |
| 215553_x_at | WDR45 | 315 | 0.137586 |
| 200598_s_at | TRA1 | 314 | −0.19349 |
| 202435_s_at | CYP1B1 | 313 | 0.056937 |
| 216206_x_at | MAP2K7 | 313 | 0.10383 |
| 212582_at | OSBPL8 | 313 | −0.17843 |
| 216509_x_at | MLLT10 | 312 | 0.123961 |
| 200908_s_at | RPLP2 | 308 | 0.136645 |
| 215108_x_at | TNRC9 | 306 | −0.1439 |
| 213872_at | C6orf62 | 302 | −0.19548 |
| 214395_x_at | EEF1D | 302 | 0.128234 |
| 222156_x_at | CCPG1 | 301 | −0.14725 |
| 201426_s_at | VIM | 301 | −0.17461 |
| 221972_s_at | Cab45 | 299 | −0.1511 |
| 219957_at | — | 298 | 0.130796 |
| 215123_at | — | 295 | 0.125434 |
| 212515_s_at | DDX3X | 295 | −0.14634 |
| 203357_s_at | CAPN7 | 295 | −0.17109 |
| 211711_s_at | PTEN | 295 | −0.12636 |
| 206165_s_at | CLCA2 | 293 | −0.17699 |
| 213959_s_at | KIAA1005 | 289 | −0.16592 |
| 215083_at | PSPC1 | 289 | 0.147348 |
| 219630_at | PDZK1IP1 | 287 | −0.15086 |
| 204018_x_at | HBA1/// | 286 | −0.08689 |
| | HBA2 | | |
| 208671_at | TDE2 | 286 | −0.17839 |
| 203427_at | ASF1A | 286 | −0.14737 |
| 215281_x_at | POGZ | 286 | 0.142825 |
| 205749_at | CYP1A1 | 285 | 0.107118 |
| 212585_at | OSBPL8 | 282 | −0.13924 |
| 211745_x_at | HBA1/// | 281 | −0.08437 |
| | HBA2 | | |
| 208078_s_at | SNF1LK | 278 | −0.14395 |
| 218041_x_at | SLC38A2 | 276 | −0.17003 |
| 212588_at | PTPRC | 270 | −0.1725 |
| 212397_at | RDX | 270 | −0.15613 |
| 208268_at | ADAM28 | 269 | 0.114996 |
| 207194_s_at | ICAM4 | 269 | 0.127304 |
| 222252_x_at | — | 269 | 0.132241 |
| 217414_x_at | HBA2 | 266 | −0.08974 |
| 207078_at | MED6 | 261 | 0.1232 |
| 215268_at | KIAA0754 | 261 | 0.13669 |
| 221387_at | GPR147 | 261 | 0.128737 |
| 201337_s_at | VAMP3 | 259 | −0.17284 |
| 220218_at | C9orf68 | 259 | 0.125851 |
| 222356_at | TBL1Y | 259 | 0.126765 |
| 208579_x_at | H2BFS | 258 | −0.16608 |
| 219161_s_at | CKLF | 257 | −0.12288 |
| 202917_s_at | S100A8 | 256 | −0.19869 |
| 204455_at | DST | 255 | −0.13072 |
| 211672_s_at | ARPC4 | 254 | −0.17791 |
| 201132_at | HNRPH2 | 254 | −0.12817 |
| 218313_s_at | GALNT7 | 253 | −0.179 |
| 218930_s_at | FLJ11273 | 251 | −0.15878 |
| 219166_at | C14orf104 | 250 | −0.14237 |
| 212805_at | KIAA0367 | 248 | −0.16649 |
| 201551_s_at | LAMP1 | 247 | −0.18035 |
| 202599_s_at | NRIP1 | 247 | −0.16226 |
| 203403_s_at | RNF6 | 247 | −0.14976 |
| 214261_s_at | ADH6 | 242 | −0.1414 |
| 202033_s_at | RB1CC1 | 240 | −0.18105 |
| 203896_s_at | PLCB4 | 237 | −0.20318 |
| 209703_x_at | DKFZP586A0 | 234 | 0.140153 |
| | 522 | | |
| 211699_x_at | HBA1/// | 232 | −0.08369 |
| | HBA2 | | |
| 210764_s_at | CYR61 | 231 | −0.13139 |
| 206391_at | RARRES1 | 230 | −0.16931 |
| 201312_s_at | SH3BGRL | 225 | −0.12265 |
| 200798_x_at | MCL1 | 221 | −0.13113 |
| 214912_at | — | 221 | 0.116262 |
| 204621_s_at | NR4A2 | 217 | −0.10896 |
| 217761_at | MTCBP-1 | 217 | −0.17558 |
| 205830_at | CLGN | 216 | −0.14737 |
| 218438_s_at | MED28 | 214 | −0.14649 |
| 207475_at | FABP2 | 214 | 0.097003 |
| 208621_s_at | VIL2 | 213 | −0.19678 |
| 202436_s_at | CYP1B1 | 212 | 0.042216 |
| 202539_s_at | HMGCR | 210 | −0.15429 |
| 210830_s_at | PON2 | 209 | −0.17184 |
| 211906_s_at | SERPINB4 | 207 | −0.14728 |
| 202241_at | TRIB1 | 207 | −0.10706 |
| 203594_at | RTCD1 | 207 | −0.13823 |
| 215863_at | TFR2 | 207 | 0.095157 |
| 221992_at | LOC283970 | 206 | 0.126744 |
| 221872_at | RARRES1 | 205 | −0.11496 |
| 219564_at | KCNJ16 | 205 | −0.13908 |
| 201329_s_at | ETS2 | 205 | −0.14994 |
| 214188_at | HIS1 | 203 | 0.1257 |
| 201667_at | GJA1 | 199 | −0.13848 |
| 201464_x_at | JUN | 199 | −0.09858 |
| 215409_at | LOC254531 | 197 | 0.094182 |
| 202583_s_at | RANBP9 | 197 | −0.13902 |
| 215594_at | — | 197 | 0.101007 |
| 214326_x_at | JUND | 196 | −0.1702 |
| 217140_s_at | VDAC1 | 196 | −0.14682 |
| 215599_at | SMA4 | 195 | 0.133438 |
| 209896_s_at | PTPN11 | 195 | −0.16258 |
| 204846_at | CP | 195 | −0.14378 |
| 222303_at | — | 193 | −0.10841 |
| 218218_at | DIP13B | 193 | −0.12136 |
| 211015_s_at | HSPA4 | 192 | −0.13489 |
| 208666_s_at | 5T13 | 191 | −0.13361 |
| 203191_at | ABCB6 | 190 | 0.096808 |
| 202731_at | PDCD4 | 190 | −0.1545 |
| 209027_s_at | ABI1 | 190 | −0.15472 |
| 205979_at | SCGB2A1 | 189 | −0.15091 |
| 216351_x_at | DAZ1 /// | 189 | 0.106368 |
| | DAZ3/// | | |
| | DAZ2/// | | |
| | DAZ4 | | |
| 220240_s_at | C13orf11 | 188 | −0.16959 |
| 204482_at | CLDN5 | 187 | 0.094134 |
| 217234_s_at | VIL2 | 186 | −0.16035 |
| 214350_at | SNTB2 | 186 | 0.095723 |
| 201693_s_at | EGR1 | 184 | −0.10732 |
| 212328_at | KIAA1102 | 182 | −0.12113 |
| 220168_at | CASC1 | 181 | −0.1105 |
| 203628_at | IGF1R | 180 | 0.067575 |
| 204622_x_at | NR4A2 | 180 | −0.11482 |
| 213246_at | C14orf109 | 180 | −0.16143 |
| 218728_s_at | HSPC163 | 180 | −0.13248 |
| 214753_at | PFAAP5 | 179 | 0.130184 |
| 206336_at | CXCL6 | 178 | −0.05634 |
| 201445_at | CNN3 | 178 | −0.12375 |
| 209886_s_at | SMAD6 | 176 | 0.079296 |
| 213376_at | ZBTB1 | 176 | −0.17777 |
| 213887_s_at | POLR2E | 175 | −0.16392 |
| 204783_at | MLF1 | 174 | −0.13409 |
| 218824_at | FLJ10781 | 173 | 0.1394 |
| 212417_at | SCAMPI | 173 | −0.17052 |
| 202437_s_at | CYP1B1 | 171 | 0.033438 |
| 217528_at | CLCA2 | 169 | −0.14179 |
| 218170_at | ISOC1 | 169 | −0.14064 |
| 206278_at | PTAFR | 167 | 0.087096 |
| 201939_at | PLK2 | 167 | −0.11049 |
| 200907_s_at | KIAA0992 | 166 | −0.18323 |
| 207480_s_at | MEIS2 | 166 | −0.15232 |
| 201417_at | SOX4 | 162 | −0.09617 |
| 213826_s_at | — | 160 | 0.097313 |
| 214953_s_at | APP | 159 | −0.1645 |
| 204897_at | PTGER4 | 159 | −0.08152 |
| 201711_x_at | RANBP2 | 158 | −0.17192 |
| 202457_s_at | PPP3CA | 158 | −0.18821 |
| 206683_at | ZNF165 | 158 | −0.08848 |
| 214581_x_at | TNFRSF21 | 156 | −0.14624 |
| 203392_s_at | CTBP1 | 155 | −0.16161 |
| 212720_at | PAPOLA | 155 | −0.14809 |
| 207758_at | PPM1F | 155 | 0.090007 |
| 220995_at | STXBP6 | 155 | 0.106749 |
| 213831_at | HLA-DQA1 | 154 | 0.193368 |
| 212044_s_at | — | 153 | 0.098889 |
| 202434_s_at | CYP1B1 | 153 | 0.049744 |
| 206166_s_at | CLCA2 | 153 | −0.1343 |
| 218343_s_at | GTF3C3 | 153 | −0.13066 |
| 202557_at | STCH | 152 | −0.14894 |
| 201133_s_at | PJA2 | 152 | −0.18481 |
| 213605_s_at | MGC22265 | 151 | 0.130895 |
| 210947_s_at | MSH3 | 151 | −0.12595 |
| 208310_s_at | C7orf28A/// | 151 | −0.15523 |
| | C7orf28B | | |
| 209307_at | — | 150 | −0.1667 |
| 215387_x_at | GPC6 | 148 | 0.114691 |
| 213705_at | MAT2A | 147 | 0.104855 |
| 213979_s_at | — | 146 | 0.121562 |
| 212731_at | LOC157567 | 146 | −0.1214 |
| 210117_at | SPAG1 | 146 | −0.11236 |
| 200641_s_at | YWHAZ | 145 | −0.14071 |
| 210701_at | CFDP1 | 145 | 0.151664 |
| 217152_at | NCOR1 | 145 | 0.130891 |
| 204224_s_at | GCH1 | 144 | −0.14574 |
| 202028_s_at | — | 144 | 0.094276 |
| 201735_s_at | CLCN3 | 144 | −0.1434 |
| 208447_s_at | PRPS1 | 143 | −0.14933 |
| 220926_s_at | C1orf22 | 142 | −0.17477 |
| 211505_s_at | STAU | 142 | −0.11618 |
| 221684_s_at | NYX | 142 | 0.102298 |
| 206906_at | ICAM5 | 141 | 0.076813 |
| 213228_at | PDE8B | 140 | −0.13728 |
| 217202_s_at | GLUL | 139 | −0.15489 |
| 211713_x_at | KIAA0101 | 138 | 0.108672 |
| 215012_at | ZNF451 | 138 | 0.13269 |
| 200806_s_at | HSPD1 | 137 | −0.14811 |
| 201466_s_at | JUN | 135 | −0.0667 |
| 211564_s_at | PDLIM4 | 134 | −0.12756 |
| 207850_at | CXCL3 | 133 | −0.17973 |
| 221841_s_at | KLF4 | 133 | −0.1415 |
| 200605_s_at | PRKAR1A | 132 | −0.15642 |
| 221198_at | SCT | 132 | 0.08221 |
| 201772_at | AZIN1 | 131 | −0.16639 |
| 205009_at | TFF1 | 130 | −0.17578 |
| 205542_at | STEAP1 | 129 | −0.08498 |
| 218195_at | C6orf211 | 129 | −0.14497 |
| 213642_at | — | 128 | 0.079657 |
| 212891_s_at | GADD45GIP1 | 128 | −0.09272 |
| 202798_at | SEC24B | 127 | −0.12621 |
| 222207_x_at | — | 127 | 0.10783 |
| 202638_s_at | ICAM1 | 126 | 0.070364 |
| 200730_s_at | PTP4A1 | 126 | −0.15289 |
| 219355_at | FLJ10178 | 126 | −0.13407 |
| 220266_s_at | KLF4 | 126 | −0.15324 |
| 201259_s_at | SYPL | 124 | −0.16643 |
| 209649_at | STAM2 | 124 | −0.1696 |
| 220094_s_at | C6orf79 | 123 | −0.12214 |
| 221751_at | PANK3 | 123 | −0.1723 |
| 200008_s_at | GDI2 | 123 | −0.15852 |
| 205078_at | PIGF | 121 | −0.13747 |
| 218842_at | FLJ21908 | 121 | −0.08903 |
| 202536_at | CHMP2B | 121 | −0.14745 |
| 220184_at | NANOG | 119 | 0.098142 |
| 201117_s_at | CPE | 118 | −0.20025 |
| 219787_s_at | ECT2 | 117 | −0.14278 |
| 206628_at | SLC5A1 | 117 | −0.12838 |
| 204007_at | FCGR3B | 116 | −0.15337 |
| 209446_s_at | — | 116 | 0.100508 |
| 211612_s_at | IL13RA1 | 115 | −0.17266 |
| 220992_s_at | C1orf25 | 115 | −0.11026 |
| 221899_at | PFAAP5 | 115 | 0.11698 |
| 221719_s_at | LZTS1 | 115 | 0.093494 |
| 201473_at | JUNB | 114 | −0.10249 |
| 221193_s_at | ZCCHC10 | 112 | −0.08003 |
| 215659_at | GSDML | 112 | 0.118288 |
| 205157_s_at | KRT17 | 111 | −0.14232 |
| 201001_s_at | UBE2V1/// | 111 | −0.16786 |
| | Kua-UEV | | |
| 216789_at | — | 111 | 0.105386 |
| 205506_at | VIL1 | 111 | 0.097452 |
| 204875_s_at | GMDS | 110 | −0.12995 |
| 207191_s_at | ISLR | 110 | 0.100627 |
| 202779_s_at | UBE2S | 109 | −0.11364 |
| 210370_s_at | LY9 | 109 | 0.096323 |
| 202842_s_at | DNAJB9 | 108 | −0.15326 |
| 201082_s_at | DCTN1 | 107 | −0.10104 |
| 215588_x_at | RIOK3 | 107 | 0.135837 |
| 211076_x_at | DRPLA | 107 | 0.102743 |
| 210230_at | — | 106 | 0.115001 |
| 206544_x_at | SMARCA2 | 106 | −0.12099 |
| 208852_s_at | CANX | 105 | −0.14776 |
| 215405_at | MYO1E | 105 | 0.086393 |
| 208653_s_at | CD164 | 104 | −0.09185 |
| 206355_at | GNAL | 103 | 0.1027 |
| 210793_s_at | NUP98 | 103 | −0.13244 |
| 215070_x_at | RABGAP1 | 103 | 0.125029 |
| 203007_x_at | LYPLA1 | 102 | −0.17961 |
| 203841_x_at | MAPRE3 | 102 | −0.13389 |
| 206759_at | FCER2 | 102 | 0.081733 |
| 202232_s_at | GA17 | 102 | −0.11373 |
| 215892_at | — | 102 | 0.13866 |
| 214359_s_at | HSPCB | 101 | −0.12276 |
| 215810_x_at | DST | 101 | 0.098963 |
| 208937_s_at | ID1 | 100 | −0.06552 |
| 213664_at | SLC1A1 | 100 | −0.12654 |
| 219338_s_at | FLJ20156 | 100 | −0.10332 |
| 206595_at | CST6 | 99 | −0.10059 |
| 207300_s_at | F7 | 99 | 0.082445 |
| 213792_s_at | INSR | 98 | 0.137962 |
| 209674_at | CRY1 | 98 | −0.13818 |
| 40665_at | FMO3 | 97 | −0.05976 |
| 217975_at | WBP5 | 97 | −0.12698 |
| 210296_s_at | PXMP3 | 97 | −0.13537 |
| 215483_at | AKAP9 | 95 | 0.125966 |
| 212633_at | KIAA0776 | 95 | −0.16778 |
| 206164_at | CLCA2 | 94 | −0.13117 |
| 216813_at | — | 94 | 0.089023 |
| 208925_at | C3orf4 | 94 | −0.1721 |
| 219469_at | DNCH2 | 94 | −0.12003 |
| 206016_at | CXorf37 | 93 | −0.11569 |
| 216745_x_at | LRCH1 | 93 | 0.117149 |
| 212999_x_at | HLA-DQB1 | 92 | 0.110258 |
| 216859_x_at | — | 92 | 0.116351 |
| 201636_at | — | 92 | −0.13501 |
| 204272_at | LGALS4 | 92 | 0.110391 |
| 215454_x_at | SFTPC | 91 | 0.064918 |
| 215972_at | — | 91 | 0.097654 |
| 220593_s_at | FLJ20753 | 91 | 0.095702 |
| 222009_at | CGI-14 | 91 | 0.070949 |
| 207115_x_at | MBTD1 | 91 | 0.107883 |
| 216922_x_at | DAZ1/// | 91 | 0.086888 |
| | DAZ3/// | | |
| | DAZ2/// | | |
| | DAZ4 | | |
| 217626_at | AKR1C1/// | 90 | 0.036545 |
| | AKR1C2 | | |
| 211429_s_at | SERPINA1 | 90 | −0.11406 |
| 209662_at | CETN3 | 90 | −0.10879 |
| 201629_s_at | ACP1 | 90 | −0.14441 |
| 201236_s_at | BTG2 | 89 | −0.09435 |
| 217137_x_at | — | 89 | 0.070954 |
| 212476_at | CENTB2 | 89 | −0.1077 |
| 218545_at | FLJ11088 | 89 | −0.12452 |
| 208857_s_at | PCMT1 | 89 | −0.14704 |
| 221931_s_at | SEH1L | 88 | −0.11491 |
| 215046_at | FLJ23861 | 88 | −0.14667 |
| 220222_at | PRO1905 | 88 | 0.081524 |
| 209737_at | AIP1 | 87 | −0.07696 |
| 203949_at | MPO | 87 | 0.113273 |
| 219290_x_at | DAPP1 | 87 | 0.111366 |
| 205116_at | LAMA2 | 86 | 0.05845 |
| 222316_at | VDP | 86 | 0.091505 |
| 203574_at | NFIL3 | 86 | −0.14335 |
| 207820_at | ADH1A | 86 | 0.104444 |
| 203751_x_at | JUND | 85 | −0.14118 |
| 202930_s_at | SUCLA2 | 85 | −0.14884 |
| 215404_x_at | FGFR1 | 85 | 0.119684 |
| 216266_s_at | ARFGEF1 | 85 | −0.12432 |
| 212806_at | KIAA0367 | 85 | −0.13259 |
| 219253_at | — | 83 | −0.14094 |
| 214605_x_at | GPR1 | 83 | 0.114443 |
| 205403_at | IL1R2 | 82 | −0.19721 |
| 222282_at | PAPD4 | 82 | 0.128004 |
| 214129_at | PDE4DIP | 82 | −0.13913 |
| 209259_s_at | CSPG6 | 82 | −0.12618 |
| 216900_s_at | CHRNA4 | 82 | 0.105518 |
| 221943_x_at | RPL38 | 80 | 0.086719 |
| 215386_at | AUTS2 | 80 | 0.129921 |
| 201990_s_at | CREBL2 | 80 | −0.13645 |
| 220145_at | FLJ21159 | 79 | −0.16097 |
| 221173_at | USH1C | 79 | 0.109348 |
| 214900_at | ZKSCAN1 | 79 | 0.075517 |
| 203290_at | HLA-DQA1 | 78 | −0.20756 |
| 215382_x_at | TPSAB1 | 78 | −0.09041 |
| 201631_s_at | IER3 | 78 | −0.12038 |
| 212188_at | KCTD12 | 77 | −0.14672 |
| 220428_at | CD207 | 77 | 0.101238 |
| 215349_at | — | 77 | 0.10172 |
| 213928_s_at | HRB | 77 | 0.092136 |
| 221228_s_at | — | 77 | 0.0859 |
| 202069_s_at | IDH3A | 76 | −0.14747 |
| 208554_at | POU4F3 | 76 | 0.107529 |
| 209504_s_at | PLEKHB1 | 76 | −0.13125 |
| 212989_at | TMEM23 | 75 | −0.11012 |
| 216197_at | ATF7IP | 75 | 0.115016 |
| 204748_at | PTGS2 | 74 | −0.15194 |
| 205221_at | HGD | 74 | 0.096171 |
| 214705_at | INADL | 74 | 0.102919 |
| 213939_s_at | RIPX | 74 | 0.091175 |
| 203691_at | P13 | 73 | −0.14375 |
| 220532_s_at | LR8 | 73 | −0.11682 |
| 209829_at | C6orf32 | 73 | −0.08982 |
| 206515_at | CYP4F3 | 72 | 0.104171 |
| 218541_s_at | C8orf4 | 72 | −0.09551 |
| 210732_s_at | LGALS8 | 72 | −0.13683 |
| 202643_s_at | TNFAIP3 | 72 | −0.16699 |
| 218963_s_at | KRT23 | 72 | −0.10915 |
| 213304_at | KIAA0423 | 72 | −0.12256 |
| 202768_at | FOSB | 71 | −0.06289 |
| 205623_at | ALDH3A1 | 71 | 0.045457 |
| 206488_s_at | CD36 | 71 | −0.15899 |
| 204319_s_at | RGS10 | 71 | −0.10107 |
| 217811_at | SELT | 71 | −0.16162 |
| 202746_at | ITM2A | 70 | −0.06424 |
| 221127_s_at | RIG | 70 | 0.110593 |
| 209821_at | C9orf26 | 70 | −0.07383 |
| 220957_at | CTAGE1 | 70 | 0.092986 |
| 215577_at | UBE2E1 | 70 | 0.10305 |
| 214731_at | DKFZp547A0 | 70 | 0.102821 |
| | 23 | | |
| 210512_s_at | VEGF | 69 | −0.11804 |
| 205267_at | POU2AF1 | 69 | 0.101353 |
| 216202_s_at | SPTLC2 | 69 | −0.11908 |
| 220477_s_at | C20orf30 | 69 | −0.16221 |
| 205863_at | D100Al2 | 68 | −0.10353 |
| 215780_s_at | SET/// | 68 | −0.10381 |
| | LOC389168 | | |
| 218197_s_at | OXR1 | 68 | −0.14424 |
| 203077_s_at | SMAD2 | 68 | −0.11242 |
| 222339_x_at | — | 68 | 0.121585 |
| 200698_at | KDELR2 | 68 | −0.15907 |
| 210540_s_at | B4GALT4 | 67 | −0.13556 |
| 217725_x_at | PAI-RBP1 | 67 | −0.14956 |
| 217082_at | — | 67 | 0.086098 |

TABLE 7
Group of 20 genes useful in prognosis and/or diagnosis of lung cancer.

| | | | Signal to noise in |
|---|---|---|---|
| | | | a cancer sample. |
| | | | Negative values |
| | | Number of runs | indicate increase |
| Affymetrix probe | | the gene is | of expression |
| ID No. that can be | | indicated in | in lung cancer, |
| used to identify | | cancer samples | positive values |
| the gene/nucleic | | as differentially | indicate decrease |
| acid sequence in | | expressed out of | of expression |
| the next column | Gene symbol | 1000 test runs | in lung cancer. |
| 207953_at | AD7C-NTP | 1000 | 0.218433 |
| 215208_x_at | RPL35A | 999 | 0.228485 |
| 215604_x_at | UBE2D2 | 998 | 0.224878 |
| 218155_x_at | FLJ10534 | 998 | 0.186425 |
| 216858_x_at | — | 997 | 0.232969 |
| 208137_x_at | — | 996 | 0.191938 |
| 214715_x_at | ZNF160 | 996 | 0.198532 |
| 217715_x_at | ZNF354A | 995 | 0.223881 |
| 220720_x_at | FLJ14346 | 989 | 0.17976 |
| 215907_at | BACH2 | 987 | 0.178338 |
| 217679_x_at | — | 987 | 0.265918 |
| 206169_x_at | RoXaN | 984 | 0.259637 |
| 208246_x_at | TK2 | 982 | 0.179058 |
| 222104_x_at | GTF2H3 | 981 | 0.186025 |
| 206056_x_at | SPN | 976 | 0.196398 |
| 217653_x_at | — | 976 | 0.270552 |
| 210679_x_at | — | 970 | 0.181718 |
| 207730_x_at | HDGF2 | 969 | 0.169108 |
| 214594_x_at | ATP8B1 | 962 | 0.284039 |

One can use the above tables to correlate or compare the expression of the transcript to the expression of the gene product. Increased expression of the transcript as shown in the table corresponds to increased expression of the gene product. Similarly, decreased expression of the transcript as shown in the table corresponds to decreased expression of the gene product

The analysis of the gene expression of one or more genes and/or transcripts of the groups or their subgroups of the present invention can be performed using any gene expression method known to one skilled in the art. Such methods include, but are not limited to expression analysis using nucleic acid chips (e.g. Affymetrix chips) and quantitative RT-PCR based methods using, for example real-time detection of the transcripts. Analysis of transcript levels according to the present invention can be made using total or messenger RNA or proteins encoded by the genes identified in the diagnostic gene groups of the present invention as a starting material. In the preferred embodiment the analysis is an immunohistochemical analysis with an antibody directed against proteins comprising at least about 10-20, 20-30, preferably at least 36, at least 36-50, 50, about 50-60, 60-70, 70-80, 80-90, 96, 100-180, 180-200, 200-250, 250-300, 300-350, 350-400, 400-450, 450-500, 500-535 proteins encoded by the genes and/or transcripts as shown in Tables 1-7.

The methods of analyzing transcript levels of the gene groups in an individual include Northern-blot hybridization, ribonuclease protection assay, and reverse transcriptase polymerase chain reaction (RT-PCR) based methods. The different RT-PCR based techniques are the most suitable quantification method for diagnostic purposes of the present invention, because they are very sensitive and thus require only a small sample size which is desirable for a diagnostic test. A number of quantitative RT-PCR based methods have been described and are useful in measuring the amount of transcripts according to the present invention. These methods include RNA quantification using PCR and complementary DNA (cDNA) arrays (Shalon et al., Genome Research 6(7):639-45, 1996; Bernard et al., Nucleic Acids Research 24(8):1435-42, 1996), real competitive PCR using a MALDI-TOF Mass spectrometry based approach (Ding et al, PNAS, 100: 3059-64, 2003), solid-phase mini-sequencing technique, which is based upon a primer extension reaction (U.S. Pat. No. 6,013,431, Suomalainen et al. Mol. Biotechnol. June; 15(2):123-31, 2000), ion-pair high-performance liquid chromatography (Doris et al. J. Chromatogr. A May 8; 806(1):47-60, 1998), and 5′ nuclease assay or real-time RT-PCR (Holland et al. Proc Natl Acad Sci USA 88: 7276-7280, 1991).

Methods using RT-PCR and internal standards differing by length or restriction endonuclease site from the desired target sequence allowing comparison of the standard with the target using gel electrophoretic separation methods followed by densitometric quantification of the target have also been developed and can be used to detect the amount of the transcripts according to the present invention (see, e.g., U.S. Pat. Nos. 5,876,978; 5,643,765; and 5,639,606.

The samples are preferably obtained from bronchial airways using, for example, endoscopic cytobrush in connection with a fiber optic bronchoscopy. In one embodiment, the cells are obtained from the individual's mouth buccal cells, using, for example, a scraping of the buccal mucosa.

In one preferred embodiment, the invention provides a prognostic and/or diagnostic immunohistochemical approach, such as a dip-stick analysis, to determine risk of developing lung disease. Antibodies against proteins, or antigenic epitopes thereof, that are encoded by the group of genes of the present invention, are either commercially available or can be produced using methods well know to one skilled in the art.

The invention contemplates either one dipstick capable of detecting all the diagnostically important gene products or alternatively, a series of dipsticks capable of detecting the amount proteins of a smaller sub-group of diagnostic proteins of the present invention.

Antibodies can be prepared by means well known in the art. The term “antibodies” is meant to include monoclonal antibodies, polyclonal antibodies and antibodies prepared by recombinant nucleic acid techniques that are selectively reactive with a desired antigen. Antibodies against the proteins encoded by any of the genes in the diagnostic gene groups of the present invention are either known or can be easily produced using the methods well known in the art. Internet sites such as Biocompare through the World Wide Web at “biocompare.com/abmatrix.asp?antibody=y” provide a useful tool to anyone skilled in the art to locate existing antibodies against any of the proteins provided according to the present invention.

Antibodies against the diagnostic proteins according to the present invention can be used in standard techniques such as Western blotting or immunohistochemistry to quantify the level of expression of the proteins of the diagnostic airway proteome. This is quantified according to the expression of the gene transcript, i.e. the increased expression of transcript corresponds to increased expression of the gene product, i.e. protein. Similarly decreased expression of the transcript corresponds to decreased expression of the gene product or protein. Detailed guidance of the increase or decrease of expression of preferred transcripts in lung disease, particularly lung cancer, is set forth in the tables. For example, Tables 5 and 6 describe a group of genes the expression of which is altered in lung cancer.

Immunohistochemical applications include assays, wherein increased presence of the protein can be assessed, for example, from a saliva or sputum sample.

The immunohistochemical assays according to the present invention can be performed using methods utilizing solid supports. The solid support can be a any phase used in performing immunoassays, including dipsticks, membranes, absorptive pads, beads, microtiter wells, test tubes, and the like. Preferred are test devices which may be conveniently used by the testing personnel or the patient for self-testing, having minimal or no previous training. Such preferred test devices include dipsticks, membrane assay systems as described in U.S. Pat. No. 4,632,901. The preparation and use of such conventional test systems is well described in the patent, medical, and scientific literature. If a stick is used, the anti-protein antibody is bound to one end of the stick such that the end with the antibody can be dipped into the solutions as described below for the detection of the protein. Alternatively, the samples can be applied onto the antibody-coated dipstick or membrane by pipette or dropper or the like.

The antibody against proteins encoded by the diagnostic airway transcriptome (the “protein”) can be of any isotype, such as IgA, IgG or IgM, Fab fragments, or the like. The antibody may be a monoclonal or polyclonal and produced by methods as generally described, for example, in Harlow and Lane, Antibodies, A Laboratory Manual, Cold Spring Harbor Laboratory, 1988, incorporated herein by reference. The antibody can be applied to the solid support by direct or indirect means. Indirect bonding allows maximum exposure of the protein binding sites to the assay solutions since the sites are not themselves used for binding to the support. Preferably, polyclonal antibodies are used since polyclonal antibodies can recognize different epitopes of the protein thereby enhancing the sensitivity of the assay.

The solid support is preferably non-specifically blocked after binding the protein antibodies to the solid support. Non-specific blocking of surrounding areas can be with whole or derivatized bovine serum albumin, or albumin from other animals, whole animal serum, casein, non-fat milk, and the like.

The sample is applied onto the solid support with bound protein-specific antibody such that the protein will be bound to the solid support through said antibodies. Excess and unbound components of the sample are removed and the solid support is preferably washed so the antibody-antigen complexes are retained on the solid support. The solid support may be washed with a washing solution which may contain a detergent such as Tween-20, Tween-80 or sodium dodecyl sulfate.

After the protein has been allowed to bind to the solid support, a second antibody which reacts with protein is applied. The second antibody may be labeled, preferably with a visible label. The labels may be soluble or particulate and may include dyed immunoglobulin binding substances, simple dyes or dye polymers, dyed latex beads, dye-containing liposomes, dyed cells or organisms, or metallic, organic, inorganic, or dye solids. The labels may be bound to the protein antibodies by a variety of means that are well known in the art. In some embodiments of the present invention, the labels may be enzymes that can be coupled to a signal producing system. Examples of visible labels include alkaline phosphatase, beta-galactosidase, horseradish peroxidase, and biotin. Many enzyme-chromogen or enzyme-substrate-chromogen combinations are known and used for enzyme-linked assays. Dye labels also encompass radioactive labels and fluorescent dyes.

Simultaneously with the sample, corresponding steps may be carried out with a known amount or amounts of the protein and such a step can be the standard for the assay. A sample from a healthy individual exposed to a similar air pollutant such as cigarette smoke, can be used to create a standard for any and all of the diagnostic gene group encoded proteins.

The solid support is washed again to remove unbound labeled antibody and the labeled antibody is visualized and quantified. The accumulation of label will generally be assessed visually. This visual detection may allow for detection of different colors, for example, red color, yellow color, brown color, or green color, depending on label used. Accumulated label may also be detected by optical detection devices such as reflectance analyzers, video image analyzers and the like. The visible intensity of accumulated label could correlate with the concentration of protein in the sample. The correlation between the visible intensity of accumulated label and the amount of the protein may be made by comparison of the visible intensity to a set of reference standards. Preferably, the standards have been assayed in the same way as the unknown sample, and more preferably alongside the sample, either on the same or on a different solid support.

The concentration of standards to be used can range from about 1 mg of protein per liter of solution, up to about 50 mg of protein per liter of solution. Preferably, two or more different concentrations of an airway gene group encoded proteins are used so that quantification of the unknown by comparison of intensity of color is more accurate.

For example, the present invention provides a method for detecting risk of developing lung cancer in a subject exposed to cigarette smoke comprising measuring the transcription profile of the proteins encoded by one or more groups of genes of the invention in a biological sample of the subject. Preferably at least about 30, still more preferably at least about 36, 40, 50, 60, 70, 80, 90, 100, 110, 120, 130, 140, 150, 160, 170, or about 180 of the proteins encoded by the airway transcriptome in a biological sample of the subject are analyzed. The method comprises binding an antibody against each protein encoded by the gene in the gene group (the “protein”) to a solid support chosen from the group consisting of dip-stick and membrane; incubating the solid support in the presence of the sample to be analyzed under conditions where antibody-antigen complexes form; incubating the support with an anti-protein antibody conjugated to a detectable moiety which produces a signal; visually detecting said signal, wherein said signal is proportional to the amount of protein in said sample; and comparing the signal in said sample to a standard, wherein a difference in the amount of the protein in the sample compared to said standard of the same group of proteins, is indicative of diagnosis of or an increased risk of developing lung cancer. The standard levels are measured to indicate expression levels in an airway exposed to cigarette smoke where no cancer has been detected.

The assay reagents, pipettes/dropper, and test tubes may be provided in the form of a kit. Accordingly, the invention further provides a test kit for visual detection of the proteins encoded by the airway gene groups, wherein detection of a level that differs from a pattern in a control individual is considered indicative of an increased risk of developing lung disease in the subject. The test kit comprises one or more solutions containing a known concentration of one or more proteins encoded by the airway transcriptome (the “protein”) to serve as a standard; a solution of a anti-protein antibody bound to an enzyme; a chromogen which changes color or shade by the action of the enzyme; a solid support chosen from the group consisting of dip-stick and membrane carrying on the surface thereof an antibody to the protein. Instructions including the up or down regulation of the each of the genes in the groups as provided by the Tables 1 and 2 are included with the kit.

The present invention also describes a novel method for prognosis and diagnosis and follow-up for lung diseases. The method is based on detecting gene expression changes of nose epithelial cells which we have discovered closely mirror the gene expression changes in the lung.

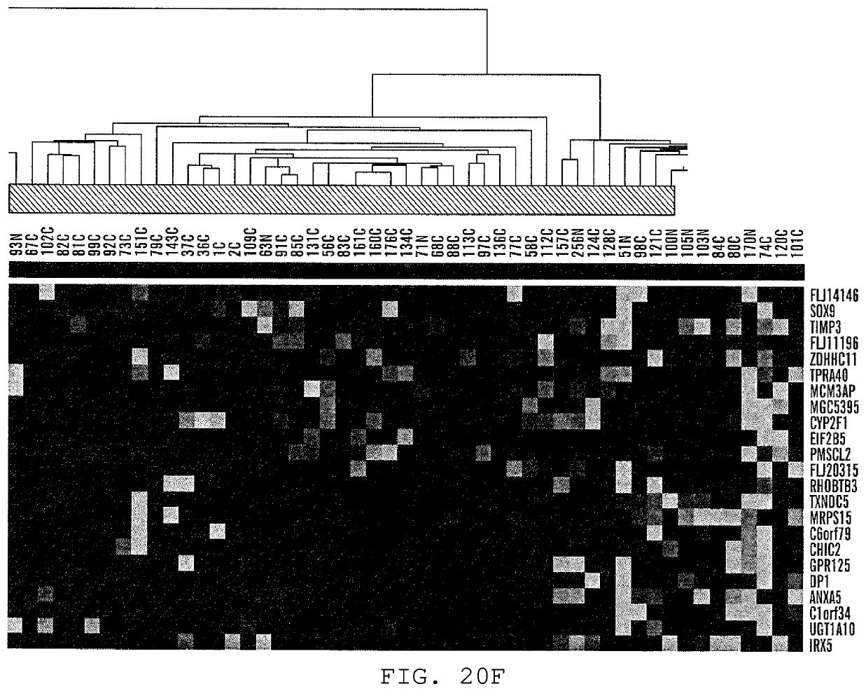

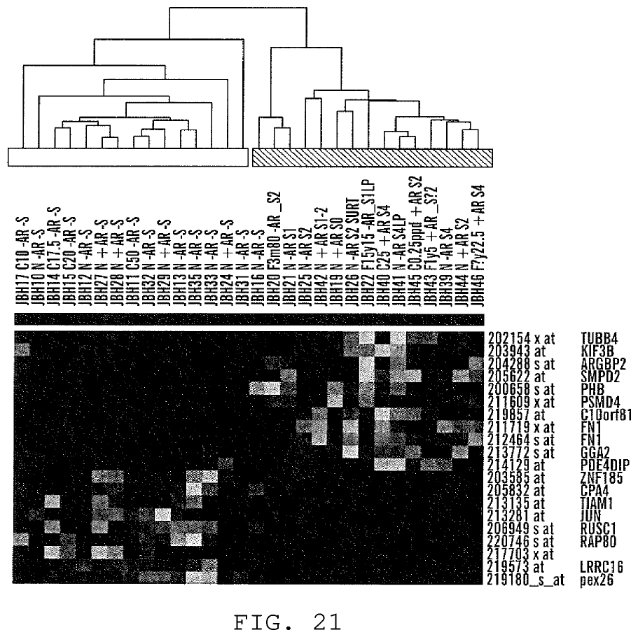

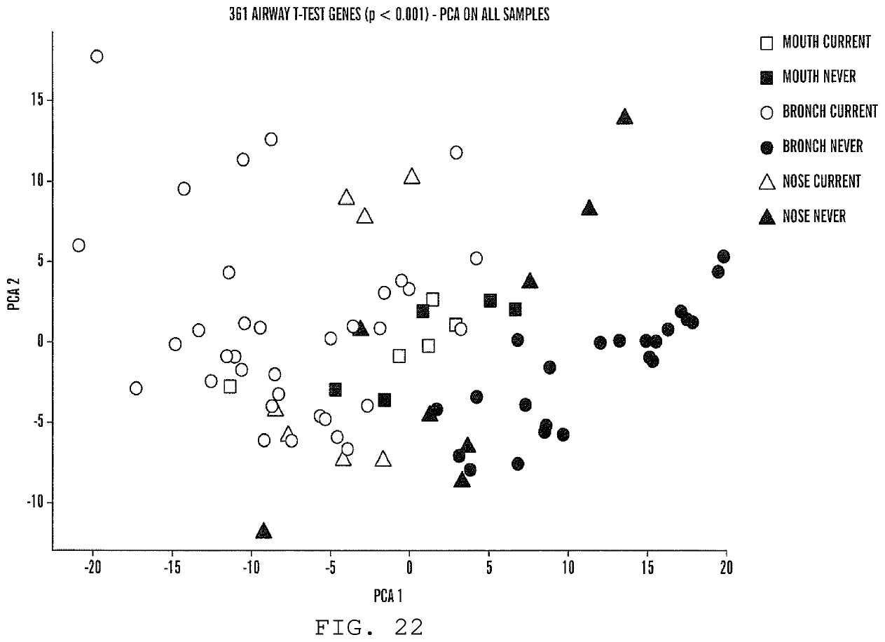

Specifically, we have discovered that similar patterns of gene expression changes can be found in the nose epithelial cells when compared to lung epithelial changes in two model systems. In one experiment, we showed that a host gene expression in response to tobacco smoke is similar whether it is measured from the lung epithelial cells or from the nasal epithelial cells (FIG. 22). Accordingly, we have discovered that we can rely on the results and data obtained with bronchial epithelial cells. This correlation is similar, typically better than 75%, even if it is not identical. Thus, by looking at the same gene groups that are diagnostic and/or prognostic for bronchial epithelial cells those groups are also diagnostic and/or prognostic for nasal epithelial cells. We also showed that gene expression changes distinguishing between individuals affected with a lung diseases, such as sarcoidosis, and from individuals not affected with that diseases.

Accordingly, the invention provides a substantially less invasive method for diagnosis, prognosis and follow-up of lung diseases using gene expression analysis of samples from nasal epithelial cells.

One can take the nose epithelial cell sample from an individual using a brush or a swab. One can collect the nose epithelial cells in any way known to one skilled in the art. For example one can use nasal brushing. For example, one can collect the nasal epithelial cells by brushing the inferior turbinate and/or the adjacent lateral nasal wall. For example, following local anesthesia with 2% lidocaine solution, a CYROBRUSH® (MedScand Medical, Malmai, Sweden) or a similar device, is inserted into the nare, for example the right nare, and under the inferior turbinate using a nasal speculum for visualization. The brush is turned a couple of times, for example 1, 2, 3, 4, 5 times, to collect epithelial cells.

To isolate nucleic acids from the cell sample, the cells can be placed immediately into a solution that prevents nucleic acids from degradation. For example, if the cells are collected using the CYTOBRUSH, and one wishes to isolate RNA, the brush is placed immediately into an RNA stabilizer solution, such as RNALATER®, AMBION®, Inc.

One can also isolate DNA. After brushing, the device can be placed in a buffer, such as phosphate buffered saline (PBS) for DNA isolation.

The nucleic acids are then subjected to gene expression analysis. Preferably, the nucleic acids are isolated and purified. However, if one uses techniques such as microfluidic devises, cells may be placed into such device as whole cells without substantial purification.

In one preferred embodiment, one analyzes gene expression from nasal epithelial cells using gene/transcript groups and methods of using the expression profile of these gene/transcript groups in diagnosis and prognosis of lung diseases.

We provide a method that is much less invasive than analysis of bronchial samples. The method provided herein not only significantly increases the diagnostic accuracy of lung diseases, such as lung cancer, but also make the analysis much less invasive and thus much easier for the patients and doctors to perform. When one combines the gene expression analysis of the present invention with bronchoscopy, the diagnosis of lung cancer is dramatically better by detecting the cancer in an earlier stage than any other available method to date, and by providing far fewer false negatives and/or false positives than any other available method.

In one embodiment, one analyzes the nasal epithelial calls for a group of gene transcripts that one can use individually and in groups or subsets for enhanced diagnosis for lung diseases, such as lung cancer, using gene expression analysis.

On one embodiment, the invention provides a group of genes useful for lung disease diagnosis from a nasal epithelial cell sample as listed in Tables 18, 19, and/or 20.

In one embodiment, one would analyze the nasal epithelial cells using at least one and no more than 361 of the genes listed in Table 18. For example, one can analyze 1, 2, 3, 4, 5, 6, 7, 8, 9, 10, 10-15, 15-20, 20-30, 30-40, 40-50, at least 10, at least 20, at least 30, at least 40 at least 50, at least 60, at least 70, at least 80, at least 90, at least 100, at least 110, at least 120, at least 130, at least 140, at least 150, at least 160, at least or at maximum of 170, at least or at maximum of 180, at least or at maximum of 190, at least or at maximum of 200, 210, 220, 230, 240, 250, 260, 270, 280, 290, 300, 310, 320, 330, 340, 350, 360, or at least 361 or at maximum of the 361 genes of genes as listed on Table 18.

In one embodiment, the invention provides genes.

One example of the gene transcript groups useful in the diagnostic/prognostic tests of the invention is set forth in Table 16. We have found that taking any group that has at least 20 of the Table 16 genes provides a much greater diagnostic capability than chance alone and that these changes are substantially the same in the nasal epithelial cells than they are in the bronchial samples as described in PCT/US2006/014132.

Preferably one would analyze the nasal epithelial cells using more than 20 of these gene transcript, for example about 20-100 and any combination between, for example, 21, 22, 23, 24, 25, 26, 27, 28, 29, 30, and so on. Our preferred groups are the groups of 96 (Table 11), 84 (Table 12), 50 (Table 13), 36 (Table 14), 80 (Table 15), 535 (Table 16) and 20 (Table 17). In some instances, we have found that one can enhance the accuracy of the diagnosis by adding additional genes to any of these specific groups.

Naturally, following the teachings of the present invention, one may also include one or more of the genes and/or transcripts presented in Tables 11-17 into a kit or a system for a multicancer screening kit. For example, any one or more genes and or transcripts from Table 17 may be added as a lung cancer marker for a gene expression analysis.

When one uses these groups, the genes in the group are compared to a control or a control group. The control groups can be non-smokers, smokers, or former smokers. Preferably, one compares the gene transcripts or their expression product in the nasal epithelial cell sample of an individual against a similar group, except that the members of the control groups do not have the lung disorder, such as emphysema or lung cancer. For example, comparing can be performed in the nasal epithelial cell sample from a smoker against a control group of smokers who do not have lung cancer. When one compares the transcripts or expression products against the control for increased expression or decreased expression, which depends upon the particular gene and is set forth in the tables—not all the genes surveyed will show an increase or decrease. However, at least 50% of the genes surveyed must provide the described pattern. Greater reliability if obtained as the percent approaches 100%. Thus, in one embodiment, one wants at least 55%, 60%, 65%, 70%, 75%, 80%, 85%, 90%, 95%, 98%, 99% of the genes surveyed to show the altered pattern indicative of lung disease, such as lung cancer, as set forth in the tables as shown below.

The presently described gene expression profile can also be used to screen for individuals who are susceptible for lung cancer. For example, a smoker, who is over a certain age, for example over 40 years old, or a smoker who has smoked, for example, a certain number of years, may wish to be screened for lung cancer. The gene expression analysis from nasal epithelial cells as described herein can provide an accurate very early diagnosis for lung cancer. This is particularly useful in diagnosis of lung cancer, because the earlier the cancer is detected, the better the survival rate is.

For example, when we analyzed the gene expression results, we found, that if one applies a less stringent threshold, the group of 80 genes as presented in Table 15 are part of the most frequently chosen genes across 1000 statistical test runs (see Examples below for more details regarding the statistical testing). Using random data, we have shown that no random gene shows up more than 67 times out of 1000. Using such a cutoff, the 535 genes of Table 16 in our data show up more than 67 times out of 1000. All the 80 genes in Table 15 form a subset of the 535 genes. Table 17 shows the top 20 genes which are subset of the 535 list. The direction of change in expression is shown using signal to noise ratio. A negative number in Tables 15, 16, and 17 means that expression of this gene or transcript is up in lung cancer samples. Positive number in Table 15, 16, and 17, indicates that the expression of this gene or transcript is down in lung cancer.

Accordingly, any combination of the genes and/or transcripts of Table 16 can be used. In one embodiment, any combination of at least 5-10, 10-20, 20-30, 30-40, 40-50, 50-60, 60-70, 70-80, 80, 80-90, 90-100, 100-120, 120-140, 140-150, 150-160, 160-170, 170-180, 180-190, 190-200, 200-210, 210-220, 220-230, 230-240, 240-250, 250-260, 260-270, 270-280, 280-290, 290-300, 300-310, 310-320, 320-330, 330-340, 340-350, 350-360, 360-370, 370-380, 380-390, 390-400, 400-410, 410-420, 420-430, 430-440, 440-450, 450-460, 460-470, 470-480, 480-490, 490-500, 500-510, 510-520, 520-530, and up to about 535 genes selected from the group consisting of genes or transcripts as shown in the Table 16.

Table 17 provides 20 of the most frequently variably expressed genes in lung cancer when compared to samples without cancer. Accordingly, in one embodiment, any combination of about 3-5, 5-10, 11, 12, 13, 14, 15, 16, 17, 18, 19, or all 20 genes and/or transcripts of Table 17, or any sub-combination thereof are used.

In one embodiment, the invention provides a gene group the expression profile of nasal epithelial cells which is useful in diagnosing lung diseases and which comprises probes that hybridize ranging from 1 to 96 and all combinations in between for example 5, 10, 15, 20, 25, 30, 35, at least about 36, at least to 40, at least to 50, at least to 60, to at least 70, to at least 80, to at least 90, or all of the following 96 gene sequences: NM_003335; NM_000918; NM_006430.1; NM_001416.1; NM_004090; NM_006406.1; NM_003001.2; NM_001319; NM_006545.1; NM_021145.1; NM_002437.1; NM_006286; NM_001003698///NM_001003699///NM_002955; NM_001123///NM_006721; NM_024824; NM_004935.1; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_001696; NM_005494///NM_058246; NM_006534///NM_181659; NM_006368; NM_002268///NM_032771; NM_014033; NM_016138; NM_007048///NM_194441; NM_006694; NM_000051///NM_138292///NM_138293; NM_000410///NM_139002///NM_139003///NM_139004///NM_139005///NM_139006///NM_139007///NM_139008///NM_139009///NM_139010///NM_139011; NM_004691; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_012394; NM_019011///NM_207111///NM_207116; NM_017646; NM_021800; NM_016049; NM_014395; NM_014336; NM_018097; NM_019014; NM_024804; NM_018260; NM_018118; NM_014128; NM_024084; NM_005294; AF077053; NM_138387; NM_024531; NM_000693; NM_018509; NM_033128; NM_020706; AI523613; and NM_014884

In one embodiment, the invention provides a gene group the expression profile of nasal epithelial cells of which is useful in diagnosing lung diseases and comprises probes that hybridize to at least, for example, 5, 10, 15, 20, 25, 30, 35, at least about 36, at least to 40, at least to 50, at least to 60, to at least 70, to at least 80, to all of the following 84 gene sequences: NM_030757.1; R83000; AK021571.1; NM_014182.1; NM_17932.1; U85430.1; AI683552; BC002642.1; AW024467; NM_030972.1; BC021135.1; AL161952.1; AK026565.1; AK023783.1; BF218804; NM_001281.1; NM_024006.1; AK023843.1; BC001602.1; BC034707.1; BC064619.1; AY280502.1; BC059387.1; AF135421.1; BC061522.1; L76200.1; U50532.1; BC006547.2; BC008797.2; BC000807.1; AL080112.1; BC033718.1///BC046176.1///BC038443.1; NM_000346.1; BC008710.1; Hs.288575 (UNIGENE ID); AF020591.1; BC000423.2; BC002503.2; BC008710.1; BC009185.2; Hs.528304 (UNIGENE ID); U50532.1; BC013923.2; BC031091; NM_007062; Hs.249591 (Unigene ID); BC075839.1///BC073760.1; BC072436.1///BC004560.2; BC001016.2; Hs.286261 (Unigene ID); AF348514.1; BC005023.1; BC066337.1///BC058736.1///BC050555.1; Hs.216623 (Unigene ID); BC072400.1; BC041073.1; U43965.1; BC021258.2; BC016057.1; BC016713.1///BC014535.1///AF237771.1; BC000360.2; BC007455.2; BC000701.2; BC010067.2; BC023528.2///BC047680.1; BC064957.1; Hs.156701 (Unigene ID); BC030619.2; BC008710.1; U43965.1; BC066329.1; Hs.438867 (Unigene ID); BC035025.2///BC050330.1; BC023976.2; BC074852.2///BC074851.2; Hs.445885 (Unigene ID); BC008591.2///BC050440.1///; BC048096.1; AF365931.1; AF257099.1; and BC028912.1.

In one embodiment, the invention provides a gene group the expression profile of nasal epithelial cells which is useful in diagnosing lung diseases and comprises probes that hybridize to at least, for example 5, 10, 15, 20, 25, 30, preferably at least about 36, still more preferably at least to 40, still more preferably at least to 45, still more preferably all of the following 50 gene sequences, although it can include any and all members, for example, 20, 21, 22, up to and including 36: NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; AB014576.1; BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1. In one preferred embodiment, one can use at least 20-30, 30-40, of the 50 genes that overlap with the individual predictor genes identified in the analysis using the t-test, and, for example, 5-9 of the non-overlapping genes, identified using the t-test analysis as individual predictor genes, and combinations thereof.

In one embodiment, the invention provides a gene group the expression profile of nasal epithelial cells which is useful in diagnosing lung diseases and comprises probes that hybridize to at least for example 5, 10, 15, 20, preferably at least about 25, still more preferably at least to 30, still more preferably all of the following 36 gene sequences: NM_007062.1; NM_001281.1; BC002642.1; NM_000346.1; NM_006545.1; BG034328; NM_019067.1; NM_017925.1; NM_017932.1; NM_030757.1; NM_030972.1; NM_002268///NM_032771; NM_007048///NM_194441; NM_006694; U85430.1; NM_004691; AB014576.1; BF218804; BE467941; R83000; AL161952.1; AK023843.1; AK021571.1; AK023783.1; AL080112.1; AW971983; AI683552; NM_024006.1; AK026565.1; NM_014182.1; NM_021800.1; NM_016049.1; NM_021971.1; NM_014128.1; AA133341; and AF198444.1. In one preferred embodiment, one can use at least 20 of the 36 genes that overlap with the individual predictors and, for example, 5-9 of the non-overlapping genes, and combinations thereof.

The expression of the gene groups in an individual sample can be analyzed using any probe specific to the nucleic acid sequences or protein product sequences encoded by the gene group members. For example, in one embodiment, a probe set useful in the methods of the present invention is selected from the nucleic acid probes of between 10-15, 15-20, 20-180, preferably between 30-180, still more preferably between 36-96, still more preferably between 36-84, still more preferably between 36-50 probes, included in the Affymetrix Inc. gene chip of the Human Genome U133 Set and identified as probe ID Nos: 208082_x_at, 214800_x_at, 215208_x_at, 218556_at, 207730_x_at, 210556_at, 217679_x_at, 202901_x_at, 213939_s_at, 208137_x_at, 214705_at, 215001_s_at, 218155_x_at, 215604_x_at, 212297_at, 201804_x_at, 217949_s_at, 215179_x_at, 211316_x_at, 217653_x_at, 266_s_at, 204718_at, 211916_s_at, 215032_at, 219920_s_at, 211996_s_at, 200075_s_at, 214753_at, 204102_s_at, 202419_at, 214715_x_at, 216859_x_at, 215529_x_at, 202936_s_at, 212130_x_at, 215204_at, 218735_s_at, 200078_s_at, 203455_s_at, 212227_x_at, 222282_at, 219678_x_at, 208268_at, 221899_at, 213721_at, 214718_at, 201608_s_at, 205684_s_at, 209008_x_at, 200825_s_at, 218160_at, 57739_at, 211921_x_at, 218074_at, 200914_x_at, 216384_x_at, 214594_x_at, 222122_s_at, 204060_s_at, 215314_at, 208238_x_at, 210705_s_at, 211184_s_at, 215418_at, 209393_s_at, 210101_x_at, 212052_s_at, 215011_at, 221932_s_at, 201239_s_at, 215553_x_at, 213351_s_at, 202021_x_at, 209442_x_at, 210131_x_at, 217713_x_at, 214707_x_at, 203272_s_at, 206279_at, 214912_at, 201729_s_at, 205917_at, 200772_x_at, 202842_s_at, 203588_s_at, 209703_x_at, 217313_at, 217588_at, 214153_at, 222155_s_at, 203704_s_at, 220934_s_at, 206929_s_at, 220459_at, 215645_at, 217336_at, 203301_s_at, 207283_at, 222168_at, 222272_x_at, 219290_x_at, 204119_s_at, 215387_x_at, 222358_x_at, 205010_at, 1316_at, 216187_x_at, 208678_at, 222310_at, 210434_x_at, 220242_x_at, 207287_at, 207953_at, 209015_s_at, 221759_at, 220856_x_at, 200654_at, 220071_x_at, 216745_x_at, 218976_at, 214833_at, 202004_x_at, 209653_at, 210858_x_at, 212041_at, 221294_at, 207020_at, 204461_x_at, 205367_at, 219203_at, 215067_x_at, 212517_at, 220215_at, 201923_at, 215609_at, 207984_s_at, 215373_x_at, 216110_x_at, 215600_x_at, 216922_x_at, 215892_at, 201530_x_at, 217371_s_at, 222231_s_at, 218265_at, 201537_s_at, 221616_s_at, 213106_at, 215336_at, 209770_at, 209061_at, 202573_at, 207064_s_at, 64371_at, 219977_at, 218617_at, 214902_x_at, 207436_x_at, 215659_at, 204216_s_at, 214763_at, 200877_at, 218425_at, 203246_s_at, 203466_at, 204247_s_at, 216012_at, 211328_x_at, 218336_at, 209746_s_at, 214722_at, 214599_at, 220113_x_at, 213212_x_at, 217671_at, 207365_x_at, 218067_s_at, 205238_at, 209432_s_at, and 213919_at. In one preferred embodiment, one can use at least, for example, 10-20, 20-30, 30-40, 40-50, 50-60, 60-70, 70-80, 80-90, 90-100, 110, 120, 130, 140, 150, 160, or 170 of the 180 genes that overlap with the individual predictor genes and, for example, 5-9 of the non-overlapping genes and combinations thereof.

Sequences for the Affymetrix probes are available from Affymetrix. Other probes and sequences that recognize the genes of interest can be easily prepared using, e.g. synthetic oligonucleotides recombinant oligonucleotides. These sequences can be selected from any, preferably unique part of the gene based on the sequence information publicly available for the genes that are indicated by their HUGO ID, GenBank No. or Unigene No.

One can analyze the expression data to identify expression patters associated with any lung disease. For example, one can analyze diseases caused by exposure to air pollutants, such as cigarette smoke, asbestos or any other pollutant. For example, the analysis can be performed as follows. One first scans a gene chip or mixture of beads comprising probes that are hybridized with a study group samples. For example, one can use samples of non-smokers and smokers, non-asbestos exposed individuals and asbestos-exposed individuals, non-smog exposed individuals and smog-exposed individuals, smokers without a lung disease and smokers with lung disease, to obtain the differentially expressed gene groups between individuals with no lung disease and individuals with lung disease. One must, of course select appropriate groups, wherein only one air pollutant can be selected as a variable. So, for example, one can compare non-smokers exposed to asbestos but not smog and non-smokers not exposed to asbestos or smog.

The obtained expression analysis, such as microarray or microbead raw data consists of signal strength and detection p-value. One normalizes or scales the data, and filters the poor quality chips/bead sets based on images of the expression data, control probes, and histograms. One also filters contaminated specimens which contain non-epithelial cells. Lastly, one filters the genes of importance using detection p-value. This results in identification of transcripts present in normal airways (normal airway transcriptome). Variability and multiple regression analysis can be used. This also results in identification of effects of smoking on airway epithelial cell transcription. For this analysis, one can use T-test and Pearson correlation analysis. One can also identify a group or a set of transcripts that are differentially expressed in samples with lung disease, such as lung cancer and samples without cancer. This analysis was performed using class prediction models.

For analysis of the data, one can use, for example, a weighted voting method. The weighted voting method ranks, and gives a weight “p” to all genes by the signal to noise ration of gene expression between two classes: P=mean_{(class 1)}−mean_{(class 2)}/sd_{(class 1)}=sd_{(class 2)}. Committees of variable sizes of the top ranked genes are used to evaluate test samples, but genes with more significant p-values can be more heavily weighed. Each committee genes in test sample votes for one class or the other, based on how close that gene expression level is to the class 1 mean or the class 2 mean. V_{(gene A)}=P_{(gene A)}, i.e. level of expression in test sample less the average of the mean expression values in the two classes. Votes for each class are tallied and the winning class is determined along with prediction strength as PS=V_{win}−Vₗₒₛₑ/V_{win}+Vₗₒₛₑ. Finally, the accuracy can be validated using cross-validation+/−independent samples.

Table 11 shows 96 genes that were identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer. In one embodiment, the exemplary probes shown in the column “Affymetrix Id in the Human Genome U133 chip” can be used.

TABLE 11
96 Gene Group

| Affymetrix | | | Expression |
|---|---|---|---|
| ID for an | | | in cancer |
| example probe | | | compared |
| identifying | | | to a sample |
| the gene | GenBank ID | Gene Name | with no cancer. |
| 1316_at | NM_003335 | UBE1L | down |
| 200654_at | NM_000918 | P4HB | up |
| 200877_at | NM_006430.1 | CCT4 | up |
| 201530_x_at | NM_001416.1 | EIF4A1 | up |
| 201537_s_at | NM_004090 | DUSP3 | up |
| 201923_at | NM_006406.1 | PRDX4 | up |
| 202004_x_at | NM_003001.2 | SDHC | up |
| 202573_at | NM_001319 | CSNK1G2 | down |
| 203246_s_at | NM_006545.1 | TUSC4 | up |
| 203301_s_at | NM_021145.1 | DMTF1 | down |
| 203466_at | NM_002437.1 | MPV17 | up |
| 203588_s_at | NM_006286 | TFDP2 | up |
| 203704_s_at | NM_001003698 /// | RREB1 | down |
| | NM_001003699 /// | | |
| | NM_002955 | | |
| 204119_s_at | NM_001123 /// | ADK | up |
| | NM_006721 | | |
| 204216_s_at | NM_024824 | FLJ11806 | up |
| 204247_s_at | NM_004935.1 | CDK5 | up |
| 204461_x_at | NM_002853.1 | RAD1 | down |
| 205010_at | NM_019067.1 | FLJ10613 | down |
| 205238_at | NM_024917.1 | CXorf34 | down |
| 205367_at | NM_020979.1 | APS | down |
| 206929_s_at | NM_005597.1 | NFIC | down |
| 207020_at | NM_007031.1 | HSF2BP | down |
| 207064_s_at | NM_009590.1 | AOC2 | down |
| 207283_at | NM_020217.1 | DKFZp547I014 | down |
| 207287_at | NM_025026.1 | FLJ14107 | down |
| 207365_x_at | NM_014709.1 | USP34 | down |
| 207436_x_at | NM_014896.1 | KIAA0894 | down |
| 207953_at | AF010144 | — | down |
| 207984_s_at | NM_005374.1 | MPP2 | down |
| 208678_at | NM_001696 | ATP6V1E1 | up |
| 209015_s_at | NM_005494 /// | DNAJB6 | up |
| | NM_058246 | | |
| 209061_at | NM_006534 /// | NCOA3 | down |
| | NM_181659 | | |
| 209432_s_at | NM_006368 | CREB3 | up |
| 209653_at | NM_002268 /// | KPNA4 | up |
| | NM_032771 | | |
| 209703_x_at | NM_014033 | DKFZP586A0522 | down |
| 209746_s_at | NM_016138 | COQ7 | down |
| 209770_at | NM_007048 /// | BTN3A1 | down |
| | NM_194441 | | |
| 210434_x_at | NM_006694 | JTB | up |
| 210858_x_at | NM_000051 /// | ATM | down |
| | NM_138292 /// | | |
| | NM_138293 | | |
| 211328_x_at | NM_000410 /// | HFE | down |
| | NM_139002 /// | | |
| | NM_139003 /// | | |
| | NM_139004 /// | | |
| | NM_139005 /// | | |
| | NM_139006 /// | | |
| | NM_139007 /// | | |
| | NM_139008 /// | | |
| | NM_139009 /// | | |
| | NM_139010 /// | | |
| | NM_139011 | | |
| 212041_at | NM_004691 | ATP6V0D1 | up |
| 212517_at | NM_012070 /// | ATRN | down |
| | NM_139321 /// | | |
| | NM_139322 | | |
| 213106_at | NM_006095 | ATP8A1 | down |
| 213212_x_at | AI632181 | — | down |
| 213919_at | AW024467 | — | down |
| 214153_at | NM_021814 | ELOVL5 | down |
| 214599_at | NM_005547.1 | IVL | down |
| 214722_at | NM_203458 | N2N | down |
| 214763_at | NM_015547 /// | THEA | down |
| | NM_147161 | | |
| 214833_at | AB007958.1 | K1AA0792 | down |
| 214902_x_at | NM_207488 | FLJ42393 | down |
| 215067_x_at | NM_005809 /// | PRDX2 | down |
| | NM_181737 /// | | |
| | NM_181738 | | |
| 215336_at | NM_016248 /// | AKAP11 | down |
| | NM_144490 | | |
| 215373_x_at | AK022213.1 | FLJ12151 | down |
| 215387_x_at | NM_005708 | GPC6 | down |
| 215600_x_at | NM_207102 | FBXW12 | down |
| 215609_at | AK023895 | — | down |
| 215645_at | NM_144606 /// | FLCN | down |
| | NM_144997 | | |
| 215659_at | NM_018530 | GSDML | down |
| 215892_at | AK021474 | — | down |
| 216012_at | U43604.1 | — | down |
| 216110_x_at | AU147017 | — | down |
| 216187_x_at | AF222691.1 | LNX1 | down |
| 216745_x_at | NM_015116 | LRCH1 | down |
| 216922_x_at | NM_001005375 /// | DAZ2 | down |
| | NM_001005785 /// | | |
| | NM_001005786 /// | | |
| | NM_004081 /// | | |
| | NM_020363 /// | | |
| | NM_020364 /// | | |
| | NM_020420 | | |
| 217313_at | AC004692 | — | down |
| 217336_at | NM_001014 | RPS10 | down |
| 217371_s_at | NM_000585 /// | IL15 | down |
| | NM_172174 /// | | |
| | NM_172175 | | |
| 217588_at | NM_054020 /// | CATSPER2 | down |
| | NM_172095 /// | | |
| | NM_172096 /// | | |
| | NM_172097 | | |
| 217671_at | BE466926 | — | down |
| 218067_s_at | NM_018011 | FLJ10154 | down |
| 218265_at | NM_024077 | SECISBP2 | down |
| 218336_at | NM_012394 | PFDN2 | up |
| 218425_at | NM_019011 /// | TRIAD3 | down |
| | NM_207111 /// | | |
| | NM_207116 | | |
| 218617_at | NM_017646 | TRIT1 | down |
| 218976_at | NM_021800 | DNAJC12 | up |
| 219203_at | NM_016049 | C14orf122 | up |
| 219290_x_at | NM_014395 | DAPP1 | down |
| 219977_at | NM_014336 | AIPL1 | down |
| 220071_x_at | NM_018097 | C15orf25 | down |
| 220113_x_at | NM_019014 | POLR1B | down |
| 220215_at | NM_024804 | FLJ12606 | down |
| 220242_x_at | NM_018260 | FLJ10891 | down |
| 220459_at | NM_018118 | MCM3APAS | down |
| 220856_x_at | NM_014128 | | down |
| 220934_s_at | NM_024084 | MGC3196 | down |
| 221294_at | NM_005294 | GPR21 | down |
| 221616_s_at | AF077053 | PGK1 | down |
| 221759_at | NM_138387 | G6PC3 | up |
| 222155_s_at | NM_024531 | GPR172A | up |
| 222168_at | NM_000693 | ALDH1A3 | down |
| 222231_s_at | NM_018509 | PRO1855 | up |
| 222272_x_at | NM_033128 | SCIN | down |
| 222310_at | NM_020706 | SFRS15 | down |
| 222358_x_at | AI523613 | — | down |
| 64371_at | NM_014884 | SFRS14 | down |

Table 12 shows one preferred 84 gene group that has been identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer. These genes were identified using traditional Student's t-test analysis.

In one embodiment, the exemplary probes shown in the column “Affymetrix Id in the Human Genome U133 chip” can be used in the expression analysis.

TABLE 12
84 Gene Group

| | | Direction | |
|---|---|---|---|
| GenBank ID | | in Cancer | |
| (unless | | compared to | |
| otherwise | Gene Name | a non-cancer | Affymetrix |
| mentioned) | Abbreviation | sample | ID |
| NM_030757.1 | MKRN4 | down | 208082_x_at |
| R83000 | BTF3 | down | 214800_x_at |
| AK021571.1 | MUC20 | down | 215208_x_at |
| NM_014182.1 | ORMDL2 | up | 218556_at |
| NM_17932.1 | FLJ20700 | down | 207730_x_at |
| U85430.1 | NFATC3 | down | 210556_at |
| AI683552 | — | down | 217679_x_at |
| BC002642.1 | CTSS | down | 202901_x_at |
| AW024467 | RIPX | down | 213939_s_at |
| NM_030972.1 | MGC5384 | down | 208137_x_at |
| BC021135.1 | INADL | down | 214705_at |
| AL161952.1 | GLUL | down | 215001_s_at |
| AK026565.1 | FLJ10534 | down | 218155_x_at |
| AK023783.1 | — | down | 215604_x_at |
| BF218804 | AFURS1 | down | 212297_at |
| NM_001281.1 | CKAP1 | up | 201804_x_at |
| NM_024006.1 | IMAGE3455200 | up | 217949_s_at |
| AK023843.1 | PGF | down | 215179_x_at |
| BC001602.1 | CFLAR | down | 211316_x_at |
| BC034707.1 | — | down | 217653_x_at |
| BC064619.1 | CD24 | down | 266_s_at |
| AY280502.1 | EPHB6 | down | 204718_at |
| BC059387.1 | MYO1A | down | 211916_s_at |
| | — | down | 215032_at |
| AF135421.1 | GMPPB | up | 219920_s_at |
| BC061522.1 | MGC70907 | down | 211996_s_at |
| L76200.1 | GUK1 | up | 200075_s_at |
| U50532.1 | CG005 | down | 214753_at |
| BC006547.2 | EEF2 | down | 204102_s_at |
| BC008797.2 | FVT1 | down | 202419_at |
| BC000807.1 | ZNF160 | down | 214715_x_at |
| AL080112.1 | — | down | 216859_x_at |
| BC033718.1 /// | C21orf106 | down | 215529_x_at |
| BC046176.1 /// | | | |
| BC038443.1 | | | |
| NM_000346.1 | SOX9 | up | 202936_s_at |
| BC008710.1 | SUI1 | up | 212130_x_at |
| Hs.288575 | — | down | 215204_at |
| (Unigene ID) | | | |
| AF020591.1 | AF020591 | down | 218735_s_at |
| BC000423.2 | ATP6V0B | up | 200078_s_at |
| BC002503.2 | SAT | down | 203455_s_at |
| BC008710.1 | SUI1 | up | 212227 x at |
| | — | down | 222282_at |
| BC009185.2 | DCLRE1C | down | 219678_x_at |
| Hs.528304 | ADAM28 | down | 208268_at |
| (UNIGENE ID) | | | |
| U50532.1 | CG005 | down | 221899_at |
| BC013923.2 | SOX2 | down | 213721_at |
| BC031091 | ODAG | down | 214718_at |
| NM_007062 | PWP1 | up | 201608_s_at |
| Hs.249591 | FLJ20686 | down | 205684_s_at |
| (Unigene ID) | | | |
| BC075839.1 /// | KRT8 | up | 209008_x_at |
| BC073760.1 | | | |
| BC072436.1 /// | HYOU1 | up | 200825_s_at |
| BC004560.2 | | | |
| BC001016.2 | NDUFA8 | up | 218160_at |
| Hs.286261 | FLJ20195 | down | 57739_at |
| (Unigene ID) | | | |
| AF348514.1 | — | down | 211921_x_at |
| BC005023.1 | CGI-128 | up | 218074_at |
| BC066337.1 /// | KTN1 | down | 200914_x_at |
| BC058736.1 /// | | | |
| BC050555.1 | | | |
| | — | down | 216384_x_at |
| Hs.216623 | ATP8B1 | down | 214594_x_at |
| (Unigene ID) | | | |
| BC072400.1 | THOC2 | down | 222122_s_at |
| BC041073.1 | PRKX | down | 204060_s_at |
| U43965.1 | ANK3 | down | 215314_at |
| | — | down | 208238_x_at |
| BC021258.2 | TRIM5 | down | 210705_s_at |
| BC016057.1 | USH1C | down | 211184_s_at |
| BC016713.1 /// | PARVA | down | 215418_at |
| BC014535.1 /// | | | |
| AF237771.1 | | | |
| BC000360.2 | EIF4EL3 | up | 209393_s_at |
| BC007455.2 | SH3GLB1 | up | 210101_x_at |
| BC000701.2 | KIAA0676 | down | 212052_s_at |
| BC010067.2 | CHC1 | down | 215011_at |
| BC023528.2 /// | C14orf87 | up | 221932_s_at |
| BC047680.1 | | | |
| BC064957.1 | KIAA0102 | up | 201239_s_at |
| Hs.156701 | — | down | 215553_x_at |
| (Unigene ID) | | | |
| BC030619.2 | KIAA0779 | down | 213351_s_at |
| BC008710.1 | SUI1 | up | 202021_x_at |
| U43965.1 | ANK3 | down | 209442_x_at |
| BC066329.1 | SDHC | up | 210131_x_at |
| Hs.438867 | — | down | 217713_x_at |
| (Unigene ID) | | | |
| BC035025.2 /// | ALMS1 | down | 214707_x_at |
| BC050330.1 | | | |
| BC023976.2 | PDAP2 | up | 203272_s_at |
| BC074852.2 /// | PRKY | down | 206279_at |
| BC074851.2 | | | |
| Hs.445885 | KIAA1217 | down | 214912_at |
| (Unigene ID) | | | |
| BC008591.2 /// | KIAA0100 | up | 201729_s_at |
| BC050440.1 /// | | | |
| BC048096.1 | | | |
| AF365931.1 | ZNF264 | down | 205917_at |
| AF257099.1 | PTMA | down | 200772_x_at |
| BC028912.1 | DNAJB9 | up | 202842_s_at |

Table 13 shows one preferred 50 gene group that was identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer.

This gene group was identified using the GenePattern server from the Broad Institute, which includes the Weighted Voting algorithm. The default settings, i.e., the signal to noise ratio and no gene filtering, were used.

In one embodiment, the exemplary probes shown in the column “Affymetrix Id in the Human Genome U133 chip” can be used in the expression analysis.

TABLE 13
50 Gene Group

| GenBank ID | Gene Name | Direction in Cancer | Affymetrix ID |
|---|---|---|---|
| NM_007062.1 | PWP1 | up in cancer | 201608_s_at |
| NM_001281.1 | CKAP1 | up in cancer | 201804_x_at |
| BC000120.1 | | up in cancer | 202355_s_at |
| NM_014255.1 | TMEM4 | up in cancer | 202857_at |
| BC002642.1 | CTSS | up in cancer | 202901_x_at |
| NM_000346.1 | SOX9 | up in cancer | 202936_s_at |
| NM_006545.1 | NPR2L | up in cancer | 203246_s_at |
| BG034328 | | up in cancer | 203588_s_at |
| NM_021822.1 | APOBEC3G | up in cancer | 204205_at |
| NM_021069.1 | ARGBP2 | up in cancer | 204288_s_at |
| NM_019067.1 | FLJ10613 | up in cancer | 205010_at |
| NM_017925.1 | FLJ20686 | up in cancer | 205684_s_at |
| NM_017932.1 | FLJ20700 | up in cancer | 207730_x_at |
| NM_030757.1 | MKRN4 | up in cancer | 208082_x_at |
| NM_030972.1 | MGC5384 | up in cancer | 208137_x_at |
| AF126181.1 | BCG1 | up in cancer | 208682_s_at |
| U93240.1 | | up in cancer | 209653_at |
| U90552.1 | | up in cancer | 209770_at |
| AF151056.1 | | up in cancer | 210434_x_at |
| U85430.1 | NFATC3 | up in cancer | 210556_at |
| U51007.1 | | up in cancer | 211609_x_at |
| BC005969.1 | | up in cancer | 211759_x_at |
| NM_002271.1 | | up in cancer | 211954_s_at |
| AL566172 | | up in cancer | 212041_at |
| AB014576.1 | KIAA0676 | up in cancer | 212052_s_at |
| BF218804 | AFURS1 | down in cancer | 212297_at |
| AK022494.1 | | down in cancer | 212932_at |
| AA114843 | | down in cancer | 213884_s_at |
| BE467941 | | down in cancer | 214153_at |
| NM_003541.1 | HIST1H4K | down in cancer | 214463_x_at |
| R83000 | BTF3 | down in cancer | 214800_x_at |
| AL161952.1 | GLUL | down in cancer | 215001_s_at |
| AK023843.1 | PGF | down in cancer | 215179_x_at |
| AK021571.1 | MUC20 | down in cancer | 215208_x_at |
| AK023783.1 | — | down in cancer | 215604_x_at |
| AU147182 | | down in cancer | 215620_at |
| AL080112.1 | — | down in cancer | 216859_x_at |
| AW971983 | | down in cancer | 217588_at |
| AI683552 | — | down in cancer | 217679_x_at |
| NM_024006.1 | IMAGE3455200 | down in cancer | 217949_s_at |
| AK026565.1 | FLJ10534 | down in cancer | 218155_x_at |
| NM_014182.1 | ORMDL2 | down in cancer | 218556_at |
| NM_021800.1 | DNAJC12 | down in cancer | 218976_at |
| NM_016049.1 | CGI-112 | down in cancer | 219203_at |
| NM_019023.1 | PRMT7 | down in cancer | 219408_at |
| NM_021971.1 | GMPPB | down in cancer | 219920_s_at |
| NM_014128.1 | — | down in cancer | 220856_x_at |
| AK025651.1 | | down in cancer | 221648_s_at |
| AA133341 | C14orf87 | down in cancer | 221932_s_at |
| AF198444.1 | | down in cancer | 222168_at |

Table 14 shows one preferred 36 gene group that was identified as a group distinguishing smokers with cancer from smokers without cancer. The difference in expression is indicated at the column on the right as either “down”, which indicates that the expression of that particular transcript was lower in smokers with cancer than in smokers without cancer, and “up”, which indicates that the expression of that particular transcript was higher in smokers with cancer than smokers without cancer.

In one embodiment, the exemplary probes shown in the column “Affymetrix Id in the Human Genome U133 chip” can be used in the expression analysis.

TABLE 14
36 Gene Group

| GenBank ID | Gene Name | Affymetrix ID |
|---|---|---|
| NM_007062.1 | PWP1 | 201608_s_at |
| NM_001281.1 | CKAP1 | 201804_x_at |
| BC002642.1 | CTSS | 202901_x_at |
| NM_000346.1 | SOX9 | 202936_s_at |
| NM_006545.1 | NPR2L | 203246_s_at |
| BG034328 | | 203588_s_at |
| NM_019067.1 | FLJ10613 | 205010_at |
| NM_017925.1 | FLJ20686 | 205684_s_at |
| NM_017932.1 | FLJ20700 | 207730_x_at |
| NM_030757.1 | MKRN4 | 208082_x_at |
| NM_030972.1 | MGC5384 | 208137_x_at |
| NM_002268///NM_032771 | KPNA4 | 209653_at |
| NM_007048///NM_194441 | BTN3A1 | 209770_at |
| NM_006694 | JBT | 210434_x_at |
| U85430.1 | NFATC3 | 210556_at |
| NM_004691 | ATP6V0D1 | 212041_at |
| AB014576.1 | KIAA0676 | 212052_s_at |
| BF218804 | AFURS1 | 212297_at |
| BE467941 | | 214153_at |
| R83000 | BTF3 | 214800_x_at |
| AL161952.1 | GLUL | 215001_s_at |
| AK023843.1 | PGF | 215179_x_at |
| AK021571.1 | MUC20 | 215208_x_at |
| AK023783.1 | — | 215604_x_at |
| AL080112.1 | — | 216859_x_at |
| AW971983 | | 217588_at |
| AI683552 | — | 217679_x_at |
| NM_024006.1 | IMAGE3455200 | 217949_s_at |
| AK026565.1 | FLJ10534 | 218155_x_at |
| NM_014182.1 | ORMDL2 | 218556_at |
| NM_021800.1 | DNAJC12 | 218976_at |
| NM_016049.1 | CGI-112 | 219203_at |
| NM_021971.1 | GMPPB | 219920_s_at |
| NM_014128.1 | — | 220856_x_at |
| AA133341 | C14orf87 | 221932_s_at |
| AF198444.1 | | 222168_at |

In one embodiment, the gene group of the present invention comprises at least, for example, 5, 10, 15, 20, 25, 30, more preferably at least 36, still more preferably at least about 40, still more preferably at least about 50, still more preferably at least about 60, still more preferably at least about 70, still more preferably at least about 80, still more preferably at least about 86, still more preferably at least about 90, still more preferably at least about 96 of the genes as shown in Tables 11-14.

In one preferred embodiment, the gene group comprises 36-180 genes selected from the group consisting of the genes listed in Tables 11-14.

In one embodiment, the invention provides group of genes the expression of which is lower in individuals with cancer.

Accordingly, in one embodiment, the invention provides of a group of genes useful in diagnosing lung diseases, wherein the expression of the group of genes is lower in individuals exposed to air pollutants with cancer as compared to individuals exposed to the same air pollutant who do not have cancer, the group comprising probes that hybridize at least 5, preferably at least about 5-10, still more preferably at least about 10-20, still more preferably at least about 20-30, still more preferably at least about 30-40, still more preferably at least about 40-50, still more preferably at least about 50-60, still more preferably at least about 60-70, still more preferably about 72 genes consisting of transcripts (transcripts are identified using their GenBank ID or Unigene ID numbers and the corresponding gene names appear in Table 11): NM_003335; NM_001319; NM_021145.1; NM_001003698///NM_001003699///; NM_002955; NM_002853.1; NM_019067.1; NM_024917.1; NM_020979.1; NM_005597.1; NM_007031.1; NM_009590.1; NM_020217.1; NM_025026.1; NM_014709.1; NM_014896.1; AF010144; NM_005374.1; NM_006534///NM_181659; NM_014033; NM_016138; NM_007048///NM_194441; NM_000051///NM_138292///NM_138293; NM_000410///NM_139002///NM_139003///NM_139004///NM_139005///NM_139006///NM_139007///NM_139008///NM_139009///NM_139010///NM_139011; NM_012070///NM_139321///NM_139322; NM_006095; AI632181; AW024467; NM_021814; NM_005547.1; NM_203458; NM_015547///NM_147161; AB007958.1; NM_207488; NM_005809///NM_181737///NM_181738; NM_016248///NM_144490; AK022213.1; NM_005708; NM_207102; AK023895; NM_144606///NM_144997; NM_018530; AK021474; U43604.1; AU147017; AF222691.1; NM_015116; NM_001005375///NM_001005785///NM_001005786///NM_004081///NM_020363///NM_020364///NM_020420; AC004692; NM_001014; NM_000585///NM_172174///NM_172175; NM_054020///NM_172095///NM_172096///NM_172097; BE466926; NM_018011; NM_024077; NM_019011///NM_207111///NM_207116; NM_017646; NM_014395; NM_014336; NM_018097; NM_019014; NM_024804; NM_018260; NM_018118; NM_014128; NM_024084; NM_005294; AF077053; NM_000693; NM_033128; NM_020706; AI523613; and NM_014884.

In another embodiment, the invention provides of a group of genes useful in diagnosing lung diseases wherein the expression of the group of genes is lower in individuals exposed to air pollutants with cancer as compared to individuals exposed to the same air pollutant who do not have cancer, the group comprising probes that hybridize at least 5, preferably at least about 5-10, still more preferably at least about 10-20, still more preferably at least about 20-30, still more preferably at least about 30-40, still more preferably at least about 40-50, still more preferably at least about 50-60, still more preferably about 63 genes consisting of transcripts (transcripts are identified using their GenBank ID or Unigene ID numbers and the corresponding gene names appear in Table 12): NM_030757.1; R83000; AK021571.1; NM_17932.1; U85430.1; AI683552; BC002642.1; AW024467; NM_030972.1; BC021135.1; AL161952.1; AK026565.1; AK023783.1; BF218804; AK023843.1; BC001602.1; BC034707.1; BC064619.1; AY280502.1; BC059387.1; BC061522.1; U50532.1; BC006547.2; BC008797.2; BC000807.1; AL080112.1; BC033718.1///BC046176.1///; BC038443.1; Hs.288575 (UNIGENE ID); AF020591.1; BC002503.2; BC009185.2; Hs.528304 (UNIGENE ID); U50532.1; BC013923.2; BC031091; Hs.249591 (Unigene ID); Hs.286261 (Unigene ID); AF348514.1; BC066337.1///BC058736.1///BC050555.1; Hs.216623 (Unigene ID); BC072400.1; BC041073.1; U43965.1; BC021258.2; BC016057.1; BC016713.1///BC014535.1///AF237771.1; BC000701.2; BC010067.2; Hs.156701 (Unigene ID); BC030619.2; U43965.1; Hs.438867 (Unigene ID); BC035025.2///BC050330.1; BC074852.2///BC074851.2; Hs.445885 (Unigene ID); AF365931.1; and AF257099.1

In another embodiment, the invention provides of a group of genes useful in diagnosing lung diseases wherein the expression of the group of genes is lower in individuals exposed to air pollutants with cancer as compared to individuals exposed to the same air pollutant who do not have cancer, the group comprising probes that hybridize at least 5, preferably at least about 5-10, still more preferably at least about 10-20, still more preferably at least about 20-25, still more preferably about 25 genes consisting of transcripts (transcripts are identified using their GenBank ID or Unigene ID numbers and the corresponding gene names appear in Table 13):BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1.

In another embodiment, the invention provides of a group of genes useful in diagnosing lung diseases wherein the expression of the group of genes is higher in individuals exposed to air pollutants with cancer as compared to individuals exposed to the same air pollutant who do not have cancer, the group comprising probes that hybridize at least to 5, preferably at least about 5-10, still more preferably at least about 10-20, still more preferably at least about 20-25, still more preferably about 25 genes consisting of transcripts (transcripts are identified using their GenBank ID or Unigene ID numbers and the corresponding gene names appear in Table 11): NM_000918; NM_006430.1; NM_001416.1; NM_004090; NM_006406.1; NM_003001.2; NM_006545.1; NM_002437.1; NM_006286; NM_001123///NM_006721; NM_024824; NM_004935.1; NM_001696; NM_005494///NM_058246; NM_006368; NM_002268///NM_032771; NM_006694; NM_004691; NM_012394; NM_021800; NM_016049; NM_138387; NM_024531; and NM_018509.

In another embodiment, the invention provides of a group of genes useful in diagnosing lung diseases wherein the expression of the group of genes is higher in individuals exposed to air pollutants with cancer as compared to individuals exposed to the same air pollutant who do not have cancer, the group comprising probes that hybridize at least to 5, preferably at least about 5-10, still more preferably at least about 10-20, still more preferably at least about 20-23, still more preferably about 23 genes consisting of transcripts (transcripts are identified using their GenBank ID or Unigene ID numbers and the corresponding gene names appear in Table 12): NM_014182.1; NM_001281.1; NM_024006.1; AF135421.1; L76200.1; NM_000346.1; BC008710.1; BC000423.2; BC008710.1; NM_007062; BC075839.1///BC073760.1; BC072436.1///BC004560.2; BC001016.2; BC005023.1; BC000360.2; BC007455.2; BC023528.2///BC047680.1; BC064957.1; BC008710.1; BC066329.1; BC023976.2; BC008591.2///BC050440.1///BC048096.1; and BC028912.1.

In another embodiment, the invention provides of a group of genes useful in diagnosing lung diseases wherein the expression of the group of genes is higher in individuals exposed to air pollutants with cancer as compared to individuals exposed to the same air pollutant who do not have cancer, the group comprising probes that hybridize at least to 5, preferably at least about 5-10, still more preferably at least about 10-20, still more preferably at least about 20-25, still more preferably about 25 genes consisting of transcripts (transcripts are identified using their GenBank ID or Unigene ID numbers and the corresponding gene names appear in Table 13): NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; and AB014576.1.

In one embodiment, the invention provides a method of diagnosing lung disease comprising the steps of measuring the expression profile of a gene group in an individual suspected of being affected or being at high risk of a lung disease (i.e. test individual), and comparing the expression profile (i.e. control profile) to an expression profile of an individual without the lung disease who has also been exposed to similar air pollutant than the test individual (i.e. control individual), wherein differences in the expression of genes when compared between the afore mentioned test individual and control individual of at least 10, more preferably at least 20, still more preferably at least 30, still more preferably at least 36, still more preferably between 36-180, still more preferably between 36-96, still more preferably between 36-84, still more preferably between 36-50, is indicative of the test individual being affected with a lung disease. Groups of about 36 genes as shown in table 14, about 50 genes as shown in table 13, about 84 genes as shown in table 12 and about 96 genes as shown in table 11 are preferred. The different gene groups can also be combined, so that the test individual can be screened for all, three, two, or just one group as shown in tables 11-14.

For example, if the expression profile of a test individual exposed to cigarette smoke is compared to the expression profile of the 50 genes shown in table 13, using the Affymetrix Inc. probe set on a gene chip as shown in table 13, the expression profile that is similar to the one shown for the individuals with cancer, is indicative that the test individual has cancer. Alternatively, if the expression profile is more like the expression profile of the individuals who do not have cancer, the test individual likely is not affected with lung cancer.

The group of 50 genes was identified using the GenePattern server from the Broad Institute, which includes the Weighted Voting algorithm. The default settings, i.e., the signal to noise ratio and no gene filtering, were used. GenePattern is available through the World Wide Wed at location broad.mit.edu/cancer/software/genepattern. This program allows analysis of data in groups rather than as individual genes. Thus, in one preferred embodiment, the expression of substantially all 50 genes of Table 13, are analyzed together. The expression profile of lower that normal expression of genes selected from the group consisting of BF218804; AK022494.1; AA114843; BE467941; NM_003541.1; R83000; AL161952.1; AK023843.1; AK021571.1; AK023783.1; AU147182; AL080112.1; AW971983; AI683552; NM_024006.1; AK026565.1; NM_014182.1; NM_021800.1; NM_016049.1; NM_019023.1; NM_021971.1; NM_014128.1; AK025651.1; AA133341; and AF198444.1, and the gene expression profile of higher than normal expression of genes selected from the group consisting of NM_007062.1; NM_001281.1; BC000120.1; NM_014255.1; BC002642.1; NM_000346.1; NM_006545.1; BG034328; NM_021822.1; NM_021069.1; NM_019067.1; NM_017925.1; NM_017932.1; NM_030757.1; NM_030972.1; AF126181.1; U93240.1; U90552.1; AF151056.1; U85430.1; U51007.1; BC005969.1; NM_002271.1; AL566172; and AB014576.1, is indicative of the individual having or being at high risk of developing lung disease, such as lung cancer. In one preferred embodiment, the expression pattern of all the genes in the Table 13 is analyzed. In one embodiment, in addition to analyzing the group of predictor genes of Table 13, 1, 2, 3, 4, 5, 6, 7, 8, 9, 10-15, 15-20, 20-30, or more of the individual predictor genes identified using the t-test analysis are analyzed. Any combination of, for example, 5-10 or more of the group predictor genes and 5-10, or more of the individual genes can also be used.

The term “expression profile” as used herein, refers to the amount of the gene product of each of the analyzed individual genes in the sample. The “expression profile” is like a signature expression map.

The term “individual”, as used herein, preferably refers to human. However, the methods are not limited to humans, and a skilled artisan can use the diagnostic/prognostic gene groupings of the present invention in, for example, laboratory test animals, preferably animals that have lungs, such as non-human primates, murine species, including, but not limited to rats and mice, dogs, sheep, pig, guinea pigs, and other model animals. Such laboratory tests can be used, for example in pre-clinical animal testing of drugs intended to be used to treat or prevent lung diseases.

In one embodiment, the group of genes the expression of which is analyzed in diagnosis and/or prognosis of lung cancer are selected from the group of 80 genes as shown in Table 15. Any combination of genes can be selected from the 80 genes. In one embodiment, the combination of 20 genes shown in Table 17 is selected. In one embodiment, a combination of genes from Table 16 is selected.

TABLE 15
Group of 80 genes for prognostic and diagnostic testing of lung cancer.

| Affymetrix | Gene symbol | Number of | Signal to noise in a |
|---|---|---|---|
| ID | (HUGO ID) | runs* | cancer sample** |
| 200729_s_at | ACTR2 | 736 | −0.22284 |
| 200760_s_at | ARL6IP5 | 483 | −0.21221 |
| 201399_s_at | TRAM1 | 611 | −0.21328 |
| 201444_s_at | ATP6AP2 | 527 | −0.21487 |
| 201635_s_at | FXR1 | 458 | −0.2162 |
| 201689_s_at | TPD52 | 565 | −0.22292 |
| 201925_s_at | DAF | 717 | −0.25875 |
| 201926_s_at | DAF | 591 | −0.23228 |
| 201946_s_at | CCT2 | 954 | −0.24592 |
| 202118_s_at | CPNE3 | 334 | −0.21273 |
| 202704_at | TOB1 | 943 | −0.25724 |
| 202833_s_at | SERPINA1 | 576 | −0.20583 |
| 202935_s_at | SOX9 | 750 | −0.25574 |
| 203413_at | NELL2 | 629 | −0.23576 |
| 203881_s_at | DMD | 850 | −0.24341 |
| 203908_at | SLC4A4 | 887 | −0.23167 |
| 204006_s_at | FCGR3A///FCGR3B | 207 | −0.20071 |
| 204403_x_at | KIAA0738 | 923 | 0.167772 |
| 204427_s_at | RNP24 | 725 | −0.2366 |
| 206056_x_at | SPN | 976 | 0.196398 |
| 206169_x_at | RoXaN | 984 | 0.259637 |
| 207730_x_at | HDGF2 | 969 | 0.169108 |
| 207756_at | — | 855 | 0.161708 |
| 207791_s_at | RAB1A | 823 | −0.21704 |
| 207953_at | AD7C-NTP | 1000 | 0.218433 |
| 208137_x_at | — | 996 | 0.191938 |
| 208246_x_at | TK2 | 982 | 0.179058 |
| 208654_s_at | CD164 | 388 | −0.21228 |
| 208892_s_at | DUSP6 | 878 | −0.25023 |
| 209189_at | FOS | 935 | −0.27446 |
| 209204_at | LMO4 | 78 | 0.158674 |
| 209267_s_at | SLC39A8 | 228 | −0.24231 |
| 209369_at | ANXA3 | 384 | −0.19972 |
| 209656_s_at | TMEM47 | 456 | −0.23033 |
| 209774_x_at | CXCL2 | 404 | −0.2117 |
| 210145_at | PLA2G4A | 475 | −0.26146 |
| 210168_at | C6 | 458 | −0.24157 |
| 210317_s_at | YWHAE | 803 | −0.29542 |
| 210397_at | DEFB1 | 176 | −0.22512 |
| 210679_x_at | — | 970 | 0.181718 |
| 211506_s_at | IL8 | 270 | −0.3105 |
| 212006_at | UBXD2 | 802 | −0.22094 |
| 213089_at | LOC153561 | 649 | 0.164097 |
| 213736_at | COX5B | 505 | 0.155243 |
| 213813_x_at | — | 789 | 0.178643 |
| 214007_s_at | PTK9 | 480 | −0.21285 |
| 214146_s_at | PPBP | 593 | −0.24265 |
| 214594_x_at | ATP8B1 | 962 | 0.284039 |
| 214707_x_at | ALMS1 | 750 | 0.164047 |
| 214715_x_at | ZNF160 | 996 | 0.198532 |
| 215204_at | SENP6 | 211 | 0.169986 |
| 215208_x_at | RPL35A | 999 | 0.228485 |
| 215385_at | FTO | 164 | 0.187634 |
| 215600_x_at | FBXW12 | 960 | 0.17329 |
| 215604_x_at | UBE2D2 | 998 | 0.224878 |
| 215609_at | STARD7 | 940 | 0.191953 |
| 215628_x_at | PPP2CA | 829 | 0.16391 |
| 215800_at | DUOX1 | 412 | 0.160036 |
| 215907_at | BACH2 | 987 | 0.178338 |
| 215978_x_at | LOC152719 | 645 | 0.163399 |
| 216834_at | — | 633 | −0.25508 |
| 216858_x_at | — | 997 | 0.232969 |
| 217446_x_at | — | 942 | 0.182612 |
| 217653_x_at | — | 976 | 0.270552 |
| 217679_x_at | — | 987 | 0.265918 |
| 217715_x_at | ZNF354A | 995 | 0.223881 |
| 217826_s_at | UBE2J1 | 812 | −0.23003 |
| 218155_x_at | FLJ10534 | 998 | 0.186425 |
| 218976_at | DNAJC12 | 486 | −0.22866 |
| 219392_x_at | FLJ11029 | 867 | 0.169113 |
| 219678_x_at | DCLRE1C | 877 | 0.169975 |
| 220199_s_at | FLJ12806 | 378 | −0.20713 |
| 220389_at | FLJ23514 | 102 | 0.239341 |
| 220720_x_at | FLJ14346 | 989 | 0.17976 |
| 221191_at | DKFZP434A0131 | 616 | 0.185412 |
| 221310_at | FGF14 | 511 | −0.19965 |
| 221765_at | — | 319 | −0.25025 |
| 222027_at | NUCKS | 547 | 0.171954 |
| 222104_x_at | GTF2H3 | 981 | 0.186025 |
| 222358_x_at | — | 564 | 0.194048 |

TABLE 16
Group of 535 genes useful in prognosis or diagnosis of lung cancer.

| | Gene symbol (HUGO | Number of | Signal to noise in a |
|---|---|---|---|
| Affymetrix ID | ID) | runs* | cancer sample** |
| 200729_s_at | ACTR2 | 736 | −0.22284 |
| 200760_s_at | ARL6IP5 | 483 | −0.21221 |
| 201399_s_at | TRAM1 | 611 | −0.21328 |
| 201444_s_at | ATP6AP2 | 527 | −0.21487 |
| 201635_s_at | FXR1 | 458 | −0.2162 |
| 201689_s_at | TPD52 | 565 | −0.22292 |
| 201925_s_at | DAF | 717 | −0.25875 |
| 201926_s_at | DAF | 591 | −0.23228 |
| 201946_s_at | CCT2 | 954 | −0.24592 |
| 202118_s_at | CPNE3 | 334 | −0.21273 |
| 202704_at | TOB1 | 943 | −0.25724 |
| 202833_s_at | SERPINA1 | 576 | −0.20583 |
| 202935_s_at | SOX9 | 750 | −0.25574 |
| 203413_at | NELL2 | 629 | −0.23576 |
| 203881_s_at | DMD | 850 | −0.24341 |
| 203908_at | SLC4A4 | 887 | −0.23167 |
| 204006_s_at | FCGR3A///FCGR3B | 207 | −0.20071 |
| 204403_x_at | KIAA0738 | 923 | 0.167772 |
| 204427_s_at | RNP24 | 725 | −0.2366 |
| 206056_x_at | SPN | 976 | 0.196398 |
| 206169_x_at | RoXaN | 984 | 0.259637 |
| 207730_x_at | HDGF2 | 969 | 0.169108 |
| 207756_at | — | 855 | 0.161708 |
| 207791_s_at | RAB1A | 823 | −0.21704 |
| 207953_at | AD7C−NTP | 1000 | 0.218433 |
| 208137_x_at | — | 996 | 0.191938 |
| 208246_x_at | TK2 | 982 | 0.179058 |
| 208654_s_at | CD164 | 388 | −0.21228 |
| 208892_s_at | DUSP6 | 878 | −0.25023 |
| 209189_at | FOS | 935 | −0.27446 |
| 209204_at | LMO4 | 78 | 0.158674 |
| 209267_s_at | SLC39A8 | 228 | −0.24231 |
| 209369_at | ANXA3 | 384 | −0.19972 |
| 209656_s_at | TMEM47 | 456 | −0.23033 |
| 209774_x_at | CXCL2 | 404 | −0.2117 |
| 210145_at | PLA2G4A | 475 | −0.26146 |
| 210168_at | C6 | 458 | −0.24157 |
| 210317_s_at | YWHAE | 803 | −0.29542 |
| 210397_at | DEFB1 | 176 | −0.22512 |
| 210679_x_at | — | 970 | 0.181718 |
| 211506_s_at | IL8 | 270 | −0.3105 |
| 212006_at | UBXD2 | 802 | −0.22094 |
| 213089_at | LOC153561 | 649 | 0.164097 |
| 213736_at | COX5B | 505 | 0.155243 |
| 213813_x_at | — | 789 | 0.178643 |
| 214007_s_at | PTK9 | 480 | −0.21285 |
| 214146_s_at | PPBP | 593 | −0.24265 |
| 214594_x_at | ATP8B1 | 962 | 0.284039 |
| 214707_x_at | ALMS1 | 750 | 0.164047 |
| 214715_x_at | ZNF160 | 996 | 0.198532 |
| 215204_at | SENP6 | 211 | 0.169986 |
| 215208_x_at | RPL35A | 999 | 0.228485 |
| 215385_at | FTO | 164 | 0.187634 |
| 215600_x_at | FBXW12 | 960 | 0.17329 |
| 215604_x_at | UBE2D2 | 998 | 0.224878 |
| 215609_at | STARD7 | 940 | 0.191953 |
| 215628_x_at | PPP2CA | 829 | 0.16391 |
| 215800_at | DUOX1 | 412 | 0.160036 |
| 215907_at | BACH2 | 987 | 0.178338 |
| 215978_x_at | LOC152719 | 645 | 0.163399 |
| 216834_at | — | 633 | −0.25508 |
| 216858_x_at | — | 997 | 0.232969 |
| 217446_x_at | — | 942 | 0.182612 |
| 217653_x_at | — | 976 | 0.270552 |
| 217679_x_at | — | 987 | 0.265918 |
| 217715_x_at | ZNF354A | 995 | 0.223881 |
| 217826_s_at | UBE2J1 | 812 | −0.23003 |
| 218155_x_at | FLJ10534 | 998 | 0.186425 |
| 218976_at | DNAJC12 | 486 | −0.22866 |
| 219392_x_at | FLJ11029 | 867 | 0.169113 |
| 219678_x_at | DCLRE1C | 877 | 0.169975 |
| 220199_s_at | FLJ12806 | 378 | −0.20713 |
| 220389_at | FLJ23514 | 102 | 0.239341 |
| 220720_x_at | FLJ14346 | 989 | 0.17976 |
| 221191_at | DKFZP434A0131 | 616 | 0.185412 |
| 221310_at | FGF14 | 511 | −0.19965 |
| 221765_at | — | 319 | −0.25025 |
| 222027_at | NUCKS | 547 | 0.171954 |
| 222104_x_at | GTF2H3 | 981 | 0.186025 |
| 222358_x_at | — | 564 | 0.194048 |
| 202113_s_at | SNX2 | 841 | −0.20503 |
| 207133_x_at | ALPK1 | 781 | 0.155812 |
| 218989_x_at | SLC30A5 | 765 | −0.198 |
| 200751_s_at | HNRPC | 759 | −0.19243 |
| 220796_x_at | SLC35E1 | 691 | 0.158199 |
| 209362_at | SURB7 | 690 | −0.18777 |
| 216248_s_at | NR4A2 | 678 | −0.19796 |
| 203138_at | HAT1 | 669 | −0.18115 |
| 221428_s_at | TBL1XR1 | 665 | −0.19331 |
| 218172_s_at | DERL1 | 665 | −0.16341 |
| 215861_at | FLJ14031 | 651 | 0.156927 |
| 209288_s_at | CDC42EP3 | 638 | −0.20146 |
| 214001_x_at | RPS10 | 634 | 0.151006 |
| 209116_x_at | HBB | 626 | −0.12237 |
| 215595_x_at | GCNT2 | 625 | 0.136319 |
| 208891_at | DUSP6 | 617 | −0.17282 |
| 215067_x_at | PRDX2 | 616 | 0.160582 |
| 202918_s_at | PREI3 | 614 | −0.17003 |
| 211985_s_at | CALM1 | 614 | −0.20103 |
| 212019_at | RSL1D1 | 601 | 0.152717 |
| 216187_x_at | KNS2 | 591 | 0.14297 |
| 215066_at | PTPRF | 587 | 0.143323 |
| 212192_at | KCTD12 | 581 | −0.17535 |
| 217586_x_at | — | 577 | 0.147487 |
| 203582_s_at | RAB4A | 567 | −0.18289 |
| 220113_x_at | POLR1B | 563 | 0.15764 |
| 217232_x_at | HBB | 561 | −0.11398 |
| 201041_s_at | DUSP1 | 560 | −0.18661 |
| 211450_s_at | MSH6 | 544 | −0.15597 |
| 202648_at | RPS19 | 533 | 0.150087 |
| 202936_s_at | SOX9 | 533 | −0.17714 |
| 204426_at | RNP24 | 526 | −0.18959 |
| 206392_s_at | RARRES1 | 517 | −0.18328 |
| 208750_s_at | ARF1 | 515 | −0.19797 |
| 202089_s_at | SLC39A6 | 512 | −0.19904 |
| 211297_s_at | CDK7 | 510 | −0.15992 |
| 215373_x_at | FLJ12151 | 509 | 0.146742 |
| 213679_at | FLJ13946 | 492 | −0.10963 |
| 201694_s_at | EGR1 | 490 | −0.19478 |
| 209142_s_at | UBE2G1 | 487 | −0.18055 |
| 217706_at | LOC220074 | 483 | 0.11787 |
| 212991_at | FBXO9 | 476 | 0.148288 |
| 201289_at | CYR61 | 465 | −0.19925 |
| 206548_at | FLJ23556 | 465 | 0.141583 |
| 202593_s_at | MIR16 | 462 | −0.17042 |
| 202932_at | YES1 | 461 | −0.17637 |
| 220575_at | FLJ11800 | 461 | 0.116435 |
| 217713_x_at | DKFZP566N034 | 452 | 0.145994 |
| 211953_s_at | RANBP5 | 447 | −0.17838 |
| 203827_at | WIPI49 | 447 | −0.17767 |
| 221997_s_at | MRPL52 | 444 | 0.132649 |
| 217662_x_at | BCAP29 | 434 | 0.116886 |
| 218519_at | SLC35A5 | 428 | −0.15495 |
| 214833_at | KIAA0792 | 428 | 0.132943 |
| 201339_s_at | SCP2 | 426 | −0.18605 |
| 203799_at | CD302 | 422 | −0.16798 |
| 211090_s_at | PRPF4B | 421 | −0.1838 |
| 220071_x_at | C15orf25 | 420 | 0.138308 |
| 203946_s_at | ARG2 | 415 | −0.14964 |
| 213544_at | ING1L | 415 | 0.137052 |
| 209908_s_at | — | 414 | 0.131346 |
| 201688_s_at | TPD52 | 410 | −0.18965 |
| 215587_x_at | BTBD14B | 410 | 0.139952 |
| 201699_at | PSMC6 | 409 | −0.13784 |
| 214902_x_at | FLJ42393 | 409 | 0.140198 |
| 214041_x_at | RPL37A | 402 | 0.106746 |
| 203987_at | FZD6 | 392 | −0.19252 |
| 211696_x_at | HBB | 392 | −0.09508 |
| 218025_s_at | PECI | 389 | −0.18002 |
| 215852_x_at | KIAA0889 | 382 | 0.12243 |
| 209458_x_at | HBA1///HBA2 | 380 | −0.09796 |
| 219410_at | TMEM45A | 379 | −0.22387 |
| 215375_x_at | — | 379 | 0.148377 |
| 206302_s_at | NUDT4 | 376 | −0.18873 |
| 208783_s_at | MCP | 372 | −0.15076 |
| 211374_x_at | — | 364 | 0.131101 |
| 220352_x_at | MGC4278 | 364 | 0.152722 |
| 216609_at | TXN | 363 | 0.15162 |
| 201942_s_at | CPD | 363 | −0.1889 |
| 202672_s_at | ATF3 | 361 | −0.12935 |
| 204959_at | MNDA | 359 | −0.21676 |
| 211996_s_at | KIAA0220 | 358 | 0.144358 |
| 222035_s_at | PAPOLA | 353 | −0.14487 |
| 208808_s_at | HMGB2 | 349 | −0.15222 |
| 203711_s_at | HIBCH | 347 | −0.13214 |
| 215179_x_at | PGF | 347 | 0.146279 |
| 213562_s_at | SQLE | 345 | −0.14669 |
| 203765_at | GCA | 340 | −0.1798 |
| 214414_x_at | HBA2 | 336 | −0.08492 |
| 217497_at | ECGF1 | 336 | 0.123255 |
| 220924_s_at | SLC38A2 | 333 | −0.17315 |
| 218139_s_at | C14orf108 | 332 | −0.15021 |
| 201096_s_at | ARF4 | 330 | −0.18887 |
| 220361_at | FLJ12476 | 325 | −0.15452 |
| 202169_s_at | AASDHPPT | 323 | −0.15787 |
| 202527_s_at | SMAD4 | 322 | −0.18399 |
| 202166_s_at | PPP1R2 | 320 | −0.16402 |
| 204634_at | NEK4 | 319 | −0.15511 |
| 215504_x_at | — | 319 | 0.145981 |
| 202388_at | RGS2 | 315 | −0.14894 |
| 215553_x_at | WDR45 | 315 | 0.137586 |
| 200598_s_at | TRA1 | 314 | −0.19349 |
| 202435_s_at | CYP1B1 | 313 | 0.056937 |
| 216206_x_at | MAP2K7 | 313 | 0.10383 |
| 212582_at | OSBPL8 | 313 | −0.17843 |
| 216509_x_at | MLLT10 | 312 | 0.123961 |
| 200908_s_at | RPLP2 | 308 | 0.136645 |
| 215108_x_at | TNRC9 | 306 | −0.1439 |
| 213872_at | C6orf62 | 302 | −0.19548 |
| 214395_x_at | EEF1D | 302 | 0.128234 |
| 222156_x_at | CCPG1 | 301 | −0.14725 |
| 201426_s_at | VIM | 301 | −0.17461 |
| 221972_s_at | Cab45 | 299 | −0.1511 |
| 219957_at | — | 298 | 0.130796 |
| 215123_at | — | 295 | 0.125434 |
| 212515_s_at | DDX3X | 295 | −0.14634 |
| 203357_s_at | CAPN7 | 295 | −0.17109 |
| 211711_s_at | PTEN | 295 | −0.12636 |
| 206165_s_at | CLCA2 | 293 | −0.17699 |
| 213959_s_at | KIAA1005 | 289 | −0.16592 |
| 215083_at | PSPC1 | 289 | 0.147348 |
| 219630_at | PDZK1IP1 | 287 | −0.15086 |
| 204018_x_at | HBA1///HBA2 | 286 | −0.08689 |
| 208671_at | TDE2 | 286 | −0.17839 |
| 203427_at | ASF1A | 286 | −0.14737 |
| 215281_x_at | POGZ | 286 | 0.142825 |
| 205749_at | CYP1A1 | 285 | 0.107118 |
| 212585_at | OSBPL8 | 282 | −0.13924 |
| 211745_x_at | HBA1///HBA2 | 281 | −0.08437 |
| 208078_s_at | SNF1LK | 278 | −0.14395 |
| 218041_x_at | SLC38A2 | 276 | −0.17003 |
| 212588_at | PTPRC | 270 | −0.1725 |
| 212397_at | RDX | 270 | −0.15613 |
| 208268_at | ADAM28 | 269 | 0.114996 |
| 207194_s_at | ICAM4 | 269 | 0.127304 |
| 222252_x_at | — | 269 | 0.132241 |
| 217414_x_at | HBA2 | 266 | −0.08974 |
| 207078_at | MED6 | 261 | 0.1232 |
| 215268_at | KIAA0754 | 261 | 0.13669 |
| 221387_at | GPR147 | 261 | 0.128737 |
| 201337_s_at | VAMP3 | 259 | −0.17284 |
| 220218_at | C9orf68 | 259 | 0.125851 |
| 222356_at | TBL1Y | 259 | 0.126765 |
| 208579_x_at | H2BFS | 258 | −0.16608 |
| 219161_s_at | CKLF | 257 | −0.12288 |
| 202917_s_at | S100A8 | 256 | −0.19869 |
| 204455_at | DST | 255 | −0.13072 |
| 211672_s_at | ARPC4 | 254 | −0.17791 |
| 201132_at | HNRPH2 | 254 | −0.12817 |
| 218313_s_at | GALNT7 | 253 | −0.179 |
| 218930_s_at | FLJ11273 | 251 | −0.15878 |
| 219166_at | C14orf104 | 250 | −0.14237 |
| 212805_at | KIAA0367 | 248 | −0.16649 |
| 201551_s_at | LAMP1 | 247 | −0.18035 |
| 202599_s_at | NRIP1 | 247 | −0.16226 |
| 203403_s_at | RNF6 | 247 | −0.14976 |
| 214261_s_at | ADH6 | 242 | −0.1414 |
| 202033_s_at | RB1CC1 | 240 | −0.18105 |
| 203896_s_at | PLCB4 | 237 | −0.20318 |
| 209703_x_at | DKFZP586A0522 | 234 | 0.140153 |
| 211699_x_at | HBA1///HBA2 | 232 | −0.08369 |
| 210764_s_at | CYR61 | 231 | −0.13139 |
| 206391_at | RARRES1 | 230 | −0.16931 |
| 201312_s_at | SH3BGRL | 225 | −0.12265 |
| 200798_x_at | MCL1 | 221 | −0.13113 |
| 214912_at | — | 221 | 0.116262 |
| 204621_s_at | NR4A2 | 217 | −0.10896 |
| 217761_at | MTCBP-1 | 217 | −0.17558 |
| 205830_at | CLGN | 216 | −0.14737 |
| 218438_s_at | MED28 | 214 | −0.14649 |
| 207475_at | FABP2 | 214 | 0.097003 |
| 208621_s_at | VIL2 | 213 | −0.19678 |
| 202436_s_at | CYP1B1 | 212 | 0.042216 |
| 202539_s_at | HMGCR | 210 | −0.15429 |
| 210830_s_at | PON2 | 209 | −0.17184 |
| 211906_s_at | SERPINB4 | 207 | −0.14728 |
| 202241_at | TRIB1 | 207 | −0.10706 |
| 203594_at | RTCD1 | 207 | −0.13823 |
| 215863_at | TFR2 | 207 | 0.095157 |
| 221992_at | LOC283970 | 206 | 0.126744 |
| 221872_at | RARRES1 | 205 | −0.11496 |
| 219564_at | KCNJ16 | 205 | −0.13908 |
| 201329_s_at | ETS2 | 205 | −0.14994 |
| 214188_at | HIS1 | 203 | 0.1257 |
| 201667_at | GJA1 | 199 | −0.13848 |
| 201464_x_at | JUN | 199 | −0.09858 |
| 215409_at | LOC254531 | 197 | 0.094182 |
| 202583_s_at | RANBP9 | 197 | −0.13902 |
| 215594_at | — | 197 | 0.101007 |
| 214326_x_at | JUND | 196 | −0.1702 |
| 217140_s_at | VDAC1 | 196 | −0.14682 |
| 215599_at | SMA4 | 195 | 0.133438 |
| 209896_s_at | PTPN11 | 195 | −0.16258 |
| 204846_at | CP | 195 | −0.14378 |
| 222303_at | — | 193 | −0.10841 |
| 218218_at | DIP13B | 193 | −0.12136 |
| 211015_s_at | HSPA4 | 192 | −0.13489 |
| 208666_s_at | ST13 | 191 | −0.13361 |
| 203191_at | ABCB6 | 190 | 0.096808 |
| 202731_at | PDCD4 | 190 | −0.1545 |
| 209027_s_at | ABI1 | 190 | −0.15472 |
| 205979_at | SCGB2A1 | 189 | −0.15091 |
| 216351_x_at | DAZ1///DAZ3/// | 189 | 0.106368 |
| | DAZ2///DAZ4 | | |
| 220240_s_at | C13orf11 | 188 | −0.16959 |
| 204482_at | CLDN5 | 187 | 0.094134 |
| 217234_s_at | VIL2 | 186 | −0.16035 |
| 214350_at | SNTB2 | 186 | 0.095723 |
| 201693_s_at | EGR1 | 184 | −0.10732 |
| 212328_at | KIAA1102 | 182 | −0.12113 |
| 220168_at | CASC1 | 181 | −0.1105 |
| 203628_at | IGF1R | 180 | 0.067575 |
| 204622_x_at | NR4A2 | 180 | −0.11482 |
| 213246_at | C14orf109 | 180 | −0.16143 |
| 218728_s_at | HSPC163 | 180 | −0.13248 |
| 214753_at | PFAAP5 | 179 | 0.130184 |
| 206336_at | CXCL6 | 178 | −0.05634 |
| 201445_at | CNN3 | 178 | −0.12375 |
| 209886_s_at | SMAD6 | 176 | 0.079296 |
| 213376_at | ZBTB1 | 176 | −0.17777 |
| 213887_s_at | POLR2E | 175 | −0.16392 |
| 204783_at | MLF1 | 174 | −0.13409 |
| 218824_at | FLJ10781 | 173 | 0.1394 |
| 212417_at | SCAMPI | 173 | −0.17052 |
| 202437_s_at | CYP1B1 | 171 | 0.033438 |
| 217528_at | CLCA2 | 169 | −0.14179 |
| 218170_at | ISOC1 | 169 | −0.14064 |
| 206278_at | PTAFR | 167 | 0.087096 |
| 201939_at | PLK2 | 167 | −0.11049 |
| 200907_s_at | KIAA0992 | 166 | −0.18323 |
| 207480_s_at | MEIS2 | 166 | −0.15232 |
| 201417_at | SOX4 | 162 | −0.09617 |
| 213826_s_at | — | 160 | 0.097313 |
| 214953_s_at | APP | 159 | −0.1645 |
| 204897_at | PTGER4 | 159 | −0.08152 |
| 201711_x_at | RANBP2 | 158 | −0.17192 |
| 202457_s_at | PPP3CA | 158 | −0.18821 |
| 206683_at | ZNF165 | 158 | −0.08848 |
| 214581_x_at | TNFRSF21 | 156 | −0.14624 |
| 203392_s_at | CTBP1 | 155 | −0.16161 |
| 212720_at | PAPOLA | 155 | −0.14809 |
| 207758_at | PPM1F | 155 | 0.090007 |
| 220995_at | STXBP6 | 155 | 0.106749 |
| 213831_at | HLA-DQA1 | 154 | 0.193368 |
| 212044_s_at | — | 153 | 0.098889 |
| 202434_s_at | CYP1B1 | 153 | 0.049744 |
| 206166_s_at | CLCA2 | 153 | −0.1343 |
| 218343_s_at | GTF3C3 | 153 | −0.13066 |
| 202557_at | STCH | 152 | −0.14894 |
| 201133_s_at | PJA2 | 152 | −0.18481 |
| 213605_s_at | MGC22265 | 151 | 0.130895 |
| 210947_s_at | MSH3 | 151 | −0.12595 |
| 208310_s_at | C7orf28A///C7orf28B | 151 | −0.15523 |
| 209307_at | — | 150 | −0.1667 |
| 215387_x_at | GPC6 | 148 | 0.114691 |
| 213705_at | MAT2A | 147 | 0.104855 |
| 213979_s_at | — | 146 | 0.121562 |
| 212731_at | LOC157567 | 146 | −0.1214 |
| 210117_at | SPAG1 | 146 | −0.11236 |
| 200641_s_at | YWHAZ | 145 | −0.14071 |
| 210701_at | CFDP1 | 145 | 0.151664 |
| 217152_at | NCOR1 | 145 | 0.130891 |
| 204224_s_at | GCH1 | 144 | −0.14574 |
| 202028_s_at | — | 144 | 0.094276 |
| 201735_s_at | CLCN3 | 144 | −0.1434 |
| 208447_s_at | PRPS1 | 143 | −0.14933 |
| 220926_s_at | C1orf22 | 142 | −0.17477 |
| 211505_s_at | STAU | 142 | −0.11618 |
| 221684_s_at | NYX | 142 | 0.102298 |
| 206906_at | ICAM5 | 141 | 0.076813 |
| 213228_at | PDE8B | 140 | −0.13728 |
| 217202_s_at | GLUL | 139 | −0.15489 |
| 211713_x_at | KIAA0101 | 138 | 0.108672 |
| 215012_at | ZNF451 | 138 | 0.13269 |
| 200806_s_at | HSPD1 | 137 | −0.14811 |
| 201466_s_at | JUN | 135 | −0.0667 |
| 211564_s_at | PDLIM4 | 134 | −0.12756 |
| 207850_at | CXCL3 | 133 | −0.17973 |
| 221841_s_at | KLF4 | 133 | −0.1415 |
| 200605_s_at | PRKAR1A | 132 | −0.15642 |
| 221198_at | SCT | 132 | 0.08221 |
| 201772_at | AZIN1 | 131 | −0.16639 |
| 205009_at | TFF1 | 130 | −0.17578 |
| 205542_at | STEAP1 | 129 | −0.08498 |
| 218195_at | C6orf211 | 129 | −0.14497 |
| 213642_at | — | 128 | 0.079657 |
| 212891_s_at | GADD45GIP1 | 128 | −0.09272 |
| 202798_at | SEC24B | 127 | −0.12621 |
| 222207_x_at | — | 127 | 0.10783 |
| 202638_s_at | ICAM1 | 126 | 0.070364 |
| 200730_s_at | PTP4A1 | 126 | −0.15289 |
| 219355_at | FLJ10178 | 126 | −0.13407 |
| 220266_s_at | KLF4 | 126 | −0.15324 |
| 201259_s_at | SYPL | 124 | −0.16643 |
| 209649_at | STAM2 | 124 | −0.1696 |
| 220094_s_at | C6orf79 | 123 | −0.12214 |
| 221751_at | PANK3 | 123 | −0.1723 |
| 200008_s_at | GDI2 | 123 | −0.15852 |
| 205078_at | PIGF | 121 | −0.13747 |
| 218842_at | FLJ21908 | 121 | −0.08903 |
| 202536_at | CHMP2B | 121 | −0.14745 |
| 220184_at | NANOG | 119 | 0.098142 |
| 201117_s_at | CPE | 118 | −0.20025 |
| 219787_s_at | ECT2 | 117 | −0.14278 |
| 206628_at | SLC5A1 | 117 | −0.12838 |
| 204007_at | FCGR3B | 116 | −0.15337 |
| 209446_s_at | — | 116 | 0.100508 |
| 211612_s_at | IL13RA1 | 115 | −0.17266 |
| 220992_s_at | C1orf25 | 115 | −0.11026 |
| 221899_at | PFAAP5 | 115 | 0.11698 |
| 221719_s_at | LZTS1 | 115 | 0.093494 |
| 201473_at | JUNB | 114 | −0.10249 |
| 221193_s_at | ZCCHC10 | 112 | −0.08003 |
| 215659_at | GSDML | 112 | 0.118288 |
| 205157_s_at | KRT17 | 111 | −0.14232 |
| 201001_s_at | UBE2V1///Kua-UEV | 111 | −0.16786 |
| 216789_at | — | 111 | 0.105386 |
| 205506_at | VIL1 | 111 | 0.097452 |
| 204875_s_at | GMDS | 110 | −0.12995 |
| 207191_s_at | ISLR | 110 | 0.100627 |
| 202779_s_at | UBE2S | 109 | −0.11364 |
| 210370_s_at | LY9 | 109 | 0.096323 |
| 202842_s_at | DNAJB9 | 108 | −0.15326 |
| 201082_s_at | DCTN1 | 107 | −0.10104 |
| 215588_x_at | RIOK3 | 107 | 0.135837 |
| 211076_x_at | DRPLA | 107 | 0.102743 |
| 210230_at | — | 106 | 0.115001 |
| 206544_x_at | SMARCA2 | 106 | −0.12099 |
| 208852_s_at | CANX | 105 | −0.14776 |
| 215405_at | MYO1E | 105 | 0.086393 |
| 208653_s_at | CD164 | 104 | −0.09185 |
| 206355_at | GNAL | 103 | 0.1027 |
| 210793_s_at | NUP98 | 103 | −0.13244 |
| 215070_x_at | RABGAP1 | 103 | 0.125029 |
| 203007_x_at | LYPLA1 | 102 | −0.17961 |
| 203841_x_at | MAPRE3 | 102 | −0.13389 |
| 206759_at | FCER2 | 102 | 0.081733 |
| 202232_s_at | GA17 | 102 | −0.11373 |
| 215892_at | — | 102 | 0.13866 |
| 214359_s_at | HSPCB | 101 | −0.12276 |
| 215810_x_at | DST | 101 | 0.098963 |
| 208937_s_at | ID1 | 100 | −0.06552 |
| 213664_at | SLC1A1 | 100 | −0.12654 |
| 219338_s_at | FLJ20156 | 100 | −0.10332 |
| 206595_at | CST6 | 99 | −0.10059 |
| 207300_s_at | F7 | 99 | 0.082445 |
| 213792_s_at | INSR | 98 | 0.137962 |
| 209674_at | CRY1 | 98 | −0.13818 |
| 40665_at | FMO3 | 97 | −0.05976 |
| 217975_at | WBP5 | 97 | −0.12698 |
| 210296_s_at | PXMP3 | 97 | −0.13537 |
| 215483_at | AKAP9 | 95 | 0.125966 |
| 212633_at | KIAA0776 | 95 | −0.16778 |
| 206164_at | CLCA2 | 94 | −0.13117 |
| 216813_at | — | 94 | 0.089023 |
| 208925_at | C3orf4 | 94 | −0.1721 |
| 219469_at | DNCH2 | 94 | −0.12003 |
| 206016_at | CXorf37 | 93 | −0.11569 |
| 216745_x_at | LRCH1 | 93 | 0.117149 |
| 212999_x_at | HLA-DQB1 | 92 | 0.110258 |
| 216859_x_at | — | 92 | 0.116351 |
| 201636_at | — | 92 | −0.13501 |
| 204272_at | LGALS4 | 92 | 0.110391 |
| 215454_x_at | SFTPC | 91 | 0.064918 |
| 215972_at | — | 91 | 0.097654 |
| 220593_s_at | FLJ20753 | 91 | 0.095702 |
| 222009_at | CGI-14 | 91 | 0.070949 |
| 207115_x_at | MBTD1 | 91 | 0.107883 |
| 216922_x_at | DAZ1///DAZ3/// | 91 | 0.086888 |
| | DAZ2///DAZ4 | | |
| 217626_at | AKR1C1///AKR1C2 | 90 | 0.036545 |
| 211429_s_at | SERPINA1 | 90 | −0.11406 |
| 209662_at | CETN3 | 90 | −0.10879 |
| 201629_s_at | ACP1 | 90 | −0.14441 |
| 201236_s_at | BTG2 | 89 | −0.09435 |
| 217137_x_at | — | 89 | 0.070954 |
| 212476_at | CENTB2 | 89 | −0.1077 |
| 218545_at | FLJ11088 | 89 | −0.12452 |
| 208857_s_at | PCMT1 | 89 | −0.14704 |
| 221931_s_at | SEH1L | 88 | −0.11491 |
| 215046_at | FLJ23861 | 88 | −0.14667 |
| 220222_at | PRO1905 | 88 | 0.081524 |
| 209737_at | AIP1 | 87 | −0.07696 |
| 203949_at | MPO | 87 | 0.113273 |
| 219290_x_at | DAPP1 | 87 | 0.111366 |
| 205116_at | LAMA2 | 86 | 0.05845 |
| 222316_at | VDP | 86 | 0.091505 |
| 203574_at | NFIL3 | 86 | −0.14335 |
| 207820_at | ADH1A | 86 | 0.104444 |
| 203751_x_at | JUND | 85 | −0.14118 |
| 202930_s_at | SUCLA2 | 85 | −0.14884 |
| 215404_x_at | FGFR1 | 85 | 0.119684 |
| 216266_s_at | ARFGEF1 | 85 | −0.12432 |
| 212806_at | KIAA0367 | 85 | −0.13259 |
| 219253_at | — | 83 | −0.14094 |
| 214605_x_at | GPR1 | 83 | 0.114443 |
| 205403_at | IL1R2 | 82 | −0.19721 |
| 222282_at | PAPD4 | 82 | 0.128004 |
| 214129_at | PDE4DIP | 82 | −0.13913 |
| 209259_s_at | CSPG6 | 82 | −0.12618 |
| 216900_s_at | CHRNA4 | 82 | 0.105518 |
| 221943_x_at | RPL38 | 80 | 0.086719 |
| 215386_at | AUTS2 | 80 | 0.129921 |
| 201990_s_at | CREBL2 | 80 | −0.13645 |
| 220145_at | FLJ21159 | 79 | −0.16097 |
| 221173_at | USH1C | 79 | 0.109348 |
| 214900_at | ZKSCAN1 | 79 | 0.075517 |
| 203290_at | HLA-DQA1 | 78 | −0.20756 |
| 215382_x_at | TPSAB1 | 78 | −0.09041 |
| 201631_s_at | IER3 | 78 | −0.12038 |
| 212188_at | KCTD12 | 77 | −0.14672 |
| 220428_at | CD207 | 77 | 0.101238 |
| 215349_at | — | 77 | 0.10172 |
| 213928_s_at | HRB | 77 | 0.092136 |
| 221228_s_at | — | 77 | 0.0859 |
| 202069_s_at | IDH3A | 76 | −0.14747 |
| 208554_at | POU4F3 | 76 | 0.107529 |
| 209504_s_at | PLEKHB1 | 76 | −0.13125 |
| 212989_at | TMEM23 | 75 | −0.11012 |
| 216197_at | ATF7IP | 75 | 0.115016 |
| 204748_at | PTGS2 | 74 | −0.15194 |
| 205221_at | HGD | 74 | 0.096171 |
| 214705_at | INADL | 74 | 0.102919 |
| 213939_s_at | RIPX | 74 | 0.091175 |
| 203691_at | PI3 | 73 | −0.14375 |
| 220532_s_at | LR8 | 73 | −0.11682 |
| 209829_at | C6orf32 | 73 | −0.08982 |
| 206515_at | CYP4F3 | 72 | 0.104171 |
| 218541_s_at | C8orf4 | 72 | −0.09551 |
| 210732_s_at | LGALS8 | 72 | −0.13683 |
| 202643_s_at | TNFAIP3 | 72 | −0.16699 |
| 218963_s_at | KRT23 | 72 | −0.10915 |
| 213304_at | KIAA0423 | 72 | −0.12256 |
| 202768_at | FOSB | 71 | −0.06289 |
| 205623_at | ALDH3A1 | 71 | 0.045457 |
| 206488_s_at | CD36 | 71 | −0.15899 |
| 204319_s_at | RGS10 | 71 | −0.10107 |
| 217811_at | SELT | 71 | −0.16162 |
| 202746_at | ITM2A | 70 | −0.06424 |
| 221127_s_at | RIG | 70 | 0.110593 |
| 209821_at | C9orf26 | 70 | −0.07383 |
| 220957_at | CTAGE1 | 70 | 0.092986 |
| 215577_at | UBE2E1 | 70 | 0.10305 |
| 214731_at | DKFZp547A023 | 70 | 0.102821 |
| 210512_s_at | VEGF | 69 | −0.11804 |
| 205267_at | POU2AF1 | 69 | 0.101353 |
| 216202_s_at | SPTLC2 | 69 | −0.11908 |
| 220477_s_at | C20orf30 | 69 | −0.16221 |
| 205863_at | S100A12 | 68 | −0.10353 |
| 215780_s_at | SET///LOC389168 | 68 | −0.10381 |
| 218197_s_at | OXR1 | 68 | −0.14424 |
| 203077_s_at | SMAD2 | 68 | −0.11242 |
| 222339_x_at | — | 68 | 0.121585 |
| 200698_at | KDELR2 | 68 | −0.15907 |
| 210540_s_at | B4GALT4 | 67 | −0.13556 |
| 217725_x_at | PAI-RBP1 | 67 | −0.14956 |
| 217082_at | — | 67 | 0.086098 |

TABLE 17
Group of 20 genes useful in
prognosis and/or diagnosis of lung cancer.

| | Gene symbol | Number | Signal to noise in a |
|---|---|---|---|
| Affymetrix ID | HUGO ID | of runs* | cancer sample* |
| 207953_at | AD7C-NTP | 1000 | 0.218433 |
| 215208_x_at | RPL35A | 999 | 0.228485 |
| 215604_x_at | UBE2D2 | 998 | 0.224878 |
| 218155_x_at | FLJ10534 | 998 | 0.186425 |
| 216858_x_at | — | 997 | 0.232969 |
| 208137_x_at | — | 996 | 0.191938 |
| 214715_x_at | ZNF160 | 996 | 0.198532 |
| 217715_x_at | ZNF354A | 995 | 0.223881 |
| 220720_x_at | FLJ14346 | 989 | 0.17976 |
| 215907_at | BACH2 | 987 | 0.178338 |
| 217679_x_at | — | 987 | 0.265918 |
| 206169_x_at | RoXaN | 984 | 0.259637 |
| 208246_x_at | TK2 | 982 | 0.179058 |
| 222104_x_at | GTF2H3 | 981 | 0.186025 |
| 206056_x_at | SPN | 976 | 0.196398 |
| 217653_x_at | — | 976 | 0.270552 |
| 210679_x_at | — | 970 | 0.181718 |
| 207730_x_at | HDGF2 | 969 | 0.169108 |
| 214594_x_at | ATP8B1 | 962 | 0.284039 |

*The number of runs when the gene is indicated in cancer samples as differentially expressed out of 1000 test runs.

**Negative values indicate increase of expression in lung cancer, positive values indicate decrease of expression in lung cancer.

One can use the above tables to correlate or compare the expression of the transcript to the expression of the gene product, i.e. protein. Increased expression of the transcript as shown in the table corresponds to increased expression of the gene product. Similarly, decreased expression of the transcript as shown in the table corresponds to decreased expression of the gene product.

In one preferred embodiment, one uses at least one, preferably at least 2, 3, 4, 5, 6, 7, 8, 9, 10 or more, of the genes as listed in Tables 18, 19 and/or 20. In one embodiment, one uses maximum of 500, 400, 300, 200, 100, or 50 of the gene that include at least 5, 6, 7, 8, 9, 10-20, 20-30, 30-40, 40-50, 50-60, 60-70, 1-70, of the genes listed in Tables 18-20.

TABLE 18
361 Airway t-test gene list

| AffyID | GeneName (HUGO ID) |
|---|---|
| 202437_s_at | CYP1B1 |
| 206561_s_at | AKR1B10 |
| 202436_s_at | CYP1B1 |
| 205749_at | CYP1A1 |
| 202435_s_at | CYP1B1 |
| 201884_at | CEACAM5 |
| 205623_at | ALDH3A1 |
| 217626_at | — |
| 209921_at | SLC7A11 |
| 209699_x_at | AKR1C2 |
| 201467_s_at | NQO1 |
| 201468_s_at | NQO1 |
| 202831_at | GPX2 |
| 214303_x_at | MUC5AC |
| 211653_x_at | AKR1C2 |
| 214385_s_at | MUC5AC |
| 216594_x_at | AKR1C1 |
| 205328_at | CLDN10 |
| 209160_at | AKR1C3 |
| 210519_s_at | NQO1 |
| 217678_at | SLC7A11 |
| 205221_at | HGD///LOC642252 |
| 204151_x_at | AKR1C1 |
| 207469_s_at | PIR |
| 206153_at | CYP4F11 |
| 205513_at | TCN1 |
| 209386_at | TM4SF1 |
| 209351_at | KRT14 |
| 204059_s_at | ME1 |
| 209213_at | CBR1 |
| 210505_at | ADH7 |
| 214404_x_at | SPDEF |
| 204058_at | ME1 |
| 218002_s_at | CXCL14 |
| 205499_at | SRPX2 |
| 210065_s_at | UPK1B |
| 204341_at | TRIM16///TRIM16L///LOC653524 |
| 221841_s_at | KLF4 |
| 208864_s_at | TXN |
| 208699_x_at | TKT |
| 210397_at | DEFB1 |
| 204971_at | CSTA |
| 211657_at | CEACAM6 |
| 201463_s_at | TALDO1 |
| 214164_x_at | CA12 |
| 203925_at | GCLM |
| 201118_at | PGD |
| 201266_at | TXNRD1 |
| 203757_s_at | CEACAM6 |
| 202923_s_at | GCLC |
| 214858_at | GPC1 |
| 205009_at | TFF1 |
| 219928_s_at | CABYR |
| 203963_at | CA12 |
| 210064_s_at | UPK1B |
| 219956_at | GALNT6 |
| 208700_s_at | TKT |
| 203824_at | TSPAN8 |
| 207126_x_at | UGT1A10///UGT1A8///UGT1A7///UGT1A6///UGT1A |
| 213441_x_at | SPDEF |
| 207430_s_at | MSMB |
| 209369_at | ANXA3 |
| 217187_at | MUC5AC |
| 209101_at | CTGF |
| 212221_x_at | IDS |
| 215867_x_at | CA12 |
| 214211_at | FTH1 |
| 217755_at | HN1 |
| 201431_s_at | DPYSL3 |
| 204875_s_at | GMDS |
| 215125_s_at | UGT1A10///UGT1A8///UGT1A7///UGT1A6///UGT1A |
| 63825_at | ABHD2 |
| 202922_at | GCLC |
| 218313_s_at | GALNT7 |
| 210297_s_at | MSMB |
| 209448_at | HTATIP2 |
| 204532_x_at | UGT1A10 ///UGT1A8///UGT1A7///UGT1A6///UGT1A |
| 200872_at | S100A10 |
| 21635 l_x_at | DAZ1///DAZ3///DAZ2///DAZ4 |
| 212223_at | IDS |
| 208680_at | PRDX1 |
| 206515_at | CYP4F3 |
| 208596_s_at | UGT1A10///UGT1A8///UGT1A7///UGT1A6///UGT1A |
| 209173_at | AGR2 |
| 204351_at | S100P |
| 202785_at | NDUFA7 |
| 204970_s_at | MAFG |
| 222016_s_at | ZNF323 |
| 200615_s_at | AP2B1 |
| 206094_x_at | UGT1A6 |
| 209706_at | NKX3-1 |
| 217977_at | SEPX1 |
| 201487_at | CTSC |
| 219508_at | GCNT3 |
| 204237_at | GULP1 |
| 213455_at | LOC283677 |
| 213624_at | SMPDL3A |
| 206770_s_at | SLC35A3 |
| 217975_at | WBP5 |
| 201263_at | TARS |
| 218696_at | EIF2AK3 |
| 212560_at | C11orf32 |
| 218885_s_at | GALNT12 |
| 212326_at | VPS13D |
| 217955_at | BCL2L13 |
| 203126_at | IMPA2 |
| 214106_s_at | GMDS |
| 209309_at | AZGP1 |
| 205112_at | PLCE1 |
| 215363_x_at | FOLH1 |
| 206302_s_at | NUDT4///NUDT4P1 |
| 200916_at | TAGLN2 |
| 205042_at | GNE |
| 217979_at | TSPAN13 |
| 203397_s_at | GALNT3 |
| 209786_at | HMGN4 |
| 211733_x_at | SCP2 |
| 207222_at | PLA2G10 |
| 204235_s_at | GULP1 |
| 205726_at | DIAPH2 |
| 203911_at | RAP1GAP |
| 200748_s_at | FTH1 |
| 212449_s_at | LYPLA1 |
| 213059_at | CREB3L1 |
| 201272_at | AKR1B1 |
| 208731_at | RAB2 |
| 205979_at | SCGB2A1 |
| 212805_at | KIAA0367 |
| 202804_at | ABCC1 |
| 218095_s_at | TPARL |
| 205566_at | ABHD2 |
| 209114_at | TSPAN1 |
| 202481_at | DHRS3 |
| 202805_s_at | ABCC1 |
| 219117_s_at | FKBP11 |
| 213172_at | TTC9 |
| 202554_s_at | GSTM3 |
| 218677_at | S100A14 |
| 203306_s_at | SLC35A1 |
| 204076_at | ENTPD4 |
| 200654_at | P4HB |
| 204500_s_at | AGTPBP1 |
| 208918_s_at | NADK |
| 221485_at | B4GALT5 |
| 221511_x_at | CCPG1 |
| 200733_s_at | PTP4A1 |
| 217901_at | DSG2 |
| 202769_at | CCNG2 |
| 202119_s_at | CPNE3 |
| 200945_s_at | SEC31L1 |
| 200924_s_at | SLC3A2 |
| 208736_at | ARPC3 |
| 221556_at | CDC14B |
| 221041_s_at | SLC17A5 |
| 215071_s_at | HIST1H2AC |
| 209682_at | CBLB |
| 209806_at | HIST1H2BK |
| 204485_s_at | TOM1L1 |
| 201666_at | TIMP1 |
| 203192_at | ABCB6 |
| 202722_s_at | GFPT1 |
| 213135_at | TIAM1 |
| 203509_at | SORL1 |
| 214620_x_at | PAM |
| 208919_s_at | NADK |
| 212724_at | RND3 |
| 212160_at | XPOT |
| 212812_at | SERINC5 |
| 200696_s_at | GSN |
| 217845_x_at | HIGD1A |
| 208612_at | PDIA3 |
| 219288_at | C3orf14 |
| 201923_at | PRDX4 |
| 211960_s_at | RAB7 |
| 64942_at | GPR153 |
| 201659_s_at | ARL1 |
| 202439_s_at | IDS |
| 209249_s_at | GHITM |
| 218723_s_at | RGC32 |
| 200087_s_at | TMED2 |
| 209694_at | PTS |
| 202320_at | GTF3C1 |
| 201193_at | IDH1 |
| 212233_at | — |
| 213891_s_at | — |
| 203041_s_at | LAMP2 |
| 202666_s_at | ACTL6A |
| 200863_s_at | RAB11A |
| 203663_s_at | COX5A |
| 211404_s_at | APLP2 |
| 201745_at | PTK9 |
| 217823_s_at | UBE2J1 |
| 202286_s_at | TACSTD2 |
| 212296_at | PSMD14 |
| 211048_s_at | PDIA4 |
| 214429_at | MTMR6 |
| 219429_at | FA2H |
| 212181_s_at | NUDT4 |
| 222116_s_at | TBC1D16 |
| 221689_s_at | PIGP |
| 209479_at | CCDC28A |
| 218434_s_at | AACS |
| 214665_s_at | CHP |
| 202085_at | TJP2 |
| 217992_s_at | EFHD2 |
| 203162_s_at | KATNB1 |
| 205406_s_at | SPA17 |
| 203476_at | TPBG |
| 201724_s_at | GALNT1 |
| 200599_s_at | HSP90B1 |
| 200929_at | TMED10 |
| 200642_at | SOD1 |
| 208946_s_at | BECN1 |
| 202562_s_at | C14orf1 |
| 201098_at | COPB2 |
| 221253_s_at | TXNDC5 |
| 201004_at | SSR4 |
| 203221_at | TLE1 |
| 201588_at | TXNL1 |
| 218684_at | LRRC8D |
| 208799_at | PSMB5 |
| 201471_s_at | SQSTM1 |
| 204034_at | ETHE1 |
| 208689_s_at | RPN2 |
| 212665_at | TIPARP |
| 200625_s_at | CAP1 |
| 213220_at | LOC92482 |
| 200709_at | FKBP1A |
| 203279_at | EDEM1 |
| 200068_s_at | CANX |
| 200620_at | TMEM59 |
| 200075_s_at | GUK1 |
| 209679_s_at | LOC57228 |
| 210715_s_at | SPINT2 |
| 209020_at | C20orf111 |
| 208091_s_at | ECOP |
| 200048_s_at | JTB |
| 218194_at | REXO2 |
| 209103_s_at | UFD1L |
| 208718_at | DDX17 |
| 219241_x_at | SSH3 |
| 216210_x_at | TRIOBP |
| 50277_at | GGA1 |
| 218023_s_at | FAM53C |
| 32540_at | PPP3CC |
| 43511_s_at | — |
| 212001_at | SFRS14 |
| 208637_x_at | ACTN1 |
| 201997_s_at | SPEN |
| 205073_at | CYP2J2 |
| 40837_at | TLE2 |
| 204447_at | ProSAPiP1 |
| 204604_at | PFTK1 |
| 210273_at | PCDH7 |
| 208614_s_at | FLNB |
| 206510_at | SIX2 |
| 200675_at | CD81 |
| 219228_at | ZNF331 |
| 209426_s_at | AMACR |
| 204000_at | GNB5 |
| 221742_at | CUGBP1 |
| 208883_at | EDD1 |
| 210166_at | TLR5 |
| 211026_s_at | MGLL |
| 220446_s_at | CHST4 |
| 207636_at | SERPINI2 |
| 212226_s_at | PPAP2B |
| 210347_s_at | BCL11A |
| 218424_s_at | STEAP3 |
| 204287_at | SYNGR1 |
| 205489_at | CRYM |
| 36129_at | RUTBC1 |
| 215418_at | PARVA |
| 213029_at | NFIB |
| 221016_s_at | TCF7L1 |
| 209737_at | MAGI2 |
| 220389_at | CCDC81 |
| 213622_at | COL9A2 |
| 204740_at | CNKSR1 |
| 212126_at | — |
| 207760_s_at | NCOR2 |
| 205258_at | INHBB |
| 213169_at | — |
| 33760_at | PEX14 |
| 220968_s_at | TSPAN9 |
| 221792_at | RAB6B |
| 205752_s_at | GSTM5 |
| 218974_at | FLJ10159 |
| 221748_s_at | TNS1 |
| 212185_x_at | MT2A |
| 209500_x_at | TNFSF13///TNFSF12-TNFSF13 |
| 215445_x_at | 1-Mar |
| 220625_s_at | ELF5 |
| 32137_at | JAG2 |
| 219747_at | FLJ23191 |
| 201397_at | PHGDH |
| 207913_at | CYP2F1 |
| 217853_at | TNS3 |
| 1598_g_at | GAS6 |
| 203799_at | CD302 |
| 203329_at | PTPRM |
| 208712_at | CCND1 |
| 210314_x_at | TNFSF13///TNFSF12-TNFSF13 |
| 213217_at | ADCY2 |
| 200953_s_at | CCND2 |
| 204326_x_at | MT1X |
| 213488_at | SNED1 |
| 213505_s_at | SFRS14 |
| 200982_s_at | ANXA6 |
| 211732_x_at | HNMT |
| 202587_s_at | AK1 |
| 396_f_at | EPOR |
| 200878_at | EPAS1 |
| 213228_at | PDE8B |
| 215785_s_at | CYFIP2 |
| 213601_at | SLIT1 |
| 37953_s_at | ACCN2 |
| 205206_at | KAL1 |
| 212859_x_at | MT1E |
| 217165_x_at | MT1F |
| 204754_at | HLF |
| 218225_at | SITPEC |
| 209784_s_at | JAG2 |
| 211538_s_at | HSPA2 |
| 211456_x_at | LOC650610 |
| 204734_at | KRT15 |
| 201563_at | SORD |
| 202746_at | ITM2A |
| 218025_s_at | PECI |
| 203914_x_at | HPGD |
| 200884_at | CKB |
| 204753_s_at | HLF |
| 207718_x_at | CYP2A6///CYP2A7///CYP2A7P1///CYP2A13 |
| 218820_at | C14orf132 |
| 204745_x_at | MT1G |
| 204379_s_at | FGFR3 |
| 207808_s_at | PROS1 |
| 207547_s_at | FAM107A |
| 20858 l_x_at | MT1X |
| 205384_at | FXYD1 |
| 213629_x_at | MT1F |
| 823_at | CX3CL1 |
| 203687_at | CX3CL1 |
| 211295_x_at | CYP2A6 |
| 204755_x_at | HLF |
| 209897_s_at | SLIT2 |
| 40093_at | BCAM |
| 211726_s_at | FMO2 |
| 206461_x_at | MT1H |
| 219250_s_at | FLRT3 |
| 210524_x_at | — |
| 220798_x_at | PRG2 |
| 219410_at | TMEM45A |
| 205680_at | MMP10 |
| 217767_at | C3///LOC653879 |
| 220562_at | CYP2W1 |
| 210445_at | FABP6 |
| 205725_at | SCGB1A1 |
| 213432_at | MUC5B///LOC649768 |
| 209074_s_at | FAM107A |
| 216346_at | SEC14L3 |

TABLE 19
107 Nose Leading Edge Genes

| AffxID | Hugo ID |
|---|---|
| 203369_x_at | — |
| 218434_s_at | AACS |
| 205566_at | ABHD2 |
| 217687_at | ADCY2 |
| 210505_at | ADH7 |
| 205623_at | ALDH3A1 |
| 200615_s_at | AP2B1 |
| 214875_x_at | APLP2 |
| 212724_at | ARHE |
| 201659_s_at | ARL1 |
| 208736_at | ARPC3 |
| 213624_at | ASM3A |
| 209309_at | AZGP1 |
| 217188_s_at | C14orf1 |
| 200620_at | C1orf8 |
| 200068_s_at | CANX |
| 213798_s_at | CAP1 |
| 200951_s_at | CCND2 |
| 202769_at | CCNG2 |
| 201884_at | CEACAM5 |
| 203757_s_at | CEACAM6 |
| 214665_s_at | CHP |
| 205328_at | CLDN10 |
| 203663_s_at | COX5A |
| 202119_s_at | CPNE3 |
| 221156_x_at | CPR8 |
| 201487_at | CTSC |
| 205749_at | CYP1A1 |
| 207913_at | CYP2F1 |
| 206153_at | CYP4F11 |
| 206514_s_at | CYP4F3 |
| 21635 l_x_at | DAZ4 |
| 203799_at | DCL-1 |
| 212665_at | DKFZP434J214 |
| 201430_s_at | DPYSL3 |
| 211048_s_at | ERP70 |
| 219118_at | FKBP11 |
| 214119_s_at | FKBP1A |
| 208918_s_at | FLJ13052 |
| 217487_x_at | FOLH1 |
| 200748_s_at | FTH1 |
| 201723_s_at | GALNT1 |
| 218885_s_at | GALNT12 |
| 203397_s_at | GALNT3 |
| 218313_s_at | GALNT7 |
| 203925_at | GCLM |
| 219508_at | GCNT3 |
| 202722_s_at | GFPT1 |
| 204875_s_at | GMDS |
| 205042_at | GNE |
| 208612_at | GRP58 |
| 214040_s_at | GSN |
| 214307_at | HGD |
| 209806_at | HIST1H2BK |
| 202579_x_at | HMGN4 |
| 207180_s_at | HTATIP2 |
| 206342_x_at | IDS |
| 203126_at | IMPA2 |
| 210927_x_at | JTB |
| 203163_at | KATNB1 |
| 204017_at | KDELR3 |
| 213174_at | KIAA0227 |
| 212806_at | KIAA0367 |
| 210616_s_at | KIAA0905 |
| 221841_s_at | KLF4 |
| 203041_s_at | LAMP2 |
| 213455_at | LOC92689 |
| 218684_at | LRRC5 |
| 204059_s_at | ME1 |
| 207430_s_at | MSMB |
| 210472_at | MT1G |
| 213432_at | MUC5B |
| 211498_s_at | NKX3-1 |
| 201467_s_at | NQO1 |
| 206303_s_at | NUDT4 |
| 213498_at | OASIS |
| 200656_s_at | P4HB |
| 213441_x_at | PDEF |
| 207469_s_at | PIR |
| 207222_at | PLA2G10 |
| 209697_at | PPP3CC |
| 201923_at | PRDX4 |
| 200863_s_at | RAB11A |
| 208734_x_at | RAB2 |
| 203911_at | RAP1GA1 |
| 218723_s_at | RGC32 |
| 200087_s_at | RNP24 |
| 200872_at | S100A10 |
| 205979_at | SCGB2A1 |
| 202481_at | SDR1 |
| 217977_at | SEPX1 |
| 221041_s_at | SLC17A5 |
| 203306_s_at | SLC35A1 |
| 207528_s_at | SLC7A11 |
| 202287_s_at | TACSTD2 |
| 210978_s_at | TAGLN2 |
| 205513_at | TCN1 |
| 201666_at | TIMP1 |
| 208699_x_at | TKT |
| 217979_at | TM4SF13 |
| 203824_at | TM4SF3 |
| 200929_at | TMP21 |
| 221253_s_at | TXNDC5 |
| 217825_s_at | UBE2J1 |
| 215125_s_at | UGT1A10 |
| 210064_s_at | UPK1B |
| 202437_s_at | CYP1B1 |

TABLE 20
70 gene list

| AFFYID | Gene Name (HUGO ID) |
|---|---|
| 213693_s_at | MUC1 |
| 211695_x_at | MUC1 |
| 207847_s_at | MUC1 |
| 208405_s_at | CD164 |
| 220196_at | MUC16 |
| 217109_at | MUC4 |
| 217110_s_at | MUC4 |
| 204895_x_at | MUC4 |
| 214385_s_at | MUC5AC |
| 1494_f_at | CYP2A6 |
| 210272_at | CYP2B7P1 |
| 206754_s_at | CYP2B7P1 |
| 210096_at | CYP4B1 |
| 208928_at | POR |
| 207913_at | CYP2F1 |
| 220636_at | DNAI2 |
| 201999_s_at | DYNLT1 |
| 205186_at | DNALI1 |
| 220125_at | DNAI1 |
| 210345_s_at | DNAH9 |
| 214222_at | DNAH7 |
| 211684_s_at | DYNC1I2 |
| 211928_at | DYNC1H1 |
| 200703_at | DYNLL1 |
| 217918_at | DYNLRB1 |
| 217917_s_at | DYNLRB1 |
| 209009_at | ESD |
| 204418_x_at | GSTM2 |
| 215333_x_at | GSTM1 |
| 217751_at | GSTK1 |
| 203924_at | GSTA1 |
| 201106_at | GPX4 |
| 200736_s_at | GPX1 |
| 204168_at | MGST2 |
| 200824_at | GSTP1 |
| 211630_s_at | GSS |
| 201470_at | GSTO1 |
| 201650_at | KRT19 |
| 209016_s_at | KRT7 |
| 209008_x_at | KRT8 |
| 201596_x_at | KRT18 |
| 210633_x_at | KRT10 |
| 207023_x_at | KRT10 |
| 212236_x_at | KRT17 |
| 201820_at | KRT5 |
| 204734_at | KRT15 |
| 203151_at | MAP1A |
| 200713_s_at | MAPRE1 |
| 204398_s_at | EML2 |
| 40016_g_at | MAST4 |
| 208634_s_at | MACF1 |
| 205623_at | ALDH3A1 |
| 212224_at | ALDH1A1 |
| 205640_at | ALDH3B1 |
| 211004_s_at | ALDH3B1 |
| 202054_s_at | ALDH3A2 |
| 205208_at | ALDH1L1 |
| 201612_at | ALDH9A1 |
| 201425_at | ALDH2 |
| 201090_x_at | K-ALPHA-1 |
| 202154_x_at | TUBB3 |
| 202477_s_at | TUBGCP2 |
| 203667_at | TBCA |
| 204141_at | TUBB2A |
| 207490_at | TUBA4 |
| 208977_x_at | TUBB2C |
| 209118_s_at | TUBA3 |
| 20925 l_x_at | TUBA6 |
| 211058_x_at | K-ALPHA-1 |
| 211072_x_at | K-ALPHA-1 |
| 211714_x_at | TUBB |
| 211750_x_at | TUBA6 |
| 212242_at | TUBA1 |
| 212320_at | TUBB |
| 212639_x_at | K-ALPHA-1 |
| 213266_at | 76P |
| 213476_x_at | TUBB3 |
| 213646_x_at | K-ALPHA-1 |
| 213726_x_at | TUBB2C |

Additionally, one can use any one or a combination of the genes listed in Table 19.

The analysis of the gene expression of one or more genes and/or transcripts of the groups or their subgroups of the present invention can be performed using any gene expression method known to one skilled in the art. Such methods include, but are not limited to expression analysis using nucleic acid chips (e.g. Affymetrix chips) and quantitative RT-PCR based methods using, for example real-time detection of the transcripts. Analysis of transcript levels according to the present invention can be made using total or messenger RNA or proteins encoded by the genes identified in the diagnostic gene groups of the present invention as a starting material. In the preferred embodiment the analysis is an immunohistochemical analysis with an antibody directed against proteins comprising at least about 10-20, 20-30, preferably at least 36, at least 36-50, 50, about 50-60, 60-70, 70-80, 80-90, 96, 100-180, 180-200, 200-250, 250-300, 300-350, 350-400, 400-450, 450-500, 500-535 proteins encoded by the genes and/or transcripts as shown in Tables 11-17.

The methods of analyzing transcript levels of the gene groups in an individual include Northern-blot hybridization, ribonuclease protection assay, and reverse transcriptase polymerase chain reaction (RT-PCR) based methods. The different RT-PCR based techniques are the most suitable quantification method for diagnostic purposes of the present invention, because they are very sensitive and thus require only a small sample size which is desirable for a diagnostic test. A number of quantitative RT-PCR based methods have been described and are useful in measuring the amount of transcripts according to the present invention. These methods include RNA quantification using PCR and complementary DNA (cDNA) arrays (Shalon et al., Genome Research 6(7):639-45, 1996; Bernard et al., Nucleic Acids Research 24(8):1435-42, 1996), real competitive PCR using a MALDI-TOF Mass spectrometry based approach (Ding et al, PNAS, 100: 3059-64, 2003), solid-phase mini-sequencing technique, which is based upon a primer extension reaction (U.S. Pat. No. 6,013,431, Suomalainen et al. Mol. Biotechnol. June; 15(2):123-31, 2000), ion-pair high-performance liquid chromatography (Doris et al. J. Chromatogr. A May 8; 806(1):47-60, 1998), and 5′ nuclease assay or real-time RT-PCR (Holland et al. Proc Natl Acad Sci USA 88: 7276-7280, 1991).

Methods using RT-PCR and internal standards differing by length or restriction endonuclease site from the desired target sequence allowing comparison of the standard with the target using gel electrophoretic separation methods followed by densitometric quantification of the target have also been developed and can be used to detect the amount of the transcripts according to the present invention (see, e.g., U.S. Pat. Nos. 5,876,978; 5,643,765; and 5,639,606.

The samples are preferably obtained from bronchial airways using, for example, endoscopic cytobrush in connection with a fiber optic bronchoscopy. In one embodiment, the cells are obtained from the individual's mouth buccal cells, using, for example, a scraping of the buccal mucosa.

In one preferred embodiment, the invention provides a prognostic and/or diagnostic immunohistochemical approach, such as a dip-stick analysis, to determine risk of developing lung disease. Antibodies against proteins, or antigenic epitopes thereof, that are encoded by the group of genes of the present invention, are either commercially available or can be produced using methods well know to one skilled in the art.

The invention contemplates either one dipstick capable of detecting all the diagnostically important gene products or alternatively, a series of dipsticks capable of detecting the amount proteins of a smaller sub-group of diagnostic proteins of the present invention.

Antibodies can be prepared by means well known in the art. The term “antibodies” is meant to include monoclonal antibodies, polyclonal antibodies and antibodies prepared by recombinant nucleic acid techniques that are selectively reactive with a desired antigen. Antibodies against the proteins encoded by any of the genes in the diagnostic gene groups of the present invention are either known or can be easily produced using the methods well known in the art. Internet sites such as Biocompare through the World Wide Web at biocompare.com at abmatrix to provide a useful tool to anyone skilled in the art to locate existing antibodies against any of the proteins provided according to the present invention.

Antibodies against the diagnostic proteins according to the present invention can be used in standard techniques such as Western blotting or immunohistochemistry to quantify the level of expression of the proteins of the diagnostic airway proteome. This is quantified according to the expression of the gene transcript, i.e. the increased expression of transcript corresponds to increased expression of the gene product, i.e. protein. Similarly decreased expression of the transcript corresponds to decreased expression of the gene product or protein. Detailed guidance of the increase or decrease of expression of preferred transcripts in lung disease, particularly lung cancer, is set forth in the tables. For example, Tables 15 and 16 describe a group of genes the expression of which is altered in lung cancer.

Immunohistochemical applications include assays, wherein increased presence of the protein can be assessed, for example, from a saliva or sputum sample.

The immunohistochemical assays according to the present invention can be performed using methods utilizing solid supports. The solid support can be a any phase used in performing immunoassays, including dipsticks, membranes, absorptive pads, beads, microtiter wells, test tubes, and the like. Preferred are test devices which may be conveniently used by the testing personnel or the patient for self-testing, having minimal or no previous training. Such preferred test devices include dipsticks, membrane assay systems as described in U.S. Pat. No. 4,632,901. The preparation and use of such conventional test systems is well described in the patent, medical, and scientific literature. If a stick is used, the anti-protein antibody is bound to one end of the stick such that the end with the antibody can be dipped into the solutions as described below for the detection of the protein. Alternatively, the samples can be applied onto the antibody-coated dipstick or membrane by pipette or dropper or the like.

The antibody against proteins encoded by the diagnostic airway transcriptome (the “protein”) can be of any isotype, such as IgA, IgG or IgM, Fab fragments, or the like. The antibody may be a monoclonal or polyclonal and produced by methods as generally described, for example, in Harlow and Lane, Antibodies, A Laboratory Manual, Cold Spring Harbor Laboratory, 1988, incorporated herein by reference. The antibody can be applied to the solid support by direct or indirect means. Indirect bonding allows maximum exposure of the protein binding sites to the assay solutions since the sites are not themselves used for binding to the support. Preferably, polyclonal antibodies are used since polyclonal antibodies can recognize different epitopes of the protein thereby enhancing the sensitivity of the assay.

The solid support is preferably non-specifically blocked after binding the protein antibodies to the solid support. Non-specific blocking of surrounding areas can be with whole or derivatized bovine serum albumin, or albumin from other animals, whole animal serum, casein, non-fat milk, and the like.

The sample is applied onto the solid support with bound protein-specific antibody such that the protein will be bound to the solid support through said antibodies. Excess and unbound components of the sample are removed and the solid support is preferably washed so the antibody-antigen complexes are retained on the solid support. The solid support may be washed with a washing solution which may contain a detergent such as Tween-20, Tween-80 or sodium dodecyl sulfate.

After the protein has been allowed to bind to the solid support, a second antibody which reacts with protein is applied. The second antibody may be labeled, preferably with a visible label. The labels may be soluble or particulate and may include dyed immunoglobulin binding substances, simple dyes or dye polymers, dyed latex beads, dye-containing liposomes, dyed cells or organisms, or metallic, organic, inorganic, or dye solids. The labels may be bound to the protein antibodies by a variety of means that are well known in the art. In some embodiments of the present invention, the labels may be enzymes that can be coupled to a signal producing system. Examples of visible labels include alkaline phosphatase, beta-galactosidase, horseradish peroxidase, and biotin. Many enzyme-chromogen or enzyme-substrate-chromogen combinations are known and used for enzyme-linked assays. Dye labels also encompass radioactive labels and fluorescent dyes.

Simultaneously with the sample, corresponding steps may be carried out with a known amount or amounts of the protein and such a step can be the standard for the assay. A sample from a healthy individual exposed to a similar air pollutant such as cigarette smoke, can be used to create a standard for any and all of the diagnostic gene group encoded proteins.

The solid support is washed again to remove unbound labeled antibody and the labeled antibody is visualized and quantified. The accumulation of label will generally be assessed visually. This visual detection may allow for detection of different colors, for example, red color, yellow color, brown color, or green color, depending on label used. Accumulated label may also be detected by optical detection devices such as reflectance analyzers, video image analyzers and the like. The visible intensity of accumulated label could correlate with the concentration of protein in the sample. The correlation between the visible intensity of accumulated label and the amount of the protein may be made by comparison of the visible intensity to a set of reference standards. Preferably, the standards have been assayed in the same way as the unknown sample, and more preferably alongside the sample, either on the same or on a different solid support.

The concentration of standards to be used can range from about 1 mg of protein per liter of solution, up to about 50 mg of protein per liter of solution. Preferably, two or more different concentrations of an airway gene group encoded proteins are used so that quantification of the unknown by comparison of intensity of color is more accurate.

For example, the present invention provides a method for detecting risk of developing lung cancer in a subject exposed to cigarette smoke comprising measuring the transcription profile in a nasal epithelial cell sample of the proteins encoded by one or more groups of genes of the invention in a biological sample of the subject. Preferably at least about 30, still more preferably at least about 36, 40, 50, 60, 70, 80, 90, 100, 110, 120, 130, 140, 150, 160, 170, or about 180 of the proteins encoded by the airway transcriptome in a biological sample of the subject are analyzed. The method comprises binding an antibody against each protein encoded by the gene in the gene group (the “protein”) to a solid support chosen from the group consisting of dip-stick and membrane; incubating the solid support in the presence of the sample to be analyzed under conditions where antibody-antigen complexes form; incubating the support with an anti-protein antibody conjugated to a detectable moiety which produces a signal; visually detecting said signal, wherein said signal is proportional to the amount of protein in said sample; and comparing the signal in said sample to a standard, wherein a difference in the amount of the protein in the sample compared to said standard of the same group of proteins, is indicative of diagnosis of or an increased risk of developing lung cancer. The standard levels are measured to indicate expression levels in an airway exposed to cigarette smoke where no cancer has been detected.

The assay reagents, pipettes/dropper, and test tubes may be provided in the form of a kit. Accordingly, the invention further provides a test kit for visual detection of the proteins encoded by the airway gene groups, wherein detection of a level that differs from a pattern in a control individual is considered indicative of an increased risk of developing lung disease in the subject. The test kit comprises one or more solutions containing a known concentration of one or more proteins encoded by the airway transcriptome (the “protein”) to serve as a standard; a solution of a anti-protein antibody bound to an enzyme; a chromogen which changes color or shade by the action of the enzyme; a solid support chosen from the group consisting of dip-stick and membrane carrying on the surface thereof an antibody to the protein. Instructions including the up or down regulation of the each of the genes in the groups as provided by the Tables 11 and 12 are included with the kit.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The methods of the present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide and protein arrays.

Nucleic acid arrays that are useful in the present invention include, but are not limited to those that are commercially available from Affymetrix (Santa Clara, Calif.) under the brand name GeneChip7. Example arrays are shown on the website at affymetrix.com.

Examples of gene expression monitoring, and profiling methods that are useful in the methods of the present invention are shown in U.S. Pat. Nos. 5,800,992, 6,013,449, 6,020,135, 6,033,860, 6,040,138, 6,177,248 and 6,309,822. Other examples of uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with expression analysis, the nucleic acid sample may be amplified by a variety of mechanisms, some of which may employ PCR. See, e.g., PCR Technology: Principles and Applications for DNA Amplification (Ed. H. A. Erlich, Freeman Press, NY, NY, 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. patent application Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (U.S. Pat. Nos. 5,409,818, 5,554,517, and 6,063,603). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described, for example, in Dong et al., Genome Research 11, 1418 (2001), in U.S. Pat. Nos. 6,361,947, 6,391,592 and U.S. patent application Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described, for example, in U.S. Pat. Nos. 5,871,928, 5,874,219, 6,045,996 and 6,386,749, 6,391,623 each of which are incorporated herein by reference.

The present invention also contemplates signal detection of hybridization between the sample and the probe in certain embodiments. See, for example, U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in provisional U.S. Patent application 60/364,731 and in PCT Application PCT/US99/06097 (published as WO99/47964).

Examples of methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964).

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001).

The present invention also makes use of various computer program products and software for a variety of purposes, such as probe design, management of data, analysis, and instrument operation. See, for example, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have embodiments that include methods for providing gene expression profile information over networks such as the Internet as shown in, for example, U.S. patent application Ser. No. 10/063,559, 60/349,546, 60/376,003, 60/394,574, 60/403,381.

Throughout this specification, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 10-20 should be considered to have specifically disclosed sub-ranges such as from 10-13, from 10-14, from 10-15, from 11-14, from 11-16, etc., as well as individual numbers within that range, for example, 10, 11, 12, 13, 14, 15, 16, 17, 18, 19, and 20. This applies regardless of the breadth of the range. In addition, the fractional ranges are also included in the exemplified amounts that are described. Therefore, for example, a range of 1-3 includes fractions such as 1.1, 1.2, 1.3, 1.4, 1.5, 1.6, etc. This applies particularly to the amount of increase or decrease of expression of any particular gene or transcript.

The present invention has many preferred embodiments and relies on many patents, applications and other references for details known to those of the art. Therefore, when a patent, application, or other reference is cited or repeated throughout the specification, it should be understood that it is incorporated by reference in its entirety for all purposes as well as for the proposition that is recited.

### EXAMPLES

### Example 1

In this study, we used three study groups: 1) normal non-smokers (n=23); 2) smokers without cancer (active v. former smokers) (n=52); 3) smokers with suspect cancer (n=98: 45 cancer, 53 no cancer).

We obtained epithelial nucleic acids (RNA/DNA) from epithelial cells in mouth and airway (bronchoscopy). We also obtained nucleic acids from blood to provide one control.

We analyzed gene expression using RNA and U133A Affymetrix array that represents transcripts from about 22,500 genes.

The microarray data analysis was performed as follows. We first scanned the Affymetrix chips that had been hybridized with the study group samples. The obtained microarray raw data consisted of signal strength and detection p-value. We normalized or scaled the data, and filtered the poor quality chips based on images, control probes, and histograms according to standard Affymetrix instructions. We also filtered contaminated specimens which contained non-epithelial cells. Lastly, the genes of importance were filtered using detection p-value. This resulted in identification of transcripts present in normal airways (normal airway transcriptome), with variability and multiple regression analysis. This also resulted in identification of effects of smoking on airway epithelial cell transcription. For this, we used T-test and Pearson correlation analysis. We also identified a group or a set of transcripts that were differentially expressed in samples with lung cancer and samples without cancer. This analysis was performed using class prediction models.

We used weighted voting method. The weighted voting method ranks, and gives a weight “p” to all genes by the signal to noise ration of gene expression between two classes: P=mean_{(class 1)}−mean_{(class 2)}/sd_{(class 1)}=sd_{(class 2)}. Committees of variable sizes of the top ranked genes were used to evaluate test samples, but genes with more significant p-values were more heavily weighed. Each committee genes in test sample votes for one class or the other, based on how close that gene expression level is to the class 1 mean or the class 2 mean. V_{(gene A)}=P_{(gene A)}, i.e. level of expression in test sample less the average of the mean expression values in the two classes. Votes for each class were tallied and the winning class was determined along with prediction strength as PS=V_{win}−Vₗₒₛₑ/V_{win}+Vₗₒₛₑ. Finally, the accuracy was validated using cross-validation+/−independent samples.

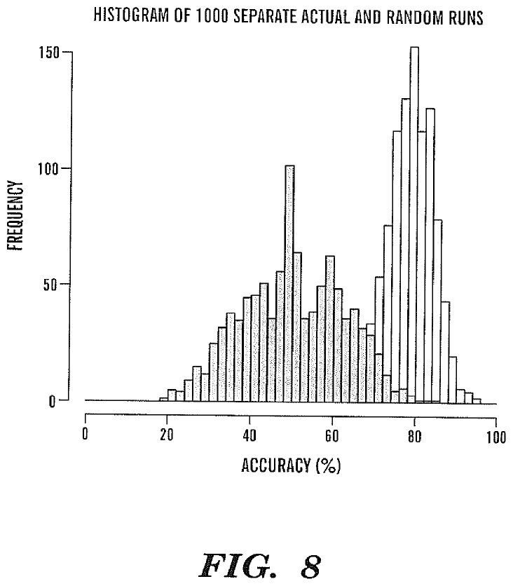

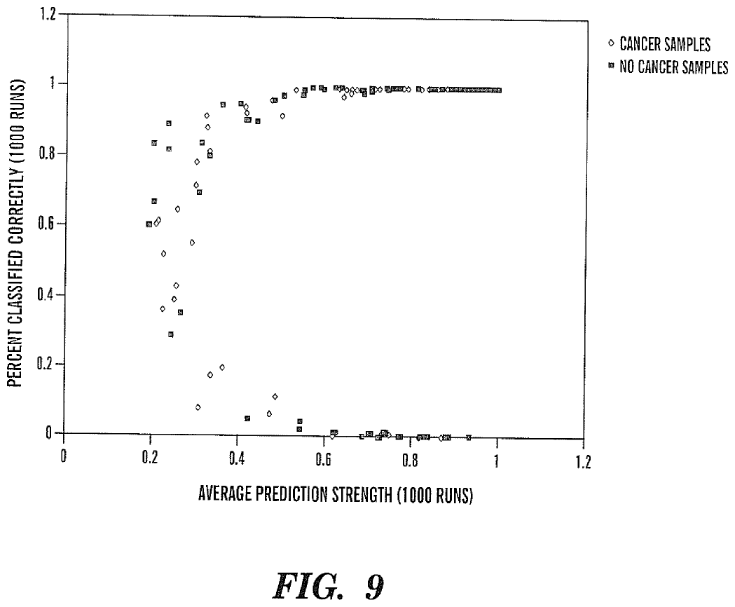

FIG. 8 shows diagrams of the class prediction model analysis used in the Example 1.

The results of the weighted voting method for a 50 gene group analysis (50 gene committee) were as follows. Cross-validation (n=74) resulted in accuracy of 81%, with sensitivity of 76% and specificity of 85%. In an independent dataset (n=24) the accuracy was 88%, with sensitivity of 75% and specificity of 100%.

We note that with sensitivity to bronchoscopy alone only 18/45 (40%) of cancers were diagnosed at the time of bronchoscopy using brushings, washings, biopsy or Wang.

We performed a gene expression analysis of the human genome using isolated nucleic acid samples comprising lung cell transcripts from individuals. The chip used was the Human Genome U133 Set. We used Microarray Suite 5.0 software to analyze raw data from the chip (i.e. to convert the image file into numerical data). Both the chip and the software are proprietary materials from Affymetrix. Bronchoscopy was performed to obtain nucleic acid samples from 98 smoker individuals.

We performed a Student's t-test using gene expression analysis of 45 smokers with lung cancer and 53 smokers without lung cancer. We identified several groups of genes that showed significant variation in their expression between smokers with cancer and smokers without cancer. We further identified at least three groups of genes that, when their expression was analyzed in combination, the results allowed us to significantly increase diagnostic power in identifying cancer carrying smokers from smokers without cancer.

The predictor groups of genes were identified using the GenePattern server from the Broad Institute, which includes the Weighted Voting algorithm. The default settings, i.e., the signal to noise ratio and no gene filtering, were used. GenePattern is available at World Wide Web from broad.mit.edu/cancer/software/genepattern. This program allows analysis of data in groups rather than as individual genes.

Table 1 shows the top 96 genes from our analysis with different expression patterns in smokers with cancer and smokers without cancer.

Table 2 shows the 84 genes that were also identified in our previous screens as individual predictors of lung cancer.

Table 4 shows a novel group of 36 genes the expression of which was different between the smokers with cancer and smokers without cancer.

Table 3 shows a group of 50 genes that we identified as most predictive of development of cancer in smokers. That is, that when the expression of these genes was analyzed and reflected the pattern (expression down or up) as shown in Table 3, we could identify the individuals who will develop cancer based on this combined expression profile of these genes. When used in combination, the expression analysis of these 50 genes was predictive of a smoker developing lung cancer in over 70% of the samples. Accuracy of diagnosis of lung cancer in our sample was 80-85% on cross-validation and independent dataset (accuracy includes both the sensitivity and specificity). The sensitivity (percent of cancer cases correctly diagnosed) was approximately 75% as compared to sensitivity of 40% using standard bronchoscopy technique. (Specificity is percent of non-cancer cases correctly diagnosed).

These data show the dramatic increase of diagnostic power that can be reached using the expression profiling of the gene groups as identified in the present study.

### Example 2

We report here a gene expression profile, derived from histologically normal large airway epithelial cells of current and former smokers with clinical suspicion of lung cancer that is highly sensitive and specific for the diagnosis of lung cancer. This airway signature is effective in diagnosing lung cancer at an early and potentially resectable stage. When combined with results from bronchoscopy (i.e. washings, brushings, and biopsies of the affected area), the expression profile is diagnostic of lung cancer in 95% of cases. We further show that the airway epithelial field of injury involves a number of genes that are differentially expressed in lung cancer tissue, providing potential information about pathways that may be involved in the genesis of lung cancer.

Patient Population: We obtained airway brushings from current and former smokers (n=208) undergoing fiber optic bronchoscopy as a diagnostic study for clinical suspicion of lung cancer between January 2003 and May 2005. Patients were recruited from 4 medical centers: Boston University Medical Center, Boston, Mass.; Boston Veterans Administration, West Roxbury, Mass.; Lahey Clinic, Burlington, Mass.; and Trinity College, Dublin, Ireland. Exclusion criteria included never smokers, cigar smokers and patients on a mechanical ventilator at the time of their bronchoscopy. Each subject was followed clinically, post-bronchoscopy, until a final diagnosis of lung cancer or an alternate benign diagnosis was made. Subjects were classified as having lung cancer if their bronchoscopy studies (brushing, bronchoalveolar lavage or endobronchial biopsy) or a subsequent lung biopsy (transthoracic biopsy or surgical lung biopsy) yielded tumor cells on pathology/cytology. Subjects were classified with an alternative benign diagnosis if the bronchoscopy or subsequent lung biopsy yielded a non-lung cancer diagnosis or if their radiographic abnormality resolved on follow up chest imaging. The study was approved by the Institutional Review Boards of all 4 medical centers and all participants provided written informed consent.

Airway epithelial cell collection: Following completion of the standard diagnostic bronchoscopy studies, bronchial airway epithelial cells were obtained from the “uninvolved” right mainstem bronchus with an endoscopic cytobrush (Cellebrity Endoscopic Cytobrush, Boston Scientific, Boston, Mass.). If a suspicious lesion (endobronchial or submucosal) was seen in the right mainstem bronchus, cells were then obtained from the uninvolved left mainstem bronchus. The brushes were immediately placed in TRIzol reagent (Invitrogen, Carlsbad, Calif.) after removal from the bronchoscope and kept at −80° C. until RNA isolation was performed. RNA was extracted from the brushes using TRIzol Reagent (Invitrogen) as per the manufacturer protocol, with a yield of 8-15 μg of RNA per patient. Integrity of the RNA was confirmed by denaturing gel electrophoresis. Epithelial cell content and morphology of representative bronchial brushing samples was quantified by cytocentrifugation (ThermoShandon Cytospin, Pittsburgh, Pa.) of the cell pellet and staining with a cytokeratin antibody (Signet, Dedham Mass.). These samples were reviewed by a pathologist who was blinded to the diagnosis of the patient.

Microarray data acquisition and preprocessing: 6-8 μg of total RNA was processed, labeled, and hybridized to Affymetrix HG-U133A GeneChips containing approximately 22,215 human transcripts as described previously (17). We obtained sufficient quantity of high quality RNA for microarray studies from 152 of the 208 samples. The quantity of RNA obtained improved during the course of the study so that 90% of brushings yielded sufficient high quality RNA during the latter half of the study. Log-normalized probe-level data was obtained from CEL files using the Robust Multichip Average (RMA) algorithm (18). A z-score filter was employed to filter out arrays of poor quality (see supplement for details), leaving 129 samples with a final diagnosis available for analysis.

Microarray Data Analysis: Class Prediction

To develop and test a gene expression predictor capable of distinguishing smokers with and without lung cancer, 60% of samples (n=77) representing a spectrum of clinical risk for lung cancer and approximately equal numbers of cancer and no cancer subjects were randomly assigned to a training set (see Supplement). Using the training set samples, the 22,215 probesets were filtered via ANCOVA using pack-years as the covariate; probesets with a p-value greater than 0.05 for the difference between the two groups were excluded. This training-set gene filter was employed to control for the potential confounding effect of cumulative tobacco exposure, which differed between subjects with and without cancer (see Table 1a).

| | Cancer | NonCancer |
|---|---|---|
| Samples | 60 | 69 |
| Age ** | 64.1 +/− 9.0 | 49.8 +/− 15.2 |
| Smoking Status | 51.7% F, 48 . . . 3% C | 37.7% F, 62 . . . 3% C |
| Gender | 80% M, 20% F | 73.9% M, 26.1% F |
| PackYears ** | 57.4 +/− 25 . . . 6 | 29.4 +/− 27 . . . 3 |
| Age Started | 15.2 +/− 4.2 | 16.7 +/− 6.8 |
| Smoking intensity | 1.3 +/− 0.45 | 0.9 +/− 0.5 |
| (PPD): Currents * | | |
| Months Quit: | 113 +/− 118 | 158 +/− 159 |
| Formers | | |

* Two classes statistically different: p < 0.05
** Two classes statistically different: p < 0.001

Table 1a shows demographic features and characteristics of the two patient classes being studied. Statistical differences between the two patient classes and associated p values were calculated using T-tests, Chi-square tests and Fisher's exact tests where appropriate.

Gene selection was conducted through internal cross-validation within the training set using the weighted voting algorithm (19). The internal cross-validation was repeated 50 times, and the top 40 up- and top 40 down-regulated probesets in cancer most frequently chosen during internal cross-validation runs were selected as the final gene committee of 80 features (see sections, infra, for details regarding the algorithm and the number of genes selected for the committee).

The accuracy, sensitivity, and specificity of the biomarker were assessed on the independent test set of 52 samples. This was accomplished by using the weighted vote algorithm to predict the class of each test set sample based on the gene expression of the 80 probesets and the probe set weights derived from the 77 samples in the training set. To assess the performance of our classifier, we first created 1000 predictors from the training set where we randomized the training set class labels. We evaluated the performance of these “class-randomized” classifiers for predicting the sample class of the test set samples and compared these to our classifier using ROC analysis. To assess whether the performance of our gene expression profile depends on the specific training and test sets from which it was derived and tested, we next created 500 new training and test sets with our 129 samples and derived new “sample-randomized” classifiers from each of these training sets which were then tested on the corresponding test set. To assess the specificity of our classifier genes, we next created 500 classifiers each composed of 80 randomly selected genes. We then tested the ability of these “gene-randomized” classifiers to predict the class of samples in the test set. To evaluate the robustness of our class prediction algorithm and data preprocessing, we also used these specific 80 genes to generate predictive models with an alternate class prediction algorithm (Prediction Analysis of Microarrays (PAM)(20)) and with MAS 5.0 generated expression data instead of RMA. Finally, the performance of our predictor was compared to the diagnostic yield of bronchoscopy.

Quantitative PCR Validation: Real time PCR (QRT-PCR) was used to confirm the differential expression of a select number of genes in our predictor. Primer sequences were designed with Primer Express software (Applied Biosystems, Foster City, Calif.). Forty cycles of amplification, data acquisition, and data analysis were carried out in an ABI Prism 7700 Sequence Detector (Applied Biosystems, Foster City, Calif.). All real time PCR experiments were carried out in triplicate on each sample (see sections infra).

Linking to lung cancer tissue microarray data: The 80-gene lung cancer biomarker derived from airway epithelium gene expression was evaluated for its ability to distinguish between normal and cancerous lung tissue using an Affymetrix HGU95Av2 dataset published by Bhattacharjee et al (21) that we processed using RMA. By mapping Unigene identifiers, 64 HGU95Av2 probesets were identified that measure the expression of genes that corresponded to the 80 probesets in our airway classifier. This resulted in a partial airway epithelium signature that was then used to classify tumor and normal samples from the dataset. In addition, PCA analysis of the lung tissue samples was performed using the expression of these 64 probesets.

To further assess the statistical significance of the relationship between datasets, Gene Set Enrichment Analysis (22) was performed to determine if the 64 biomarker genes are non-randomly distributed within the HGU95Av2 probesets ordered by differential expression between normal and tumor tissue. Finally, a two-tailed Fisher Exact Test was used to test if the proportion of biomarker genes among the genes differentially expressed between normal and tumor lung tissue is different from the overall proportion of differentially expressed genes (see sections, infra).

Statistical Analysis: RMA was performed in BioConductor. The upstream gene filtering by ANCOVA, and the implementation of the weighted voted algorithm and internal cross validation used to generate the data were executed through an R script we wrote for this purpose. The PAM algorithm was carried out using the ‘pamr’ library in R. All other statistical analyses including Student's T-Tests, Fisher's exact tests, ROC curves and PCA were performed using the R statistical package.

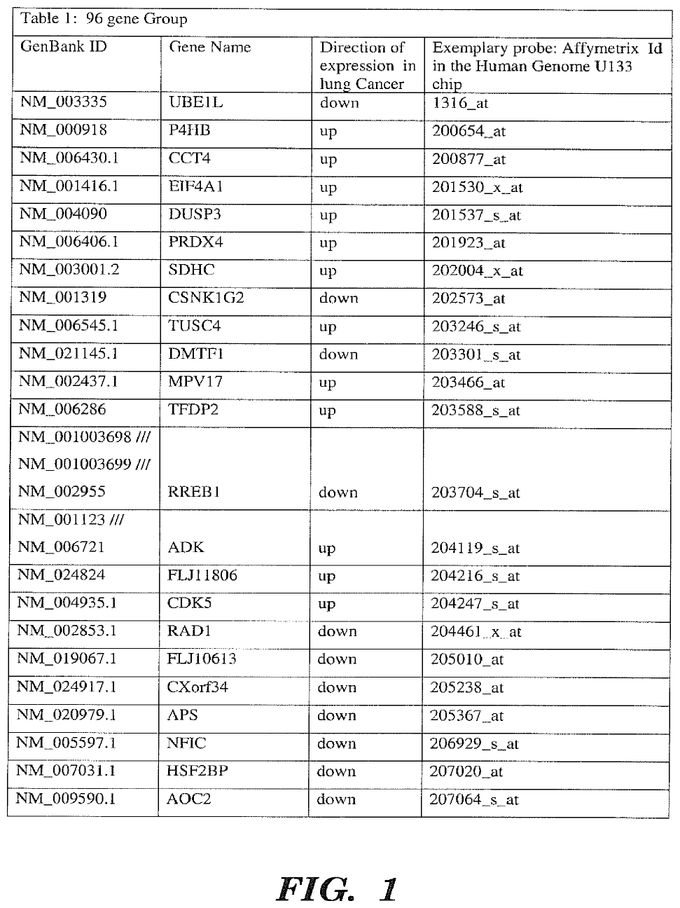

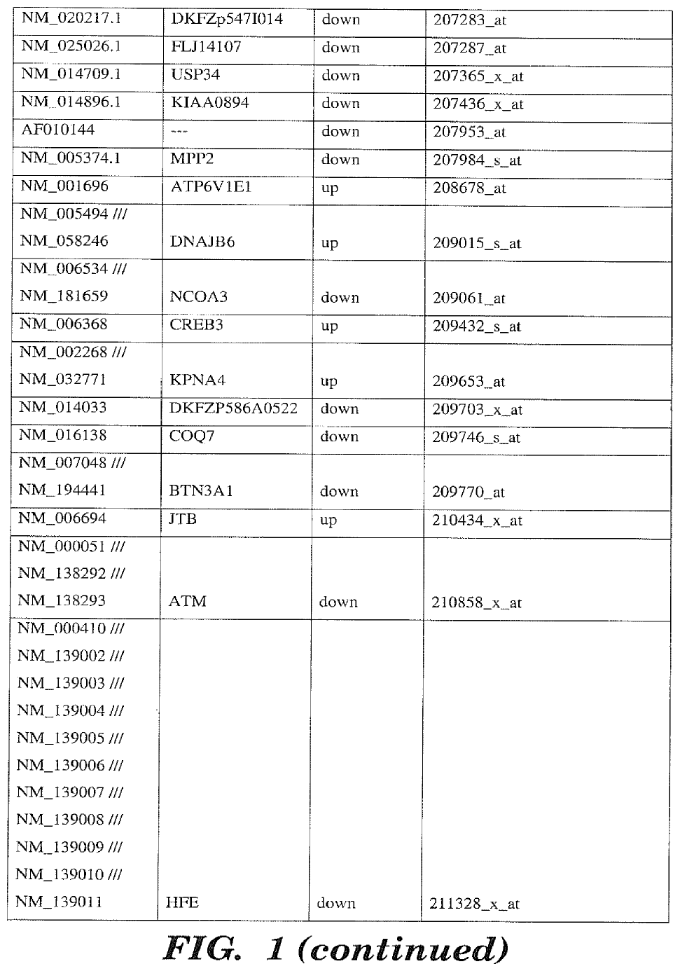

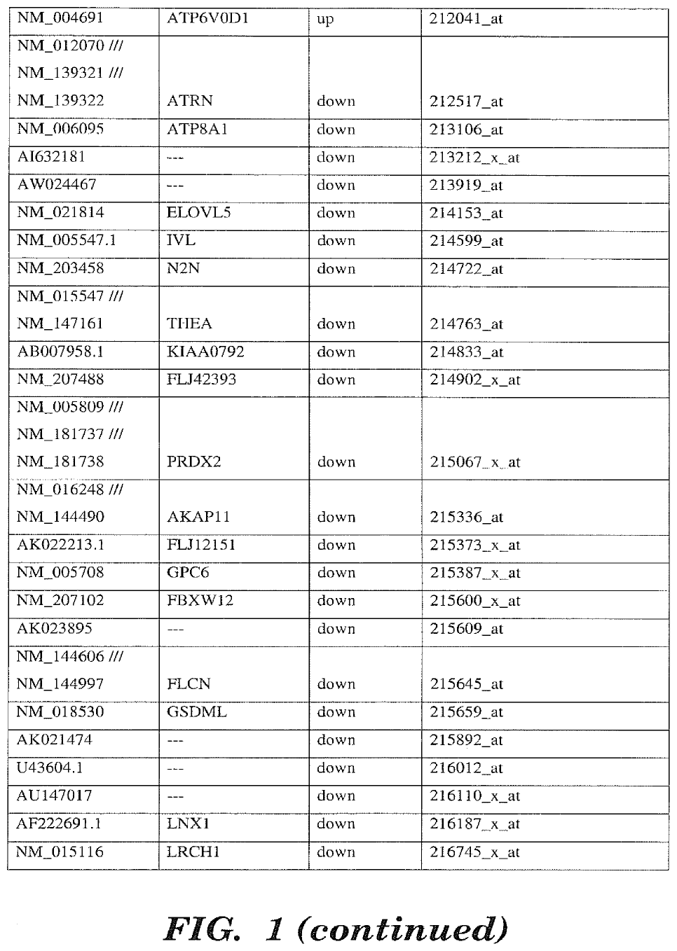

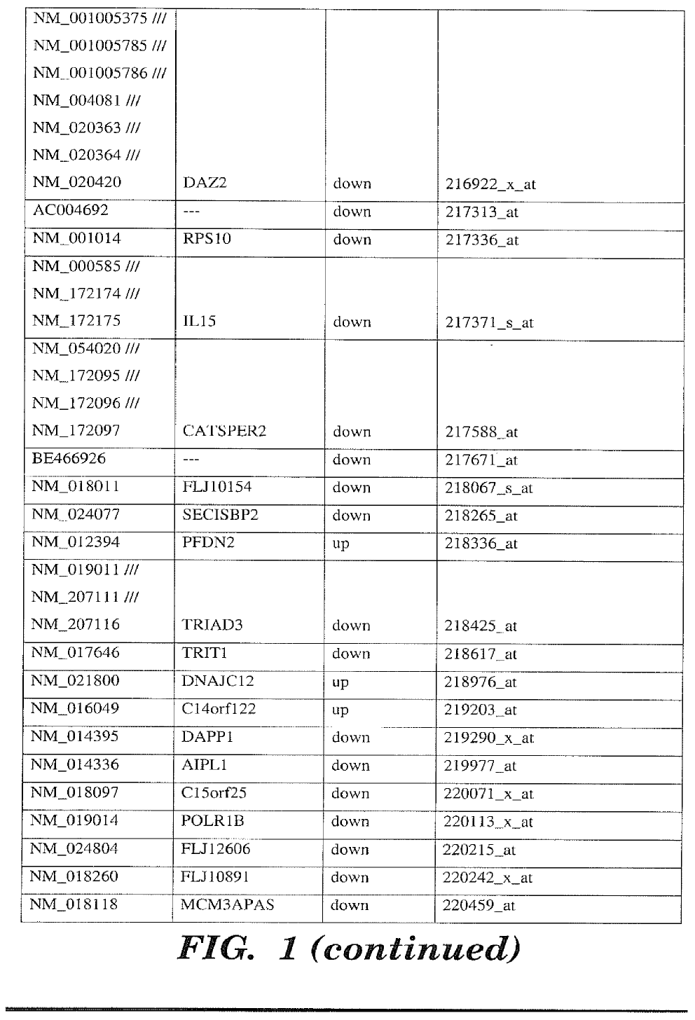

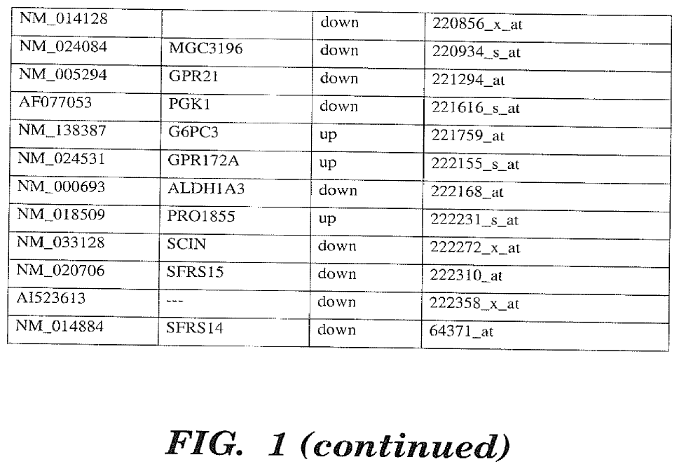

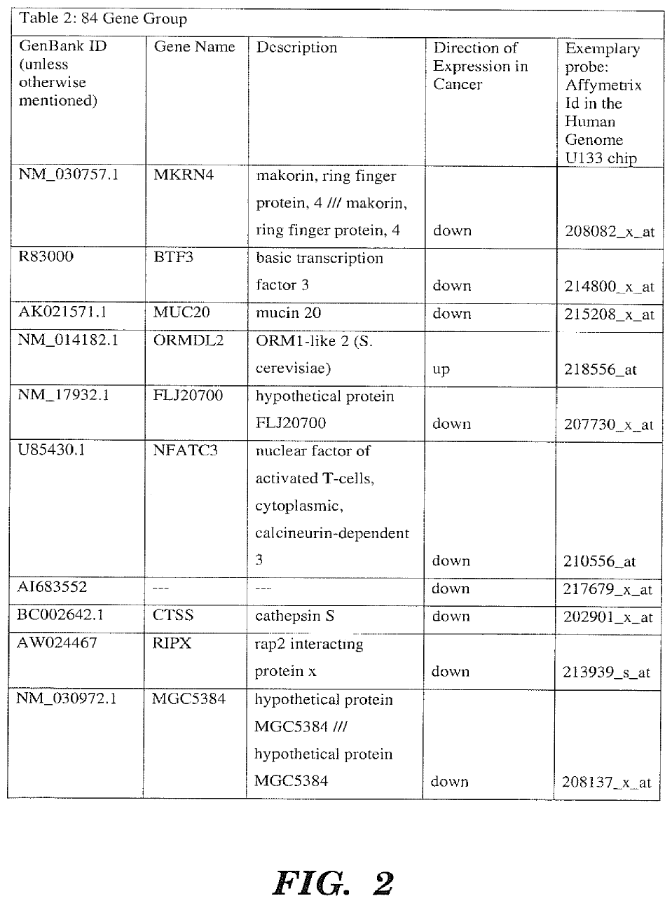

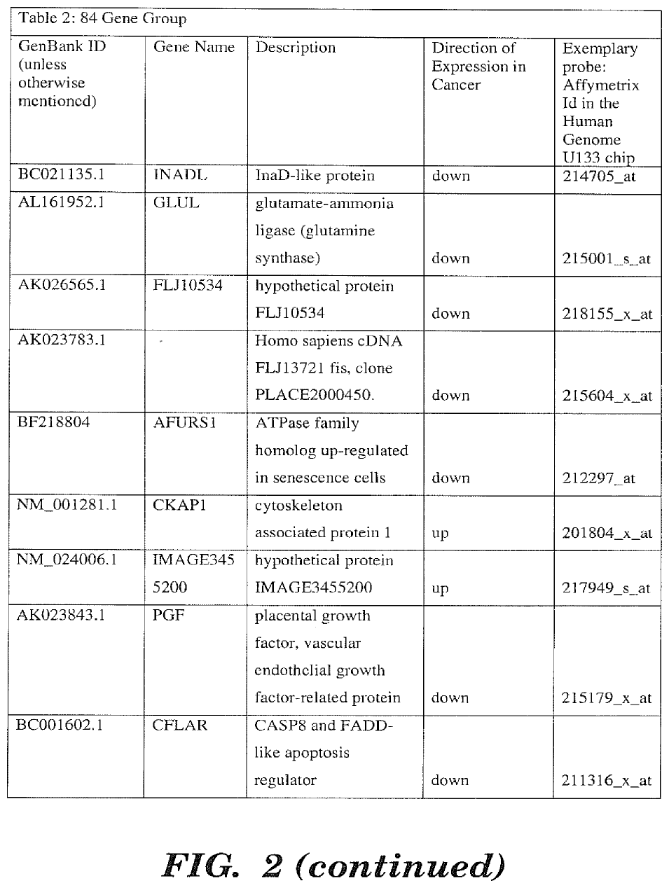

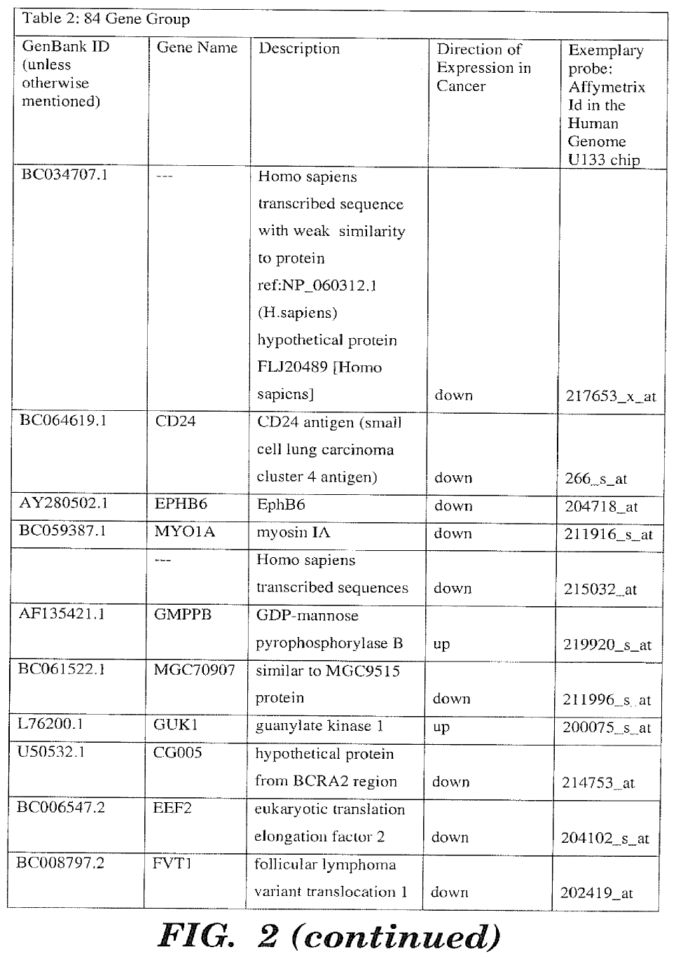

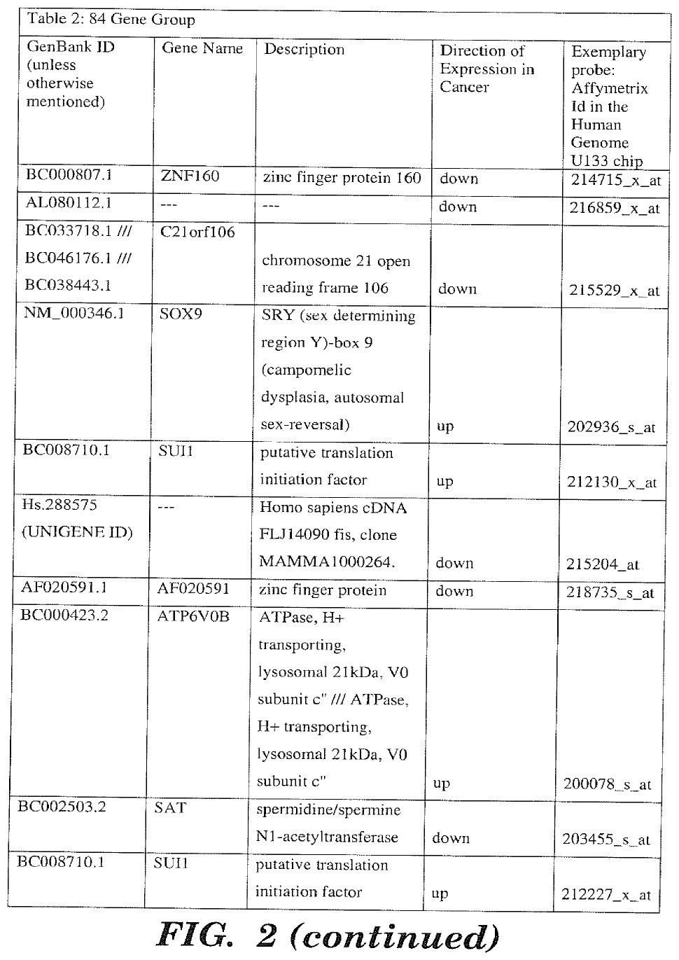

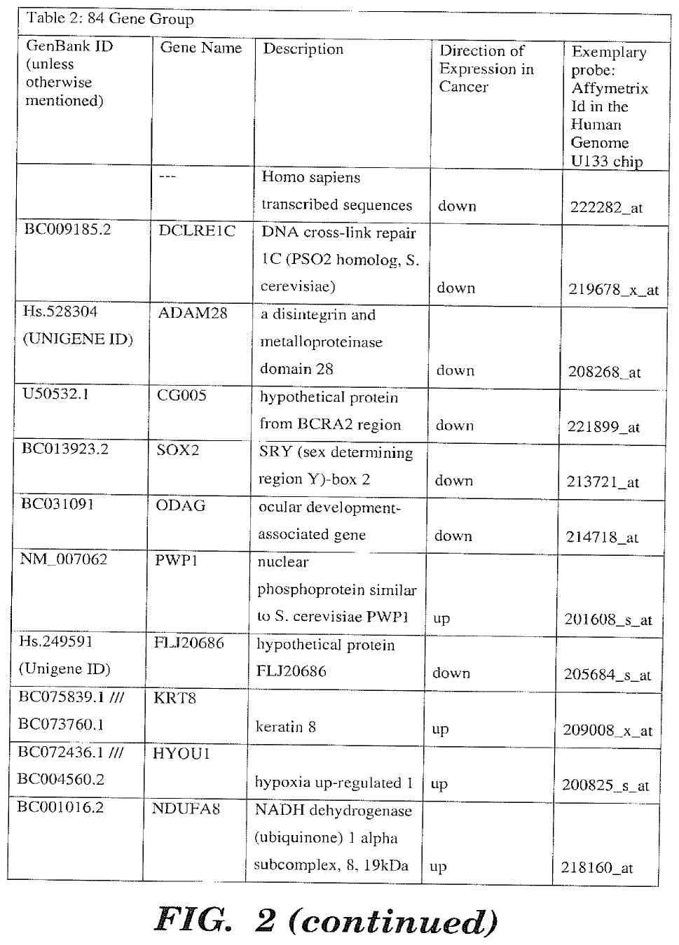

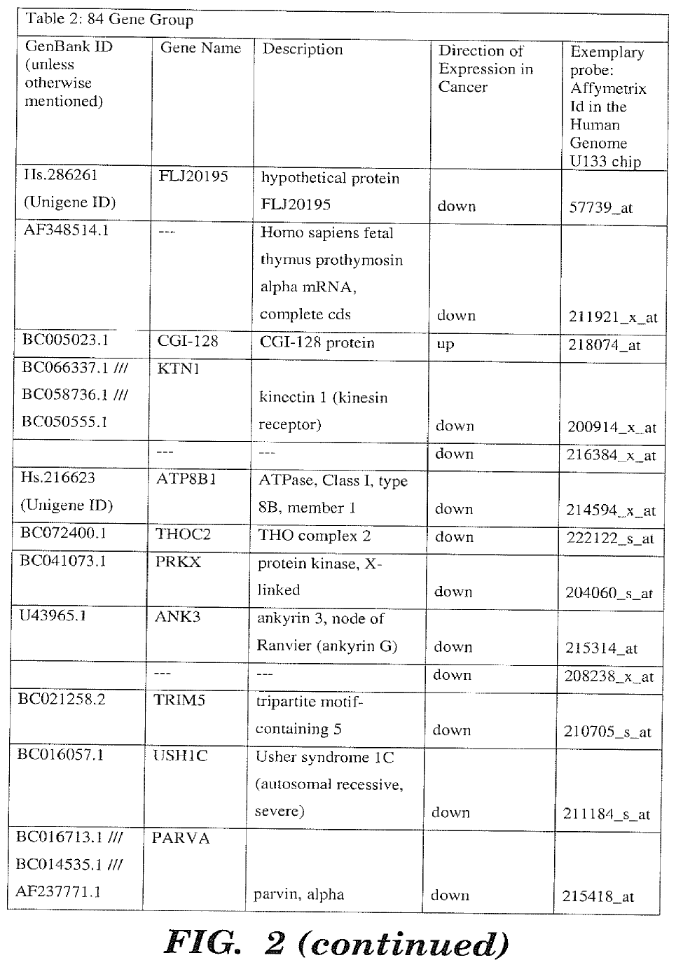

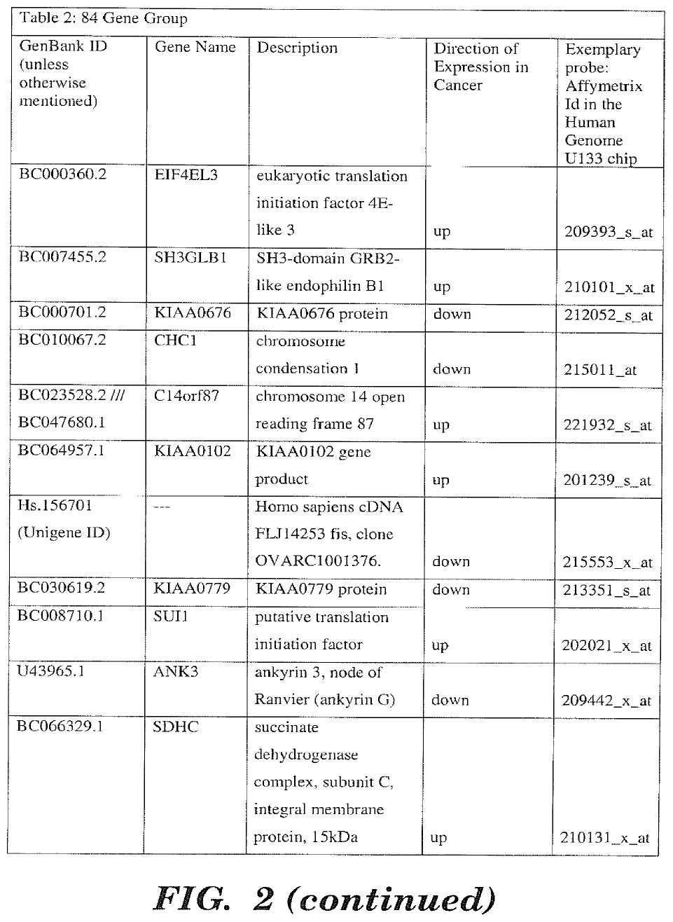

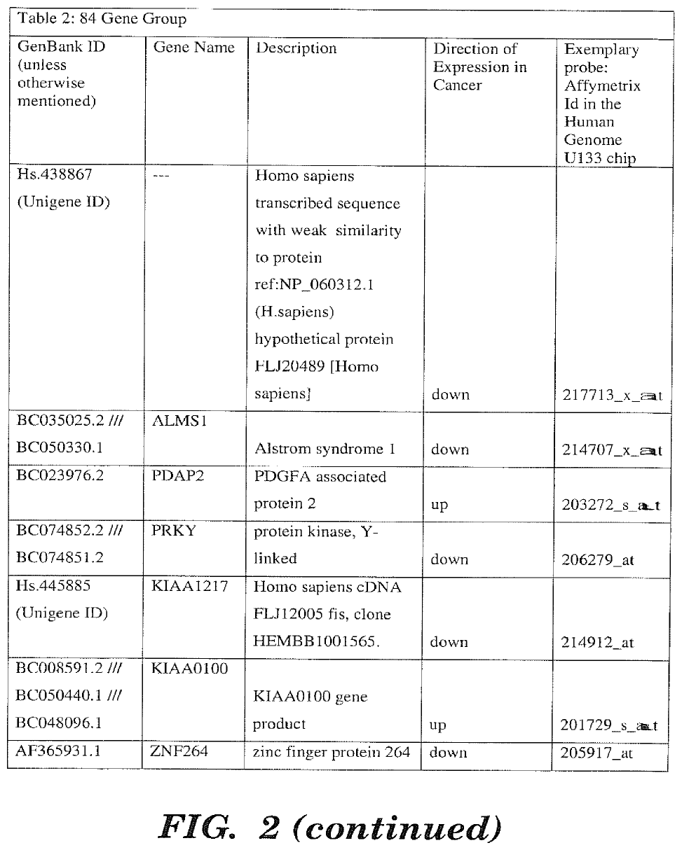

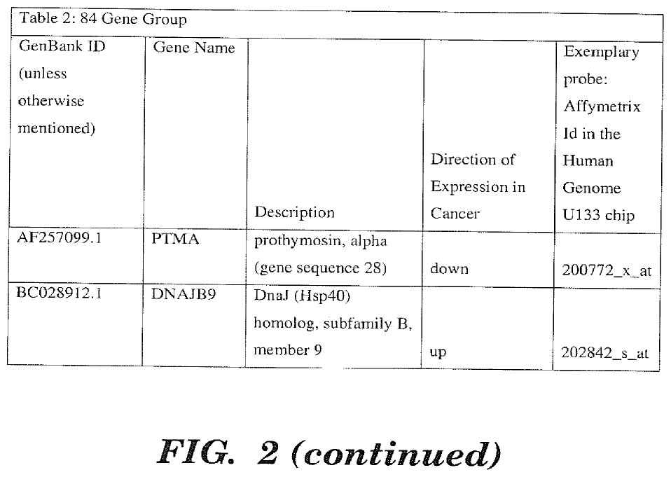

Study Population and Epithelial samples: 129 subjects that had microarrays passing the quality control filter described above were included in the class prediction analysis (see Supplemental FIG. 1). Demographic data on these subjects, including 60 smokers with primary lung cancer and 69 smokers without lung cancer is presented in Table 1. Cell type and stage information for all cancer patients is shown in Supplemental Table 1. Bronchial brushings yielded 90% epithelial cells, as determined by cytokeratin staining, with the majority being ciliated cells with normal bronchial airway morphology. No dysplastic or cancer cells were seen on any representative brushings obtained from smokers with or without cancer.

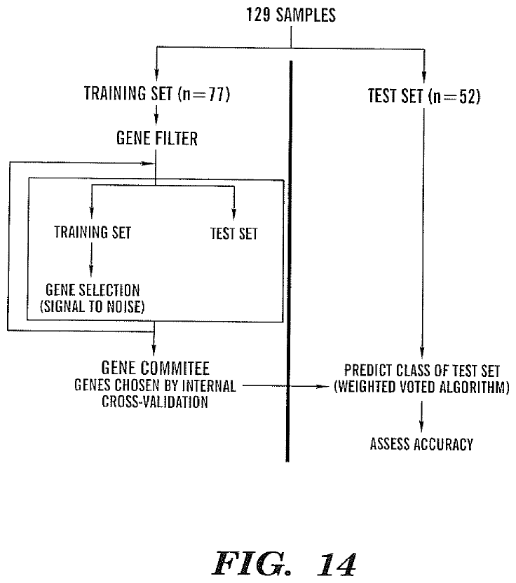

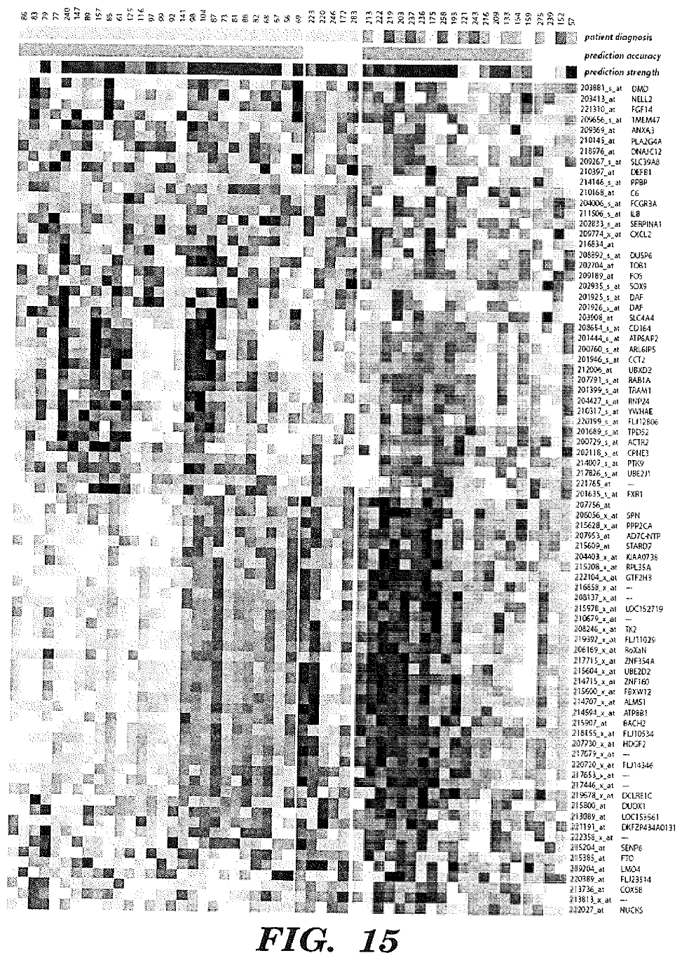

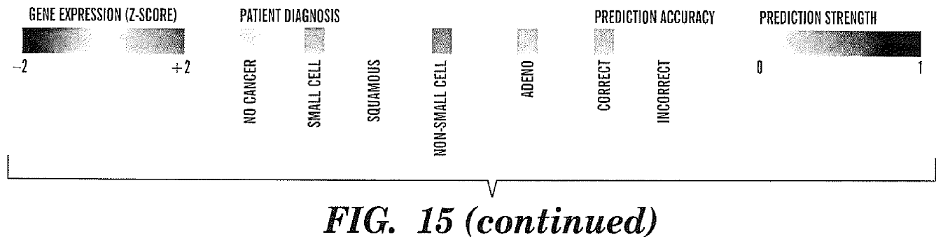

Class Prediction analysis: Comparison of demographic features for 77 subjects in the training set vs. the 52 samples in the test set is shown in Supplemental Table 2. An 80 gene class prediction committee capable of distinguishing smokers with and without cancer was built on the training set of 77 samples and tested on the independent sample set (FIG. 14). The accuracy, sensitivity and specificity of this model was 83%(43/52), 80% (16/20) and 84% (27/32) respectively. When samples predicted with a low degree of confidence (as defined by a Prediction Strength metric<0.3; see Supplement for details) were considered non-diagnostic, the overall accuracy of the model on the remaining 43 samples in the test set increased to 88% (93% sensitivity, 86% specificity). Hierarchical clustering of the 80 genes selected for the diagnostic biomarker in the test set samples is shown in FIG. 15. Principal Component Analysis of all cancer samples according to the expression of these 80 genes did not reveal grouping by cell type (FIG. 10). The accuracy of this 80-gene classifier was similar when microarray data was preprocessed in MAS 5.0 and when the PAM class prediction algorithm was used (see Supplemental Table 3).

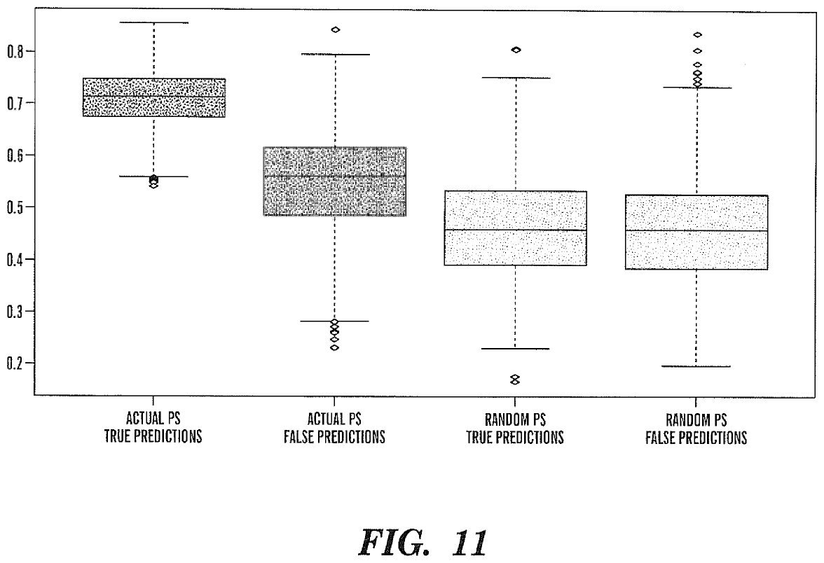

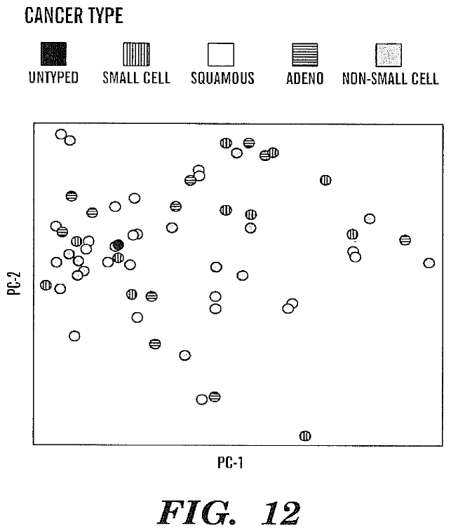

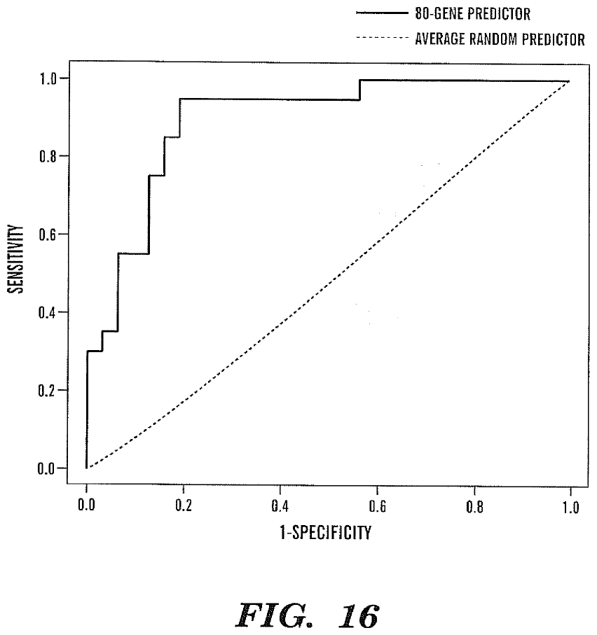

The 80-gene predictor's accuracy, sensitivity and specificity on the 52 sample test set was significantly better than the performance of classifiers derived from randomizing the class labels of the training set (p=0.004; empiric p-value for random classifier AUC>true classifier AUC; FIG. 16). The performance of the classifier was not dependent on the particular composition of the training and test set on which it was derived and tested: 500 training and test sets (derived from the 129 samples) resulted in classifiers with similar accuracy as the classifier derived from our training set (FIG. 11). Finally, we demonstrated that the classifier is better able to distinguish the two sample classes than 500 classifiers derived by randomly selecting genes (see FIG. 12).

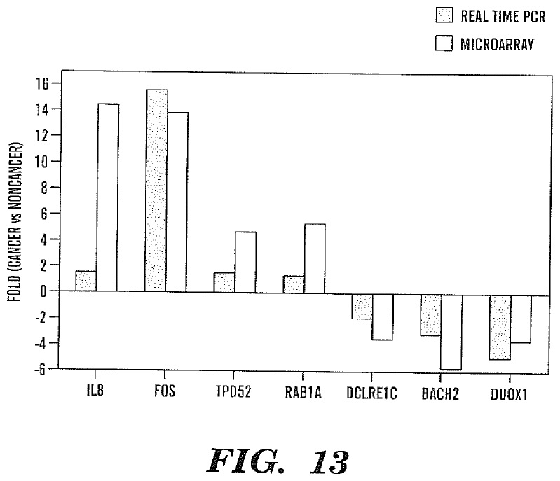

Real time PCR: Differential expression of select genes in our diagnostic airway profile was confirmed by real time PCR (see FIG. 13).

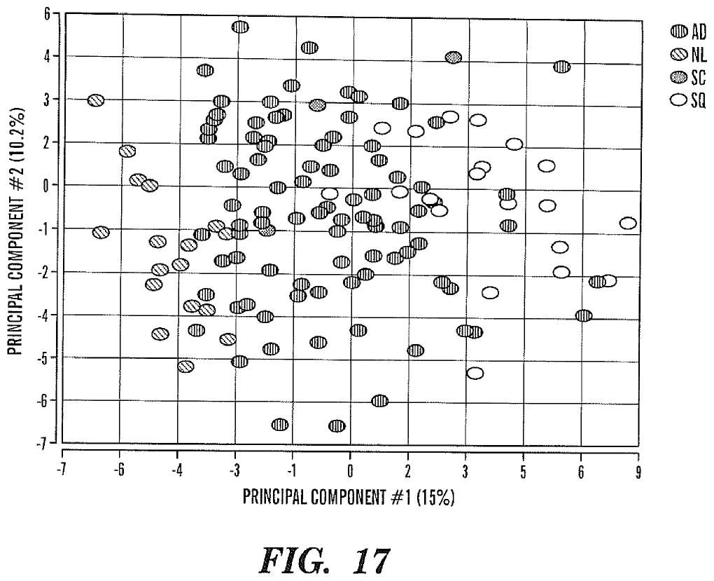

Linking to lung cancer tissue: Our airway biomarker was also able to correctly classify lung cancer tissue from normal lung tissue with 98% accuracy. Principal Component Analysis demonstrated separation of non-cancerous samples from cancerous samples in the Bhattacharjee dataset according to the expression of our airway signature (see FIG. 17). Furthermore, our class prediction genes were statistically overrepresented among genes differentially expressed between cancer vs. no cancer in the Bhattacharjee dataset by Fisher exact test (p<0.05) and Gene Enrichment Analysis (FDR<0.25, see Supplement for details).

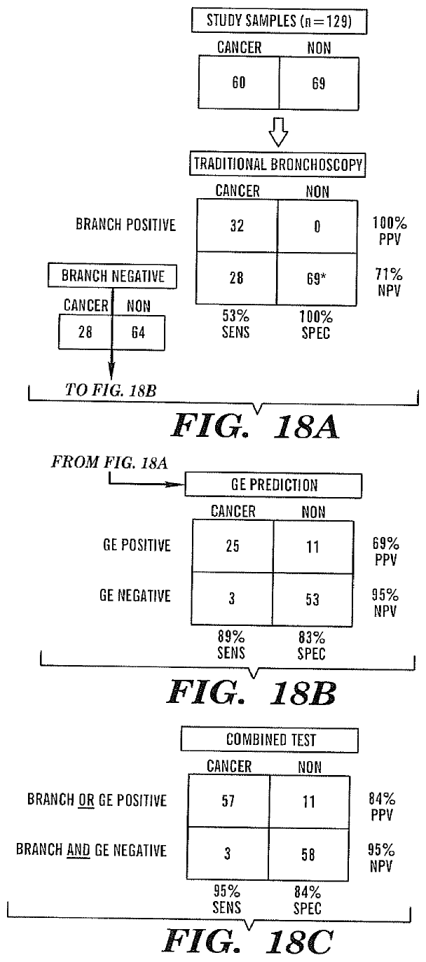

Synergy with Bronchoscopy: Bronchoscopy was diagnostic (via endoscopic brushing, washings or biopsy of the affected region) in 32/60 (53%) of lung cancer patients and 5/69 non-cancer patients. Among non-diagnostic bronchoscopies (n=92), our class prediction model had an accuracy of 85% with 89% sensitivity and 83% specificity. Combining bronchoscopy with our gene expression signature resulted in a 95% diagnostic sensitivity (57/60) across all cancer subjects. Given the approximate 50% disease prevalence in our cohort, a negative bronchoscopy and negative gene expression signature for lung cancer resulted in a 95% negative predictive value (NPV) for disease (FIG. 18). In patients with a negative bronchoscopy, the positive predictive value of our gene expression profile for lung cancer was approximately 70% (FIG. 18).

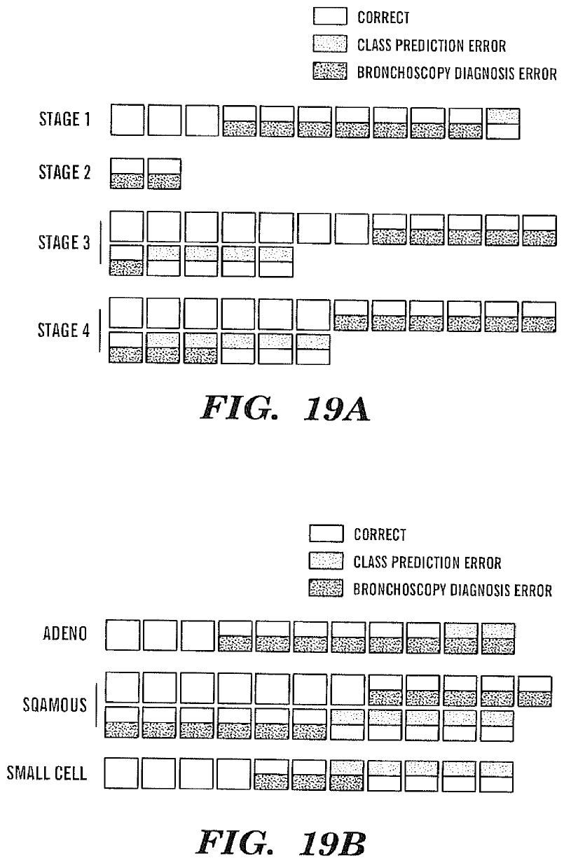

Stage and cell type subgroup analysis: The diagnostic yield of our airway gene expression signature vs. bronchoscopy according to stage and cell type of the lung cancer samples is shown in FIG. 19.

Lung cancer is the leading cause of death from cancer in the United States, in part because of the lack of sensitive and specific diagnostic tools that are useful in early-stage disease. With approximately 90 million former and current smokers in the U.S., physicians increasingly encounter smokers with clinical suspicion for lung cancer on the basis of an abnormal radiographic imaging study and/or respiratory symptoms. Flexible bronchoscopy represents a relatively noninvasive initial diagnostic test to employ in this setting. This study was undertaken in order to develop a gene expression-based diagnostic, that when combined with flexible bronchoscopy, would provide a sensitive and specific one-step procedure for the diagnosis of lung cancer. Based on the concept that cigarette smoking creates a respiratory tract “field defect”, we examined the possibility that profiles of gene expression in relatively easily accessible large airway epithelial cells would serve as an indicator of the amount and type of cellular injury induced by smoking and might provide a diagnostic tool in smokers who were being evaluated for the possibility of lung cancer.

We have previously shown that smoking induces a number of metabolizing and anti-oxidant genes, induces expression of several putative oncogenes and suppresses expression of several potential tumor suppressor genes in large airway epithelial cells (17). We show here that the pattern of airway gene expression in smokers with lung cancer differs from smokers without lung cancer, and the expression profile of these genes in histologically normal bronchial epithelial cells can be used as a sensitive and specific predictor of the presence of lung cancer. We found that the expression signature was particularly useful in early stage disease where bronchoscopy was most often negative and where most problems with diagnosis occur. Furthermore, combining the airway gene expression signature with bronchoscopy results in a highly sensitive diagnostic approach capable of identifying 95% of lung cancer cases.

Given the unique challenges to developing biomarkers for disease using DNA microarrays (23), we employed a rigorous computational approach in the evaluation of our dataset. The gene expression biomarker reported in this paper was derived from a training set of samples obtained from smokers with suspicion of lung cancer and was tested on an independent set of samples obtained from four tertiary medical centers in the US and Ireland. The robust nature of this approach was confirmed by randomly assigning samples into separate training and test sets and demonstrating a similar overall accuracy (FIG. 11). In addition, the performance of our biomarker was significantly better than biomarkers obtained via randomization of class labels in the training set (FIG. 16) or via random 80 gene committees (FIG. 8). Finally, the performance of our 80-gene profile remained unchanged when microarray data was preprocessed via a different algorithm or when a second class prediction algorithm was employed.

In terms of limitations, our study was not designed to assess performance as a function of disease stage or subtype. Our gene expression predictor, however, does appear robust in early stage disease compared with bronchoscopy (see FIG. 19). Our profile was able to discriminate between cancer and no cancer across all subtypes of lung cancer (see FIG. 10). 80% of the cancers in our dataset were NSCLC and our biomarker was thus trained primarily on events associated with that cell type. However, given the high yield for bronchoscopy alone in the diagnosis of small cell lung cancer, this does not limit the sensitivity and negative predictive value of the combined bronchoscopy and gene expression signature approach. A large-scale clinical trial is needed to validate our signature across larger numbers of patients and establish its efficacy in early stage disease as well as its ability to discriminate between subtypes of lung cancer.

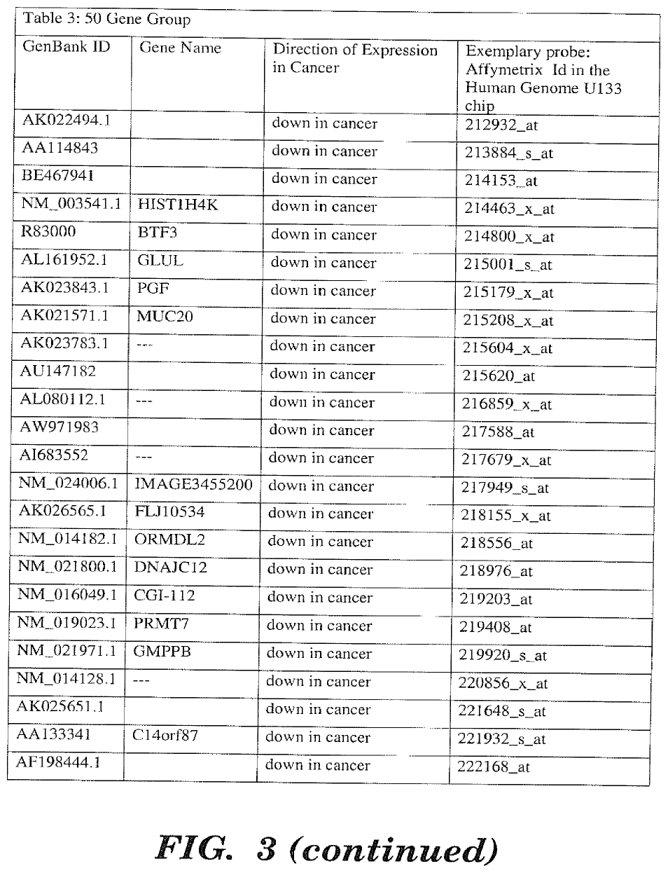

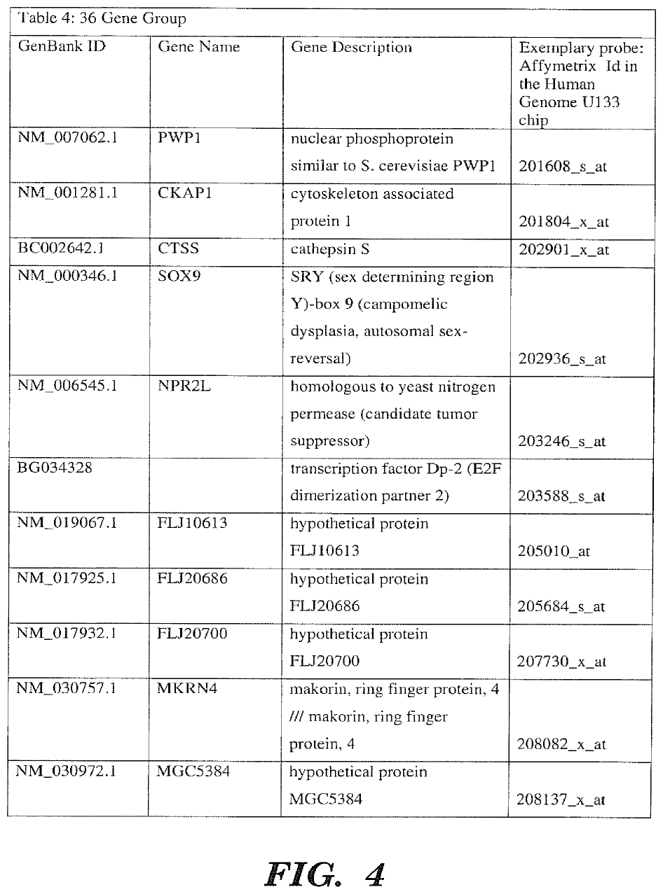

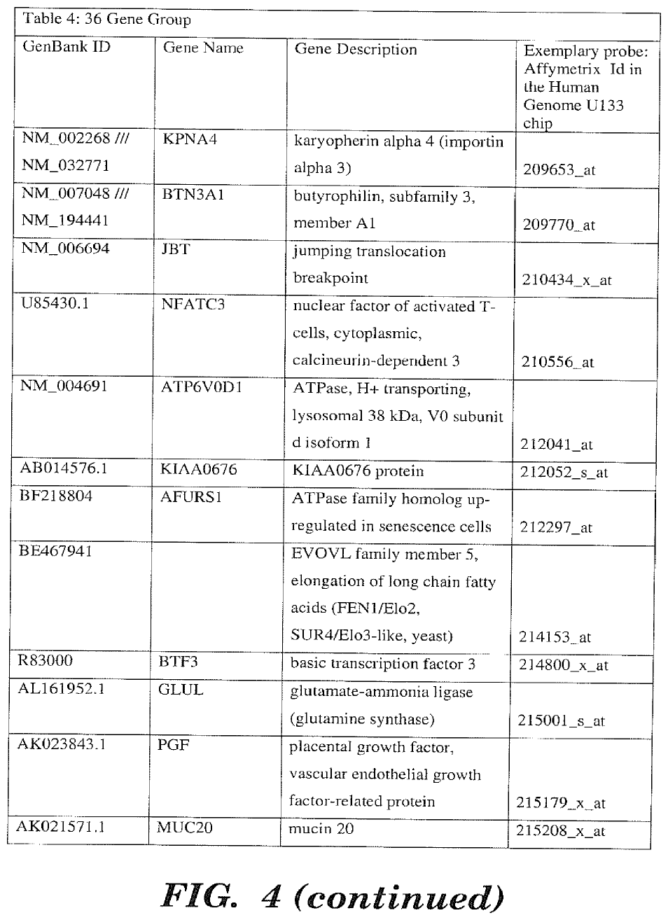

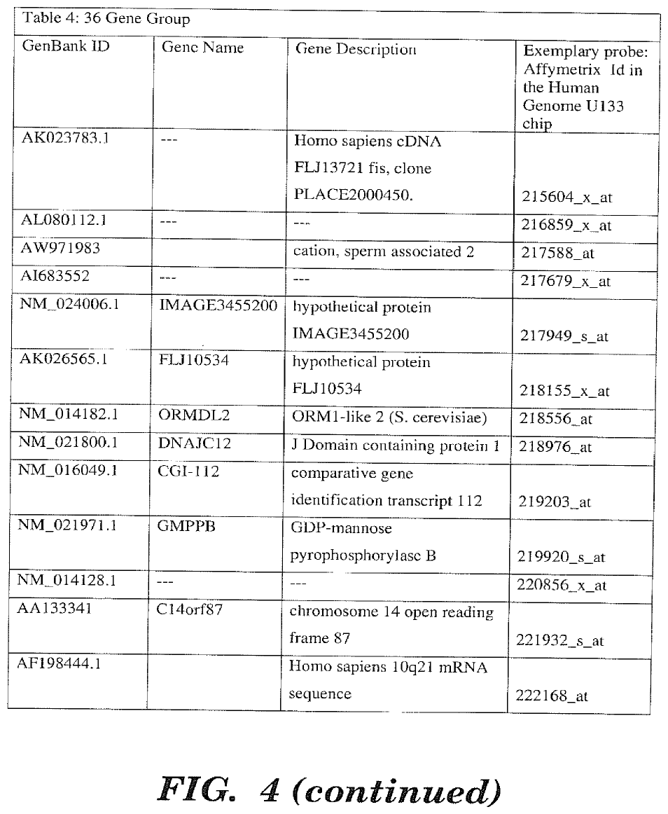

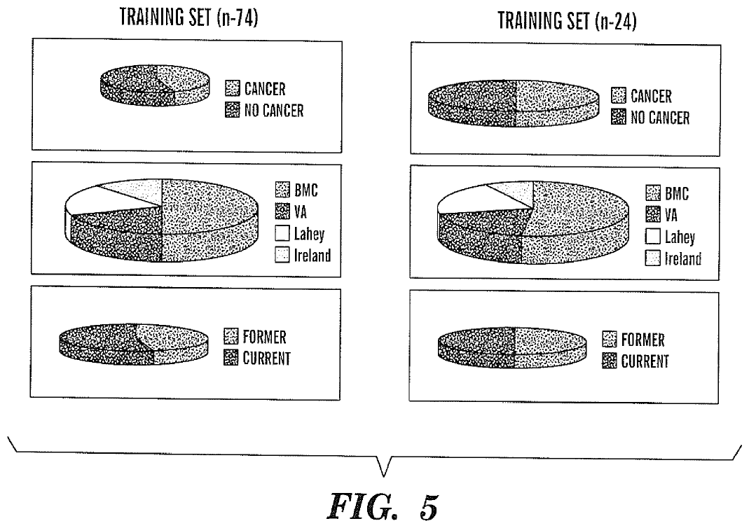

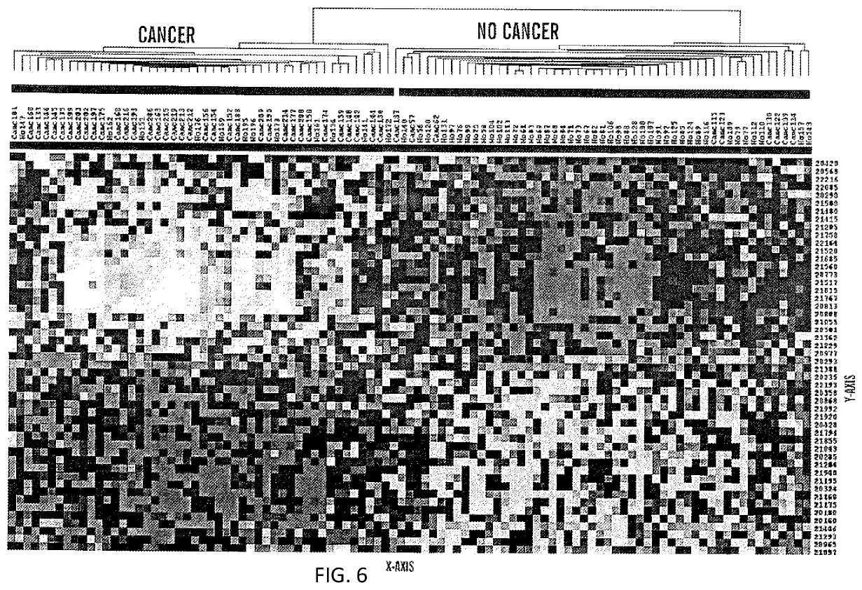

In addition to serving as a diagnostic biomarker, profiling airway gene expression across smokers with and without lung cancer can also provide insight into the nature of the “field of injury” reported in smokers and potential pathways implicated in lung carcinogenesis. Previous studies have demonstrated allelic loss and methylation of tumor suppressor genes in histologically normal bronchial epithelial cells from smokers with and without lung cancer (12; 13; 15). Whether these changes are random mutational effects or are directly related to lung cancer has been unclear. The finding that our airway gene signature was capable of distinguishing lung cancer tissue from normal lung (FIG. 4) suggests that the airway biomarker is, at least in part, reflective of changes occurring in the cancerous tissue and may provide insights into lung cancer biology.

Among the 80 genes in our diagnostic signature, a number of genes associated with the RAS oncogene pathway, including Rab 1a and FOS, are up regulated in the airway of smokers with lung cancer. Rab proteins represent a family of at least 60 different Ras-like GTPases that have crucial roles in vesicle trafficking, signal transduction, and receptor recycling, and dysregulation of RAB gene expression has been implicated in tumorigenesis (24). A recent study by Shimada et al. (25) found a high prevalence of Rab1A-overexpression in head and neck squamous cell carcinomas and also in premalignant tongue lesions, suggesting that it may be an early marker of smoking-related respiratory tract carcinogenesis.

In addition to these RAS pathway genes, the classifier contained several pro-inflammatory genes, including Interleukin-8 (IL-8) and beta-defensin 1 that were up regulated in smokers with lung cancer. IL-8, originally discovered as a chemotactic factor for leukocytes, has been shown to contribute to human cancer progression through its mitogenic and angiogenic properties (26; 27). Beta defensins, antimicrobial agents expressed in lung epithelial cells, have recently found to be elevated in the serum of patients with lung cancer as compared to healthy smokers or patients with pneumonia (28). Higher levels of these mediators of chronic inflammation in response to tobacco exposure may result in increased oxidative stress and contribute to tumor promotion and progression in the lung (29; 30)

A number of key antioxidant defense genes were found to be decreased in airway epithelial cells of subjects with lung cancer, including BACH2 and dual oxidase 1, along with a DNA repair enzyme, DNA repair protein 1C. BACH-2, a transcription factor, promotes cell apoptosis in response to high levels of oxidative-stress (31). We have previously found that a subset of healthy smokers respond differently to tobacco smoke, failing to induce a set of detoxification enzymes in their normal airway epithelium, and that these individuals may be predisposed to its carcinogenic effects (17). Taken together, these data suggest that a component of the airway “field defect” may reflect whether a given smoker is appropriately increasing expression of protective genes in response to the toxin. This inappropriate response may reflect a genetic susceptibility to lung cancer or alternatively, epigenetic silencing or deletion of that gene by the carcinogen.

In summary, our study has identified an airway gene expression biomarker that has the potential to directly impact the diagnostic evaluation of smokers with suspect lung cancer. These patients usually undergo fiberoptic bronchoscopy as their initial diagnostic test. Gene expression profiling can be performed on normal-appearing airway epithelial cells obtained in a simple, non-invasive fashion at the time of the bronchoscopy, prolonging the procedure by only 3-5 minutes, without adding significant risks. Our data strongly suggests that combining results from bronchoscopy with the gene expression biomarker substantially improves the diagnostic sensitivity for lung cancer (from 53% to 95%). In a setting of 50% disease prevalence, a negative bronchoscopy and negative gene expression signature for lung cancer results in a 95% negative predictive value (NPV), allowing these patients to be followed non-aggressively with repeat imaging studies. For patients with a negative bronchoscopy and positive gene expression signature, the positive predictive value is ˜70%, and these patients would likely require further invasive testing (i.e. transthoracic needle biopsy or open lung biopsy) to confirm the presumptive lung cancer diagnosis. However, this represents a substantial reduction in the numbers of patients requiring further invasive diagnostic testing compared to using bronchoscopy alone. In our study, 92/129 patients were bronchoscopy negative and would have required further diagnostic work up. However, the negative predictive gene expression profile in 56 of these 92 negative bronchoscopy subjects would leave only 36 subjects who would require further evaluation (see FIG. 18).

The cross-sectional design of our study limits interpretation of the false positive rate for our signature. Given that the field of injury may represent whether a smoker is appropriately responding to the toxin, derangements in gene expression could precede the development of lung cancer or indicate a predisposition to the disease. Long-term follow-up of the false positive cases is needed (via longitudinal study) to assess whether they represent smokers who are at higher risk for developing lung cancer in the future. If this proves to be true, our signature could serve as a screening tool for lung cancer among healthy smokers and have the potential to identify candidates for chemoprophylaxis trials.

### Study Patients and Sample Collection

A. Primary sample set: We recruited current and former smokers undergoing flexible bronchoscopy for clinical suspicion of lung cancer at four tertiary medical centers. All subjects were older than 21 years of age and had no contraindications to flexible bronchoscopy including hemodynamic instability, severe obstructive airway disease, unstable cardiac or pulmonary disease (i.e. unstable angina, congestive heart failure, respiratory failure) inability to protect airway or altered level of consciousness and inability to provide informed consent. Never smokers and subjects who only smoked cigars were excluded from the study. For each consented subject, we collected data regarding their age, gender, race, and a detailed smoking history including age started, age quit, and cumulative tobacco exposure. Former smokers were defined as patients who had not smoked a cigarette for at least one month prior to entering our study. All subjects were followed, post-bronchoscopy, until a final diagnosis of lung cancer or an alternative diagnosis was made (mean follow-up time=52 days). For those patients diagnosed with lung cancer, the stage and cell type of their tumor was recorded. The clinical data collected from each subject in this study can be accessed in a relational database at http://pulm.bumc.bu.edu/CancerDx/. The stage and cell type of the 60 cancer samples used to train and test the class prediction model is shown in Supplemental Table 1 below.

| | | Stage | |
|---|---|---|---|
| Cell Type | | NSCLC staging | |
| NSCLC | 48 | IA | 2 |
| Squamous Cell | 23 | IB | 9 |
| Adenocarcinoma | 11 | IIA | 2 |
| Large Cell | 4 | IIB | 0 |
| Not classified | 10 | IIIA | 9 |
| Small Cell | 11 | IIIE | 9 |
| Unknown | 1 | IV | 17 |

Supplemental Table 1 above shows cell type and staging information for 60 lung cancer patients in the 129 primary sample set used to build and test the class prediction model. Staging information limited to the 48 non-small cell samples.

The demographic features of the samples in training and test shown are shown in Supplemental Table 2 below. The Table shows patient demographics for the primary dataset (n=129) according to training and test set status. Statistical differences between the two patient classes and associated p values were calculated using T-tests, Chi-square tests and Fisher's exact tests where appropriate. PPD=packs per day, F=former smokers, C=current smokers, M=male,

| | Training set | Test set |
|---|---|---|
| Samples | 77 | 52 |
| Age | 59.3 +/− 13.1 | 52.1 +/− 15.6 |
| Smoking Status | 41.6% F, 58.4% C | 48.1% F, 51.9% C |
| Gender* | 83.1% M, 16.9% F | 67.3% M, 32.7% F |
| PackYears | 45.6 +/− 31 | 37.7 +/− 27.8 |
| Age Started | 16.2 +/− 6.3 | 15.8 +/− 5.3 |
| Smoking intensity | 1.1 +/− 0.53 | 1 +/− 0.5 |
| (PPD): Currents | | |
| Months Quit: | 128 +/− 139 | 139 +/− 141 |
| Formers | | |

*Two classes statistically different: p < 0.05

F=female.

While our study recruited patients whose indication for bronchoscopy included a suspicion for lung cancer, each patient's clinical pre-test probability for disease varied. In order to ensure that our class prediction model was trained on samples representing a spectrum of lung cancer risk, three independent pulmonary clinicians, blinded to the final diagnoses, evaluated each patient's clinical history (including age, smoking status, cumulative tobacco exposure, co-morbidities, symptoms/signs and radiographic findings) and assigned a pre-bronchoscopy probability for lung cancer. Each patient was classified into one of three risk groups: low (<10% probability of lung cancer), medium (10-50% probability of lung cancer) and high (>50% probability of lung cancer). The final risk assignment for each patient was decided by the majority opinion.

### Prospective Sample Set:

After completion of the primary study, a second set of samples was collected from smokers undergoing flexible bronchoscopy for clinical suspicion of lung cancer at 5 medical centers (St. Elizabeth's Hospital in Boston, Mass. was added to the 4 institutions used for the primary dataset). Inclusion and exclusion criteria were identical to the primary sample set. Forty additional subjects were included in this second validation set. Thirty-five subjects had microarrays that passed our quality-control filter. Demographic data on these subjects, including 18 smokers with primary lung cancer and 17 smokers without lung cancer, is presented in Supplemental Table 3. There was no statistical difference in age or cumulative tobacco exposure between case and controls in this prospective cohort (as opposed to the primary dataset; see Table 1a).

Supplemental Table 3 below shows patient demographics for the prospective validation set (n=35) by cancer status. Statistical differences between the two patient classes and associated p values were calculated using T-tests, Chi-square tests and Fisher's exact tests where appropriate. PPD=packs per day, F=former smokers, C=current smokers, M=male, F=female.

| | Cancer | No Cancer |
|---|---|---|
| Samples | 18 | 17 |
| Age | 66.1+/− 11.4 | 62.2 +/− 11.1 |
| Smoking Status | 66.7% F, 33.3% C | 52.9% F, 47.1% C |
| Gender* | 66.6% M, 33.3% F | 70.6% M, 29.4% F |
| PackYears | 46.7 +/− 28.8 | 60 +/− 44.3 |
| Age Started | 16.4 +/− 7.3 | 14.2 +/− 3.8 |
| Smoking intensity | 1.1 +/− 0.44 | 1.2 +/− 0.9 |
| (PPD): Currents | | |
| Months Quit: | 153 +/− 135 | 93 +/− 147 |
| Formers | | |

*Two classes statistically different: p < 0.05

### Airway Epithelial Cell Collection:

Bronchial airway epithelial cells were obtained from the subjects described above via flexible bronchoscopy. Following local anesthesia with 2% topical lidocaine to the oropharynx, flexible bronchoscopy was performed via the mouth or nose. Following completion of the standard diagnostic bronchoscopy studies (i.e. bronchoalveolar lavage, brushing and endo/transbronchial biopsy of the affected region), brushings were obtained via three endoscopic cytobrushes from the right mainstem bronchus. The cytobrush was rubbed over the surface of the airway several times and then retracted from the bronchoscope so that epithelial cells could be placed immediately in TRIzol solution and kept at −80° C. until RNA isolation was performed.

Given that these patients were undergoing bronchoscopy for clinical indications, the risks from our study were minimal, with less than a 5% risk of a small amount of bleeding from these additional brushings. The clinical bronchoscopy was prolonged by approximately 3-4 minutes in order to obtain the research samples. All participating subjects were recruited by IRB-approved protocols for informed consent, and participation in the study did not affect subsequent treatment. Patient samples were given identification numbers in order to protect patient privacy.

### Microarray Data Acquisition and Preprocessing

Microarray data acquisition: 6-8 μg of total RNA from bronchial epithelial cells were converted into double-stranded cDNA with SuperScript II reverse transcriptase (Invitrogen) using an oligo-dT primer containing a T7 RNA polymerase promoter (Genset, Boulder, Colo.). The ENZO Bioarray RNA transcript labeling kit (Enzo Life Sciences, Inc, Farmingdale, N.Y.) was used for in vitro transcription of the purified double stranded cDNA. The biotin-labeled cRNA was then purified using the RNeasy kit (Qiagen) and fragmented into fragments of approximately 200 base pairs by alkaline treatment. Each cRNA sample was then hybridized overnight onto the Affymetrix HG-U133A array followed by a washing and staining protocol. Confocal laser scanning (Agilent) was then performed to detect the streptavidin-labeled fluor.

Preprocessing of array data via RMA: The Robust Multichip Average (RMA) algorithm was used for background adjustment, normalization, and probe-level summarization of the microarray samples in this study (Irizarry R A, et al., Summaries of Affymetrix GeneChip probe level data. Nucleic Acids Res 2003; 31(4):e15.). RMA expression measures were computed using the R statistical package and the justRMA function in the Affymetrix Bioconductor package. A total of 296 CEL files from airway epithelial samples included in this study as well as those previously processed in our lab were analyzed using RMA. RMA was chosen for probe-level analysis instead of Microarray Suite 5.0 because it maximized the correlation coefficients observed between 7 pairs of technical replicates (Supplemental Table 4).

SUPPLEMENTAL TABLE 4
Pearson Correlation Coefficients (22,215 probe-sets)

| | Affy | log2Affy | RMA |
|---|---|---|---|
| Average | 0.972 | 0.903 | 0.985 |
| SD | 0.017 | 0.029 | 0.009 |
| Median | 0.978 | 0.912 | 0.987 |

Supplemental Table 4 shows the Average Pearson Correlations between 7 pairs of replicate samples where probe-set gene expression values were determined using Microarray Suite 5.0 (Affy), logged data from Microarray Suite 5.0 (log 2 Affy), and RMA. RMA maximizes the correlation between replicate samples.

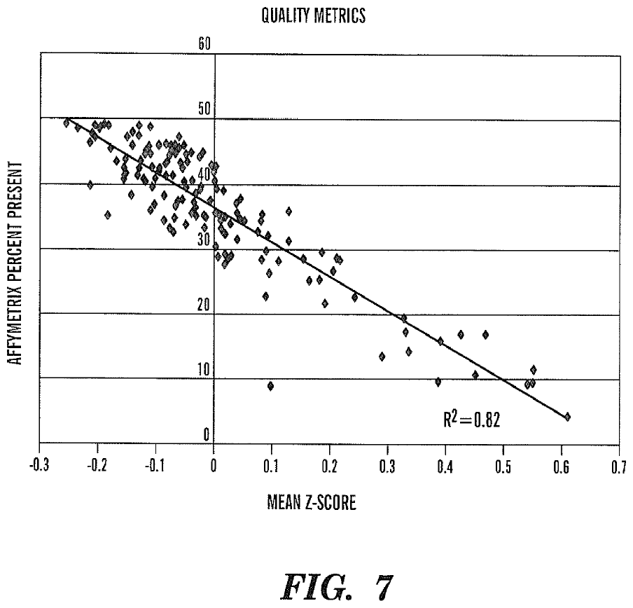

Sample filter: To filter out arrays of poor quality, each probeset on the array was z-score normalized to have a mean of zero and a standard deviation of 1 across all 152 samples. These normalized gene-expression values were averaged across all probe-sets for each sample. The assumption explicit in this analysis is that poor-quality samples will have probeset intensities that consistently trend higher or lower across all samples and thus have an average z-score that differs from zero. This average z-score metric correlates with Affymetrix MAS 5.0 quality metrics such as percent present (FIG. 7) and GAPDH 3′/5′ ratio. Microarrays that had an average z-score with a value greater than 0.129 (˜15% of the 152 samples) were filtered out. The resulting sample set consisted of 60 smokers with cancer and 69 smokers without cancer.

Prospective validation test set: CEL files for the additional 40 samples were added to the collection of airway epithelial CEL files described above, and the entire set was analyzed using RMA to derive expression values for the new samples. Microarrays that had an average z-score with a value greater than 0.129 (5 of the 40 samples) were filtered out. Class prediction of the 35 remaining prospective samples was conducted using the vote weights for the 80-predictive probesets derived from the training set of 77 samples using expression values computed in the section above.

### Microarray Data Analysis

Class Prediction Algorithm: The 129-sample set (60 cancer samples, 69 no cancer samples) was used to develop a class-prediction algorithm capable of distinguishing between the two classes. One potentially confounding difference between the two groups is a difference in cumulative tobacco-smoke exposure as measured by pack-years. To insure that the genes chosen for their ability to distinguish patients with and without cancer in the training set were not simply distinguishing this difference in tobacco smoke exposure, the pack-years each patient smoked was included as a covariate in the training set ANCOVA gene filter.

In addition, there are differences in the pre-bronchoscopy clinical risk for lung cancer among the 129 patients. Three physicians reviewed each patient's clinical data (including demographics, smoking histories, and radiographic findings) and divided the patients into three groups: high, medium, and low pre-bronchoscopy risk for lung cancer (as described above). In order to control for differences in pre-bronchoscopy risk for lung cancer between the patients with and without a final diagnosis of lung cancer, the training set was constructed with roughly equal numbers of cancer and no cancer samples from a spectrum of lung cancer risk.

The weighted voting algorithm (Golub T R, et al. Molecular classification of cancer: class discovery and class prediction by gene expression monitoring. Science 1999; 286(5439):531-537) was implemented as the class prediction method, with several modifications to the gene-selection methodology. Genes that varied between smokers with and without cancer in the training set samples after adjusting for tobacco-smoke exposure (p<0.05) were identified using an ANCOVA with pack-years as the covariate. Further gene selection was performed using the signal to noise metric and internal cross-validation where the 40 most consistently up- and the 40 most consistently down-regulated probesets were identified. The internal cross validation involved leaving 30% of the training samples out of each round of cross-validation, and selecting genes based on the remaining 70% of the samples. The final gene committee consisted of eighty probesets that were identified as being most frequently up-regulated or down-regulated across 50 rounds of internal cross-validation. The parameters of this gene-selection algorithm were chosen to maximize the average accuracy, sensitivity and specificity obtained from fifty runs. This algorithm was implemented in R and yields results that are comparable to the original implementation of the weighted-voted algorithm in GenePattern when a specific training, test, and gene set are given as input.

After determination of the optimal gene-selection parameters, the algorithm was run using a training set of 77 samples to arrive at a final set of genes capable of distinguishing between smokers with and without lung cancer. The accuracy, sensitivity and specificity of this classifier were tested against 52 samples that were not included in the training set. The performance of this classifier in predicting the class of each test-set sample was assessed by comparing it to runs of the algorithm where either: 1) different training/tests sets were used; 2) the cancer status of the training set of 77 samples were randomized; or 3) the genes in the classifier were randomly chosen (see randomization section below for details).

Randomization: The accuracy, sensitivity, specificity, and area under the ROC curve (using the signed prediction strength as a continuous cancer predictor) for the 80-probeset predictor (above) were compared to 1000 runs of the algorithm using three different types of randomization. First, the class labels of the training set of 77 samples were permuted and the algorithm, including gene selection, was re-run 1000 times (referred to in Supplemental Table 5 as Random 1).

Supplemental Table 5 below shows results of a comparison between the actual classifier and random runs (explained above). Accur=Accuracy, Sens=Sensitivity, Spec=Specificity, AUC=area under the curve, and sd=standard deviation. All p-value are empirically derived.

SUPPLEMENTAL TABLE 5

| | Accur | sd (Accur) | p-value | Sens | sd (Sens) | p-value | Spec | sd (Spec) | p-value | AUC | sd (AUC) | p-value |
|---|---|---|---|---|---|---|---|---|---|---|---|---|
| Actual | 0.827 | | | 0.8 | | | 0.844 | | | 0.897 | | |
| Classifier | | | | | | | | | | | | |
| Random1 | 0.491 | 0.171 | 0.018 | 0.487 | 0.219 | 0.114 | 0.493 | 0.185 | 0.015 | 0.487 | 0.223 | 0.004 |
| Random 2 | 0.495 | 0.252 | 0.078 | 0.496 | 0.249 | 0.173 | 0.495 | 0.263 | 0.073 | 0.495 | 0.309 | 0.008 |
| Random 3 | 0.495 | 0.193 | 0.021 | 0.491 | 0.268 | 0.217 | 0.498 | 0.17 | 0.006 | 0.492 | 0.264 | 0.007 |

The second randomization used the 80 genes in the original predictor but permuted the class labels of the training set samples over 1000 runs to randomize the gene weights used in the classification step of the algorithm (referred to in Supplemental Table 5 as Random 2).

In both of these randomization methods, the class labels were permuted such that half of the training set samples was labeled correctly. The third randomization method involved randomly selecting 80 probesets for each of 1000 random classifiers (referred to in Supplemental Table 5 as Random 3).

The p-value for each metric and randomization method shown indicate the percentage of 1000 runs using that randomization method that exceeded or was equal to the performance of the actual classifier.

In addition to the above analyses, the actual classifier was compared to 1000 runs of the algorithm where different training/test sets were chosen but the correct sample labels were retained. Empirically derived p-values were also computed to compare the actual classifier to the 1000 runs of the algorithm (see Supplemental Table 6). These data indicate that the actual classifier was derived using a representative training and test set.

SUPPLEMENTAL TABLE 6

| | Accur | sd(Accur) | p-value | Sens | sd(Sens) | p-value | Spec | sd(Spec) | p-value | AUC | sd(AUC) | p-value |
|---|---|---|---|---|---|---|---|---|---|---|---|---|
| Actual | 0.827 | | | 0.8 | | | 0.844 | | | 0.897 | | |
| Classifier | | | | | | | | | | | | |
| 1000 Runs | 0.784 | 0.054 | 0.283 | 0.719 | 0.104 | 0.245 | 0.83 | 0.06 | 0.407 | 0.836 | 0.053 | 0.108 |

Supplemental Table 6 above shows a comparison of actual classifier to 1000 runs of the algorithm with different training/test sets.

Finally, these 1000 runs of the algorithm were also compared to 1000 runs were the class labels of different training sets were randomized in the same way as described above. Empirically derived p-values were computed to compare 1000 runs to 1000 random runs (Supplemental Table 7).

SUPPLEMENTAL TABLE 7

| | Accur | sd(Accur) | p-value | Sens | sd(Sens) | p-value | Spec | sd(Spec) | p-value | AUC | sd(AUC) | p-value |
|---|---|---|---|---|---|---|---|---|---|---|---|---|
| 1000 Runs | 0.784 | 0.054 | | 0.719 | 0.104 | | 0.83 | 0.06 | | 0.836 | 0.053 | |
| 1000 Random | 0.504 | 0.126 | 0.002 | 0.501 | 0.154 | 0.025 | 0.506 | 0.154 | 0.003 | 0.507 | 0.157 | 0.001 |
| Runs | | | | | | | | | | | | |

Supplemental Table 7 above shows comparison of runs of the algorithm using different training/test sets to runs where the class labels of the training sets were randomized (1000 runs were conducted).

The distribution of the prediction accuracies summarized in Supplemental Tables 6 and 7 is shown in FIG. 8.

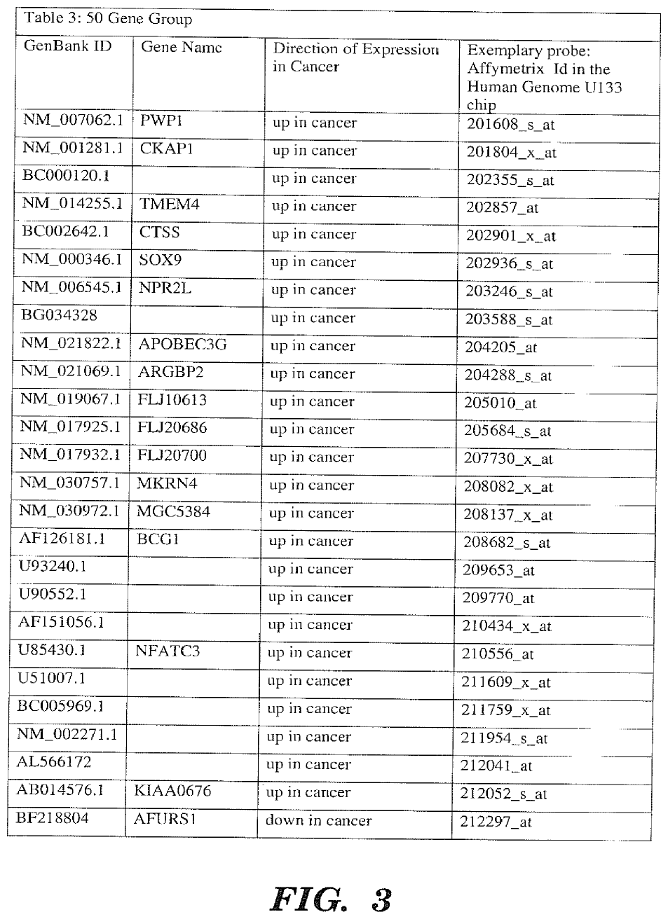

Characteristics of the 1000 additional runs of the algorithm: The number of times a sample in the test set was classified correctly and its average prediction strength was computed across the 1000 runs of the algorithm. The average prediction strength when a sample was classified correctly was 0.54 for cancers and 0.61 for no cancers, and the average prediction strength when a sample was misclassified was 0.31 for cancer and 0.37 for no cancers. The slightly higher prediction strength for smokers without cancer is reflective of the fact that predictors have a slightly higher specificity on average. Supplemental FIG. 3 shows that samples that are consistently classified correctly or classified incorrectly are classified with higher confidence higher average prediction strength). Interestingly, 64% of the samples that are consistently classified incorrectly (incorrect greater than 95% of the time, n=22 samples) are samples from smokers that do not currently have a final diagnosis of cancer. This significantly higher false-positive rate might potentially reflect the ability of the biomarker to predict future cancer occurrence or might indicate that a subset of smokers with a cancer-predisposing gene-expression phenotype are protected from developing cancer through some unknown mechanism.

In order to further assess the stability of the biomarker gene committee, the number of times the 80-predictive probesets used in the biomarker were selected in each of the 1000 runs (Supplemental Table 6) was examined. (See FIG. 10A) The majority of the 80-biomarker probesets were chosen frequently over the 1000 runs (37 probesets were present in over 800 runs, and 58 of the probesets were present in over half of the runs). For purposes of comparison, when the cancer status of the training set samples are randomized over 1000 runs (Supplemental Table 7), the most frequently selected probeset is chosen 66 times, and the average is 7.3 times. (See FIG. 10B).

Comparison of RMA vs. MAS 5.0 and weighted voting vs. PAM: To evaluate the robustness of our ability to use airway gene expression to classify smokers with and without lung cancer, we examined the effect of different class-prediction and data preprocessing algorithms. We tested the 80-probesets in our classifier to generate predictive models using the Prediction Analysis of Microarrays (PAM) algorithm (Tibshirani R, et al., Diagnosis of multiple cancer types by shrunken centroids of gene expression. Proc Natl Acad Sci USA 2002; 99(10):6567-6572), and we also tested the ability of the WV algorithm to use probeset level data that had been derived using the MAS 5.0 algorithm instead of RMA. The accuracy of the classifier was similar when microarray data was preprocessed in MAS 5.0 and when the PAM class prediction algorithm was used (see Supplemental Table 8).

SUPPLEMENTAL TABLE 8

| | Accuracy | Sensitivity | Specificity |
|---|---|---|---|
| WV - RMA data | 82.69% | 80% | 84.38% |
| PAM - RMA data | 86.54% | 90% | 84.38% |
| WV - MAS5 data | 82.69% | 80% | 84.38% |
| PAM - MAS5 data | 86.54% | 95% | 81.25% |

Supplemental Table 8 shows a comparison of accuracy, sensitivity and specificity for our 80 probeset classifier on the 52 sample test set using alternative microarray data preprocessing algorithms and class prediction algorithms.

Prediction strength: The Weighted voting algorithm predicts a sample's class by summing the votes each gene on the class prediction committee gives to one class versus the other. The level of confidence with which a prediction is made is captured by the Prediction Strength (PS) and is calculated as follows:

$$PS = \frac{{V}_{winning} - {V}_{losing}}{{V}_{winning} + {V}_{losing}}$$

V_{winning} refers to the total gene committee votes for the winning class and V_{losing} refers to the total gene committee votes for the losing class. Since V_{winning} is always greater than V_{losing}, PS confidence varies from 0 (arbitrary) to 1 (complete confidence) for any given sample.

In our test set, the average PS for our gene profile's correct predictions (43/52 test samples) is 0.73 (+/−0.27), while the average PS for the incorrect predictions (9/52 test samples) is much lower: 0.49 (+/−0.33; p<z; Student T-Test). This result shows that, on average, the Weighted Voting algorithm is more confident when it is making a correct prediction than when it is making an incorrect prediction. This result holds across 1000 different training/test set pairs (FIG. 11):

Cancer cell type: To determine if the tumor cell subtype affects the expression of genes that distinguish airway epithelium from smokers with and without lung cancer, Principal Component Analysis (PCA) was performed on the gene-expression measurements for the 80 probesets in our predictor and all of the airway epithelium samples from patients with lung cancer (FIG. 12). Gene expression measurements were Z(0,1) normalized prior to PCA. There is no apparent separation of the samples with regard to cancer subtype.

### Link to Lung Cancer Tissue Microarray Dataset

Preprocessing of Bhattacharjee data: The 254 CEL files from HgU95Av2 arrays used by Bhattacharjee et al. (Classification of human lung carcinomas by mRNA expression profiling reveals distinct adenocarcinoma subclasses. Proc Natl Acad Sci USA 2001; 98(24):13790-13795) were downloaded from the MIT Broad Institute's database available through internet (broad.mit.edu/mpr/lung). RMA-derived expression measurements were computed using these CEL files as described above. Technical replicates were filtered by choosing one at random to represent each patient. In addition, arrays from carcinoid samples and patients who were indicated to have never smoked were excluded, leaving 151 samples. The z-score quality filter described above was applied to this data set resulting in 128 samples for further analysis (88 adenocarcinomas, 3 small cell, 20 squamous, and 17 normal lung samples).

Probesets were mapped between the HGU133A array and HGU95Av2 array using Chip Comparer at the Duke University's database available through the world wide web at tenero.duhs.duke.edu/genearray/perl/chip/chipcomparer.pl. 64 probesets on the HGU95Av2 array mapped to the 80-predictive probesets. The 64 probesets on the HGU95Av2 correspond to 48 out of the 80 predictive probesets (32/80 predictive probesets have no clear corresponding probe on the HGU95Av2 array).

Analyses of Bhattacharjee dataset: In order to explore the expression of genes that we identified as distinguishing large airway epithelial cells from smokers with and without lung cancer in lung tumors profiled by Bhattacharjee, two different analyses were performed. Principal component analysis was used to organize the 128 Bhattacharjee samples according to the expression of the 64 mapped probesets. Principal component analysis was conducted in R using the package prcomp on the z-score normalized 128 samples by 64 probeset matrix. The normal and malignant samples in the Bhattacharjee dataset appear to separate along principal component 1 (see FIG. 17). To assess the significance of this result, the principal component analysis was repeated using the 128 samples and 1000 randomly chosen sets of 64 probesets. The mean difference between normal and malignant samples was calculated based on the projected values for principal component 1 for the actual 64 probesets and for each of the 1000 random sets of 64 probesets. The mean difference between normal and malignant from the 1000 random gene sets was used to generate a null distribution. The observed difference between the normal and malignant samples using the biomarker probesets was greater than the difference observed using randomly selected genes (p=0.026 for mean difference and p=0.034 for median difference).

The second analysis involved using the weighted voted algorithm to predict the class of 108 samples in the Bhattacharjee dataset using the 64 probesets and a training set of 10 randomly chosen normal tissues and 10 randomly chosen tumor tissues. The samples were classified with 89.8% accuracy, 89.1% sensitivity, and 100% specificity (see Supplemental Table 9 below, Single Run). To examine the significance of these results, the weighted voted algorithm was re-run using two types of data randomization. First, the class labels of the training set of 20 samples were permuted and the algorithm, including gene selection, was re-run 1000 times (referred to in Supplemental Table 9 as Random 1). The second randomization involved permuting the class labels of the training set of 20 samples and re-running the algorithm 1000 times keeping the list of 64-probsets constant (referred to in Supplemental Table 9 as Random 2). In the above two types of randomization, the class labels were permuted such that half the samples were correctly labeled. The p-value for each metric and randomization method shown indicate the percentage of 1000 runs using that randomization method that exceeded or were equal to the performance of the actual classifier. Genes that distinguish between large airway epithelial cells from smokers with and without cancer are significantly better able to distinguish lung cancer tissue from normal lung tissue than any random run where the class labels of the training set are randomized.

SUPPLEMENTAL TABLE 9

| | Accur | sd(Accur) | p-value | Sens | sd(Sens) | p-value | Spec | sd(Spec) | p-value | AUC | sd(AUC) | p-value |
|---|---|---|---|---|---|---|---|---|---|---|---|---|
| Single Run | 0.898 | | | 0.891 | | | 1 | | | 0.984 | | |
| Random 1 | 0.486 | 0.218 | 0.007 | 0.486 | 0.217 | 0.008 | 0.484 | 0.352 | 0.131 | 0.481 | 0.324 | 0.005 |
| Random 2 | 0.498 | 0.206 | 0.009 | 0.499 | 0.201 | 0.011 | 0.494 | 0.344 | 0.114 | 0.494 | 0.324 | 0.014 |

Supplemental Table 9 above shows results of a comparison between the predictions of the Bhattacharjee samples using the 64 probesets that map to a subset of the 80-predictive probesets and random runs (explained above). Accur=Accuracy, Sens=Sensitivity, Spec=Specificity, AUC=area under the curve, and sd=standard deviation.

Real Time PCR: Quantitative RT-PCR analysis was used to confirm the differential expression of a seven genes from our classifier. Primer sequences for the candidate genes and a housekeeping gene, the 18S ribosomal subunit, were designed with PRIMER EXPRESS® software (Applied Biosystems) (see Supplemental Table 10).

Supplemental TABLE 10
Candidate and housekeeping gene primers for real time PCR assay

| Gene | | | | | |
|---|---|---|---|---|---|
| Symbol | Affy ID | Ensembl ID | Name | Forward Primer | Reverse Primer |
| BACH2 | 215907_at | ENSG00000112182 | BTB and CNC | TGGCAAAACCGCATCTCT | ACCACCATGCCCAGCTAA |
| | | | homology 1, | AC (SEQ ID No. 1) | (SEQ ID No. 2) |
| | | | basic | | |
| | | | leucine zipper | | |
| | | | transcription | | |
| | | | factor 2 | | |
| DCLRE1C | 219678_x_at | ENSG00000152457 | DNA cross-link | GCACTTTGAGGTGGGCAA | CCAGGCTGGTGTCGAACTC |
| | | | repair 1C | T (SEQ ID No. 3) | (SEQ ID No. 4) |
| DUOX1 | 215800_at | ENSG00000137857 | dual oxidase 1 | GAGAGAAAGCAAAGGAG | CATGTGAGTCTGAAATTACAGCATT |
| | | | | TGAACTT (SEQ ID No. 5) | (SEQ ID No. 6) |
| FOS | 209189_at | ENSG00000170345 | v-fos FBJ | AGATGTAGCAAAACGCAT | CTCTGAAGTGTCACTGGGAACA |
| | | | murine | GGA | (SEQ ID No. 8) |
| | | | osteosarcoma | (SEQ ID No. 7) | |
| | | | viral oncogene | | |
| | | | homolog | | |
| IL8 | 211506_s_at | ENSG00000169429 | interleukin 8 | GCTAAAGAACTTAGATGT | GGTGGAAAGGTTTGGAGTATGTC |
| | | | | CAGTGCAT (SEQ ID No. 9) | (SEQ ID No. 10) |
| RAB1A | 207791_s_at | ENSG00000138069 | RAB1A, member | GGAGCCCATGGCATCATA | TTGAAGGACTCCTGATCTGTCA |
| | | | RAS oncogene | (SEQ ID No. 11) | (SEQ ID No. 12) |
| | | | family | | |
| TPD52 | 201689_s_at | ENSG00000076554 | tumor protein | TGACTTGAGAGTGGAACC | TTACTGTCACAAACGGTGCTAAA |
| | | | D52 | TCCTA (SEQ ID No. 13) | (SEQ ID No. 14) |
| 18S | | | | TTTCGGAACTGAGGCCAT | TTTCGCTCTGGTCCGTCTT |
| | | | | G | (SEQ ID No. 16) |
| | | | | (SEQ ID No. 15) | |
| GAPDH | | | | TGCACCACCAACTGCTTA | GGCATGGACTGTGGTCATGAG |
| | | | | GC | (SEQ ID No. 18) |
| | | | | (SEQ ID No. 17) | |
| HPRT1 | | | | TGACACTGGCAAAACAAT | GGTCCTTTTCACCAGCAAGCT |
| | | | | GCA | (SEQ ID No. 20) |
| | | | | (SEQ ID No. 19) | |
| SDHA | | | | TGGGAACAAGAGGGCATC | CCACCACTGCATCAAATTCATG |
| | | | | TG | (SEQ ID No. 22) |
| | | | | (SEQ ID No. 21) | |
| TBP | | | | TGCACAGGAGCCAAGAGT | CACATCACAGCTCCCCACCA |
| | | | | GAA | (SEQ ID No. 24) |
| | | | | (SEQ ID No. 23) | |
| YWHAZ | | | | ACTTTTGGTACATTGTGG | CCGCCAGGACAAACCAGTAT |
| | | | | CTTCAA (SEQ ID No. 25) | (SEQ ID No. 26) |

Primer sequences for five other housekeeping genes (HPRT1, SDHA, YWHAZ, GAPDH, and TBP) were adopted from Vandesompele et al. (Accurate normalization of real-time quantitative RT-PCR data by geometric averaging of multiple internal control genes. Genome Biol 2002; 3(7)). RNA samples (1 μg of the RNA used in the microarray experiment) were treated with DNAfree (Ambion, Austin, Tex.), according to the manufacturer's protocol, to remove contaminating genomic DNA. Total RNA was reverse-transcribed using random hexamers (Applied Biosystems) and SuperScript II reverse transcriptase (Invitrogen). The resulting first-strand cDNA was diluted with nuclease-free water (Ambion) to 5 ng/μl. PCR amplification mixtures (25 μl) contained 10 ng template cDNA, 12.5 μl of 2×SYBR Green PCR master mix (Applied Biosystems) and 300 nM forward and reverse primers. Forty cycles of amplification and data acquisition were carried out in an Applied Biosystems 7500 Real Time PCR System. Threshold determinations were automatically performed by Sequence Detection Software (version 1.2.3) (Applied Biosystems) for each reaction. All real-time PCR experiments were carried out in triplicate on each sample (6 samples total; 3 smokers with lung cancer and 3 smokers without lung cancer).

Data analysis was performed using the geNorm tool (Id.). Three genes (YWHAZ, GAPDH, and TBP) were determined to be the most stable housekeeping genes and were used to normalize all samples. Data from the QRT-PCR for 7 genes along with the microarray results for these genes is shown in FIG. 13.

### REFERENCES

(1) Parkin D M, et al., CA Cancer J Clin 2005; 55(2):74-108.
(2) Shields P G. Ann Oncol 1999; 10 Suppl 5:S7-11.
(3) Hirsch F R, et al., Clin Cancer Res 2001; 7(1):5-22.
(4) Jett J R. Clin Cancer Res 2005; 11(13 Pt 2):4988s-4992s.
(5) Macredmond R, et al., Thorax 2006; 61(1):54-56.
(6) Postmus P E. Chest 2005; 128(1):16-18.
(7) Mazzone P, et al., Clin Chest Med 2002; 23(1):137-58, ix.
(8) Schreiber G, and McCrory D C. Chest 2003; 123(1 Suppl):115S-128S.
(9) Janssen-Heijnen M L, et al., Epidemiology 2001; 12(2):256-258.
(10) Salomaa E R, et al., Chest 2005; 128(4):2282-2288.
(11) Auerbach O, et al., Arch Environ Health 1970; 21(6):754-768.
(12) Powell C A, et al., Clin Cancer Res 1999; 5(8):2025-2034.
(13) Wistuba I I, et al., J Natl Cancer Inst 1997; 89(18):1366-1373.
(14) Franklin W A, et al., J Clin Invest 1997; 100(8):2133-2137.
(15) Guo M, et al., Clin Cancer Res 2004; 10(15):5131-5136.
(16) Miyazu Y M, et al., Cancer Res 2005; 65(21):9623-9627.
(17) Spira A, et al., Proc Natl Acad Sci USA 2004; 101(27):10143-10148.
(18) Bolstad B M, et al., Bioinformatics 2003; 19(2):185-193.
(19) Golub T R, et al, Science 1999; 286(5439):531-537.
(20) Tibshirani R, et al., Proc Natl Acad Sci USA 2002; 99(10):6567-6572.
(21) Bhattacharjee A, et al., Proc Natl Acad Sci USA 2001; 98(24):13790-13795.
(22) Subramanian A, et al., Proc Natl Acad Sci USA 2005; 102(43):15545-15550.
(23) Simon R, et al., J Natl Cancer Inst 2003; 95(1):14-18.
(24) Cheng K W, et al., Cancer Res 2005; 65(7):2516-2519.
(25) Shimada K, et al., Br J Cancer 2005; 92(10):1915-1921.
(26) Xie K. Cytokine Growth Factor Rev 2001; 12(4):375-391.
(27) Campa D, et al., Cancer Epidemiol Biomarkers Prev 2005; 14(10):2457-2458.
(28) Arimura Y, et al., Anticancer Res. 24, 4051-4057. 2004.
(29) Coussens L M, and Werb Z. Nature 2002; 420(6917):860-867.
(30) Godschalk R, et al., Carcinogenesis 2002; 23(12):2081-2086.
(31) Kamio T, et al., Blood 2003; 102(9):3317-3322

### Example 3

In this study, we obtained nucleic acid samples (RNA/DNA) from nose epithelial cells. We also obtained nucleic acids from blood to provide one control. We used our findings in the PCT/US2006/014132 to compare the gene expression profile in the bronchial epithelial cells as disclosed in the PCT/US2006/014132 to the gene expression pattern discovered in this example from the nasal epithelial cells.

We have explored the concept that inhaled toxic substances create a epithelial cell “field of injury” that extends throughout the respiratory tract. We have developed the hypothesis that this “field of injury”, measured most recently in our laboratory with high density gene expression arrays, provides information about the degree of airway exposure to a toxin and the way in which an individual has responded to that toxin. Our studies have been focused on cigarette smoke, the major cause of lung cancer and of COPD, although it is likely that most inhaled toxins result in a change in gene expression of airway epithelial cells.

We began our studies by examining allelic loss in bronchial epithelial cells brushed from airways during diagnostic bronchcoscopy. We showed, as have others, that allelic loss occurs throughout the intra-pulmonary airways in smokers with lung cancer, on the side of the cancer as well as the opposite side from the cancer. Allelic loss also occurs, but to a lesser extent, in airway epithelial cells of smokers without cancer (Clinical Cancer Research 5:2025, 1999). We expended these studies to adenocarcinomas from smokers and non-smokers and showed that there was a “field of injury” in non-cancerous lung tissue of smokers, but not in non-smokers (Lung Cancer. 39:23, 2003, Am. J. Respir. Cell. Mol. Biol. 29:157, 2003).

We have progressed to using high density arrays to explore patterns of gene expression that occur in large airway epithelial cells of smokers and non-smokers. We have defined the types of genes that are induced by cigarette smoke, the relation to the amount smoked, racial differences (ATS) in how individuals respond to cigarette smoke, the changes that are reversible and not reversible in individuals who stop smoking (PNAS. 101:10143-10148, 2004). In addition, we have recently documented changes that occur in smokers who develop lung cancer (submitted and AACR), and changes that occur in smokers who develop COPD (Am. J. Respir. Cell Mol. Biol. 31: 601, 2004). All of these studies are ongoing in our laboratory and all depend on obtaining large airway epithelial cells at bronchoscopy, a process that does not lend itself to surveying large populations in epidemiologic studies.

In order to develop a tool that could assay airway epithelial gene expression without bronchoscopy in large numbers of smokers, we begun to explore the potential of using epithelial cells obtained from the oral mucosa. We developed a method of obtaining RNA from mouth epithelial cells and could measure expression levels of a few genes that changed in the bronchial epithelium of smokers, but problems with the quality and quantity of RNA obtained from the mouth has limited widespread application of this method (Biotechniques 36:484-87, 2004).

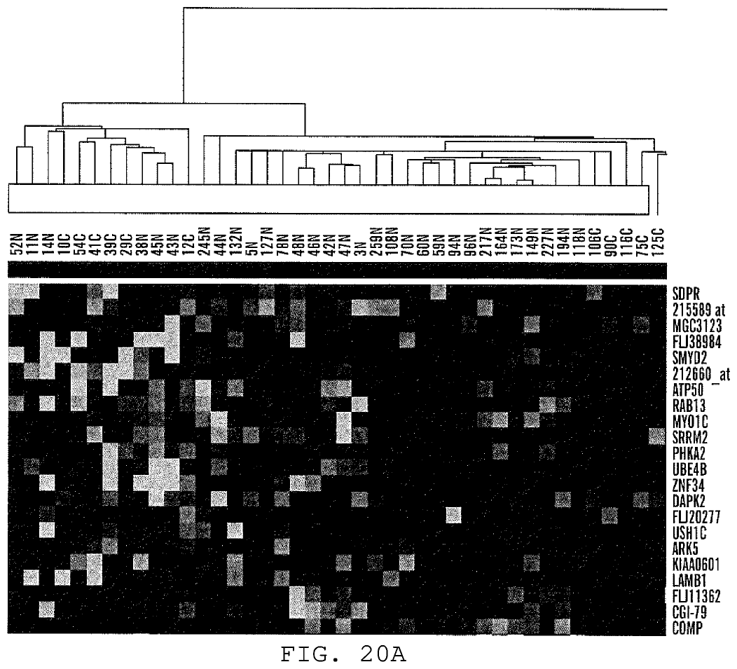

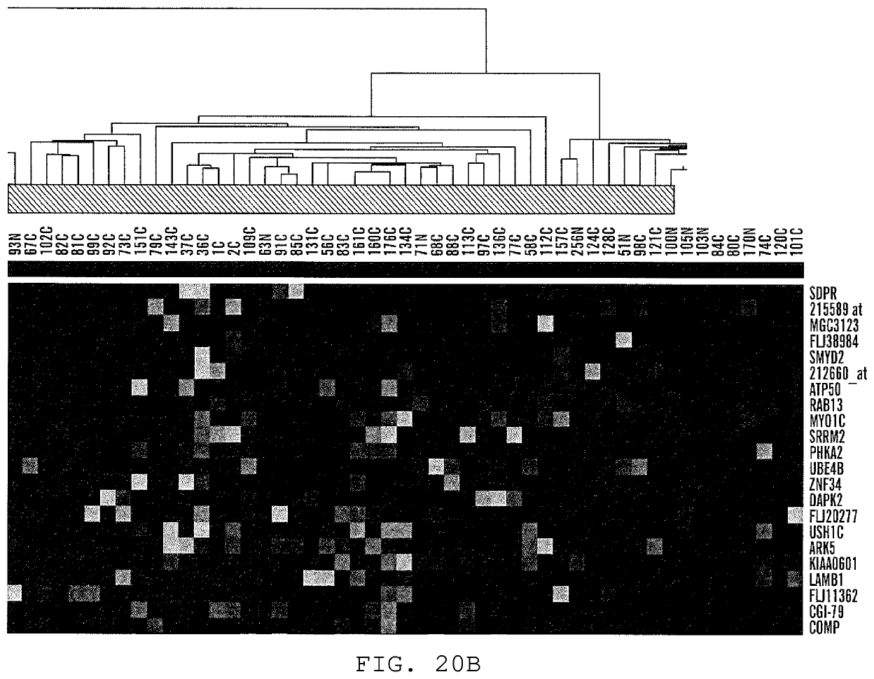

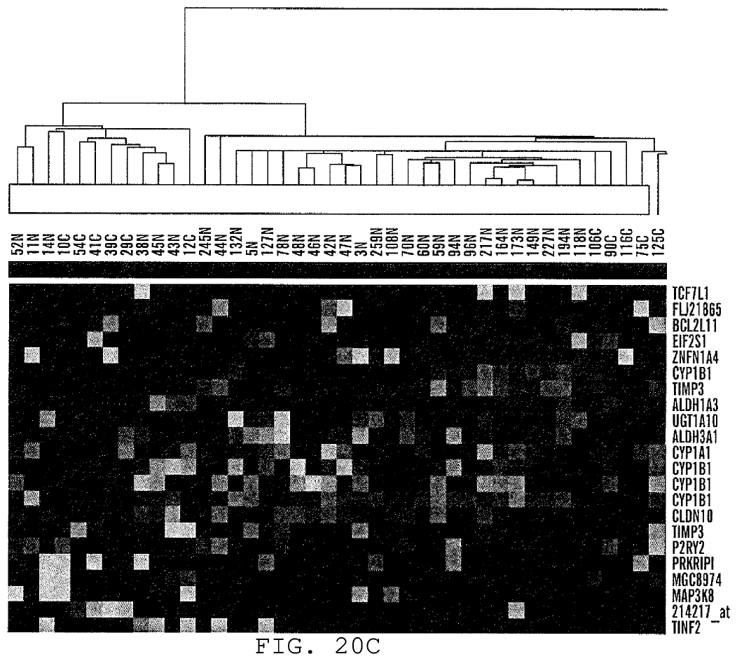

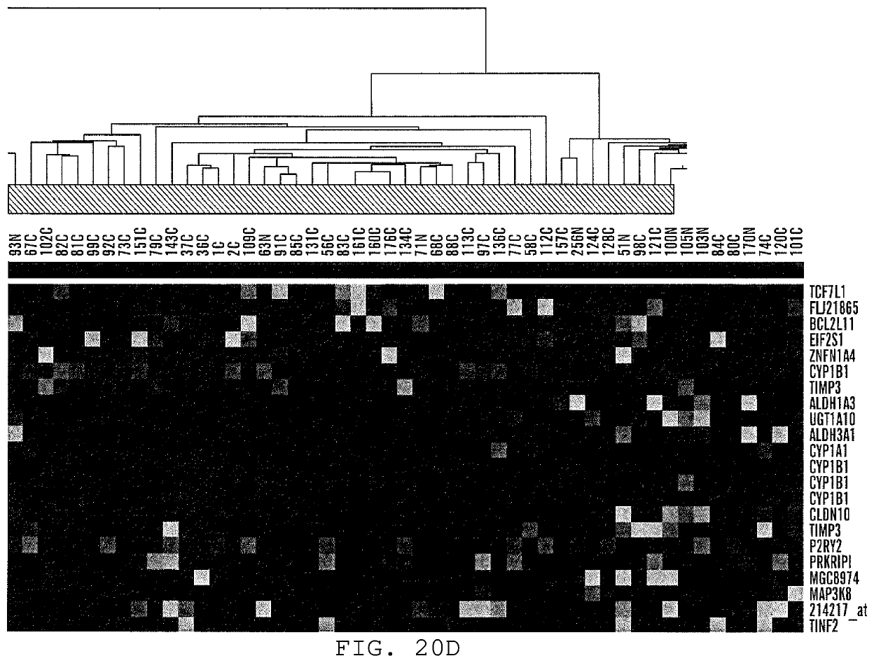

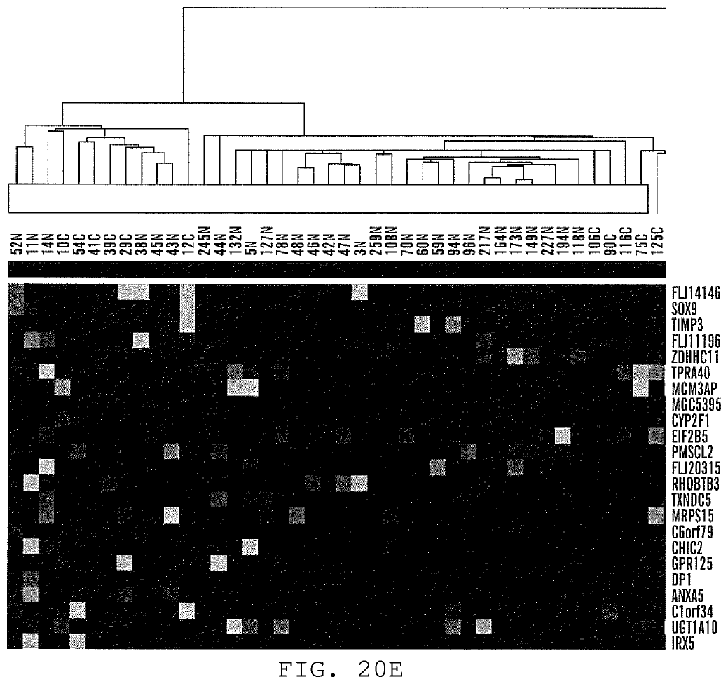

We have now shown that epithelial cells obtained by brushing the nasal mucosa could be used as a diagnostic and prognostic tool for lung disorders. Preliminary results show that we can obtain abundant amounts of high quality RNA and DNA from the nose with ease (see protocol below), that we can measure gene expression using this RNA and high density microarrays and that many of the genes that change with smoking in the bronchial epithelium also change in the nose (see FIG. 20A-20F). We have further shown that gene expression in nasal epithelium can be used to define a potentially diagnostic and clinical stage-specific pattern of gene expression in subjects with sarcoidosis, even when the sarcoidosis does not clinically involve the lung (see FIG. 21). We can also obtain DNA from these same specimens allowing us to assess gene methylation patterns and genetic polymorphisms that explain changes in gene expression.

These studies show that gene expression in nasal epithelial cells, obtained in a non-invasive fashion, can indicate individual responses to a variety of inhaled toxins such as cigarette smoke, and can provide diagnostic, and possibly prognostic and pathogenetic information about a variety of diseases that involve the lung.

Accordingly, based on our studies we have now developed the method of analyzing nasal epithelial cells as a technique and as a screening tool that can be used to evaluate individual and population responses to a variety of environmental toxins and as a diagnostic/prognostic tool for a variety of lung diseases, including lung cancer. While our initial studies utilize “discovery-based” genome-wide expression profiling, it is likely that initial studies will ultimately lead to a simpler “defined-gene” platform that will be less complicated and costly and might be used in the field.

### Protocol for Noninvasive Nasal Epithelium RNA and DNA Isolation:

Following local anesthesia with 2% lidocaine solution, a Cytosoft brush is inserted into the right nare and under the inferior turbinate using a nasal speculum for visualization. The brush is turned 3 times to collect epithelial cells and immediately placed into RNA Later. Repeat brushing is performed and the 2nd brush is placed in PBS for DNA isolation.

### Extending the Airway ‘Field of Injury’ to the Mouth and Nose

While we have demonstrated gene expression differences in bronchial epithelium associated with current, cumulative and past tobacco exposure, the relatively invasive nature of bronchoscopy makes the collection of these tissue samples challenging for large scale population studies and for studies of low-disease-risk individuals. Given our hypothesis that the field of tobacco injury extends to epithelial cells lining the entire respiratory tract, we performed a pilot study to explore the relationship between bronchial, mouth and nasal gene expression in response to tobacco exposure as nasal and oral buccal epithelium are exposed to cigarette smoke and can be obtained using noninvasive methods. In our pilot study, we collected 15 nasal epithelial samples (8 never smokers, 7 current smokers) via brushing the right inferior turbinate as described in our Research Methods and Design section. In addition, we collected buccal mucosa epithelial samples from 10 subjects (5 never smokers, 5 current smokers) using a scraping device that we have described previously [38] (see Appendix). All samples were run on Affymetrix HG-U133A arrays. Due to the small amounts (1-2 ug) of partially degraded RNA obtained from the mouth, samples were collected serially on each subject monthly and pooled to yield sufficient RNA (6-8 ug), Low transcript detection rates were observed for mouth samples, likely as a result of lower levels of intact full-length mRNA in the mouth samples

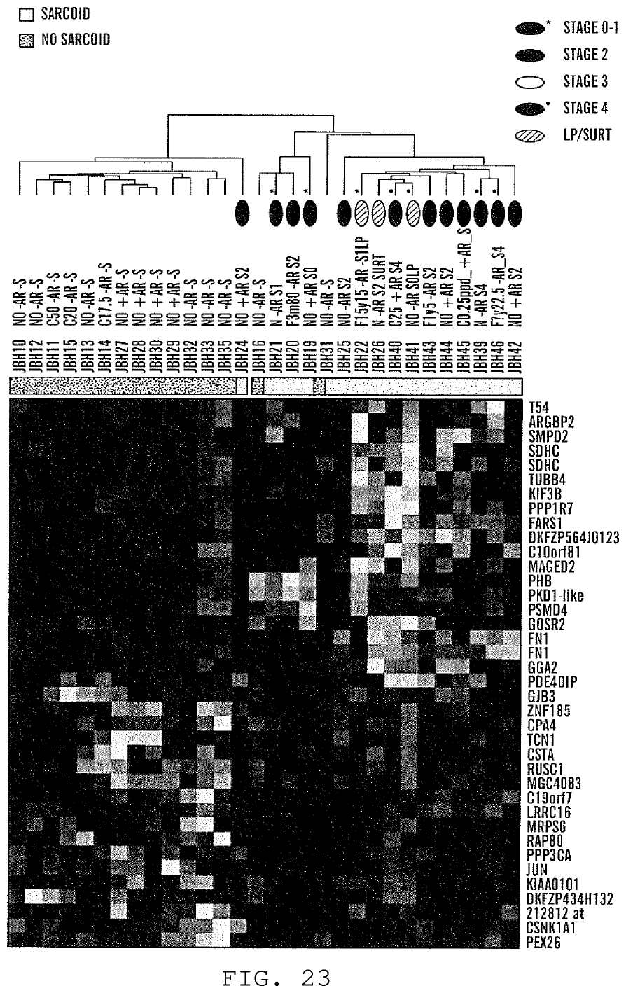

A relationship between the tobacco-smoke induced pattern of gene expression in all three tissues was first identified by Gene Set Enrichment Analysis (GSEA; [39]) which demonstrates that genes differentially expressed in the bronchus are similarly changed in both the mouth and nose (GSEA p<0.01). We next performed a 2 way ANOVA to identify 365 genes are differentially expressed with smoking across all three tissues at p<0.001. PCA of all samples normalized within each tissue for these 365 genes is shown in FIG. 24.

Finally, while this pilot study in the nose and mouth was not well powered for class prediction, we explored the possibility of using these tissues to identify biomarkers for smoke exposure. The genes with the 20 highest and 20 lowest signal-to-noise ratios between smokers and never-smokers were identified in both the nose and mouth. A classifier was then trained using these genes in bronchial epithelial samples (15 current and 15 never smokers), and tested on an independent test set of 41 samples. Genes selected from mouth and nose classify bronchial epithelium of current vs. never-smokers with high accuracy:

| | Genes | Genes | Genes | Random |
|---|---|---|---|---|
| | selected | selected | selected | sselected |
| | from Nose | from Mouth | from Bronch | Genes |
| Bronchus | 82.8% | 79.2% | 93.2% | 64.2 ± 8.1 |
| Classification | | | | |
| Accuracy | | | | |

This pilot study established the feasibility of obtaining significant quantities of good quality RNA from brushings of the nasal mucosa suitable for DNA microarray studies and has demonstrated a relationship between previously defined smoking-related changes in the bronchial airway and those occurring in the nasal epithelium. While the quality and quantity of RNA obtained from buccal mucosa complicates analysis on the U133A platform, pooled studies suggest a gene-expression relationship to the bronchial airway in the setting of tobacco exposure. These results support the central hypothesis that gene expression profiles in the upper airway reflect host response to exposure. By using a novel array platform with the potential to measure gene expression in setting of partially degraded RNA, we propose to more fully explore the ability to create biomarkers of tobacco exposure with samples from nose and mouth epithelium.

### Example 4

A Comparison of the Genomic Response to Smoking in Buccal, Nasal and Airway Epithelium

Approximately 1.3 billion people smoke cigarettes worldwide which accounts for almost 5 million preventable deaths per year (1). Smoking is a significant risk factor for lung cancer, the leading cause of cancer-related death in the United States, and chronic obstructive pulmonary disease (COPD), the fourth leading cause of death overall. Approximately 90% of lung cancer can be attributed to cigarette smoking, yet only 10-15% of smokers actually develop this disease (2). Despite the well-established causal role of cigarette smoke in lung cancer and COPD, the molecular epidemiology explaining why only a minority of smokers develop them is still poorly understood.

Cigarette smoking has been found to induce a number of changes in both the upper and lower respiratory tract epithelia including cellular atypia (3, 4), aberrant gene expression, loss of heterozygosity (3, 5) and promoter hypermethylation. Several authors have reported molecular and genetic changes such as LOH or microsatellitle alterations dispersed throughout the airway epithelium of smokers including areas that are histologically normal (4, 6). We previously have characterized the effect of smoking on the normal human airway epithelial transcriptome and found that smoking induces expression of airway genes involved in regulation of oxidant stress, xenobiotic metabolism, and oncogenesis while suppressing those involved in regulation of inflammation and tumor suppression (7). While this bronchoscopy-based study elucidated some potential candidates for biomarkers of smoking related lung damage, there is currently a significant impetus to develop less invasive clinical specimens to serve as surrogates for smoking related lung damage.

Oral and nasal mucosa are attractive candidates for a biomarkers since they are exposed to high concentrations of inhaled carcinogens and are definitively linked to smoking-related diseases (8). We have previously shown that it is feasible to obtain sufficient RNA from both nasal (9) and buccal mucosa for gene expression analysis (10) despite the high level of RNAses in saliva and nasal secretions (11, 12). Few studies have characterized global gene expression in either of these tissues, and none has attempted to establish a link between upper and lower airway gene expression changes that occur with smoking. A pilot study by Smith et. al. used brush biopsies of buccal mucosa from smokers and nonsmokers to obtain RNA for cDNA microarrays and found approximately 100 genes that could distinguish the two groups in training and test sets. While the study provided encouraging evidence that buccal gene expression changes with smoking, many of these genes were undefined ESTs, and the study did not address any potential relationship between genetic responses in the upper and lower airways. Spivak et. al. found a qualitative relationship via PCR (i.e. detected or not detected) between patient matched buccal mucosa and laser-dissected lung epithelial cells across nine carcinogen or oxidant-metabolizing genes (13) in 11 subjects being evaluated for lung cancer. However, quantitative real-time PCR of these genes in buccal mucosa was not able to reliably predict lung cancer vs. control cases. While global gene expression profiling on nasal brushing has been done recently on children with asthma (14) and cystic fibrosis (15), we are unaware of any studies addressing the effects of smoking on nasal epithelial gene expression.

In the current study, we report for the first time, a genome wide expression assay of buccal and nasal mucosa on normal healthy individuals, which herein are referred to as the “normal buccal and nasal transcriptomes”. We then evaluate the effects of smoking on these transcriptomes and compare them to a previous bronchial epithelial gene expression dataset. By comparing these smoking-induced changes in the mouth, nose, and bronchus we establish a relationship between the lower and upper airway genetic responses to cigarette smoke and further advance the concept of a smoking-induced “field defect” on a global gene expression level. Lastly, we validate the use of mass spectrometry as a feasible method for multiplexed gene expression studies using small amounts of degraded RNA from buccal mucosa scrapings.

### Study Population

Microarrays were performed on total of 25 subjects and mass spectrometry validation on 14 additional subjects. Demographic data for the microarray and mass spectrometry validation groups are presented in Table 21.

Microarray analysis of normal tissue samples was performed on previously published datasets collected from the Gene Expression Omnibus (GEO). Ninety two samples spanning 10 different tissues types were analyzed altogether, including 12 nasal and buccal epithelial samples of non-smokers collected for this study. Additional microarray data from normal nasal epithelial samples were also collected to determine the reproducibility of gene expression patterns in nasal tissue collected from a different study. A detailed breakdown of the different tissues analyzed and number of samples within each tissue type are shown in Table 22.

### The Relationship Between Normal Airway Epithelial Cells

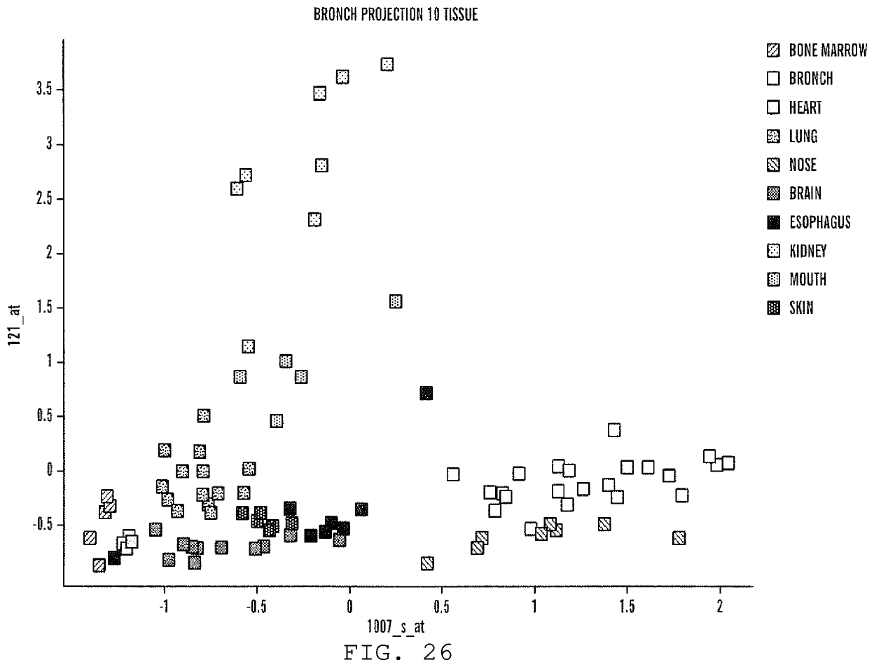

Principal component analysis (PCA) of the normal tissue samples spanning 10 tissue types (n=92 total samples) was performed across the 2382 genes comprising the normal airway transcriptome, which has been previously characterized (Spira et. al, 2004, PNAS). FIG. 26 shows bronchial and nasal epithelial samples clearly grouped together based on the expression of these 2382 genes.

Overrepresented sets of functional gene categories (“functional sets”) among the 2382 normal airway transcriptome genes were determined by EASE analysis. Table 23 lists the 16 functional sets that were significantly overrepresented among the normal airway transcriptome. On average there were approximately 109 probe sets per functional cluster. A variability metric was used to determine those functional sets that were most different across the 10 tissue types. Ahdehyde dehydrogenase, antigen processing and presentation, and microtubule and cytoskeletal complex were the most variable functional sets. The least variable sets included ribosomal subunits, and nuclear and protein transport. Two dimensional hierarchical clustering was also performed on each of these 16 functional sets to determine which tissues showed similar expression patterns across all the genes in each set. Among the top three most variable functional sets listed above, bronchial and nasal epithelial samples always grouped together (data not shown).

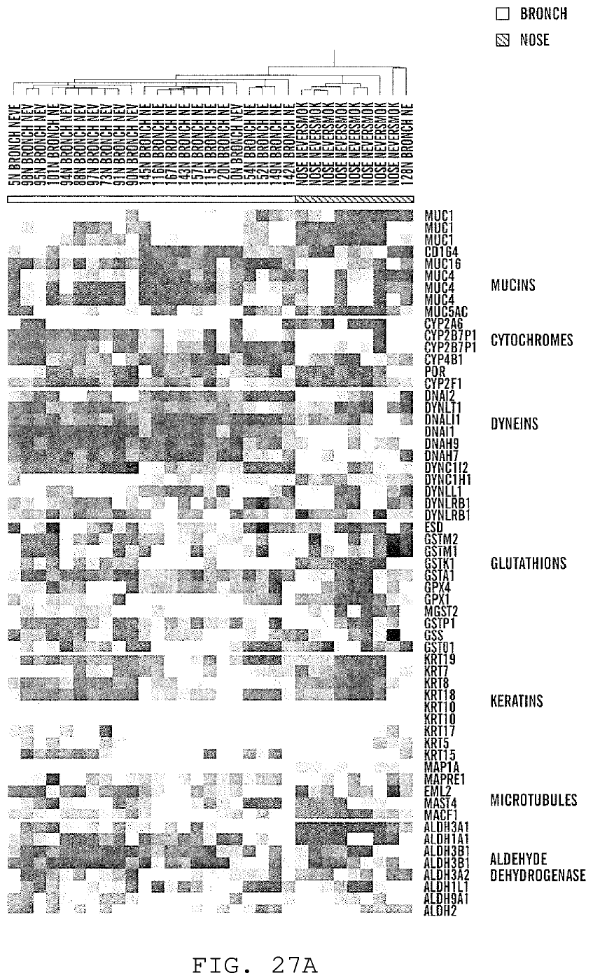

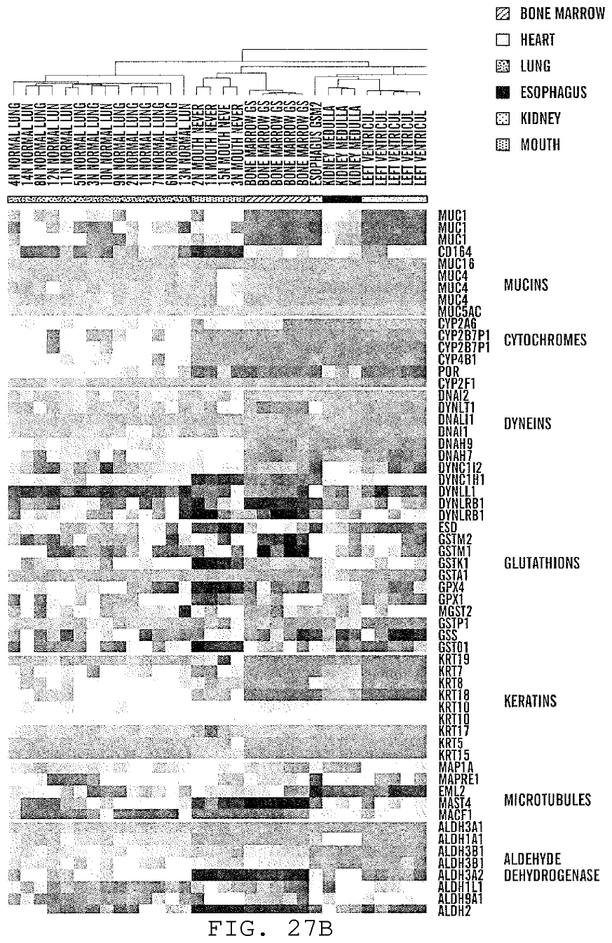

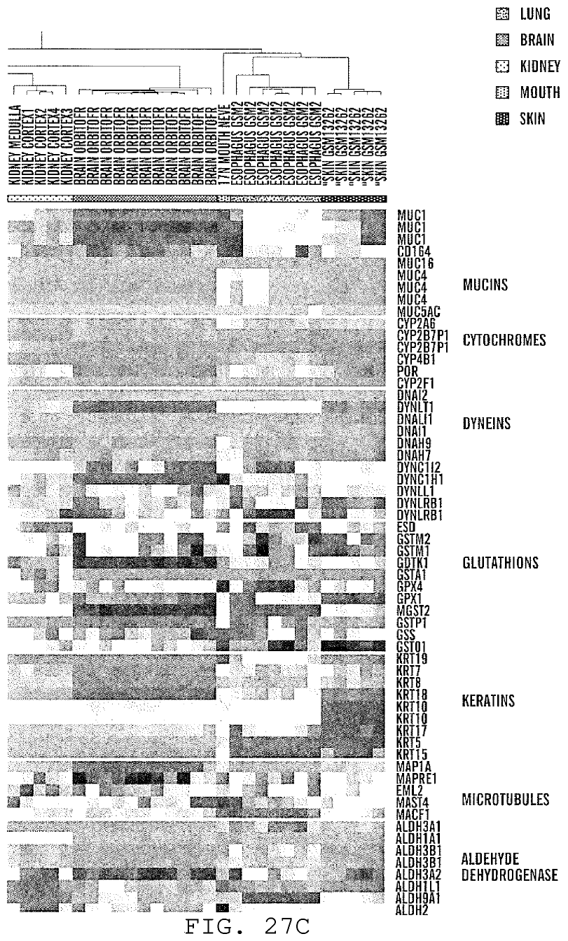

To further examine the relationship between bronchial epithelial tissues and other tissues, genes from functional groups commonly expressed in airway epithelium were selected from among the normal airway transcriptome. Genes from the mucin, dynein, microtubule, keratin, glutathione, cytochrome P450, and aldehyde dehydrogenase functional groups were selected from among the 2382 genes in the normal airway transcriptome, based on their gene annotations. Fifty-nine genes from these functional groups were present among the normal airway transcriptome and analyzed using supervised hierarchical clustering, as shown in FIG. 27. Bronchial and nasal epithelial samples clustered together based on the expression of these 59 genes, with many being expressed at higher levels in these two tissues. Genes highly expressed in bronchial and nasal epithelium were generally evenly distributed among the five functional groups. Several dynein, cytochrome P450, and aldehyde dehydrogenase genes were expressed highly in bronchial and nasal epithelium compared to other tissues. Buccal mucosa samples clustered mainly with lung tissue, with specific keratin genes being highly expressed. While some keratins were expressed specifically in skin and esophageal epithelium, other keratins, such as KRT7, KRT8, KRT18, and KRT19 were expressed primarily in bronchial and nasal epithelium. The same pattern was seen with mucin genes, with MUC4, MUC5AC, and MUC16 being expressed primarily in bronchial and nasal epithelium, while MUC1 was expressed in other epithelial tissues. Glutathione genes were expressed highly in bronchial and nasal epithelium as well as other tissues. Microtubule expression was fairly even across all tissues.

To explore the similar expression pattern between bronchial and nasal epithelium, a metagene was created by selected a subset of the 59 functionally relevant normal transcriptome genes with highly correlated expression in between bronchial and nasal samples. All genes which were highly correlated to the metagene (R>0.6, p<0.001) were selected and analyzed using EASE to determine sets functionally overrepresented categories. The microtubule and cytoskeletal complex functional set was significantly enriched among the genes most highly correlated with the expression pattern of the metagene.

A separate set of normal nasal epithelial samples run on the same microarray platform (16) was used in place of our nasal epithelial dataset to determine the reproducibility of the relationships in gene expression between bronchial and nasal epithelium. This separate nasal epithelial dataset consisted of 11 normal epithelial samples run on Affymetrix HG133A microarrays. These samples were first examined with the 92 normal tissue samples from previous analysis. A correlation matrix was created to determine the average pearson correlation of each set of samples within a tissue type with samples from other tissue types. The two nasal epithelial datasets had the highest correlation with each other, with the next highest correlation being between nasal and bronchial epithelial samples. These 11 nasal epithelial samples also clustered together with bronchial epithelial samples across the entire normal transcriptome and the subset of 59 functionally relevant genes from the transcriptome when used in place of our original 8 nasal epithelial samples.

### Effect of Cigarette Smoking on the Airway Epithelial

To examine the effect of cigarette smoke on airway epithelial cells, current and never smokers samples from buccal and nasal epithelial cell samples were analyzed together with current and never smokers from bronchial epithelial samples published previously (Spira et. al, 2004, PNAS). In total there were 82 samples across these three tissue types (57 bronch, 10 buccal, 15 nasal). To determine the relationship in the response to cigarette smoke between these three tissues, expression of 361 genes previously reported to distinguish smokers from non-smokers in bronchial epithelial cells (Spira et. al, 2004, PNAS) was examined across all 82 samples from bronchial, nasal, and buccal epithelium.

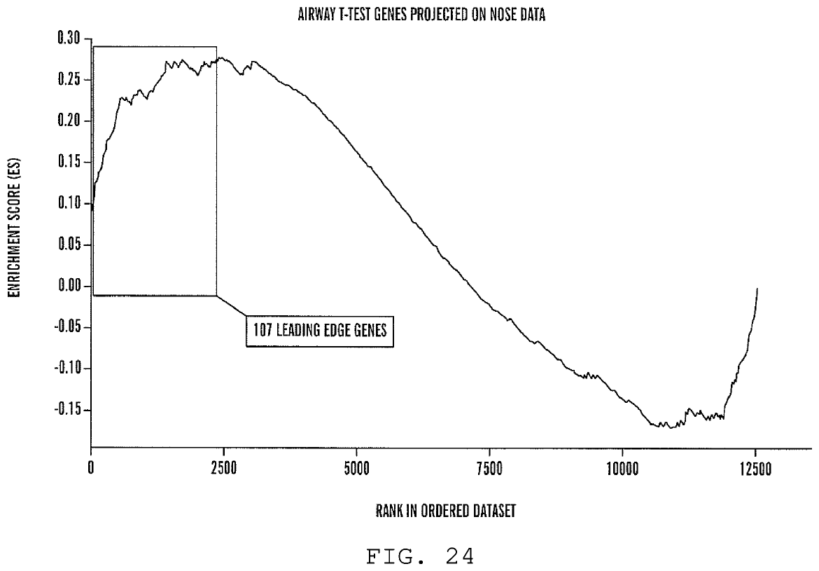

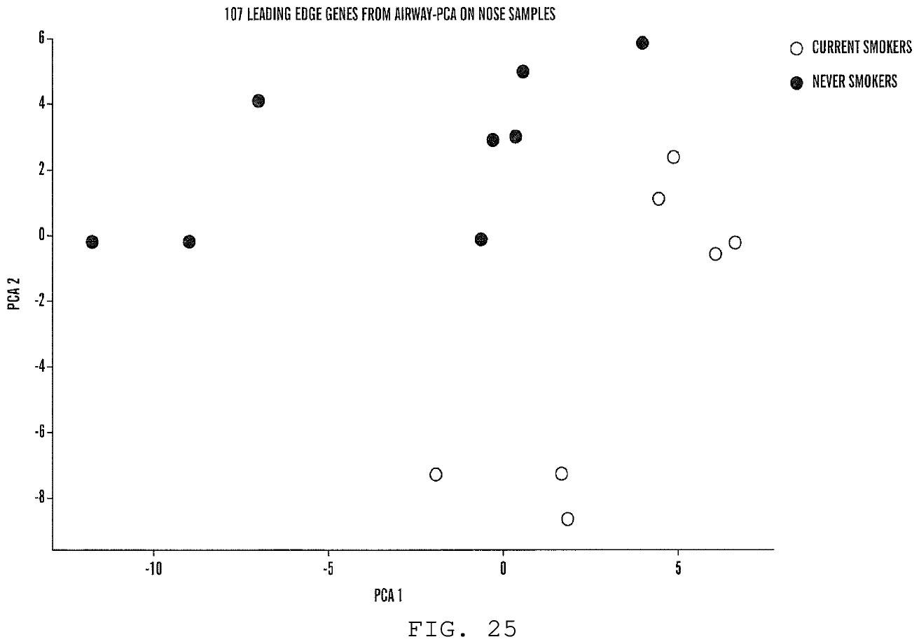

The 361 genes as shown in Table 18 most differently expressed in the airway epithelial cells of current and never smokers were generally able to distinguish bronchial, nasal, and buccal epithelial samples based on smoking status using principal component analysis, with few exceptions among buccal mucosa samples (FIG. 22). This finding suggests a relationship between gene expression profiles in epithelial cells in the bronchus and upper airway epithelium in response to cigarette smoke. To further establish this connection across airway epithelial cells, gene set enrichment analysis (GSEA) was performed to determine if genes most differentially expressed in bronchial epithelium based on smoking status were overrepresented among the genes that change with smoking in both nasal and buccal epithelium. We showed that smoking-induced airway genes are significantly enriched among the genes most affected by smoking in buccal mucosa, with 101 genes composing the “leading edge subset” (p<0.001). The leading edge subset consists of the genes that contribute most to the enrichment of airway genes in buccal mucosa samples. FIG. 25 similarly shows that the genes differing most across the bronchial epithelium of smokers were also significantly enriched among the genes most affected by smoking in nasal epithelial cell samples, with 107 genes comprising the leading edge subset (p<0.001). PCA of the leading edge genes show that they are able to separate buccal mucosa samples and nasal epithelial samples (FIG. 26) based on smoking status, suggesting a global relationship in gene expression across airway epithelial cells in response to smoking. EASE analysis of the leading edge subsets from FIG. 24 reveals that overrepresented functional categories from these gene lists include oxidoreductase activity, metal-ion binding, and electron transport activity (see Table 23).

### Study Population

We recruited current and never smoker volunteers from Boston Medical Center for a buccal microarray study (n=11), nasal microarray study (n=15) and subsequent prospective buccal epithelial cell mass spectrometry validation (n=14). Current smokers in each group had smoked at least 10 cigarettes per day in the past month, with at least a cumulative 10 pack-year history. Non-smoking volunteers with significant environmental cigarette exposure and subjects with respiratory symptoms, known respiratory, nasal or oral diseases or regular use of inhaled medications were excluded. For each subject, a detailed smoking history was obtained including number of pack-years, number of packs per day, age started, and environmental tobacco exposure. Current and never smokers were matched for age, race and sex. The study was approved by the Institutional Review Board of Boston Medical Center and all subjects provided written informed consent.

### Buccal Epithelial Cell Collection

Buccal epithelial cells were collected on 25 subjects (11 for the buccal microarray study, 14 for the mass spectrometry validation) as previously reported (Spira et. al. 2004, Biotechniques). Briefly, we developed a non-invasive method for obtaining small amounts of RNA from the mouth using a concave plastic tool with serrated edges. Using gentle pressure, the serrated edge was scraped 5 times against the buccal mucosa on the inside left cheek and placed immediately into 1 mL of RNALATER (Qiagen, Valencia, Calif.). The procedure was repeated for the inside right cheek and the cellular material was combined into one tube. After storage at room temperature for up to 24 hours, total RNA was isolated from the cell pellet using TRIZOL® reagent (Invitrogen, Carlsbad, Calif.) according to the manufacturer's protocol. The integrity of the RNA was confirmed on an RNA denaturing gel. Epithelial cell content was quantified by cytocentrifugation at 700×g (Cytospin, ThermoShandon, Pittsburgh, Pa.) of the cell pellet and staining with a cytokeratin antibody (Signet, Dedham, Mass.). Using this protocol, we were able to obtain an average of 1823 ng+/−1243 ng of total RNA per collection. Buccal epithelial cells were collected serially over 6 weeks in order to obtain a minimum of 8 ug of RNA per subject. For the 14 subjects included in the mass spectrometry validation, a single collection was sufficient. Nasal epithelial cell collection

Nasal epithelial cells were collected by first anesthesizing the right nare with 1 cc of 1% lidocaine. A nasal speculum (Bionix, Toledo Ohio) was use to spread the nare while a standard cytology brush (Cytosoft Brush, Medical Packaging Corporation, Camarillo Calif.) was inserted underneath the inferior nasal turbinate. The brush was rotated in place once, removed, and immediately placed in 1 mL RNA Later (Qiagen, Valencia, Calif.). After storage at 4 degrees overnight, RNA was isolated via Qiagen RNEASY® Mini Kits per manufacturer's protocol. As above, the integrity of RNA was confirmed with an RNA denaturing gel and epithelial cell content was quantified by cytocentrifugation.

### Bronchial Epithelial Cell Collection

Bronchial epithelial cells were also obtained on a subset of patients in the mass spectrometry study (N=6 of the 14) from brushings of the right mainstem during fibertoptic bronchoscopy with three endoscopic cytobrushes (Cellebrity Endoscopic Cytobrush, Boston Scientific, Boston). After removal of the brush, it was immediately placed in TRIZOL® reagent (Invitrogen), and kept at −80° C. until RNA isolation was performed. RNA was extracted from the brush using the TRIZOL® reagent (Invitrogen, Carlsbad, Calif.) according to the manufacturer's protocol with an average yield of 8-15 ug of RNA per patient. Integrity of RNA was confirmed by running an RNA-denaturing gel and epithelial cell content was quantified by cytocentrifugation and cytokeratin staining.

### Microarray Data Acquisition and Preprocessing

Eight micrograms of total RNA from buccal epithelial cells (N=11) and nasal epithelial cells (N=15) was processed, labelled, and hybridized to Affymetrix HG-U133A GeneChips containing 22,215 probe sets as previously described (Spira et. al, 2004, PNAS). A single weighted mean expression level for each gene was derived using MICROARRAY SUITE 5.0 (MAS 5.0) software (Affymetrix, Santa Clara, Calif.). The MAS 5.0 software also generated a detection P value [P(detection)] using a one-sided Wilcoxon sign-ranked test, which indicated whether the transcript was reliably detected. One buccal mucosa microarray sample was excluded from further analysis based on the percentage of genes detected being lower than two standard deviations from the median percentage detected across all buccal mucosa microarray samples, leaving 10 samples for further analysis. All 15 nasal epithelial cell microarray samples contained sufficiently high percentages of genes detected based on the same criteria, and were all included for further analysis. Microarray data from 57 bronchial epithelial cell samples was obtained from previously published data (Spira et. al, 2004, PNAS).

Microarray data from 7 additional normal human tissues was obtained from datasets in the Gene Expression Omnibus (GEO). The samples were selected from normal, non-diseased tissue, where there were at least 5 samples per tissue type. All samples were run on either Affymetrix HGU133A or HGU133 Plus 2.0 microarrays. Array data from normal tissue samples from the following 7 tissues were used (GEO accession number included): lung (GSE1650), skin (GSE5667), esophagus (GSE1420), kidney (GSE3526), bone marrow (GSE3526), heart (GSE2240), and brain (GSE5389). A detailed breakdown of the array data obtained for these tissues can be seen in Table 12.

Microarray data from buccal mucosa, nasal epithelium, and bronchial epithelial cell samples, as well at normal tissue samples from the 8 datasets listed above were each normalized using MAS 5.0, where the mean intensity for each array (excluding the top and bottom 2% of genes) was corrected using a scaling factor to set the average target intensity of all probes on the chip to 100. For tissue samples run on the HGU133 Plus 2.0 arrays, only those probe sets in common with the HGU133A array were selected and normalized using MATLAB Student Version 7.1 (The Mathworks, Inc.), where the mean intensity of the selected probes (excluding the top and bottom 2% of genes) was corrected using a scaling factor to set the average target intensity of the remaining probes to 100.

### Microarray Data Analysis

Clinical information, array data, and gene annotations are stored in an interactive MYSQL database coded in PERL (37). All statistical analyses described below and within the database were performed using the R v. 2.2.0 software (38). The gene annotations used for each probe set were from the December 2004 NetAffx HG-U133A annotation files.

Principal component analysis (PCA) was performed using the Spotfire DecisionSite software package (39) on the following normal non-smoker tissue samples from 10 different tissue types: bronchial (n=23), nasal (n=8), buccal mucosa (n=5), lung (n=14), skin (n=5), esophagus (n=8), kidney (n=8), bone marrow (n=5), heart (n=5), and brain (n=11). PCA analysis was used to determine relationships in the gene expression of these tissue types across the normal airway transcriptome, which has been previously characterized (Spira et. al, 2004, PNAS).

Functional annotation clustering was performed using the EASE software package (40) to determine overrepresented sets of functional groups (“functional sets”) among the normal airway transcriptome. Each functional group within a cluster was given a p-value, determined by a Fisher-Exact test. The significance of the functional cluster was then determined by taking the geometric mean of the p-values of each functional group in the cluster. To limit the number of functional sets returned by EASE, only functional groups from the Gene Ontology (GO) database below the 5th hierarchical node were used.

To determine the variability of the functional sets across the 10 different tissue types, the following formula was used:

V=X⁻(1 . . . i)[COV(X⁻G1 . . . X⁻Gk))]

Where Gk is the expression of gene G across all the samples in tissue type k, i is the total number of genes in a functional cluster, and COV is the coefficient of variation (standard deviation divided by mean) of the average expression of gene G across all tissue types. This produced one variability metric (V) for each functional cluster. All the genes in each functional cluster were then analyzed using 2D hierarchical clustering performed by using log-transformed z-score normalized data with a Pearson correlation (uncentered) similarity metric and average linkage clustering with CLUSTER and TREEVIEW software (41).

To further analyze the relationship between airway epithelium and other tissue types, genes from the normal airway transcriptome included in functional categories commonly expressed in airway epithelial cells were examined. The functional categories explored were mucin, dynein, microtubule, cytochrome p450, glutathione, aldehyde dehydrogenase, and keratin. Genes from these categories were determined by selecting all those genes from the normal airway transcriptome that were also included in any of these functional groups based on their gene annotation. Fifty-nine genes from the normal airway transcriptome which also spanned the functional categories of interest were further analyzed across the 10 tissues types using supervised hierarchical clustering.

To assess whether genes outside of the normal airway transcriptome were expressed at similar levels in bronchial and nasal epithelium, we created a metagene by taking a subset of the 59 genes from the normal airway transcriptome spanning the specified functional categories which were highly expressed in bronchial and nasal epithelial samples, based on the Pearson correlation similarity metric for these genes. A correlation matrix was then generated between the average expression of the metagene across all 10 tissues and each probe set on the HGU133A array (22215 total probe sets) across all 10 tissues, to determine genes with a similar expression pattern to bronchial and nasal epithelium (a detailed protocol for this analysis can be found in the supplement).

A second nasal epithelial dataset (Wright et. al, 2006, Am J Respir Cell Mol Biol.) was included for further analysis to determine the reproducibility of the expression patterns observed in nasal epithelium compared to other tissues. In all there were 11 nasal epithelial samples from this second dataset (GSE2395) which were used in place of our original 8 nasal samples to determine the reproducibility of gene expression patterns and relationships between nasal epithelium and other tissues.

To determine the relationship in the response to cigarette smoke by bronchial, buccal, and nasal epithelial cells, PCA was performed across 82 smoker and non-smoker samples (57 bronchial, 10 buccal, 15 nasal) using 361 genes differentially expressed between smokers and non-smokers in bronchial epithelial cells (p<0.001), as determined from a prior study (Spira et. al, 2004, PNAS). Gene set enrichment analysis (GSEA) (42) was then used to further establish a global relationship between gene expression profiles from these three tissue types in response to cigarette smoke. Our goal was to determine if the genes most differentially expressed with smoking in bronchial epithelial cells were significantly enriched among the top smoking-induced buccal and nasal epithelial genes based on signal-to-noise ratios. P-values were generated in GSEA by permuting ranked gene labels and generating empirical p-values to determine significant enrichment. The airway genes most significantly enriched among ranked lists of nasal epithelial and buccal mucosa samples (leading edge subsets), were further analyzed using PCA to determine the ability of the leading edge subsets to distinguish samples in the nasal and buccal epithelial datasets based on smoking status.

Table 21 below shows Patient demographic data. Demographic data for patient samples used for microarray analysis (n=10) and mass spectrometry analysis (n=14). *P-values calculated by Fisher Extact test

| | Buccal Microarray | | | Nasal Microarray | | | MS Validation | | |
|---|---|---|---|---|---|---|---|---|---|
| | (N = 10) | | | (N = 15) | | | (N = 14) | | |
| | Smokers | Never | P-Value | Smokers | Never | P-Value | Smokers | Never | P-Value |
| Sex | 1M, 4F | 2M, 3F | (p = 0.42*) | 6 M, 1 F | 5 M, 2 | (p = .58) | 6 M, 1 F | 4 M, 3 F | (p = .24*) |
| | | | | | F, 1 U | | | | |
| Age | 36 (+/−8) | 31 (+/−9) | (p = 0.36) | 47 +/− 12 | 43 +/− 18 | | 59 (+/−15) | 41 (+/−17) | (p = 0.06) |
| Race | 3 CAU, 2 AFA | 2 CAU, 3 AFA | (p = 0.40*) | 3 CAU, 3 | 5 CAU, 2 | | 5 CAU, 2 AFA | 4 CAU, 3AFA | (p = .37*) |
| | | | | AFA, 1 HIS | AFA, 1 HIS | | | | |

Table 22 below shows breakdown of all microarray datasets analyzed in this study.

| Category | Tissue | # Samples | Platform | GEO reference | Sample Description |
|---|---|---|---|---|---|
| epithelial | Mouth | 5 | U133A | n/a | 5 never smokers |
| epithelial | Bronch | 23 | U133A | GSE994 | 23 never smokers |
| epithelial | Nose | 8 | U133A | n/a | 8 never smokers |
| epithelial | Nose | 11 | U133A | GSE2395 | normal nasal epithelium, |
| | | | | | from cystic fibrosis study |
| epithelial | Lung | 14 | U133A | GSE1650 | from COPD study, no/mild |
| | | | | | emphezyma patients |
| epithelial | Skin | 5 | U133A | GSE5667 | normal skin tissue |
| Epithelial | Esophagus | 8 | U133A | GSE1420 | normal esophageal |
| | | | | | epithelium |
| mostly | Kidney | 8 | U133 + 2.0 | GSE3526 | 4 kidney cortex, 4 kidney |
| epithelial | | | | | medulla (post-mortem) |
| non epithelial | Bone | 5 | U133 + 2.0 | GSE3526 | 5 bone marrow (post- |
| | marrow | | | | mortem) |
| non epithelial | Heart | 5 | U133A | GSE2240 | left ventricular |
| | | | | | myocardium, non-failing |
| non epithelial | Brain | 11 | U133A | GSE5389 | postmortem orbitofrontal |
| | | | | | cortex |

Table 23 below shows Significantly overrepresented “functional sets” among the normal airway transcriptome. Sixteen functional sets significantly overrepresented among the normal airway transcriptome, ranked by the variability of each cluster across 10 tissue types.

| Functional Category | Average COV | P-value |
|---|---|---|
| Aldehyde Dehydrogenase | 108.7083218 | 0.052807847 |
| Antigen processing and presentation | 83.83536768 | 0.003259035 |
| Microtubule and Cytoskeletal complex | 74.77767675 | 0.018526945 |
| Carbohydrate and Alcohol catabolism/metabolism | 67.69528886 | 0.025158044 |
| Oxidative phosphorylation, protein/ion transport, | 66.99814067 | 4.53E−07 |
| metabolism | | |
| ATPase Activity | 62.97844577 | 7.96E−08 |
| Apoptosis | 61.75272195 | 0.005467272 |
| Mitochondrial components and activity | 61.34998026 | 3.65E−09 |
| NADH Dehydrogenase | 58.28368171 | 4.77E−11 |
| Regulation of protein synthesis and metabolism | 55.93424773 | 0.002257705 |
| NF-kB | 55.70796256 | 0.011130609 |
| Protein/macromolecule catabolism | 55.62842326 | 6.74E−05 |
| Intracellular and protein transport | 53.51411018 | 8.10E−09 |
| Protein/Macromolecule Biosynthesis | 52.28818306 | 1.62E−25 |
| Vesicular Transport | 49.6560062 | 0.019136042 |
| Nuclear Transport | 44.88736037 | 0.003807797 |
| Ribosomal Subunits | 42.57469554 | 5.42E−15 |

Table 24 below shows Common overrepresented functional categories among “leading edge subsets” from GSEA analysis. Common EASE molecular functions of leading edge genes from GSEA analysis. P-values were calculated using EASE software.

| Molecular Function | P-value (calculated in EASE) |
|---|---|
| Oxidoreductase activity | p < 1.36 × 10-6 |
| Electron transporter activity | p < 4.67 × 10-5 |
| Metal ion binding | p < .02 |
| Monooxygenase activity | p < .02 |

### REFERENCES

All references cited herein and throughout the specification are herein incorporated by reference in their entirety.
1. WHO: The Facts About Smoking and Health, 30 May 2006 [http://www.wpro.who.int/media_centre/fact_sheets/fs_20060530.htm]
. Shields, P. G.: Molecular Epidemiology of lung cancer. Ann. Oncol, 1999, Suppl. 5:7-11.
2. Franklin W A, Gazdar A F, Haney J, Wistuba I I, LaRosa F G, Kennedy T, Ritchey D M, and Miller Y E.: Widely Dispersed p53 mutation in respiratory epithelium. A Novel mechanism for field carcinogenesis. Journal of Clinical Investigation, 1997, 100:2133-2137.
3. Wistuba I I, Lam S, Behrens C, Virmani A K, Fong K M, LeRiche J, Samet J M, Srivastava S, Minna J D, and Gazdar A F: Molecular damage in the bronchial epithelium of current and former smokers. Journal of the National Cancer Institute, 1997, 89:1366-1373.
4. Powell C A, Klares S, O'Connor G, Brody J S: Loss of Heterozygosity in Epithelial Cells Obtained by Bronchial Brushing: Clinical Utility in Lung Cancer. Clinical Cancer Research, 1999, 5: 2025-2034.
5. Thiberville L, Payne P, Vielkinds J, LeRiche J, Horsman D, Nouvet G, Palcic B, Lam S: Evidence of cumulative gene losses with progression of premalignant epithelial lesions to carcinoma of the bronchus. Cancer Res, 1995, 55: 5133-9.
6. Spira A S, Beane J, Shah V, Schembri F, Yang X, Palma J and Brody J S: Effects of cigarette smoke on the human airway epithelial transcriptome. PNAS, 2004, 101:10143-10148.
7. Phillips D E, Hill L, Weller M, Willett M, and Bakewell R. R Tobacco smoke and the upper airway. Clin. Otoloaryngol. 2003, 28, 492-496.
8. 7.5 Immunophenotype of the Nasal Mucosa in Sarcoidosis, [Publication Page: A795]
9. D. M. Serlin, M D, X. F. Li, PhD, J. Spiegel, M D, K. Steiling, M D, C. J. O'Hara, M D, A. Spira, M D, A. W. O'Regan, M D, J. S. Berman, M D, Boston, Mass., Galway, Ireland. Abrstact, A T S 2006
10. Spira A, Beane J, Schembri F, Liu G, Ding C, Gilman S, Yang X, Cantor C and Brody J S: Noninvasive method for obtaining RNA from buccal mucosa epithelial cells for gene expression profiling. Biotechniques, 2004, 36:484-497.
11. Kharchenko S V, Shpakov A A: Regulation of the RNase activity of saliva in healthy subjects and in stomach cancer. Inz Akad Nauk SSSR Biol, 1989, 1:58-63.
12. Ceder O, van Dijken J, Ericson T, Kollberg J: Ribonuclease in different types of saliva from cystic fibrosis patients. Acta Paediatr. Scand, 1985, 74:102-104.
13. Spivak S, Hurteau G, Jain R, Kumar S, Aldous K, Gierthy J, Kaminsky L S: Gene-Environment Interaction Signatures by Quantitative mRNA Profiling of Exfoliated Buccal Mucosal Cells. Cancer Research, 2004, 64:6805-6813.
14. Guajardo J R, Schleifer K W, Daines M O, Ruddy R M, Aronow B J, Wills-Karp M, Hershey G K, Altered gene expression profiles in nasal respiratory epithelium reflext stable versus acute childhood asthma. J Allergy Clin Immunol. 2005,
15. Wright J M, Merlo C A, Reynolds J B, Zeitlin P L, Garcia J N, Guggino W B, Boyle M P. Respiratory epithelial gene expression in patients with mild and severe cystic fibrosis lung disease. Am. J. Resp. Cell Biology, 2006, 35: 327-336.
16. Wright J M, Merlo C A, Reynolds J B, Zeitlin P L, Garcia J G N, Guggino W B, Boyle M P. Respiratory Epithelial Gene Expression in Patients with Mild and Severe Cystic Fibrosis Lung Disease. Am J Respir Cell Mol Biol, 2006, 35(3):327-336.
17. Slaughter D P, Southwick H W, Smejkal W: Field cancerization in oral stratified squamous epithelium; clinical implications of multicentric origin. Cancer, 1953, 6:963-968.
18. Wistuba I, Lam S, Behrens C, Virmani A, Fong K W, LeRiche J, Samet J, Srivastava S, Minna J, Gazdar A. Molecular damage in the bronchial epithelium of current and former smokers. JNCI. 89: 18. 1366-1373.
19. Partridge M, Emilion G, Pateromichelakis S, Phillips E, Langdon J: Field cancerisation of the oral cavity: Comparison of the spectrum of molecular alterations in cases presenting with both dysplastic and malignant lesions. Oral Oncol, 1997, 33:332-337.
20. Bosatra A, Bussani R, Silvestri F: From epithelial dysplasia to squamous carcinoma in the head and neck region: an epidemiological assessment. Acta Otolaryngol Suppl, 1997, 527:49-51.
21. Sudbo J, Kildal W, Risberg B, Koppang H S, Danielsen H E, Reith A: DNA content as a prognostic marker in patients with oral leukoplakia. N Engl J Med, 2001, 344(17):1270-1278.
22. Demedts I K, Demoor T, Bracke K R, Joos G F, Brusselle G G: Role of apoptosis in the pathogenesis of COPD and pulmonary emphysema. Respir Res., 2006, 7:53.
23. Loro L L, Johannessen A C, Vintermyr O K: Decreased expression of bcl-2 in moderate and severe oral epithelia dysplasias. Oral Oncol., 2002, 38(7):691-698.
24. Yang S R, Chida A S, Bauter M R, Shafiq N, Seweryniak K, Maggirwar S B, Kilty I, Rahman I: Cigarette smoke induces proinflammatory cytokine release by activation of NF-kappaB and posttranslational modifications of histone deacetylase in macrophages. Am J Physiol Lung Cell Mol Physiol., 2006, 291(1):L46-57.
25. Sasaki H, Moriyama S, Nakashima Y, Kobayashi Y, Kiriyama M, Fukai I, Yamakawa Y, Fujii Y: Histone deacetylase 1 mRNA expression in lung cancer. Lung Cancer, 2004, 46(2):171-178.
26. Balciunaite E, Spektor A, Lents N H, Cam H, Te Riele H, Scime A, Rudnicki M A, Young R, Dynlacht B D: Pocket protein complexes are recruited to distinct targets in quiescent and proliferating cells. Mol Cell Biol, 2005, 25(18):8166-8178.
27. Soni S, Kaur J, Kumar A, Chakravarti N, Mathur M, Bahadur S, Shukla N K, Deo S V, Ralhan R: Alterations of rb pathway components are frequent events in patients with oral epithelial dysplasia and predict clinical outcome in patients with squamous cell carcinoma. Oncology, 2005, 68(4-6):314-325.
28. Xue Jun H, Gemma A, Hosoya Y, Matsuda K, Nara M, Hosomi Y, Okano T, Kurimoto F, Seike M, Takenaka K, Yoshimura A, Toyota M, Kudoh S. Reduced transcription of the RB2/p130 gene in human lung cancer. Mol Carcinog, 2003, 38(3):124-129.
29. Mishina T, Dosaka-Akita H, Hommura F, Nishi M, Kojima T, Ogura S, Shimizu M, Katoh H, Kawakami Y: Cyclin E expression, a potential prognostic marker for non-small cell lung cancers. Clin Cancer Res, 2000, 6(1):11-16.
30. Shintani S, Mihara M, Nakahara Y, Kiyota A, Ueyama Y, Matsumura T, Wong D T. Expression of cell cycle control proteins in normal epithelium, premalignant and malignant lesions of oral cavity. Oral Oncol, 2002, 38(3):235-243.
31. Kim J H, Sherman M E, Curriero F C, Guengerich F P, Strickland P T, Sutter T R: Expression of cytochromes P450 1A1 and 1B1 in human lung from smokers, non-smokers, and ex-smokers. Toxicol Appl Pharmacol, 2004, 299:210-219
32. Rusznak C, Mills P, Devalia J, Sapsford R, Davies R Lozewicz S: Effect of cigarette smoke on the permeability and IL-1beta and sICAM-1 release from cultured human bronchial epithelial cells of never-smokers, smokers, and patients with chronic obstructive pulmonary disease. American Journal of Respiratory and Molecular Cell Biology, 2000, 23:530-536.
33. Katsuragi H, Hasegawa A, Saito K: Distribution of metallothionein in cigarette smokers and nonsmokers in advanced periodontitis patients. Journal of Periodontology, 1997, 68(10):1005-9
34. Cardosa S V, Barbosa H M, Candellori I M, Loyola A M, Aguiar M C: Prognostic impact of metallothionein on oral squamous cell cancer. Virchows Archive, 2002, 441(2):174-178.
35. Li Y, Maie A, Zhou X, Kim Y, Sinha U, Jordan R, Eisele D, Abemayor E, Elashoff D, Park N, Wong D: Salivary Transcriptome Diagnostics for Oral Cancer Detection. Clinical Cancer Research, 2004, 10:8442-8450.
36. Li Y, Zhou X, St. John M A R, Wong D T W: RNA profiling of cell-free saliva using microarray technology. J Dent Res, 2004, 83(3):199-203.
37. The Mouth Database at the World Wide Web address pulm.bumc.bu.edu/MouthDB/index.
38. The R-project for Statistical Computing at the World Wide Web address r-project.org.
39. Spotfire at the World Wide Web address spotfire.com.
40. EASE at the World Wide Web address david.abcc.ncifcrf.gov/tools.jsp.
41. CLUSTER, TREVIEW at the World Wide Web address rana.lbl.gov/EisenSoftware.
43. Subramanian A, Tamayo P, Mootha V K, Mukherjee S, Ebert B L, Gillette M A, Paulovich A, Pomeroy S L, Golub T R, Lander E S, Mesirov J P: Gene set enrichment analysis: A knowledge-based approach for interpreting genome-wide expression profiles. PNAS, 2005, 102(43):15545-15550.
44. Ding, C, Cantor, CR: A high-throughput gene expression analysis technique using competitive PCR and matrix-assisted laser desorption ionization time-of-flight MS. PNAS, 2003, 100(6):3059-3064.
45. Vandesompele J, De Preter K, Pattyn F, Poppe B, Van Roy N, De Paepe A, Speleman F: Accurate normalization of real-time quantitative RT-PCR data by geometric averaging of multiple internal control genes. Genome Biol, 2002, 3(7).

We claim:

1. A method of diagnosing lung cancer in an individual comprising the steps of:
a) measuring a biological sample comprising lung epithelial tissue from the individual for the expression of at least 20 gene transcripts from Table 6;
b) comparing the expression of the at least 20 gene transcripts to a control sample of those transcripts from individuals without cancer,
wherein increased expression of the gene transcripts as indicated by a negative score in the last column of Table 6 and/or decreased expression of the gene transcripts as indicated by a positive score in the last column of Table 6 is indicative of the individual having lung cancer.

2. The method of claim 1, wherein at least 40 gene transcripts are measured.

3. The method of claim 1, wherein at least 60 gene transcripts are measured.

4. The method of claim 1, wherein at least 70 gene transcripts are measured.

5. The method of claim 1, wherein the gene transcript measured is set forth in Table 5.

6. The method of claim 1, wherein the gene transcript measured is set forth in Table 7.

7. The method of claim 1, wherein the gene transcript measured is set forth in Table 1 wherein the measurement of the gene transcript relative to the control uses the third column of Table 1 setting forth direction of expression in lung cancer to determine if the individual has lung cancer.

8. The method of claim 7, wherein the transcript measured is at least Table 3.

9. The method of claim 7, wherein the transcript used is at least the transcripts set forth in Table 4.
